(19) United States
(12) Patent Application Publication
(10) Pub. No.: US 2026/0090994 A1
(43) Pub. Date: Apr. 2, 2026

(54) TABLET FORMULATIONS

(71) Applicants: Gilead Sciences, Inc., Foster City, CA (US); Merck Sharp & Dohme LLC, Rahway, NJ (US)

(72) Inventors: Benjamin Chal, Milbrae, CA (US); Jay Poorna Reddy, San Jose, CA (US); Dhananjay Devidas Marathe, Basking Ridge, NJ (US); Marjorie Z. Imperial, Wapato, WA (US); James C. DiNunzio, Flemington, NJ (US); Pavithra Sundararajan, South Plainfield, NJ (US); Ryan Vargo, Carmel, IN (US); Diane M. Longo, Cleveland, OH (US); Michelle Pham, Brooklyn, NY (US); Diana Sperger, Fanwood, NJ (US)

(21) Appl. No.: 19/314,157

(22) Filed: Aug. 29, 2025

Related U.S. Application Data
(60) Provisional application No. 63/689,587, filed on Aug. 30, 2024.
(60) Provisional application No. 63/768,353, filed on Mar. 7, 2025.

(51) Int. Cl. A61K 9/20; A61K 31/4439; A61K 31/7076

(52) U.S. Cl. CPC A61K 9/2077; A61K 9/2018; A61K 9/2027; A61K 9/2031; A61K 9/2054; A61K 31/4439; A61K 31/7076

(57) ABSTRACT

The present disclosure relates to pharmaceutical compositions (e.g., tablets) comprising an human immunodeficiency virus (HIV) capsid inhibitor and a nucleoside reverse transcriptase translocation inhibitor (NRTTI), useful for the treatment of an HIV infection in a patient.

### TECHNICAL FIELD

The present disclosure relates to pharmaceutical compositions (e.g., tablets) comprising an HIV capsid inhibitor and a nucleoside reverse transcriptase translocation inhibitor (NRTTI), which are useful for the treatment of a human immunodeficiency virus (HIV) infection in a patient.

### BACKGROUND

Advances in combination antiretroviral therapy (ART) for HIV have led to significant improvements in morbidity and mortality by suppressing viral replication, preserving immunologic function, and averting disease progression to AIDS. Standard of care for the treatment of HIV-1 infection involves the use of a combination of ARV drugs to suppress viral replication to below detectable limits, increase CD4+ T-cell counts, and delay disease progression.

While currently available combination ART for the treatment of HIV-1 infection is efficacious and well tolerated, all current oral regimens require daily adherence to minimize the emergence of drug resistant variants. In addition, “treatment fatigue” can occur, defined as “decreased desire and motivation to maintain vigilance in adhering to a treatment regimen” among people with HIV-1 (PWH) prescribed chronic or life-long treatment (see e.g., Claborn et al, Psychol. Health Med. 2015; 20 (3):1-11), which can lead to nonadherence and treatment failure. As such, there remains a significant medical need for ARVs that can be administered less frequently (i.e., long acting drug products), thereby providing alternative treatment options for PWH.

### SUMMARY

The present application provides, inter alia, a tablet comprising a compound of Formula Ia:

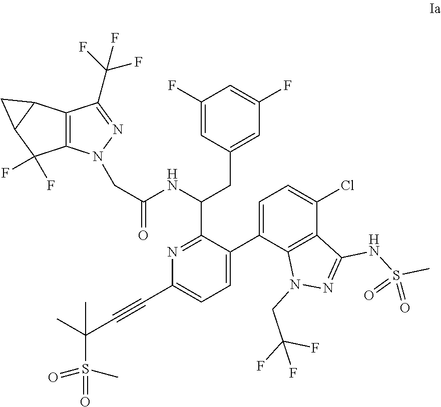 [SMILES: CC(C)(C#Cc1ccc(-c2ccc(Cl)c3c(NS(C)(=O)=O)nn(CC(F)(F)F)c23)c(C(Cc2cc(F)cc(F)c2)NC(=O)Cn2nc(C(F)(F)F)c3c2C(F)(F)C2CC32)n1)S(C)(=O)=O]

or a pharmaceutically acceptable salt, co-crystal, solvate, or combination thereof;
a compound of Formula IIa:

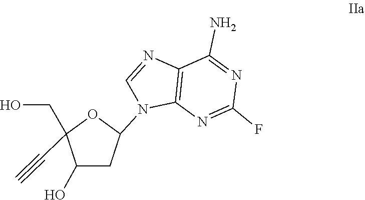 [SMILES: C#CC1(CO)OC(n2cnc3c(N)nc(F)nc32)CC1O]

or a pharmaceutically acceptable salt, co-crystal, solvate, or combination thereof;
and one or more pharmaceutically acceptable excipients

The present disclosure further provides a method of treating HIV in a patient, comprising administering to the patient a tablet provided herein.

The present disclosure further provides a tablet provided herein, for use in any of the methods described herein.

The present disclosure further provides use of a tablet provided herein, for the preparation of a medicament for use in any of the methods described herein.

### DESCRIPTION OF DRAWINGS

FIGS. 1A-1B show mean (SD) islatravir (ISL) plasma concentration-time profiles by cohort. The horizontal reference line indicates the lower limit of quantitation (LLOQ), defined as 0.02 ng/mL for ISL. Value below the limit of quantitation were treated as 0 for pre-dose and post-dose summary statistics.

FIG. 2 shows mean (SD) islatravir (ISL) plasma concentration-time profiles (through 24-hour time after dose on Day 1) by cohort. The horizontal reference line indicates the lower limit of quantitation (LLOQ), defined as 0.02 ng/mL for ISL. Value below the limit of quantitation were treated as 0 for pre-dose and post-dose summary statistics.

FIGS. 3A-3B show mean (SD) lenacapavir (LEN) plasma concentration-time profiles by cohort. The horizontal reference line indicates the lower limit of quantitation (LLOQ), defined as 0.1 ng/mL for LEN. Value below the limit of quantitation were treated as 0 for pre-dose and post-dose summary statistics.

FIG. 4 shows mean (SD) lenacapavir (LEN) plasma concentration-time profiles (through 24-hour time after dose on Day 1) by cohort. The horizontal reference line indicates the lower limit of quantitation (LLOQ), defined as 0.1 ng/mL for LEN. Value below the limit of quantitation were treated as 0 for pre-dose and post-dose summary statistics.

FIGS. 5A-5B show representative schemes for preparing the fixed-dose combination (FDC) tablets of Examples 1-2.

FIG. 6 shows a representative scheme for preparing the lenacapavir spray dried dispersion (LEN SDD) described in Example 3.

FIGS. 7A-7B show structural PopPK models for ISL (FIG. 7A) and LEN (FIG. 7B).

FIG. 8 shows simulated dosing scenarios of ISL and LEN. Dosing scenarios under steady-state conditions following missed oral QW doses (Scenarios 2a-4) were compared against the reference scenario of no missed doses (Scenario 1), as well as efficacy and clinically established safety exposure thresholds, as appropriate.

FIGS. 9A-9E show dose simulation plots for ISL under simulated dosing Scenario 1 (FIG. 9A), Scenario 2a (FIG. 9B), Scenario 2b (FIG. 9C), Scenario 3 (FIG. 9D), and Scenario 4 (FIG. 9E). Left-most arrow indicates a missed dose; right most arrow(s) indicate the next dose(s) taken (e.g., in Scenario 4, the leftmost two arrows indicate missed dose and the rightmost two arrows indicate the next dose(s) taken). ^{a}Maintenance FDC (2 mg ISL) was used for Scenarios 2a, 2b, and 3. ^{b} Starting FDC (1 mg ISL) was used for Scenario 4.

FIGS. 10A-10E show dose simulation plots for LEN under simulated dosing Scenario 1 (FIG. 10A), Scenario 2a (FIG. 10B), Scenario 2b (FIG. 10C), Scenario 3 (FIG. 10D), and Scenario 4 (FIG. 10E). Left-most arrow indicates a missed dose; right-most arrow(s) indicate the next dose(s) taken (e.g., in Scenario 4, the leftmost two arrows indicate missed dose and the rightmost two arrows indicate the next dose(s) taken). ^{a} Maintenance FDC (300 mg LEN) was used for Scenarios 2a, 2b, and 3. ^{b} Starting FDC (600 mg LEN) was used for Scenario 4.

### DETAILED DESCRIPTION

Lenacapavir (LEN), a human immunodeficiency virus type 1 (HIV-1) capsid inhibitor, has the potential to provide an additional option for populations at risk of HIV acquisition. Lenacapavir can help address substantial existing PrEP barriers including 1) requirement for daily adherence, 2) stigma and concerns about disclosure and discrimination or other social harms, 3) oral medication-associated adverse events (AEs), including gastrointestinal tolerability, and 4) challenges with access to health care providers in overburdened health systems or in geographical PrEP deserts. Thus, LEN has the potential to increase the uptake of, adherence to, and thereby the scalability of PrEP, thus contributing to the overarching goal of ending the HIV-1 epidemic.

As used herein, the term “lenacapavir” or “LEN” refers to the compound N—((S)-1-(3-(4-chloro-3-(methylsulfonamido)-1-(2,2,2-trifluoroethyl)-1H-indazol-7-yl)-6-(3-methyl-3-(methylsulfonyl)but-1-yn-1-yl)pyridin-2-yl)-2-(3,5-difluorophenyl)ethyl)-2-((3bS,4aR)-5,5-difluoro-3-(trifluoromethyl)-3b,4,4a,5-tetrahydro-1H-cyclopropa[3,4]cyclopenta[1,2-c]pyrazol-1-yl)acetamide, having the following structure:

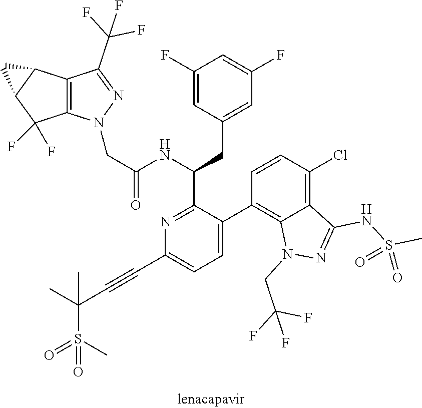 [SMILES: CC(C)(C#Cc1ccc(-c2ccc(Cl)c3c(NS(C)(=O)=O)nn(CC(F)(F)F)c23)c([C@H](Cc2cc(F)cc(F)c2)NC(=O)Cn2nc(C(F)(F)F)c3c2C(F)(F)[C@@H]2C[C@H]32)n1)S(C)(=O)=O]

Islatravir (ISL) is a novel deoxyadenosine analog with potent ARV activity, including activity against common nucleoside reverse transcriptase inhibitor-resistant variants, and favorable nonclinical toxicity and pharmacokinetic (PK) profiles. ISL is converted intracellularly to the active ISL-triphosphate (ISL-TP) that potently inhibits HIV-1 replication by preventing translocation of reverse transcriptase on the viral primer template. Lenacapavir (LEN) is a novel, first-in-class, multistage selective inhibitor of HIV-1 capsid function. It inhibits HIV-1 at multiple points in the viral lifecycle, including interfering with capsid-mediated nuclear uptake of preintegration complexes and impairing virion production and proper capsid core formation. It has 2 dosage forms (oral tablets for initial loading and 6-monthly subcutaneous injection) both of which are approved in adults in combination with other ARVs for the treatment of multidrug resistant HIV-1 infection in several countries. Oral administration of ISL and LEN as a 2-drug combination regimen has the potential to offer a safe, efficacious, and well tolerated once-weekly regimen for the treatment of PWH.

As used herein, the term “islatravir” or “ISL” refers to 4′-ethynyl-2-fluoro-2′-deoxyadenosine (i.e., (2R,3S,5R)-5-(6-amino-2-fluoro-9H-purin-9-yl)-2-ethynyl-2-(hydroxymethyl)tetrahydrofuran-3-ol), having the following structure:

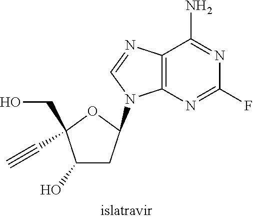 [SMILES: C#C[C@]1(CO)O[C@@H](n2cnc3c(N)nc(F)nc32)C[C@@H]1O]

One skilled in the art understands that a compound structure may be named or identified using commonly recognized nomenclature systems and symbols. By way of example, the compound may be named or identified with common names, systematic or non-systematic names. The nomenclature systems and symbols that are commonly recognized in the art of chemistry including but not limited to Chemical Abstract Service (CAS) and International Union of Pure and Applied Chemistry (IUPAC).

### Pharmaceutical Compositions

Accordingly, the present application provides a tablet comprising a compound of Formula Ia:

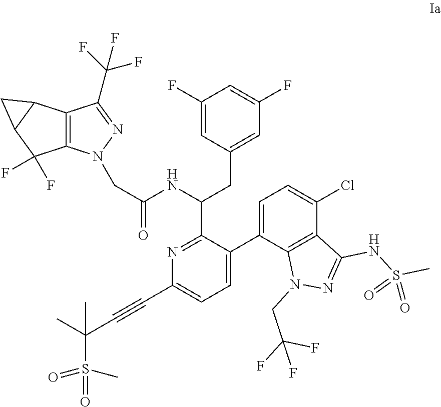 [SMILES: CC(C)(C#Cc1ccc(-c2ccc(Cl)c3c(NS(C)(=O)=O)nn(CC(F)(F)F)c23)c(C(Cc2cc(F)cc(F)c2)NC(=O)Cn2nc(C(F)(F)F)c3c2C(F)(F)C2CC32)n1)S(C)(=O)=O]

or a pharmaceutically acceptable salt, co-crystal, solvate, or combination thereof;
a compound of Formula IIa:

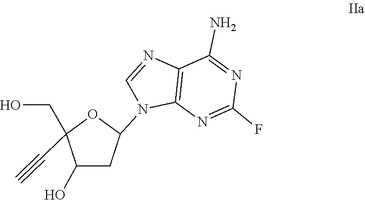 [SMILES: C#CC1(CO)OC(n2cnc3c(N)nc(F)nc32)CC1O]

or a pharmaceutically acceptable salt, co-crystal, solvate, or combination thereof;
and one or more pharmaceutically acceptable excipients.

In some embodiments, the tablets disclosed herein consist essentially of the components described herein. In some embodiments, the tablets disclosed herein consist of the components described herein.

Tablets of the disclosure are formulated so as to allow the active ingredients contained therein to be bioavailable upon administration of the tablet to a patient. The tablet to be administered will, in any event, contain a therapeutically effective amount of the compound of Formula Ia or Ib and the compound of Formula IIa or IIb, or a pharmaceutically acceptable salt, co-crystal, solvate, or combination thereof, for treating an HIV infection as described herein. In some embodiments, the tablet provided herein is suitable for oral administration. In some embodiments, the tablet provided herein is a fixed-dose combination tablet (i.e., a fixed dose combination of the compound of Formula Ia or Ib and the compound of Formula IIa or IIb, or a pharmaceutically acceptable salt, co-crystal, solvate, or combination thereof).

In some embodiments, the compound of Formula Ia or Ib is administered as a salt, such as a pharmaceutically acceptable salt. A salt generally refers to a derivative of a disclosed compound wherein the parent compound is modified by converting an existing acid or base moiety to its salt form. A pharmaceutically acceptable salt is one that, within the scope of sound medical judgment, is suitable for use in contact with the tissues of human beings and animals without excessive toxicity, irritation, allergic response, or other problem or complication, commensurate with a reasonable benefit/risk ratio. Examples of pharmaceutically acceptable salts include, but are not limited to, mineral or organic acid salts of basic residues such as amines, alkali or organic salts of acidic residues such as carboxylic acids, and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid. Lists of suitable salts are found in Remington's Pharmaceutical Sciences, 17^{th} ed., Mack Publishing Company, Easton, Pa., 1985, p. 1418 and Journal of Pharmaceutical Science, 66, 2 (1977), each of which is incorporated herein by reference in its entirety. In some embodiments, the salt is a sodium salt. In some embodiments, the pharmaceutically acceptable salt of the compound of Formula Ia or Ib provided herein is a sodium salt. In some embodiments, the pharmaceutically acceptable salt of the compound of Formula Ib provided herein is a sodium salt.

In some embodiments, the compound of Formula Ia or Ib, or a pharmaceutically acceptable salt, co-crystal, solvate, or combination thereof (e.g., lenacapavir sodium) is present in a tablet provided herein as a spray dried dispersion (SDD).

In some embodiments, the tablet comprises a spray dried dispersion (SDD) of the compound of Formula Ia, or a pharmaceutically acceptable salt, co-crystal, solvate, or combination thereof. In some embodiments, the tablet comprises a spray dried dispersion (SDD) of the compound of Formula Ia, or a pharmaceutically acceptable salt thereof. In some embodiments, the tablet comprises a spray dried dispersion (SDD) of the sodium salt of the compound of Formula Ia.

In some embodiments, the tablet comprises a spray dried dispersion (SDD) of the compound of Formula Ib, or a pharmaceutically acceptable salt, co-crystal, solvate, or combination thereof. In some embodiments, the tablet comprises a spray dried dispersion (SDD) of the compound of Formula Ib, or a pharmaceutically acceptable salt thereof. In some embodiments, the tablet comprises a spray dried dispersion (SDD) of the sodium salt of the compound of Formula Ib (i.e., “the lenacapavir spray dried dispersion” or “LEN SDD” or “lenacapavir spray dried dispersion”).

In some embodiments, the spray dried dispersion comprises the compound of Formula Ia or Ib, or a pharmaceutically acceptable salt, co-crystal, solvate, or combination thereof, copovidone, and poloxamer 407.

In some embodiments, the spray dried dispersion comprises the compound of Formula Ia, or a pharmaceutically acceptable salt, co-crystal, solvate, or combination thereof, copovidone, and poloxamer 407. In some embodiments, the spray dried dispersion comprises the compound of Formula Ia, or a pharmaceutically acceptable salt thereof, copovidone, and poloxamer 407. In some embodiments, the spray dried dispersion comprises the sodium salt of the compound of Formula Ia, copovidone, and poloxamer 407.

In some embodiments, the spray dried dispersion comprises the compound of Formula Ib, or a pharmaceutically acceptable salt, co-crystal, solvate, or combination thereof, copovidone, and poloxamer 407. In some embodiments, the spray dried dispersion comprises the compound of Formula Ib, or a pharmaceutically acceptable salt thereof, copovidone, and poloxamer 407. In some embodiments, the spray dried dispersion comprises the sodium salt of the compound of Formula Ib, copovidone, and poloxamer 407.

In some embodiments, the spray dried dispersion comprises about 76 w/w % to about 77 w/w % of the compound of Formula Ia or Ib, or a pharmaceutically acceptable salt, co-crystal, solvate, or combination thereof, about 18 w/w % to about 19 w/w % copovidone, and about 4 w/w % to about 6 w/w % poloxamer 407. In some embodiments, the spray dried dispersion comprises about 76 w/w % to about 77 w/w % of the compound of Formula Ia or Ib, or a pharmaceutically acceptable salt thereof; about 18 w/w % to about 19 w/w % copovidone, and about 4 w/w % to about 6 w/w % poloxamer 407. In some embodiments, the spray dried dispersion comprises about 76 w/w % to about 77 w/w % of the sodium salt of the compound of Formula Ia or Ib, about 18 w/w % to about 19 w/w % copovidone, and about 4 w/w % to about 6 w/w % poloxamer 407. In some embodiments, the spray dried dispersion comprises about 76 w/w % to about 77 w/w % of the sodium salt of the compound of Formula Ia, about 18 w/w % to about 19 w/w % copovidone, and about 4 w/w % to about 6 w/w % poloxamer 407. In some embodiments, the spray dried dispersion comprises about 76 w/w % to about 77 w/w % of the sodium salt of the compound of Formula Ib, about 18 w/w % to about 19 w/w % copovidone, and about 4 w/w % to about 6 w/w % poloxamer 407.

In some embodiments, the spray dried dispersion comprises about 76.7 w/w % of the compound of Formula Ia or Ib, or a pharmaceutically acceptable salt, co-crystal, solvate, or combination thereof, about 18.3 w/w % copovidone, and about 5.0 w/w % poloxamer 407. In some embodiments, the spray dried dispersion comprises about 76.7 w/w % of the compound of Formula Ia or Ib, or a pharmaceutically acceptable salt thereof, about 18.3 w/w % copovidone, and about 5.0 w/w % poloxamer 407. In some embodiments, the spray dried dispersion comprises about 76.7 w/w % of the sodium salt of the compound of Formula Ia or Ib, about 18.3 w/w % copovidone, and about 5.0 w/w % poloxamer 407. In some embodiments, the spray dried dispersion comprises about 76.7 w/w % of the sodium salt of the compound of Formula Ia, about 18.3 w/w % copovidone, and about 5.0 w/w % poloxamer 407. In some embodiments, the spray dried dispersion comprises about 76.7 w/w % of the sodium salt of the compound of Formula Ib, about 18.3 w/w % copovidone, and about 5.0 w/w % poloxamer 407.

As used herein, the terms “lenacapavir spray dried dispersion” and “LEN SDD” refers to the spray dried dispersion described in Table 17.

As used herein, the term “co-crystal” refers to a crystalline material formed by combining a compound (e.g., a compound of Formula Ia, Ib, IIa, or IIb) or any Formula disclosed herein and one or more co-crystal formers (i.e., a molecule, ion, or atom). In certain instances, co-crystals may have improved properties as compared to the parent form (i.e., the free molecule, zwitterion, etc.) or a salt of the parent compound. Improved properties can be increased solubility, increased dissolution, increased bioavailability, increased dose response, decreased hygroscopicity, a crystalline form of a normally amorphous compound, a crystalline form of a difficult to salt or unsaltable compound, decreased form diversity, more desired morphology, and the like. Methods for making and characterizing co-crystals are known to those of skill in the art.

As used herein, the term “hydrate” refers to the complex formed by the combining of a compound (e.g., a compound of Formula IIa or IIb) or any Formula disclosed herein, and water.

As used herein, the term “solvate” refers to a complex formed by combining a compound (e.g., a compound of Formula IIa or IIb) or any other Formula as disclosed herein, and a solvent or a crystalline solid containing amounts of a solvent incorporated within the crystal structure. As used herein, the term “solvate” includes hydrates.

Any formula or structure given herein, including Formulas Ia, Ib, IIa, or IIb, is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the disclosure include isotopes of hydrogen, carbon, nitrogen, oxygen, phosphorous, fluorine and chlorine, such as, but not limited to ²H (deuterium, D), ³H (tritium), ¹¹C, ¹³C, ¹⁴C, ¹⁵N, ¹⁸F, ³¹P, ³²P, ³⁵S, ³⁶Cl and ¹²⁵I. Various isotopically labeled compounds of the present disclosure, for example those into which radioactive isotopes such as ³H, ¹³C and ¹⁴C are incorporated. Such isotopically labeled compounds may be useful in metabolic studies, reaction kinetic studies, detection or imaging techniques, such as positron emission tomography (PET) or single-photon emission computed tomography (SPECT) including drug or substrate tissue distribution assays or in radioactive treatment of patients.

The modifier “about” used in connection with a quantity is inclusive of the stated value and has the meaning dictated by the context (e.g., includes the degree of error associated with measurement of the particular quantity).

Except as otherwise noted, the methods and techniques of the present disclosure are generally performed according to conventional methods well known in the art and as described in various general and more specific references that are cited and discussed throughout the present specification. See, e.g., Loudon, Organic Chemistry, 5^{th} edition, New York: Oxford University Press, 2009; Smith, March's Advanced Organic Chemistry: Reactions, Mechanisms, and Structure, 7^{th} edition, Wiley-Interscience, 2013.

Compounds as described herein can be purified by any of the means known in the art, including chromatographic means, such as high performance liquid chromatography (HPLC), preparative thin layer chromatography, flash column chromatography, supercritical fluid chromatography (SFC), and ion exchange chromatography. Any suitable stationary phase can be used, including normal and reversed phases as well as ionic resins. Most typically the disclosed compounds are purified via silica gel and/or alumina chromatography. See, e.g., Introduction to Modern Liquid Chromatography, 2^{nd} ed., ed. L. R. Snyder and J. J. Kirkland, John Wiley and Sons, 1979; and Thin Layer Chromatography, E. Stahl (ed.), Springer-Verlag, New York, 1969.

As used herein, “pharmaceutically acceptable carrier” is meant to refer to any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

In some embodiments, the tablet comprises the compound of Formula Ia, or a pharmaceutically acceptable salt thereof. In some embodiments, the tablet comprises a pharmaceutically acceptable salt of the compound of Formula Ia. In some embodiments, the tablet comprises a sodium salt of the compound of Formula Ia.

In some embodiments, the compound of Formula Ia, or a pharmaceutically acceptable salt, co-crystal, solvate, or combination thereof, is a compound of Formula Ib:

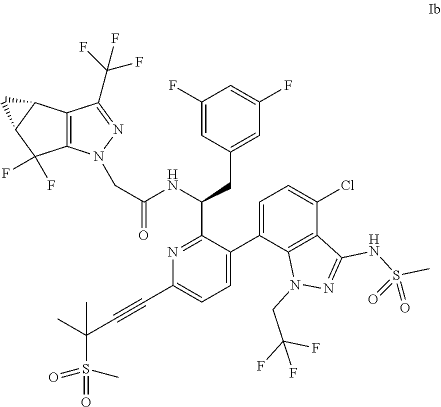 [SMILES: CC(C)(C#Cc1ccc(-c2ccc(Cl)c3c(NS(C)(=O)=O)nn(CC(F)(F)F)c23)c([C@H](Cc2cc(F)cc(F)c2)NC(=O)Cn2nc(C(F)(F)F)c3c2C(F)(F)[C@@H]2C[C@H]32)n1)S(C)(=O)=O]

or a pharmaceutically acceptable salt, co-crystal, solvate, or combination thereof.

In some embodiments, the tablet comprises the compound of Formula IIa, or a pharmaceutically acceptable salt thereof. In some embodiments, the tablet comprises a pharmaceutically acceptable salt of the compound of Formula Ib. In some embodiments, the tablet comprises a sodium salt of the compound of Formula Ib.

In some embodiments, the tablet comprises a compound of Formula IIa, or a solvate thereof. In some embodiments, the tablet comprises a solvate of the compound of Formula IIa. In some embodiments, the tablet comprises a hydrate of the compound of Formula IIa. In some embodiments, the tablet comprises a monohydrate of the compound of Formula IIa.

In some embodiments, the compound of Formula IIa, or a pharmaceutically acceptable salt, co-crystal, solvate, or combination thereof, is a compound of Formula IIb:

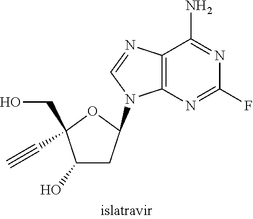 [SMILES: C#C[C@]1(CO)O[C@@H](n2cnc3c(N)nc(F)nc32)C[C@@H]1O]

or a pharmaceutically acceptable salt, co-crystal, solvate, or combination thereof. In some embodiments, the tablet comprises a compound of Formula IIb, or a solvate thereof. In some embodiments, the tablet comprises a solvate of the compound of Formula IIb. In some embodiments, the tablet comprises a hydrate of the compound of Formula IIb. In some embodiments, the tablet comprises a monohydrate of the compound of Formula IIb.

In some embodiments, the tablet comprises about 18 w/w % to about 30 w/w % of the compound of Formula Ia or Ib, or a pharmaceutically acceptable salt, co-crystal, solvate, or combination thereof, for example, about 18 w/w %, about 19 w/w %, about 20 w/w %, about 20.1 w/w %, about 20.2 w/w %, about 20.3 w/w %, about 20.4 w/w %, about 20.5 w/w %, about 20.6 w/w %, about 20.7 w/w %, about 20.8 w/w %, about 20.9 w/w %, about 21 w/w %, about 22 w/w %, about 23 w/w %, about 24 w/w %, about 25 w/w %, about 26 w/w %, about 27 w/w %, about 28 w/w %, about 29 w/w %, or about 30 w/w % of the compound of Formula Ia or Ib, or a pharmaceutically acceptable salt, co-crystal, solvate, or combination thereof.

In some embodiments, the tablet comprises about 20 w/w % to about 30 w/w % of the spray dried dispersion of the compound of Formula Ia or Ib, or a pharmaceutically acceptable salt, co-crystal, solvate, or combination thereof, for example, about 20 w/w %, about 21 w/w %, about 22 w/w %, about 23 w/w %, about 24 w/w %, about 25 w/w %, about 26 w/w %, about 27 w/w %, about 28 w/w %, about 29 w/w %, or about 30 w/w % of the spray dried dispersion of the compound of Formula Ia or Ib, or a pharmaceutically acceptable salt, co-crystal, solvate, or combination thereof. In some embodiments, the tablet comprises about 26 w/w % to about 27 w/w % of the spray dried dispersion of the compound of Formula Ia or Ib, or a pharmaceutically acceptable salt, co-crystal, solvate, or combination thereof. In some embodiments, the tablet comprises about 26.6 w/w % to about 26.7 w/w % of the spray dried dispersion of the compound of Formula Ia or Ib, or a pharmaceutically acceptable salt, co-crystal, solvate, or combination thereof. In some embodiments, the tablet comprises about 26.67 w/w % of the spray dried dispersion of the compound of Formula Ia or Ib, or a pharmaceutically acceptable salt, co-crystal, solvate, or combination thereof.

In some embodiments, the tablet comprises about 20 w/w % to about 30 w/w % of the spray dried dispersion of the compound of Formula Ia or Ib, or a pharmaceutically acceptable salt thereof, for example, about 20 w/w %, about 21 w/w %, about 22 w/w %, about 23 w/w %, about 24 w/w %, about 25 w/w %, about 26 w/w %, about 27 w/w %, about 28 w/w %, about 29 w/w %, or about 30 w/w % of the spray dried dispersion of the compound of Formula Ia or Ib, or a pharmaceutically acceptable salt thereof. In some embodiments, the tablet comprises about 26 w/w % to about 27 w/w % of the spray dried dispersion of the compound of Formula Ia or Ib, or a pharmaceutically acceptable salt thereof. In some embodiments, the tablet comprises about 26.6 w/w % to about 26.7 w/w % of the spray dried dispersion of the compound of Formula Ia or Ib, or a pharmaceutically acceptable salt thereof. In some embodiments, the tablet comprises about 26.67 w/w % of the spray dried dispersion of the compound of Formula Ia or Ib, or a pharmaceutically acceptable salt thereof.

In some embodiments, the tablet comprises about 20 w/w % to about 30 w/w % of the spray dried dispersion of the sodium salt of the compound of Formula Ia or Ib, for example, about 20 w/w %, about 21 w/w %, about 22 w/w %, about 23 w/w %, about 24 w/w %, about 25 w/w %, about 26 w/w %, about 27 w/w %, about 28 w/w %, about 29 w/w %, or about 30 w/w % of the spray dried dispersion of the sodium salt of the compound of Formula Ia or Ib. In some embodiments, the tablet comprises about 26 w/w % to about 27 w/w % of the spray dried dispersion of the sodium salt of the compound of Formula Ia or Ib. In some embodiments, the tablet comprises about 26.6 w/w % to about 26.7 w/w % of the spray dried dispersion of the sodium salt of the compound of Formula Ia or Ib. In some embodiments, the tablet comprises about 26.67 w/w % of the spray dried dispersion of the sodium salt of the compound of Formula Ia or Ib.

In some embodiments, the tablet comprises about 20 w/w % to about 30 w/w % of the spray dried dispersion of the sodium salt of the compound of Formula Ia, for example, about 20 w/w %, about 21 w/w %, about 22 w/w %, about 23 w/w %, about 24 w/w %, about 25 w/w %, about 26 w/w %, about 27 w/w %, about 28 w/w %, about 29 w/w %, or about 30 w/w % of the spray dried dispersion of the sodium salt of the compound of Formula Ia. In some embodiments, the tablet comprises about 26 w/w % to about 27 w/w % of the spray dried dispersion of the sodium salt of the compound of Formula Ia. In some embodiments, the tablet comprises about 26.6 w/w % to about 26.7 w/w % of the spray dried dispersion of the sodium salt of the compound of Formula Ia. In some embodiments, the tablet comprises about 26.67 w/w % of the spray dried dispersion of the sodium salt of the compound of Formula Ia.

In some embodiments, the tablet comprises about 20 w/w % to about 30 w/w % of the spray dried dispersion of the sodium salt of the compound of Formula Ib, for example, about 20 w/w %, about 21 w/w %, about 22 w/w %, about 23 w/w %, about 24 w/w %, about 25 w/w %, about 26 w/w %, about 27 w/w %, about 28 w/w %, about 29 w/w %, or about 30 w/w % of the spray dried dispersion of the sodium salt of the compound of Formula Ib. In some embodiments, the tablet comprises about 26 w/w % to about 27 w/w % of the spray dried dispersion of the sodium salt of the compound of Formula Ib. In some embodiments, the tablet comprises about 26.6 w/w % to about 26.7 w/w % of the spray dried dispersion of the sodium salt of the compound of Formula Ib. In some embodiments, the tablet comprises about 26.67 w/w % of the spray dried dispersion of the sodium salt of the compound of Formula Ib.

In some embodiments, the tablet comprises about 20 w/w % to about 30 w/w % of the lenacapavir spray dried dispersion as described herein, for example, about 20 w/w %, about 21 w/w %, about 22 w/w %, about 23 w/w %, about 24 w/w %, about 25 w/w %, about 26 w/w %, about 27 w/w %, about 28 w/w %, about 29 w/w %, or about 30 w/w % of the lenacapavir spray dried dispersion. In some embodiments, the tablet comprises about 26 w/w % to about 27 w/w % of the lenacapavir spray dried dispersion. In some embodiments, the tablet comprises about 26.6 w/w % to about 26.7 w/w % of the lenacapavir spray dried dispersion. In some embodiments, the tablet comprises about 26.67 w/w % of the lenacapavir spray dried dispersion.

In some embodiments, the tablet comprises about 18 w/w % to about 30 w/w % of the compound of Formula Ia or Ib, or a pharmaceutically acceptable salt thereof, for example, about 18 w/w %, about 19 w/w %, about 20 w/w %, about 21 w/w %, about 22 w/w %, about 23 w/w %, about 24 w/w %, about 25 w/w %, about 26 w/w %, about 27 w/w %, about 28 w/w %, about 29 w/w %, or about 30 w/w % of the compound of Formula Ia or Ib, or a pharmaceutically acceptable salt thereof. In some embodiments, the tablet comprises about 19 w/w % to about 21 w/w % of the compound of Formula Ia or Ib, or a pharmaceutically acceptable salt thereof. In some embodiments, the tablet comprises about 19.8 w/w % to about 20.8 w/w % of the compound of Formula Ia or Ib, or a pharmaceutically acceptable salt thereof.

In some embodiments, the tablet comprises about 18 w/w % to about 30 w/w % of the sodium salt of the compound of Formula Ia or Ib, for example, about 18 w/w %, about 19 w/w %, about 20 w/w %, about 21 w/w %, about 22 w/w %, about 23 w/w %, about 24 w/w %, about 25 w/w %, about 26 w/w %, about 27 w/w %, about 28 w/w %, about 29 w/w %, or about 30 w/w % of the sodium salt of the compound of Formula Ia or Ib. In some embodiments, the tablet comprises about 20 w/w % to about 21 w/w % of the sodium salt of the compound of Formula Ia or Ib. In some embodiments, the tablet comprises about 20.2 w/w % to about 20.7 w/w % of the sodium salt of the compound of Formula Ia or Ib. In some embodiments, the tablet comprises about 20.45 w/w % of the sodium salt of the compound of Formula Ia or Ib. In some embodiments, the tablet comprises about 20.46 w/w % of the sodium salt of the compound of Formula Ia or Ib.

In some embodiments, the tablet comprises about 18 w/w % to about 30 w/w % of the compound of Formula Ia or Ib free acid, for example, about 18 w/w %, about 19 w/w %, about 20 w/w %, about 21 w/w %, about 22 w/w %, about 23 w/w %, about 24 w/w %, about 25 w/w %, about 26 w/w %, about 27 w/w %, about 28 w/w %, about 29 w/w %, or about 30 w/w % of the compound of Formula Ia or Ib free acid. In some embodiments, the tablet comprises about 19 w/w % to about 21 w/w % of the compound of Formula Ia or Ib free acid. In some embodiments, the tablet comprises about 19.8 w/w % to about 20.2 w/w % of the compound of Formula Ia or Ib free acid. In some embodiments, the tablet comprises about 20.0 w/w % of the compound of Formula Ia or Ib free acid.

In some embodiments, the tablet comprises about 18 w/w % to about 30 w/w % of the compound of Formula Ia free acid, for example, about 18 w/w %, about 19 w/w %, about 20 w/w %, about 21 w/w %, about 22 w/w %, about 23 w/w %, about 24 w/w %, about 25 w/w %, about 26 w/w %, about 27 w/w %, about 28 w/w %, about 29 w/w %, or about 30 w/w % of the compound of Formula Ia free acid. In some embodiments, the tablet comprises about 19 w/w % to about 21 w/w % of the compound of Formula Ia free acid. In some embodiments, the tablet comprises about 19.80 w/w % to about 20.2 w/w % of the compound of Formula Ia free acid. In some embodiments, the tablet comprises about 20.0 w/w % of the compound of Formula Ia free acid.

In some embodiments, the tablet comprises about 18 w/w % to about 30 w/w % of the compound of Formula Ib free acid, for example, about 18 w/w %, about 19 w/w %, about 20 w/w %, about 21 w/w %, about 22 w/w %, about 23 w/w %, about 24 w/w %, about 25 w/w %, about 26 w/w %, about 27 w/w %, about 28 w/w %, about 29 w/w %, or about 30 w/w % of the compound of Formula Ib free acid. In some embodiments, the tablet comprises about 19 w/w % to about 21 w/w % of the compound of Formula Ib free acid. In some embodiments, the tablet comprises about 19.8 w/w % to about 20.2 w/w % of the compound of Formula Ib free acid. In some embodiments, the tablet comprises about 20.0 w/w % of the compound of Formula Ib free acid.

In some embodiments, the tablet comprises about 0.01 w/w % to about 2.0 w/w % of the compound of Formula IIa or IIb, or a pharmaceutically acceptable salt, co-crystal, solvate, or combination thereof, for example, about 0.01 w/w %, about 0.02 w/w %, about 0.03 w/w %, about 0.04 w/w %, about 0.05 w/w %, about 0.06 w/w %, about 0.07 w/w %, about 0.08 w/w %, about 0.09 w/w %, about 0.10 w/w %, about 0.30 w/w %, about 0.40 w/w %, about 0.50 w/w %, about 0.60 w/w %, about 0.70 w/w %, about 0.80 w/w %, about 0.90 w/w %, about 1.0 w/w %, about 1.1 w/w %, about 1.2 w/w %, about 1.3 w/w %, about 1.4 w/w %, about 1.5 w/w %, about 1.6 w/w %, about 1.7 w/w %, about 1.8 w/w %, about 1.9 w/w %, or about 2.0 w/w % of the compound of Formula IIa or IIb, or a pharmaceutically acceptable salt, co-crystal, solvate, or combination thereof.

In some embodiments, the tablet comprises about 0.02 w/w % to about 0.2 w/w % of the compound of Formula IIa or IIb, or a pharmaceutically acceptable salt, co-crystal, solvate, or combination thereof.

In some embodiments, the tablet comprises about 0.03 w/w % to about 0.15 w/w % of the compound of Formula IIa or IIb, or a pharmaceutically acceptable salt, co-crystal, solvate, or combination thereof.

In some embodiments, the tablet comprises about 0.14 w/w % to about 0.15 w/w % of the compound of Formula IIa or IIb, or a pharmaceutically acceptable salt, co-crystal, solvate, or combination thereof. In some embodiments, the tablet comprises about 0.14 w/w % of the compound of Formula IIa or IIb, or a pharmaceutically acceptable salt, co-crystal, solvate, or combination thereof. In some embodiments, the tablet comprises about 0.142 w/w % of the compound of Formula IIa or IIb, or a pharmaceutically acceptable salt, co-crystal, solvate, or combination thereof.

In some embodiments, the tablet comprises about 0.03 w/w % to about 0.04 w/w % of the compound of Formula IIa or IIb, or a pharmaceutically acceptable salt, co-crystal, solvate, or combination thereof. In some embodiments, the tablet comprises about 0.035 w/w % to about 0.036 w/w % of the compound of Formula IIa or IIb, or a pharmaceutically acceptable salt, co-crystal, solvate, or combination thereof. In some embodiments, the tablet comprises about 0.0355 w/w % of the compound of Formula IIa or IIb, or a pharmaceutically acceptable salt, co-crystal, solvate, or combination thereof.

In some embodiments, the tablet comprises about 0.01 w/w % to about 2.0 w/w % of the compound of Formula IIa or IIb, or a solvate thereof, for example, about 0.01 w/w %, about 0.02 w/w %, about 0.03 w/w %, about 0.04 w/w %, about 0.05 w/w %, about 0.06 w/w %, about 0.07 w/w %, about 0.08 w/w %, about 0.09 w/w %, about 0.10 w/w %, about 0.30 w/w %, about 0.40 w/w %, about 0.50 w/w %, about 0.60 w/w %, about 0.70 w/w %, about 0.80 w/w %, about 0.90 w/w %, about 1.0 w/w %, about 1.1 w/w %, about 1.2 w/w %, about 1.3 w/w %, about 1.4 w/w %, about 1.5 w/w %, about 1.6 w/w %, about 1.7 w/w %, about 1.8 w/w %, about 1.9 w/w %, or about 2.0 w/w % of the compound of Formula IIa or IIb, or a solvate thereof.

In some embodiments, the tablet comprises about 0.02 w/w % to about 0.2 w/w % of the compound of Formula IIa or IIb, or a solvate thereof. In some embodiments, the tablet comprises about 0.03 w/w % to about 0.15 w/w % of the compound of Formula IIa or IIb, or a solvate thereof.

In some embodiments, the tablet comprises about 0.14 w/w % to about 0.15 w/w % of the compound of Formula IIa or IIb, or a solvate thereof. In some embodiments, the tablet comprises about 0.14 w/w % of the compound of Formula IIa or IIb, or a solvate thereof. In some embodiments, the tablet comprises about 0.142 w/w % of the compound of Formula IIa or IIb, or a solvate thereof.

In some embodiments, the tablet comprises about 0.03 w/w % to about 0.04 w/w % of the compound of Formula IIa or IIb, or a solvate thereof. In some embodiments, the tablet comprises about 0.035 w/w % to about 0.036 w/w % of the compound of Formula IIa or IIb, or a solvate thereof. In some embodiments, the tablet comprises about 0.0355 w/w % of the compound of Formula IIa or IIb, or a solvate thereof.

In some embodiments, the tablet comprises about 0.01 w/w % to about 2.0 w/w % of the monohydrate of the compound of Formula IIa or IIb, for example, about 0.01 w/w %, about 0.02 w/w %, about 0.03 w/w %, about 0.04 w/w %, about 0.05 w/w %, about 0.06 w/w %, about 0.07 w/w %, about 0.08 w/w %, about 0.09 w/w %, about 0.10 w/w %, about 0.30 w/w %, about 0.40 w/w %, about 0.50 w/w %, about 0.60 w/w %, about 0.70 w/w %, about 0.80 w/w %, about 0.90 w/w %, about 1.0 w/w %, about 1.1 w/w %, about 1.2 w/w %, about 1.3 w/w %, about 1.4 w/w %, about 1.5 w/w %, about 1.6 w/w %, about 1.7 w/w %, about 1.8 w/w %, about 1.9 w/w %, or about 2.0 w/w % of the monohydrate of the compound of Formula IIa or IIb.

In some embodiments, the tablet comprises about 0.02 w/w % to about 0.2 w/w % of the monohydrate of the compound of Formula IIa or IIb. In some embodiments, the tablet comprises about 0.03 w/w % to about 0.15 w/w % of the monohydrate of the compound of Formula IIa or IIb.

In some embodiments, the tablet comprises about 0.14 w/w % to about 0.15 w/w % of the monohydrate of the compound of Formula IIa or IIb. In some embodiments, the tablet comprises about 0.14 w/w % of the monohydrate of the compound of Formula IIa or IIb. In some embodiments, the tablet comprises about 0.142 w/w % of the monohydrate of the compound of Formula IIa or IIb.

In some embodiments, the tablet comprises about 0.03 w/w % to about 0.04 w/w % of the monohydrate of the compound of Formula IIa or IIb. In some embodiments, the tablet comprises about 0.035 w/w % to about 0.036 w/w % of the monohydrate of the compound of Formula IIa or IIb. In some embodiments, the tablet comprises about 0.0355 w/w % of the monohydrate of the compound of Formula IIa or IIb.

In some embodiments, the tablet disclosed herein comprises a compound of Formula Ia or Ib, or a pharmaceutically acceptable salt, co-crystal, solvate, or combination thereof, a compound of Formula IIa or IIb, or a pharmaceutically acceptable salt, co-crystal, solvate, or combination thereof, and one or more pharmaceutically acceptable excipients selected from the group consisting of a filler, a disintegrant, and a lubricant.

In some embodiments, the one or more fillers comprise microcrystalline cellulose and/or mannitol. In some embodiments, the one or more fillers comprise microcrystalline cellulose and mannitol. In some embodiments, the disintegrant comprises croscarmellose sodium. In some embodiments, the lubricant comprises magnesium stearate.

In some embodiments, the tablet comprises one or more pharmaceutically acceptable excipients selected from mannitol, microcrystalline cellulose, croscarmellose sodium, and magnesium stearate, or any combination thereof. In some embodiments, the tablet comprises mannitol, microcrystalline cellulose, croscarmellose sodium, and magnesium stearate.

In some embodiments, the tablet disclosed herein comprises a compound of Formula Ia or Ib, or a pharmaceutically acceptable salt, co-crystal, solvate, or combination thereof, a compound of Formula IIa or IIb, or a pharmaceutically acceptable salt, co-crystal, solvate, or combination thereof, mannitol, microcrystalline cellulose, croscarmellose sodium, and magnesium stearate.

In some embodiments, the tablet disclosed herein comprises a compound of Formula Ia or Ib, or a pharmaceutically acceptable salt thereof, a compound of Formula IIa or IIb, or a solvate thereof, mannitol, microcrystalline cellulose, croscarmellose sodium, and magnesium stearate.

In some embodiments, the tablet disclosed herein comprises a sodium salt of the Formula Ia or Ib, or a pharmaceutically acceptable salt thereof, a monohydrate of the compound of Formula IIa or IIb, mannitol, microcrystalline cellulose, croscarmellose sodium, and magnesium stearate.

In some embodiments, the tablet comprises about 35 w/w % to about 45 w/w % of mannitol, for example, about 35 w/w %, about 36 w/w %, about 37 w/w %, about 38 w/w %, about 39 w/w %, about 40 w/w %, about 41 w/w %, about 42 w/w %, about 43 w/w %, about 44 w/w %, or about 45 w/w % of mannitol. In some embodiments, the tablet comprises about 40 w/w % to about 42 w/w % of mannitol. In some embodiments, the tablet comprises about 41.3 w/w % to about 41.5 w/w % of mannitol. In some embodiments, the tablet comprises about 41.4 w/w % of mannitol.

In some embodiments, the tablet comprises about 20 w/w % to about 25 w/w % of microcrystalline cellulose, for example, about 20 w/w %, about 21 w/w %, about 22 w/w %, about 23 w/w %, about 24 w/w %, or about 25 w/w % microcrystalline cellulose. In some embodiments, the tablet comprises about 22 w/w % to about 23 w/w % of microcrystalline cellulose. In some embodiments, the tablet comprises about 22.2 w/w % to about 22.4 w/w % of microcrystalline cellulose.

In some embodiments, the tablet comprises about 22.2 w/w % to about 22.3 w/w % microcrystalline cellulose. In some embodiments, the tablet comprises about 22.29 w/w % microcrystalline cellulose. In some embodiments, the tablet comprises about 22.288 w/w % microcrystalline cellulose.

In some embodiments, the tablet comprises about 22.3 w/w % to about 22.4 w/w % microcrystalline cellulose. In some embodiments, the tablet comprises about 22.39 w/w % microcrystalline cellulose. In some embodiments, the tablet comprises about 22.3945 w/w % microcrystalline cellulose.

In some embodiments, the tablet comprises about 5 w/w % to about 10 w/w % of croscarmellose sodium, for example, about 5 w/w %, about 6 w/w %, about 7 w/w %, about 8 w/w %, about 9 w/w %, or about 10 w/w % croscarmellose sodium. In some embodiments, the tablet comprises about 7 w/w % to about 9 w/w % of croscarmellose sodium. In some embodiments, the tablet comprises about 7.5 w/w % to about 8.5 w/w % of croscarmellose sodium. In some embodiments, the tablet comprises about 7.9 w/w % to about 8.1 w/w % of croscarmellose sodium. In some embodiments, the tablet comprises about 8 w/w % of croscarmellose sodium.

In some embodiments, the tablet comprises about 0.1 w/w % to about 2.0 w/w % of magnesium stearate, for example, 0.1 w/w %, 0.2 w/w %, 0.3 w/w %, 0.4 w/w %, 0.5 w/w %, 0.6 w/w %, 0.7 w/w %, 0.8 w/w %, 0.9 w/w %, 1.0 w/w %, 1.1 w/w %, 1.2 w/w %, 1.3 w/w %, 1.4 w/w %, 1.5 w/w %, 1.6 w/w %, 1.7 w/w %, 1.8 w/w %, 1.9 w/w %, or about 2.0 w/w % magnesium stearate. In some embodiments, the tablet comprises about 1.0 w/w % to about 2.0 w/w % of magnesium stearate. In some embodiments, the tablet comprises about 1.4 w/w % to about 1.6 w/w % of magnesium stearate. In some embodiments, the tablet comprises about 1.5 w/w % of magnesium stearate.

In some embodiments, the tablet comprises:
about 20 w/w % to about 30 w/w % of the compound of Formula Ia or Ib, or a pharmaceutically acceptable salt, co-crystal, solvate, or combination thereof;
about 0.01 w/w % to about 2.0 w/w % of the compound of Formula IIa or IIb, or a pharmaceutically acceptable salt, co-crystal, solvate, or combination thereof; and
one or more pharmaceutically acceptable excipients.

In some embodiments, the tablet comprises:
about 26 w/w % to about 27 w/w % of the spray dried dispersion of the compound of Formula Ia or Ib, or a pharmaceutically acceptable salt, co-crystal, solvate, or combination thereof;
about 0.02 w/w % to about 0.2 w/w % of the compound of Formula IIa or IIb, or a pharmaceutically acceptable salt, co-crystal, solvate, or combination thereof; and one or more pharmaceutically acceptable excipients.

In some embodiments, the tablet comprises:
about 26.6 w/w % to about 26.7 w/w % of the spray dried dispersion of the compound of Formula Ia or Ib, or a pharmaceutically acceptable salt, co-crystal, solvate, or combination thereof;
about 0.14 w/w % to about 0.15 w/w % of the compound of Formula IIa or IIb, or a pharmaceutically acceptable salt, co-crystal, solvate, or combination thereof; and one or more pharmaceutically acceptable excipients.

In some embodiments, the tablet comprises:
about 26.67 w/w % of the spray dried dispersion of the compound of Formula Ia or Ib, or a pharmaceutically acceptable salt, co-crystal, solvate, or combination thereof;
about 0.142 w/w % of the compound of Formula IIa or IIb, or a pharmaceutically acceptable salt, co-crystal, solvate, or combination thereof; and
one or more pharmaceutically acceptable excipients.

In some embodiments, the tablet comprises:
about 26.6 w/w % to about 26.7 w/w % of the spray dried dispersion of the compound of Formula Ia or Ib, or a pharmaceutically acceptable salt, co-crystal, solvate, or combination thereof;
about 0.03 w/w % to about 0.04 w/w % of the compound of Formula IIa or IIb, or a pharmaceutically acceptable salt, co-crystal, solvate, or combination thereof; and
one or more pharmaceutically acceptable excipients.

In some embodiments, the tablet comprises:
about 26.67 w/w % of the spray dried dispersion of the compound of Formula Ia or Ib, or a pharmaceutically acceptable salt, co-crystal, solvate, or combination thereof;
about 0.0355 w/w % of the compound of Formula IIa or IIb, or a pharmaceutically acceptable salt, co-crystal, solvate, or combination thereof; and
one or more pharmaceutically acceptable excipients.

In some embodiments, the tablet comprises:
about 26 w/w % to about 27 w/w % of the spray dried dispersion of the compound of Formula Ia or Ib, or a pharmaceutically acceptable salt thereof;
about 0.02 w/w % to about 0.2 w/w % of the compound of Formula IIa or IIb, or a pharmaceutically acceptable salt, co-crystal, solvate, or combination thereof; and
one or more pharmaceutically acceptable excipients.

In some embodiments, the tablet comprises:
about 26.6 w/w % to about 26.7 w/w % of the spray dried dispersion of the compound of Formula Ia or Ib, or a pharmaceutically acceptable salt thereof;
about 0.14 w/w % to about 0.15 w/w % of the compound of Formula IIa or IIb, or a pharmaceutically acceptable salt, co-crystal, solvate, or combination thereof; and
one or more pharmaceutically acceptable excipients.

In some embodiments, the tablet comprises:
about 26.67 w/w % of the spray dried dispersion of the compound of Formula Ia or Ib, or a pharmaceutically acceptable salt thereof;
about 0.142 w/w % of the compound of Formula IIa or IIb, or a pharmaceutically acceptable salt, co-crystal, solvate, or combination thereof; and
one or more pharmaceutically acceptable excipients.

In some embodiments, the tablet comprises:
about 26.6 w/w % to about 26.7 w/w % of the spray dried dispersion of the compound of Formula Ia or Ib, or a pharmaceutically acceptable salt thereof;
about 0.03 w/w % to about 0.04 w/w % of the compound of Formula IIa or IIb, or a pharmaceutically acceptable salt, co-crystal, solvate, or combination thereof; and
one or more pharmaceutically acceptable excipients.

In some embodiments, the tablet comprises:
about 26.67 w/w % of the spray dried dispersion of the compound of Formula Ia or Ib, or a pharmaceutically acceptable salt thereof;
about 0.0355 w/w % of the compound of Formula IIa or IIb, or a pharmaceutically acceptable salt, co-crystal, solvate, or combination thereof; and
one or more pharmaceutically acceptable excipients.

In some embodiments, the tablet comprises:
about 26 w/w % to about 27 w/w % of the spray dried dispersion of the sodium salt of the compound of Formula Ia or Ib;
about 0.02 w/w % to about 0.2 w/w % of the compound of Formula IIa or IIb, or a pharmaceutically acceptable salt, co-crystal, solvate, or combination thereof; and
one or more pharmaceutically acceptable excipients.

In some embodiments, the tablet comprises:
about 26.6 w/w % to about 26.7 w/w % of the spray dried dispersion of the sodium salt of the compound of Formula Ia or Ib;
about 0.14 w/w % to about 0.15 w/w % of the compound of Formula IIa or IIb, or a pharmaceutically acceptable salt, co-crystal, solvate, or combination thereof; and
one or more pharmaceutically acceptable excipients.

In some embodiments, the tablet comprises:
about 26.67 w/w % of the spray dried dispersion of the sodium salt of the compound of Formula Ia or Ib;
about 0.142 w/w % of the compound of Formula IIa or IIb, or a pharmaceutically acceptable salt, co-crystal, solvate, or combination thereof; and
one or more pharmaceutically acceptable excipients.

In some embodiments, the tablet comprises:
about 26.6 w/w % to about 26.7 w/w % of the spray dried dispersion of the sodium salt of the compound of Formula Ia or Ib;
about 0.03 w/w % to about 0.04 w/w % of the compound of Formula IIa or IIb, or a pharmaceutically acceptable salt, co-crystal, solvate, or combination thereof; and
one or more pharmaceutically acceptable excipients.

In some embodiments, the tablet comprises:
about 26.67 w/w % of the spray dried dispersion of the sodium salt of the compound of Formula Ia or Ib;
about 0.0355 w/w % of the compound of Formula IIa or IIb, or a pharmaceutically acceptable salt, co-crystal, solvate, or combination thereof; and
one or more pharmaceutically acceptable excipients.

In some embodiments, the tablet comprises:
about 26 w/w % to about 27 w/w % of the spray dried dispersion of the sodium salt of the compound of Formula Ia;
about 0.02 w/w % to about 0.2 w/w % of the compound of Formula IIa or IIb, or a pharmaceutically acceptable salt, co-crystal, solvate, or combination thereof; and
one or more pharmaceutically acceptable excipients.

In some embodiments, the tablet comprises:
about 26.6 w/w % to about 26.7 w/w % of the spray dried dispersion of the sodium salt of the compound of Formula Ia;
about 0.14 w/w % to about 0.15 w/w % of the compound of Formula IIa or IIb, or a pharmaceutically acceptable salt, co-crystal, solvate, or combination thereof; and
one or more pharmaceutically acceptable excipients.

In some embodiments, the tablet comprises:
about 26.67 w/w % of the spray dried dispersion of the sodium salt of the compound of Formula Ia;
about 0.142 w/w % of the compound of Formula IIa or IIb, or a pharmaceutically acceptable salt, co-crystal, solvate, or combination thereof; and
one or more pharmaceutically acceptable excipients.

In some embodiments, the tablet comprises:
about 26.6 w/w % to about 26.7 w/w % of the spray dried dispersion of the sodium salt of the compound of Formula Ia;
about 0.03 w/w % to about 0.04 w/w % of the compound of Formula IIa or IIb, or a pharmaceutically acceptable salt, co-crystal, solvate, or combination thereof; and
one or more pharmaceutically acceptable excipients.

In some embodiments, the tablet comprises:
about 26.67 w/w % of the spray dried dispersion of the sodium salt of the compound of Formula Ia;
about 0.0355 w/w % of the compound of Formula IIa or IIb, or a pharmaceutically acceptable salt, co-crystal, solvate, or combination thereof; and
one or more pharmaceutically acceptable excipients.

In some embodiments, the tablet comprises:
about 26 w/w % to about 27 w/w % of the lenacapavir spray dried dispersion; about 0.02 w/w % to about 0.2 w/w % of the compound of Formula IIa or IIb, or a pharmaceutically acceptable salt, co-crystal, solvate, or combination thereof; and
one or more pharmaceutically acceptable excipients.

In some embodiments, the tablet comprises:
about 26.6 w/w % to about 26.7 w/w % of the lenacapavir spray dried dispersion;
about 0.14 w/w % to about 0.15 w/w % of the compound of Formula IIa or IIb, or a pharmaceutically acceptable salt, co-crystal, solvate, or combination thereof; and
one or more pharmaceutically acceptable excipients.

In some embodiments, the tablet comprises:
about 26.67 w/w % of the lenacapavir spray dried dispersion; about 0.142 w/w % of the compound of Formula IIa or IIb, or a pharmaceutically acceptable salt, co-crystal, solvate, or combination thereof; and
one or more pharmaceutically acceptable excipients.

In some embodiments, the tablet comprises:
about 26.6 w/w % to about 26.7 w/w % of the lenacapavir spray dried dispersion;
about 0.03 w/w % to about 0.04 w/w % of the compound of Formula IIa or IIb, or a pharmaceutically acceptable salt, co-crystal, solvate, or combination thereof; and
one or more pharmaceutically acceptable excipients.

In some embodiments, the tablet comprises:
about 26.67 w/w % of the lenacapavir spray dried dispersion;
about 0.0355 w/w % of the compound of Formula IIa or IIb, or a pharmaceutically acceptable salt, co-crystal, solvate, or combination thereof; and
one or more pharmaceutically acceptable excipients.

In some embodiments, the tablet comprises:
about 18 w/w % to about 30 w/w % of the compound of Formula Ia or Ib, or a pharmaceutically acceptable salt thereof;
about 0.01 w/w % to about 2.0 w/w % of the compound of Formula IIa or IIb, or a solvate thereof, and
one or more pharmaceutically acceptable excipients.

In some embodiments, the tablet comprises:
about 19 w/w % to about 21 w/w % of the compound of Formula Ia or Ib, or a pharmaceutically acceptable salt thereof;
about 0.02 w/w % to about 0.2 w/w % of the compound of Formula IIa or IIb, or a solvate thereof, and
one or more pharmaceutically acceptable excipients.

In some embodiments, the tablet comprises:
about 19.8 w/w % to about 20.8 w/w % of the compound of Formula Ia or Ib, or a pharmaceutically acceptable salt thereof;
about 0.14 w/w % to about 0.15 w/w % of the compound of Formula IIa or IIb, or a solvate thereof, and
one or more pharmaceutically acceptable excipients.

In some embodiments, the tablet comprises:
about 20.46 w/w % of the compound of Formula Ia or Ib, or a pharmaceutically acceptable salt thereof;
about 0.142 w/w % of the compound of Formula IIa or IIb, or a solvate thereof; and
one or more pharmaceutically acceptable excipients.

In some embodiments, the tablet comprises:
about 20.45 w/w % of the compound of Formula Ia or Ib, or a pharmaceutically acceptable salt thereof;
about 0.142 w/w % of the compound of Formula IIa or IIb, or a solvate thereof; and
one or more pharmaceutically acceptable excipients.

In some embodiments, the tablet comprises:
about 20.0 w/w % of the compound of Formula Ia or Ib, or a pharmaceutically acceptable salt thereof;
about 0.142 w/w % of the compound of Formula IIa or IIb, or a solvate thereof; and
one or more pharmaceutically acceptable excipients.

In some embodiments, the tablet comprises:
about 19 w/w % to about 21 w/w % of the compound of Formula Ia or Ib, or a pharmaceutically acceptable salt thereof;
about 0.03 w/w % to about 0.04 w/w % of the compound of Formula IIa or IIb, or a solvate thereof, and
one or more pharmaceutically acceptable excipients.

In some embodiments, the tablet comprises:
about 19.8 w/w % to about 20.8 w/w % of the compound of Formula Ia or Ib, or a pharmaceutically acceptable salt thereof;
about 0.03 w/w % to about 0.04 w/w % of the compound of Formula IIa or IIb, or a solvate thereof, and
one or more pharmaceutically acceptable excipients.

In some embodiments, the tablet comprises:
about 20.46 w/w % of the compound of Formula Ia or Ib, or a pharmaceutically acceptable salt thereof;
about 0.0355 w/w % of the compound of Formula IIa or IIb, or a solvate thereof, and
one or more pharmaceutically acceptable excipients.

In some embodiments, the tablet comprises:
about 20.45 w/w % of the compound of Formula Ia or Ib, or a pharmaceutically acceptable salt thereof;
about 0.0355 w/w % of the compound of Formula IIa or IIb, or a solvate thereof, and
one or more pharmaceutically acceptable excipients.

In some embodiments, the tablet comprises:
about 20.0 w/w % of the compound of Formula Ia or Ib, or a pharmaceutically acceptable salt thereof;
about 0.0355 w/w % of the compound of Formula IIa or IIb, or a solvate thereof, and
one or more pharmaceutically acceptable excipients.

In some embodiments, the tablet comprises:
about 18 w/w % to about 30 w/w % of the sodium salt of the compound of Formula Ia or Ib;
about 0.01 w/w % to about 2.0 w/w % of the monohydrate of the compound of Formula IIa or IIb; and
one or more pharmaceutically acceptable excipients.

In some embodiments, the tablet comprises:
about 20 w/w % to about 21 w/w % of the sodium salt of the compound of Formula Ia or Ib;
about 0.02 w/w % to about 0.2 w/w % of the monohydrate of the compound of Formula IIa or IIb; and
one or more pharmaceutically acceptable excipients.

In some embodiments, the tablet comprises:
about 20.2 w/w % to about 20.7 w/w % of the sodium salt of the compound of Formula Ia or Ib;
about 0.14 w/w % to about 0.15 w/w % of the monohydrate of the compound of Formula IIa or IIb; and
one or more pharmaceutically acceptable excipients.

In some embodiments, the tablet comprises:
about 20.46 w/w % of the sodium salt of the compound of Formula Ia or Ib;
about 0.142 w/w % of the monohydrate of the compound of Formula IIa or IIb; and
one or more pharmaceutically acceptable excipients.

In some embodiments, the tablet comprises:
about 20.45 w/w % of the sodium salt of the compound of Formula Ia or Ib;
about 0.142 w/w % of the monohydrate of the compound of Formula IIa or IIb; and
one or more pharmaceutically acceptable excipients.

In some embodiments, the tablet comprises:
about 20.2 w/w % to about 20.7 w/w % of the sodium salt of the compound of Formula Ia or Ib;
about 0.03 w/w % to about 0.04 w/w % of the monohydrate of the compound of Formula IIa or IIb; and
one or more pharmaceutically acceptable excipients.

In some embodiments, the tablet comprises:
about 20.46 w/w % of the sodium salt of the compound of Formula Ia or Ib;
about 0.0355 w/w % of the monohydrate of the compound of Formula IIa or IIb; and
one or more pharmaceutically acceptable excipients.

In some embodiments, the tablet comprises:
about 20.45 w/w % of the sodium salt of the compound of Formula Ia or Ib;
about 0.0355 w/w % of the monohydrate of the compound of Formula IIa or IIb; and
one or more pharmaceutically acceptable excipients.

In some embodiments, the tablet comprises:
about 35 w/w % to about 45 w/w % of mannitol;
about 20 w/w % to about 25 w/w % of microcrystalline cellulose;
about 5 w/w % to about 10 w/w % of croscarmellose sodium; and
about 0.1 w/w % to about 2.0 w/w % of magnesium stearate.

In some embodiments, the tablet comprises:
about 40 w/w % to about 42 w/w % of mannitol;
about 22 w/w % to about 23 w/w % of microcrystalline cellulose;
about 7 w/w % to about 9 w/w % of croscarmellose sodium; and
about 1.0 w/w % to about 2.0 w/w % of magnesium stearate

In some embodiments, the tablet comprises:
about 41 w/w % to about 42 w/w % of mannitol;
about 22.2 w/w % to about 22.3 w/w % of microcrystalline cellulose;
about 7.9 w/w % to about 8.1 w/w % of croscarmellose sodium; and
about 1.4 w/w % to about 1.6 w/w % of magnesium stearate.

In some embodiments, the tablet comprises:
about 41.40 w/w % of mannitol;
about 22.288 w/w % microcrystalline cellulose;
about 8.0 w/w % of croscarmellose sodium; and
about 1.5 w/w % of magnesium stearate.

In some embodiments, the tablet comprises:
about 41 w/w % to about 42 w/w % of mannitol;
about 22.3 w/w % to about 22.4 w/w % of microcrystalline cellulose;
about 7.9 w/w % to about 8.1 w/w % of croscarmellose sodium; and
about 1.4 w/w % to about 1.6 w/w % of magnesium stearate.

In some embodiments, the tablet comprises:
about 41.40 w/w % of mannitol;
about 22.3945 w/w % microcrystalline cellulose;
about 8.0 w/w % of croscarmellose sodium; and
about 1.5 w/w % of magnesium stearate.

In some embodiments, the tablet comprises:
about 20 w/w % to about 30 w/w % of the compound of Formula Ia or Ib, or a pharmaceutically acceptable salt, co-crystal, solvate, or combination thereof;
about 0.01 w/w % to about 2.0 w/w % of the compound of Formula IIa or IIb, or a pharmaceutically acceptable salt, co-crystal, solvate, or combination thereof;
about 35 w/w % to about 45 w/w % of mannitol;
about 20 w/w % to about 25 w/w % of microcrystalline cellulose;
about 5 w/w % to about 10 w/w % of croscarmellose sodium; and
about 0.1 w/w % to about 2.0 w/w % of magnesium stearate.

In some embodiments, the tablet comprises:
about 26 w/w % to about 27 w/w % of the lenacapavir spray dried dispersion;
about 0.02 w/w % to about 0.2 w/w % of the compound of Formula IIa or IIb, or a pharmaceutically acceptable salt, co-crystal, solvate, or combination thereof;
about 40 w/w % to about 42 w/w % of mannitol;
about 22 w/w % to about 23 w/w % of microcrystalline cellulose;
about 7 w/w % to about 9 w/w % of croscarmellose sodium; and
about 1.0 w/w % to about 2.0 w/w % of magnesium stearate.

In some embodiments, the tablet comprises:
about 26.6 w/w % to about 26.7 w/w % of the lenacapavir spray dried dispersion;
about 0.14 w/w % to about 0.15 w/w % of the compound of Formula IIa or IIb, or a pharmaceutically acceptable salt, co-crystal, solvate, or combination thereof;
about 41 w/w % to about 42 w/w % of mannitol;
about 22.2 w/w % to about 22.3 w/w % of microcrystalline cellulose;
about 7.9 w/w % to about 8.1 w/w % of croscarmellose sodium; and
about 1.4 w/w % to about 1.6 w/w % of magnesium stearate.

In some embodiments, the tablet comprises:
about 26.67 w/w % of the lenacapavir spray dried dispersion;
about 0.142 w/w % of the compound of Formula IIa or IIb, or a pharmaceutically acceptable salt, co-crystal, solvate, or combination thereof;
about 41.40 w/w % of mannitol;
about 22.288 w/w % microcrystalline cellulose;
about 8.0 w/w % of croscarmellose sodium; and
about 1.5 w/w % of magnesium stearate.

In some embodiments, the tablet comprises:
about 26.6 w/w % to about 26.7 w/w % of the lenacapavir spray dried dispersion;
about 0.03 w/w % to about 0.04 w/w % of the compound of Formula IIa or IIb, or a pharmaceutically acceptable salt, co-crystal, solvate, or combination thereof;
about 41 w/w % to about 42 w/w % of mannitol;
about 22.3 w/w % to about 22.4 w/w % of microcrystalline cellulose;
about 7.9 w/w % to about 8.1 w/w % of croscarmellose sodium; and
about 1.4 w/w % to about 1.6 w/w % of magnesium stearate.

In some embodiments, the tablet comprises:
about 26.67 w/w % of the lenacapavir spray dried dispersion;
about 0.0355 w/w % of the compound of Formula IIa or IIb, or a pharmaceutically acceptable salt, co-crystal, solvate, or combination thereof;
about 41.40 w/w % of mannitol;
about 22.3945 w/w % microcrystalline cellulose;
about 8.0 w/w % of croscarmellose sodium; and
about 1.5 w/w % of magnesium stearate.

In some embodiments, the tablet comprises:
about 26 w/w % to about 27 w/w % of the spray dried dispersion of the compound of Formula Ia or Ib, or a pharmaceutically acceptable salt, co-crystal, solvate, or combination thereof;
about 0.02 w/w % to about 0.2 w/w % of the compound of Formula IIa or IIb, or a pharmaceutically acceptable salt, co-crystal, solvate, or combination thereof;
about 40 w/w % to about 42 w/w % of mannitol;
about 22 w/w % to about 23 w/w % of microcrystalline cellulose;
about 7 w/w % to about 9 w/w % of croscarmellose sodium; and
about 1.0 w/w % to about 2.0 w/w % of magnesium stearate.

In some embodiments, the tablet comprises:
about 26.6 w/w % to about 26.7 w/w % of the spray dried dispersion of the compound of Formula Ia or Ib, or a pharmaceutically acceptable salt, co-crystal, solvate, or combination thereof;
about 0.14 w/w % to about 0.15 w/w % of the compound of Formula IIa or IIb, or a pharmaceutically acceptable salt, co-crystal, solvate, or combination thereof;
about 41 w/w % to about 42 w/w % of mannitol;
about 22.2 w/w % to about 22.3 w/w % of microcrystalline cellulose;
about 7.9 w/w % to about 8.1 w/w % of croscarmellose sodium; and
about 1.4 w/w % to about 1.6 w/w % of magnesium stearate.

In some embodiments, the tablet comprises:
about 26.67 w/w % of the spray dried dispersion of the compound of Formula Ia or Ib, or a pharmaceutically acceptable salt, co-crystal, solvate, or combination thereof;
about 0.142 w/w % of the compound of Formula IIa or IIb, or a pharmaceutically acceptable salt, co-crystal, solvate, or combination thereof;
about 41.40 w/w % of mannitol;
about 22.288 w/w % microcrystalline cellulose;
about 8.0 w/w % of croscarmellose sodium; and
about 1.5 w/w % of magnesium stearate.

In some embodiments, the tablet comprises:
about 26.6 w/w % to about 26.7 w/w % of the spray dried dispersion of the compound of Formula Ia or Ib, or a pharmaceutically acceptable salt, co-crystal, solvate, or combination thereof;
about 0.03 w/w % to about 0.04 w/w % of the compound of Formula IIa or IIb, or a pharmaceutically acceptable salt, co-crystal, solvate, or combination thereof;
about 41 w/w % to about 42 w/w % of mannitol;
about 22.3 w/w % to about 22.4 w/w % of microcrystalline cellulose;
about 7.9 w/w % to about 8.1 w/w % of croscarmellose sodium; and
about 1.4 w/w % to about 1.6 w/w % of magnesium stearate.

In some embodiments, the tablet comprises:
about 26.67 w/w % of the spray dried dispersion of the compound of Formula Ia or Ib, or a pharmaceutically acceptable salt, co-crystal, solvate, or combination thereof;
about 0.0355 w/w % of the compound of Formula IIa or IIb, or a pharmaceutically acceptable salt, co-crystal, solvate, or combination thereof;
about 41.40 w/w % of mannitol;
about 22.3945 w/w % microcrystalline cellulose;
about 8.0 w/w % of croscarmellose sodium; and
about 1.5 w/w % of magnesium stearate.

In some embodiments, the tablet comprises:
about 26 w/w % to about 27 w/w % of the spray dried dispersion of the compound of Formula Ia or Ib, or a pharmaceutically acceptable salt thereof;
about 0.02 w/w % to about 0.2 w/w % of the compound of Formula IIa or IIb, or a pharmaceutically acceptable salt, co-crystal, solvate, or combination thereof;
about 40 w/w % to about 42 w/w % of mannitol;
about 22 w/w % to about 23 w/w % of microcrystalline cellulose;
about 7 w/w % to about 9 w/w % of croscarmellose sodium; and
about 1.0 w/w % to about 2.0 w/w % of magnesium stearate.

In some embodiments, the tablet comprises:
about 26.6 w/w % to about 26.7 w/w % of the spray dried dispersion of the compound of Formula Ia or Ib, or a pharmaceutically acceptable salt thereof;
about 0.14 w/w % to about 0.15 w/w % of the compound of Formula IIa or IIb, or a pharmaceutically acceptable salt, co-crystal, solvate, or combination thereof;
about 41 w/w % to about 42 w/w % of mannitol;
about 22.2 w/w % to about 22.3 w/w % of microcrystalline cellulose;
about 7.9 w/w % to about 8.1 w/w % of croscarmellose sodium; and
about 1.4 w/w % to about 1.6 w/w % of magnesium stearate.

In some embodiments, the tablet comprises:
about 26.67 w/w % of the spray dried dispersion of the compound of Formula Ia or Ib, or a pharmaceutically acceptable salt thereof;
about 0.142 w/w % of the compound of Formula IIa or IIb, or a pharmaceutically acceptable salt, co-crystal, solvate, or combination thereof;
about 41.40 w/w % of mannitol;
about 22.288 w/w % microcrystalline cellulose;
about 8.0 w/w % of croscarmellose sodium; and
about 1.5 w/w % of magnesium stearate.

In some embodiments, the tablet comprises:
about 26.6 w/w % to about 26.7 w/w % of the spray dried dispersion of the compound of Formula Ia or Ib, or a pharmaceutically acceptable salt thereof;
about 0.03 w/w % to about 0.04 w/w % of the compound of Formula IIa or IIb, or a pharmaceutically acceptable salt, co-crystal, solvate, or combination thereof;
about 41 w/w % to about 42 w/w % of mannitol;
about 22.3 w/w % to about 22.4 w/w % of microcrystalline cellulose;
about 7.9 w/w % to about 8.1 w/w % of croscarmellose sodium; and
about 1.4 w/w % to about 1.6 w/w % of magnesium stearate.

In some embodiments, the tablet comprises:
about 26.67 w/w % of the spray dried dispersion of the compound of Formula Ia or Ib, or a pharmaceutically acceptable salt thereof;
about 0.0355 w/w % of the compound of Formula IIa or IIb, or a pharmaceutically acceptable salt, co-crystal, solvate, or combination thereof;
about 41.40 w/w % of mannitol;
about 22.3945 w/w % microcrystalline cellulose;
about 8.0 w/w % of croscarmellose sodium; and
about 1.5 w/w % of magnesium stearate.

In some embodiments, the tablet comprises:
about 26 w/w % to about 27 w/w % of the spray dried dispersion of the sodium salt of the compound of Formula Ia or Ib;
about 0.02 w/w % to about 0.2 w/w % of the compound of Formula IIa or IIb, or a pharmaceutically acceptable salt, co-crystal, solvate, or combination thereof;
about 40 w/w % to about 42 w/w % of mannitol;
about 22 w/w % to about 23 w/w % of microcrystalline cellulose;
about 7 w/w % to about 9 w/w % of croscarmellose sodium; and
about 1.0 w/w % to about 2.0 w/w % of magnesium stearate.

In some embodiments, the tablet comprises:
about 26.6 w/w % to about 26.7 w/w % of the spray dried dispersion of the sodium salt of the compound of Formula Ia or Ib;
about 0.14 w/w % to about 0.15 w/w % of the compound of Formula IIa or IIb, or a pharmaceutically acceptable salt, co-crystal, solvate, or combination thereof;
about 41 w/w % to about 42 w/w % of mannitol;
about 22.2 w/w % to about 22.3 w/w % of microcrystalline cellulose;
about 7.9 w/w % to about 8.1 w/w % of croscarmellose sodium; and
about 1.4 w/w % to about 1.6 w/w % of magnesium stearate.

In some embodiments, the tablet comprises:
about 26.67 w/w % of the spray dried dispersion of the sodium salt of the compound of Formula Ia or Ib;
about 0.142 w/w % of the compound of Formula IIa or IIb, or a pharmaceutically acceptable salt, co-crystal, solvate, or combination thereof;
about 41.40 w/w % of mannitol;
about 22.288 w/w % microcrystalline cellulose;
about 8.0 w/w % of croscarmellose sodium; and
about 1.5 w/w % of magnesium stearate.

In some embodiments, the tablet comprises:
about 26.6 w/w % to about 26.7 w/w % of the spray dried dispersion of the sodium salt of the compound of Formula Ia or Ib;
about 0.03 w/w % to about 0.04 w/w % of the compound of Formula IIa or IIb, or a pharmaceutically acceptable salt, co-crystal, solvate, or combination thereof;
about 41 w/w % to about 42 w/w % of mannitol;
about 22.3 w/w % to about 22.4 w/w % of microcrystalline cellulose;
about 7.9 w/w % to about 8.1 w/w % of croscarmellose sodium; and
about 1.4 w/w % to about 1.6 w/w % of magnesium stearate.

In some embodiments, the tablet comprises:
about 26.67 w/w % of the spray dried dispersion of the sodium salt of the compound of Formula Ia or Ib;
about 0.0355 w/w % of the compound of Formula IIa or IIb, or a pharmaceutically acceptable salt, co-crystal, solvate, or combination thereof;
about 41.40 w/w % of mannitol;
about 22.3945 w/w % microcrystalline cellulose;
about 8.0 w/w % of croscarmellose sodium; and
about 1.5 w/w % of magnesium stearate.

In some embodiments, the tablet comprises:
about 26 w/w % to about 27 w/w % of the spray dried dispersion of the sodium salt of the compound of Formula Ia;
about 0.02 w/w % to about 0.2 w/w % of the compound of Formula IIa or IIb, or a pharmaceutically acceptable salt, co-crystal, solvate, or combination thereof;
about 40 w/w % to about 42 w/w % of mannitol;
about 22 w/w % to about 23 w/w % of microcrystalline cellulose;
about 7 w/w % to about 9 w/w % of croscarmellose sodium; and
about 1.0 w/w % to about 2.0 w/w % of magnesium stearate.

In some embodiments, the tablet comprises:
about 26.6 w/w % to about 26.7 w/w % of the spray dried dispersion of the sodium salt of the compound of Formula Ia;
about 0.14 w/w % to about 0.15 w/w % of the compound of Formula IIa or IIb, or a pharmaceutically acceptable salt, co-crystal, solvate, or combination thereof;
about 41 w/w % to about 42 w/w % of mannitol;
about 22.2 w/w % to about 22.3 w/w % of microcrystalline cellulose;
about 7.9 w/w % to about 8.1 w/w % of croscarmellose sodium; and
about 1.4 w/w % to about 1.6 w/w % of magnesium stearate.

In some embodiments, the tablet comprises:
about 26.67 w/w % of the spray dried dispersion of the sodium salt of the compound of Formula Ia;
about 0.142 w/w % of the compound of Formula IIa or IIb, or a pharmaceutically acceptable salt, co-crystal, solvate, or combination thereof;
about 41.40 w/w % of mannitol;
about 22.288 w/w % microcrystalline cellulose;
about 8.0 w/w % of croscarmellose sodium; and
about 1.5 w/w % of magnesium stearate.

In some embodiments, the tablet comprises:
about 26.6 w/w % to about 26.7 w/w % of the spray dried dispersion of the sodium salt of the compound of Formula Ia;
about 0.03 w/w % to about 0.04 w/w % of the compound of Formula IIa or IIb, or a pharmaceutically acceptable salt, co-crystal, solvate, or combination thereof;
about 41 w/w % to about 42 w/w % of mannitol;
about 22.3 w/w % to about 22.4 w/w % of microcrystalline cellulose;
about 7.9 w/w % to about 8.1 w/w % of croscarmellose sodium; and
about 1.4 w/w % to about 1.6 w/w % of magnesium stearate.

In some embodiments, the tablet comprises:
about 26.67 w/w % of the spray dried dispersion of the sodium salt of the compound of Formula Ia;
about 0.0355 w/w % of the compound of Formula IIa or IIb, or a pharmaceutically acceptable salt, co-crystal, solvate, or combination thereof;
about 41.40 w/w % of mannitol;
about 22.3945 w/w % microcrystalline cellulose;
about 8.0 w/w % of croscarmellose sodium; and
about 1.5 w/w % of magnesium stearate.

In some embodiments, the tablet comprises:
about 19 w/w % to about 21 w/w % of the compound of Formula Ia or Ib, or a pharmaceutically acceptable salt thereof;
about 0.02 w/w % to about 0.2 w/w % of the compound of Formula IIa or IIb, or a pharmaceutically acceptable salt, co-crystal, solvate, or combination thereof;
about 40 w/w % to about 42 w/w % of mannitol;
about 22 w/w % to about 23 w/w % of microcrystalline cellulose;
about 7 w/w % to about 9 w/w % of croscarmellose sodium; and
about 1.0 w/w % to about 2.0 w/w % of magnesium stearate.

In some embodiments, the tablet comprises:
about 19.8 w/w % to about 20.8 w/w % of the compound of Formula Ia or Ib, or a pharmaceutically acceptable salt thereof;
about 0.14 w/w % to about 0.15 w/w % of the compound of Formula IIa or IIb, or a pharmaceutically acceptable salt, co-crystal, solvate, or combination thereof;
about 41 w/w % to about 42 w/w % of mannitol;
about 22.2 w/w % to about 22.3 w/w % of microcrystalline cellulose;
about 7.9 w/w % to about 8.1 w/w % of croscarmellose sodium; and
about 1.4 w/w % to about 1.6 w/w % of magnesium stearate.

In some embodiments, the tablet comprises:
about 20.45 w/w % of the compound of Formula Ia or Ib, or a pharmaceutically acceptable salt thereof;
about 0.142 w/w % of the compound of Formula IIa or IIb, or a pharmaceutically acceptable salt, co-crystal, solvate, or combination thereof;
about 41.40 w/w % of mannitol;
about 22.288 w/w % microcrystalline cellulose;
about 8.0 w/w % of croscarmellose sodium; and
about 1.5 w/w % of magnesium stearate.

In some embodiments, the tablet comprises:
about 19.8 w/w % to about 20.8 w/w % of the compound of Formula Ia or Ib, or a pharmaceutically acceptable salt thereof;
about 0.03 w/w % to about 0.04 w/w % of the compound of Formula IIa or IIb, or a pharmaceutically acceptable salt, co-crystal, solvate, or combination thereof;
about 41 w/w % to about 42 w/w % of mannitol;
about 22.3 w/w % to about 22.4 w/w % of microcrystalline cellulose;
about 7.9 w/w % to about 8.1 w/w % of croscarmellose sodium; and
about 1.4 w/w % to about 1.6 w/w % of magnesium stearate.

In some embodiments, the tablet comprises:
about 20.45 w/w % of the compound of Formula Ia or Ib, or a pharmaceutically acceptable salt thereof;
about 0.0355 w/w % of the compound of Formula IIa or IIb, or a pharmaceutically acceptable salt, co-crystal, solvate, or combination thereof;
about 41.40 w/w % of mannitol;
about 22.3945 w/w % microcrystalline cellulose;
about 8.0 w/w % of croscarmellose sodium; and
about 1.5 w/w % of magnesium stearate.

In some embodiments, the tablet comprises:
about 20 w/w % to about 21 w/w % of the sodium salt of the compound of Formula Ia or Ib;
about 0.02 w/w % to about 0.2 w/w % of the compound of Formula IIa or IIb, or a pharmaceutically acceptable salt, co-crystal, solvate, or combination thereof;
about 40 w/w % to about 42 w/w % of mannitol;
about 22 w/w % to about 23 w/w % of microcrystalline cellulose;
about 7 w/w % to about 9 w/w % of croscarmellose sodium; and
about 1.0 w/w % to about 2.0 w/w % of magnesium stearate.

In some embodiments, the tablet comprises:
about 20.2 w/w % to about 20.7 w/w % of the sodium salt of the compound of Formula Ia or Ib;
about 0.14 w/w % to about 0.15 w/w % of the compound of Formula IIa or IIb, or a pharmaceutically acceptable salt, co-crystal, solvate, or combination thereof;
about 41 w/w % to about 42 w/w % of mannitol;
about 22.2 w/w % to about 22.3 w/w % of microcrystalline cellulose;
about 7.9 w/w % to about 8.1 w/w % of croscarmellose sodium; and
about 1.4 w/w % to about 1.6 w/w % of magnesium stearate.

In some embodiments, the tablet comprises:
about 20.45 w/w % of the sodium salt of the compound of Formula Ia or Ib;
about 0.142 w/w % of the compound of Formula IIa or IIb, or a pharmaceutically acceptable salt, co-crystal, solvate, or combination thereof;
about 41.40 w/w % of mannitol;
about 22.288 w/w % microcrystalline cellulose;
about 8.0 w/w % of croscarmellose sodium; and
about 1.5 w/w % of magnesium stearate.

In some embodiments, the tablet comprises:
about 20.2 w/w % to about 20.7 w/w % of the sodium salt of the compound of Formula Ia or Ib;
about 0.03 w/w % to about 0.04 w/w % of the compound of Formula IIa or IIb, or a pharmaceutically acceptable salt, co-crystal, solvate, or combination thereof;
about 41 w/w % to about 42 w/w % of mannitol;
about 22.3 w/w % to about 22.4 w/w % of microcrystalline cellulose;
about 7.9 w/w % to about 8.1 w/w % of croscarmellose sodium; and
about 1.4 w/w % to about 1.6 w/w % of magnesium stearate.

In some embodiments, the tablet comprises:
about 20.45 w/w % of the sodium salt of the compound of Formula Ia or Ib;
about 0.0355 w/w % of the compound of Formula IIa or IIb, or a pharmaceutically acceptable salt, co-crystal, solvate, or combination thereof;
about 41.40 w/w % of mannitol;
about 22.3945 w/w % microcrystalline cellulose;
about 8.0 w/w % of croscarmellose sodium; and
about 1.5 w/w % of magnesium stearate.

In some embodiments, the tablet comprises:
about 19 w/w % to about 21 w/w % of the compound of Formula Ia or Ib free acid;
about 0.02 w/w % to about 0.2 w/w % of the compound of Formula IIa or IIb, or a pharmaceutically acceptable salt, co-crystal, solvate, or combination thereof;
about 40 w/w % to about 42 w/w % of mannitol;
about 22 w/w % to about 23 w/w % of microcrystalline cellulose;
about 7 w/w % to about 9 w/w % of croscarmellose sodium; and
about 1.0 w/w % to about 2.0 w/w % of magnesium stearate.

In some embodiments, the tablet comprises:
about 19.8 w/w % to about 20.2 w/w % of the compound of Formula Ia or Ib free acid;
about 0.14 w/w % to about 0.15 w/w % of the compound of Formula IIa or IIb, or a pharmaceutically acceptable salt, co-crystal, solvate, or combination thereof;
about 41 w/w % to about 42 w/w % of mannitol;
about 22.2 w/w % to about 22.3 w/w % of microcrystalline cellulose;
about 7.9 w/w % to about 8.1 w/w % of croscarmellose sodium; and
about 1.4 w/w % to about 1.6 w/w % of magnesium stearate.

In some embodiments, the tablet comprises:
about 20.0 w/w % of the compound of Formula Ia or Ib free acid;
about 0.142 w/w % of the compound of Formula IIa or IIb, or a pharmaceutically acceptable salt, co-crystal, solvate, or combination thereof;
about 41.40 w/w % of mannitol;
about 22.288 w/w % microcrystalline cellulose;
about 8.0 w/w % of croscarmellose sodium; and
about 1.5 w/w % of magnesium stearate.

In some embodiments, the tablet comprises:
about 19.8 w/w % to about 20.2 w/w % of the compound of Formula Ia or Ib free acid;
about 0.03 w/w % to about 0.04 w/w % of the compound of Formula IIa or IIb, or a pharmaceutically acceptable salt, co-crystal, solvate, or combination thereof;
about 41 w/w % to about 42 w/w % of mannitol;
about 22.3 w/w % to about 22.4 w/w % of microcrystalline cellulose;
about 7.9 w/w % to about 8.1 w/w % of croscarmellose sodium; and
about 1.4 w/w % to about 1.6 w/w % of magnesium stearate.

In some embodiments, the tablet comprises:
about 20.0 w/w % of the compound of Formula Ia or Ib free acid;
about 0.0355 w/w % of the compound of Formula IIa or IIb, or a pharmaceutically acceptable salt, co-crystal, solvate, or combination thereof;
about 41.40 w/w % of mannitol;
about 22.3945 w/w % microcrystalline cellulose;
about 8.0 w/w % of croscarmellose sodium; and
about 1.5 w/w % of magnesium stearate.

In some embodiments, the tablet comprises:
about 19 w/w % to about 21 w/w % of the compound of Formula Ia or Ib, or a pharmaceutically acceptable salt thereof;
about 0.14 w/w % to about 0.15 w/w % of the compound of Formula IIa or IIb, or a solvate thereof;
about 4 w/w % to about 6 w/w % of copovidone;
about 0.1 w/w % to about 2 w/w % of poloxamer 407;
about 40 w/w % to about 42 w/w % of mannitol;
about 22 w/w % to about 23 w/w % of microcrystalline cellulose;
about 7 w/w % to about 9 w/w % of croscarmellose sodium; and
about 1.0 w/w % to about 2.0 w/w % of magnesium stearate.

In some embodiments, the tablet comprises:
about 19.8 w/w % to about 20.8 w/w % of the compound of Formula Ia or Ib, or a pharmaceutically acceptable salt thereof;
about 0.14 w/w % to about 0.15 w/w % of the compound of Formula IIa or IIb, or a solvate thereof;
about 4 w/w % to about 5 w/w % of copovidone;
about 1 w/w % to about 2 w/w % of poloxamer 407;
about 41 w/w % to about 42 w/w % of mannitol;
about 22.2 w/w % to about 22.3 w/w % of microcrystalline cellulose;
about 7.9 w/w % to about 8.1 w/w % of croscarmellose sodium; and
about 1.4 w/w % to about 1.6 w/w % of magnesium stearate.

In some embodiments, the tablet comprises:
about 20.46 w/w % of the compound of Formula Ia or Ib, or a pharmaceutically acceptable salt thereof;
about 0.142 w/w % of the compound of Formula IIa or IIb, or a solvate thereof;
about 4.88 w/w % of copovidone;
about 1.33 w/w % of poloxamer 407;
about 41.40 w/w % of mannitol;
about 22.288 w/w % of microcrystalline cellulose;
about 8.0 w/w % of croscarmellose sodium; and
about 1.5 w/w % of magnesium stearate.

In some embodiments, the tablet comprises:
about 19 w/w % to about 21 w/w % of the sodium salt of the compound of Formula Ia or Ib;
about 0.14 w/w % to about 0.15 w/w % of the monohydrate of the compound of Formula IIa or IIb;
about 4 w/w % to about 6 w/w % of copovidone;
about 0.1 w/w % to about 2 w/w % of poloxamer 407;
about 40 w/w % to about 42 w/w % of mannitol;
about 22 w/w % to about 23 w/w % of microcrystalline cellulose;
about 7 w/w % to about 9 w/w % of croscarmellose sodium; and
about 1.0 w/w % to about 2.0 w/w % of magnesium stearate.

In some embodiments, the tablet comprises:
about 19.8 w/w % to about 20.8 w/w % of the sodium salt of the compound of Formula Ia or Ib;
about 0.14 w/w % to about 0.15 w/w % of the monohydrate of the compound of Formula IIa or IIb;
about 4 w/w % to about 5 w/w % of copovidone;
about 1 w/w % to about 2 w/w % of poloxamer 407;
about 41 w/w % to about 42 w/w % of mannitol;
about 22.2 w/w % to about 22.3 w/w % of microcrystalline cellulose;
about 7.9 w/w % to about 8.1 w/w % of croscarmellose sodium; and
about 1.4 w/w % to about 1.6 w/w % of magnesium stearate.

In some embodiments, the tablet comprises:
about 20.46 w/w % of the sodium salt of the compound of Formula Ia or Ib;
about 0.142 w/w % of the monohydrate of the compound of Formula IIa or IIb;
about 4.88 w/w % of copovidone;
about 1.33 w/w % of poloxamer 407;
about 41.40 w/w % of mannitol;
about 22.288 w/w % of microcrystalline cellulose;
about 8.0 w/w % of croscarmellose sodium; and
about 1.5 w/w % of magnesium stearate.

In some embodiments, the tablet comprises:
about 19 w/w % to about 21 w/w % of the compound of Formula Ia or Ib, or a pharmaceutically acceptable salt thereof;
about 0.14 w/w % to about 0.15 w/w % of the compound of Formula IIa or IIb, or a solvate thereof;
about 4 w/w % to about 6 w/w % of copovidone;
about 0.1 w/w % to about 2 w/w % of poloxamer 407;
about 40 w/w % to about 42 w/w % of mannitol;
about 22 w/w % to about 23 w/w % of microcrystalline cellulose;
about 7 w/w % to about 9 w/w % of croscarmellose sodium;
about 1.0 w/w % to about 2.0 w/w % of magnesium stearate; and
an outer film coat.

In some embodiments, the tablet comprises:
about 19.8 w/w % to about 20.8 w/w % of the compound of Formula Ia or Ib, or a pharmaceutically acceptable salt thereof;
about 0.14 w/w % to about 0.15 w/w % of the compound of Formula IIa or IIb, or a solvate thereof;
about 4 w/w % to about 5 w/w % of copovidone;
about 1 w/w % to about 2 w/w % of poloxamer 407;
about 41 w/w % to about 42 w/w % of mannitol;
about 22.2 w/w % to about 22.3 w/w % of microcrystalline cellulose;
about 7.9 w/w % to about 8.1 w/w % of croscarmellose sodium;
about 1.4 w/w % to about 1.6 w/w % of magnesium stearate; and
an outer film coat.

In some embodiments, the tablet comprises:
about 20.46 w/w % of the compound of Formula Ia or Ib, or a pharmaceutically acceptable salt thereof;
about 0.142 w/w % of the compound of Formula IIa or IIb, or a solvate thereof;
about 4.88 w/w % of copovidone;
about 1.33 w/w % of poloxamer 407;
about 41.40 w/w % of mannitol;
about 22.288 w/w % of microcrystalline cellulose;
about 8.0 w/w % of croscarmellose sodium;
about 1.5 w/w % of magnesium stearate; and
an outer film coat.

In some embodiments, the tablet comprises:
about 19 w/w % to about 21 w/w % of the sodium salt of the compound of Formula Ia or Ib;
about 0.14 w/w % to about 0.15 w/w % of the monohydrate of the compound of Formula IIa or IIb;
about 4 w/w % to about 6 w/w % of copovidone;
about 0.1 w/w % to about 2 w/w % of poloxamer 407;
about 40 w/w % to about 42 w/w % of mannitol;
about 22 w/w % to about 23 w/w % of microcrystalline cellulose;
about 7 w/w % to about 9 w/w % of croscarmellose sodium;
about 1.0 w/w % to about 2.0 w/w % of magnesium stearate; and
an outer film coat.

In some embodiments, the tablet comprises:
about 19.8 w/w % to about 20.8 w/w % of the sodium salt of the compound of Formula Ia or Ib;
about 0.14 w/w % to about 0.15 w/w % of the monohydrate of the compound of Formula IIa or IIb;
about 4 w/w % to about 5 w/w % of copovidone;
about 1 w/w % to about 2 w/w % of poloxamer 407;
about 41 w/w % to about 42 w/w % of mannitol;
about 22.2 w/w % to about 22.3 w/w % of microcrystalline cellulose;
about 7.9 w/w % to about 8.1 w/w % of croscarmellose sodium;
about 1.4 w/w % to about 1.6 w/w % of magnesium stearate; and
an outer film coat.

In some embodiments, the tablet comprises:
about 20.46 w/w % of the sodium salt of the compound of Formula Ia or Ib;
about 0.142 w/w % of the monohydrate of the compound of Formula IIa or IIb;
about 4.88 w/w % of copovidone;
about 1.33 w/w % of poloxamer 407;
about 41.40 w/w % of mannitol;
about 22.288 w/w % of microcrystalline cellulose;
about 8.0 w/w % of croscarmellose sodium;
about 1.5 w/w % of magnesium stearate; and
an outer film coat.

In some embodiments, the tablet consists essentially of:
about 19 w/w % to about 21 w/w % of the compound of Formula Ia or Ib, or a pharmaceutically acceptable salt thereof;
about 0.14 w/w % to about 0.15 w/w % of the compound of Formula IIa or IIb, or a solvate thereof;
about 4 w/w % to about 6 w/w % of copovidone;
about 0.1 w/w % to about 2 w/w % of poloxamer 407;
about 40 w/w % to about 42 w/w % of mannitol;
about 22 w/w % to about 23 w/w % of microcrystalline cellulose;
about 7 w/w % to about 9 w/w % of croscarmellose sodium; and
about 1.0 w/w % to about 2.0 w/w % of magnesium stearate.

In some embodiments, the tablet consists essentially of:
about 19.8 w/w % to about 20.8 w/w % of the compound of Formula Ia or Ib, or a pharmaceutically acceptable salt thereof;
about 0.14 w/w % to about 0.15 w/w % of the compound of Formula IIa or IIb, or a solvate thereof;
about 4 w/w % to about 5 w/w % of copovidone;
about 1 w/w % to about 2 w/w % of poloxamer 407;
about 41 w/w % to about 42 w/w % of mannitol;
about 22.2 w/w % to about 22.3 w/w % of microcrystalline cellulose;
about 7.9 w/w % to about 8.1 w/w % of croscarmellose sodium; and
about 1.4 w/w % to about 1.6 w/w % of magnesium stearate.

In some embodiments, the tablet consists essentially of:
about 20.46 w/w % of the compound of Formula Ia or Ib, or a pharmaceutically acceptable salt thereof;
about 0.142 w/w % of the compound of Formula IIa or IIb, or a solvate thereof;
about 4.88 w/w % of copovidone;
about 1.33 w/w % of poloxamer 407;
about 41.40 w/w % of mannitol;
about 22.288 w/w % of microcrystalline cellulose;
about 8.0 w/w % of croscarmellose sodium; and
about 1.5 w/w % of magnesium stearate.

In some embodiments, the tablet consists essentially of:
about 19 w/w % to about 21 w/w % of the sodium salt of the compound of Formula Ia or Ib;
about 0.14 w/w % to about 0.15 w/w % of the monohydrate of the compound of Formula Ila or IIb;
about 4 w/w % to about 6 w/w % of copovidone;
about 0.1 w/w % to about 2 w/w % of poloxamer 407;
about 40 w/w % to about 42 w/w % of mannitol;
about 22 w/w % to about 23 w/w % of microcrystalline cellulose;
about 7 w/w % to about 9 w/w % of croscarmellose sodium; and
about 1.0 w/w % to about 2.0 w/w % of magnesium stearate.

In some embodiments, the tablet consists essentially of:
about 19.8 w/w % to about 20.8 w/w % of the sodium salt of the compound of Formula Ia or Ib;
about 0.14 w/w % to about 0.15 w/w % of the monohydrate of the compound of Formula IIa or IIb;
about 4 w/w % to about 5 w/w % of copovidone;
about 1 w/w % to about 2 w/w % of poloxamer 407;
about 41 w/w % to about 42 w/w % of mannitol;
about 22.2 w/w % to about 22.3 w/w % of microcrystalline cellulose;
about 7.9 w/w % to about 8.1 w/w % of croscarmellose sodium; and
about 1.4 w/w % to about 1.6 w/w % of magnesium stearate.

In some embodiments, the tablet consists essentially of:
about 20.46 w/w % of the sodium salt of the compound of Formula Ia or Ib;
about 0.142 w/w % of the monohydrate of the compound of Formula IIa or IIb;
about 4.88 w/w % of copovidone;
about 1.33 w/w % of poloxamer 407;
about 41.40 w/w % of mannitol;
about 22.288 w/w % of microcrystalline cellulose;
about 8.0 w/w % of croscarmellose sodium; and
about 1.5 w/w % of magnesium stearate.

In some embodiments, the tablet consists essentially of:
about 19 w/w % to about 21 w/w % of the compound of Formula Ia or Ib, or a pharmaceutically acceptable salt thereof;
about 0.14 w/w % to about 0.15 w/w % of the compound of Formula IIa or IIb, or a solvate thereof;
about 4 w/w % to about 6 w/w % of copovidone;
about 0.1 w/w % to about 2 w/w % of poloxamer 407;
about 40 w/w % to about 42 w/w % of mannitol;
about 22 w/w % to about 23 w/w % of microcrystalline cellulose;
about 7 w/w % to about 9 w/w % of croscarmellose sodium;
about 1.0 w/w % to about 2.0 w/w % of magnesium stearate; and
an outer film coat.

In some embodiments, the tablet consists essentially of:
about 19.8 w/w % to about 20.8 w/w % of the compound of Formula Ia or Ib, or a pharmaceutically acceptable salt thereof;
about 0.14 w/w % to about 0.15 w/w % of the compound of Formula IIa or IIb, or a solvate thereof;
about 4 w/w % to about 5 w/w % of copovidone;
about 1 w/w % to about 2 w/w % of poloxamer 407;
about 41 w/w % to about 42 w/w % of mannitol;
about 22.2 w/w % to about 22.3 w/w % of microcrystalline cellulose;
about 7.9 w/w % to about 8.1 w/w % of croscarmellose sodium;
about 1.4 w/w % to about 1.6 w/w % of magnesium stearate; and
an outer film coat.

In some embodiments, the tablet consists essentially of:
about 20.46 w/w % of the compound of Formula Ia or Ib, or a pharmaceutically acceptable salt thereof;
about 0.142 w/w % of the compound of Formula IIa or IIb, or a solvate thereof;
about 4.88 w/w % of copovidone;
about 1.33 w/w % of poloxamer 407;
about 41.40 w/w % of mannitol;
about 22.288 w/w % of microcrystalline cellulose;
about 8.0 w/w % of croscarmellose sodium;
about 1.5 w/w % of magnesium stearate; and
an outer film coat.

In some embodiments, the tablet consists essentially of:
about 19 w/w % to about 21 w/w % of the sodium salt of the compound of Formula Ia or Ib;
about 0.14 w/w % to about 0.15 w/w % of the monohydrate of the compound of Formula IIa or IIb;
about 4 w/w % to about 6 w/w % of copovidone;
about 0.1 w/w % to about 2 w/w % of poloxamer 407;
about 40 w/w % to about 42 w/w % of mannitol;
about 22 w/w % to about 23 w/w % of microcrystalline cellulose;
about 7 w/w % to about 9 w/w % of croscarmellose sodium;
about 1.0 w/w % to about 2.0 w/w % of magnesium stearate; and
an outer film coat.

In some embodiments, the tablet consists essentially of:
about 19.8 w/w % to about 20.8 w/w % of the sodium salt of the compound of Formula Ia or Ib;
about 0.14 w/w % to about 0.15 w/w % of the monohydrate of the compound of Formula IIa or IIb;
about 4 w/w % to about 5 w/w % of copovidone;
about 1 w/w % to about 2 w/w % of poloxamer 407;
about 41 w/w % to about 42 w/w % of mannitol;
about 22.2 w/w % to about 22.3 w/w % of microcrystalline cellulose;
about 7.9 w/w % to about 8.1 w/w % of croscarmellose sodium;
about 1.4 w/w % to about 1.6 w/w % of magnesium stearate; and
an outer film coat.

In some embodiments, the tablet consists essentially of:
about 20.46 w/w % of the sodium salt of the compound of Formula Ia or Ib;
about 0.142 w/w % of the monohydrate of the compound of Formula IIa or IIb;
about 4.88 w/w % of copovidone;
about 1.33 w/w % of poloxamer 407;
about 41.40 w/w % of mannitol;
about 22.288 w/w % of microcrystalline cellulose;
about 8.0 w/w % of croscarmellose sodium;
about 1.5 w/w % of magnesium stearate; and
an outer film coat.

In some embodiments, the tablet consists of:
about 19 w/w % to about 21 w/w % of the compound of Formula Ia or Ib, or a pharmaceutically acceptable salt thereof;
about 0.14 w/w % to about 0.15 w/w % of the compound of Formula IIa or IIb, or a solvate thereof;
about 4 w/w % to about 6 w/w % of copovidone;
about 0.1 w/w % to about 2 w/w % of poloxamer 407;
about 40 w/w % to about 42 w/w % of mannitol;
about 22 w/w % to about 23 w/w % of microcrystalline cellulose;
about 7 w/w % to about 9 w/w % of croscarmellose sodium; and
about 1.0 w/w % to about 2.0 w/w % of magnesium stearate.

In some embodiments, the tablet consists of:
about 19.8 w/w % to about 20.8 w/w % of the compound of Formula Ia or Ib, or a pharmaceutically acceptable salt thereof;
about 0.14 w/w % to about 0.15 w/w % of the compound of Formula IIa or IIb, or a solvate thereof;
about 4 w/w % to about 5 w/w % of copovidone;
about 1 w/w % to about 2 w/w % of poloxamer 407;
about 41 w/w % to about 42 w/w % of mannitol;
about 22.2 w/w % to about 22.3 w/w % of microcrystalline cellulose;
about 7.9 w/w % to about 8.1 w/w % of croscarmellose sodium; and
about 1.4 w/w % to about 1.6 w/w % of magnesium stearate.

In some embodiments, the tablet consists of:
about 20.46 w/w % of the compound of Formula Ia or Ib, or a pharmaceutically acceptable salt thereof;
about 0.142 w/w % of the compound of Formula IIa or IIb, or a solvate thereof;
about 4.88 w/w % of copovidone;
about 1.33 w/w % of poloxamer 407;
about 41.40 w/w % of mannitol;
about 22.288 w/w % of microcrystalline cellulose;
about 8.0 w/w % of croscarmellose sodium; and
about 1.5 w/w % of magnesium stearate.

In some embodiments, the tablet consists of:
about 19 w/w % to about 21 w/w % of the sodium salt of the compound of Formula Ia or Ib;
about 0.14 w/w % to about 0.15 w/w % of the monohydrate of the compound of Formula IIa or IIb;
about 4 w/w % to about 6 w/w % of copovidone;
about 0.1 w/w % to about 2 w/w % of poloxamer 407;
about 40 w/w % to about 42 w/w % of mannitol;
about 22 w/w % to about 23 w/w % of microcrystalline cellulose;
about 7 w/w % to about 9 w/w % of croscarmellose sodium; and
about 1.0 w/w % to about 2.0 w/w % of magnesium stearate.

In some embodiments, the tablet consists of:
about 19.8 w/w % to about 20.8 w/w % of the sodium salt of the compound of Formula Ia or Ib;
about 0.14 w/w % to about 0.15 w/w % of the monohydrate of the compound of Formula IIa or IIb;
about 4 w/w % to about 5 w/w % of copovidone;
about 1 w/w % to about 2 w/w % of poloxamer 407;
about 41 w/w % to about 42 w/w % of mannitol;
about 22.2 w/w % to about 22.3 w/w % of microcrystalline cellulose;
about 7.9 w/w % to about 8.1 w/w % of croscarmellose sodium; and
about 1.4 w/w % to about 1.6 w/w % of magnesium stearate.

In some embodiments, the tablet consists of:
about 20.46 w/w % of the sodium salt of the compound of Formula Ia or Ib;
about 0.142 w/w % of the monohydrate of the compound of Formula IIa or IIb;
about 4.88 w/w % of copovidone;
about 1.33 w/w % of poloxamer 407;
about 41.40 w/w % of mannitol;
about 22.288 w/w % of microcrystalline cellulose;
about 8.0 w/w % of croscarmellose sodium; and
about 1.5 w/w % of magnesium stearate.

In some embodiments, the tablet consists of:
about 19 w/w % to about 21 w/w % of the compound of Formula Ia or Ib, or a pharmaceutically acceptable salt thereof;
about 0.14 w/w % to about 0.15 w/w % of the compound of Formula IIa or IIb, or a solvate thereof;
about 4 w/w % to about 6 w/w % of copovidone;
about 0.1 w/w % to about 2 w/w % of poloxamer 407;
about 40 w/w % to about 42 w/w % of mannitol;
about 22 w/w % to about 23 w/w % of microcrystalline cellulose;
about 7 w/w % to about 9 w/w % of croscarmellose sodium;
about 1.0 w/w % to about 2.0 w/w % of magnesium stearate; and
an outer film coat.

In some embodiments, the tablet consists of:
about 19.8 w/w % to about 20.8 w/w % of the compound of Formula Ia or Ib, or a pharmaceutically acceptable salt thereof;
about 0.14 w/w % to about 0.15 w/w % of the compound of Formula IIa or IIb, or a solvate thereof;
about 4 w/w % to about 5 w/w % of copovidone;
about 1 w/w % to about 2 w/w % of poloxamer 407;
about 41 w/w % to about 42 w/w % of mannitol;
about 22.2 w/w % to about 22.3 w/w % of microcrystalline cellulose;
about 7.9 w/w % to about 8.1 w/w % of croscarmellose sodium;
about 1.4 w/w % to about 1.6 w/w % of magnesium stearate; and
an outer film coat.

In some embodiments, the tablet consists of:
about 20.46 w/w % of the compound of Formula Ia or Ib, or a pharmaceutically acceptable salt thereof;
about 0.142 w/w % of the compound of Formula IIa or IIb, or a solvate thereof;
about 4.88 w/w % of copovidone;
about 1.33 w/w % of poloxamer 407;
about 41.40 w/w % of mannitol;
about 22.288 w/w % of microcrystalline cellulose;
about 8.0 w/w % of croscarmellose sodium;
about 1.5 w/w % of magnesium stearate; and
an outer film coat.

In some embodiments, the tablet consists of:
about 19 w/w % to about 21 w/w % of the sodium salt of the compound of Formula Ia or Ib;
about 0.14 w/w % to about 0.15 w/w % of the monohydrate of the compound of Formula IIa or IIb;
about 4 w/w % to about 6 w/w % of copovidone;
about 0.1 w/w % to about 2 w/w % of poloxamer 407;
about 40 w/w % to about 42 w/w % of mannitol;
about 22 w/w % to about 23 w/w % of microcrystalline cellulose;
about 7 w/w % to about 9 w/w % of croscarmellose sodium;
about 1.0 w/w % to about 2.0 w/w % of magnesium stearate; and
an outer film coat.

In some embodiments, the tablet consists of:
about 19.8 w/w % to about 20.8 w/w % of the sodium salt of the compound of Formula Ia or Ib;
about 0.14 w/w % to about 0.15 w/w % of the monohydrate of the compound of Formula IIa or IIb;
about 4 w/w % to about 5 w/w % of copovidone;
about 1 w/w % to about 2 w/w % of poloxamer 407;
about 41 w/w % to about 42 w/w % of mannitol;
about 22.2 w/w % to about 22.3 w/w % of microcrystalline cellulose;
about 7.9 w/w % to about 8.1 w/w % of croscarmellose sodium;
about 1.4 w/w % to about 1.6 w/w % of magnesium stearate; and
an outer film coat.

In some embodiments, the tablet consists of:
about 20.46 w/w % of the sodium salt of the compound of Formula Ia or Ib;
about 0.142 w/w % of the monohydrate of the compound of Formula IIa or IIb;
about 4.88 w/w % of copovidone;
about 1.33 w/w % of poloxamer 407;
about 41.40 w/w % of mannitol;
about 22.288 w/w % of microcrystalline cellulose;
about 8.0 w/w % of croscarmellose sodium;
about 1.5 w/w % of magnesium stearate; and
an outer film coat.

In some embodiments, the tablet comprises:
about 19 w/w % to about 21 w/w % of the compound of Formula Ia or Ib, or a pharmaceutically acceptable salt thereof;
about 0.03 w/w % to about 0.04 w/w % of the compound of Formula IIa or IIb, or a solvate thereof;
about 4 w/w % to about 6 w/w % of copovidone;
about 0.1 w/w % to about 2 w/w % of poloxamer 407;
about 40 w/w % to about 42 w/w % of mannitol;
about 22 w/w % to about 23 w/w % of microcrystalline cellulose;
about 7 w/w % to about 9 w/w % of croscarmellose sodium; and
about 1.0 w/w % to about 2.0 w/w % of magnesium stearate.

In some embodiments, the tablet comprises:
about 19.8 w/w % to about 20.8 w/w % of the compound of Formula Ia or Ib, or a pharmaceutically acceptable salt thereof;
about 0.03 w/w % to about 0.04 w/w % of the compound of Formula IIa or IIb, or a solvate thereof;
about 4 w/w % to about 5 w/w % of copovidone;
about 1 w/w % to about 2 w/w % of poloxamer 407;
about 41 w/w % to about 42 w/w % of mannitol;
about 22.3 w/w % to about 22.4 w/w % of microcrystalline cellulose;
about 7.9 w/w % to about 8.1 w/w % of croscarmellose sodium; and
about 1.4 w/w % to about 1.6 w/w % of magnesium stearate.

In some embodiments, the tablet comprises:
about 20.46 w/w % of the compound of Formula Ia or Ib, or a pharmaceutically acceptable salt thereof;
about 0.0355 w/w % of the compound of Formula IIa or IIb, or a solvate thereof;
about 4.88 w/w % of copovidone;
about 1.33 w/w % of poloxamer 407;
about 41.40 w/w % of mannitol;
about 22.395 w/w % of microcrystalline cellulose;
about 8.0 w/w % of croscarmellose sodium; and
about 1.5 w/w % of magnesium stearate.

In some embodiments, the tablet comprises:
about 19 w/w % to about 21 w/w % of the sodium salt of the compound of Formula Ia or Ib;
about 0.03 w/w % to about 0.04 w/w % of the monohydrate of the compound of Formula IIa or IIb;
about 4 w/w % to about 6 w/w % of copovidone;
about 0.1 w/w % to about 2 w/w % of poloxamer 407;
about 40 w/w % to about 42 w/w % of mannitol;
about 22 w/w % to about 23 w/w % of microcrystalline cellulose;
about 7 w/w % to about 9 w/w % of croscarmellose sodium; and
about 1.0 w/w % to about 2.0 w/w % of magnesium stearate.

In some embodiments, the tablet comprises:
about 19.8 w/w % to about 20.8 w/w % of the sodium salt of the compound of Formula Ia or Ib;
about 0.03 w/w % to about 0.04 w/w % of the monohydrate of the compound of Formula IIa or IIb;
about 4 w/w % to about 5 w/w % of copovidone;
about 1 w/w % to about 2 w/w % of poloxamer 407;
about 41 w/w % to about 42 w/w % of mannitol;
about 22.3 w/w % to about 22.4 w/w % of microcrystalline cellulose;
about 7.9 w/w % to about 8.1 w/w % of croscarmellose sodium; and
about 1.4 w/w % to about 1.6 w/w % of magnesium stearate.

In some embodiments, the tablet comprises:
about 20.46 w/w % of the sodium salt of the compound of Formula Ia or Ib;
about 0.0355 w/w % of the monohydrate of the compound of Formula IIa or IIb;
about 4.88 w/w % of copovidone;
about 1.33 w/w % of poloxamer 407;
about 41.40 w/w % of mannitol;
about 22.395 w/w % of microcrystalline cellulose;
about 8.0 w/w % of croscarmellose sodium; and
about 1.5 w/w % of magnesium stearate.

In some embodiments, the tablet comprises:
about 19 w/w % to about 21 w/w % of the compound of Formula Ia or Ib, or a pharmaceutically acceptable salt thereof;
about 0.03 w/w % to about 0.04 w/w % of the compound of Formula IIa or IIb, or a solvate thereof;
about 4 w/w % to about 6 w/w % of copovidone;
about 0.1 w/w % to about 2 w/w % of poloxamer 407;
about 40 w/w % to about 42 w/w % of mannitol;
about 22 w/w % to about 23 w/w % of microcrystalline cellulose;
about 7 w/w % to about 9 w/w % of croscarmellose sodium;
about 1.0 w/w % to about 2.0 w/w % of magnesium stearate; and
an outer film coat.

In some embodiments, the tablet comprises:
about 19.8 w/w % to about 20.8 w/w % of the compound of Formula Ia or Ib, or a pharmaceutically acceptable salt thereof;
about 0.03 w/w % to about 0.04 w/w % of the compound of Formula IIa or IIb, or a solvate thereof;
about 4 w/w % to about 5 w/w % of copovidone;
about 1 w/w % to about 2 w/w % of poloxamer 407;
about 41 w/w % to about 42 w/w % of mannitol;
about 22.3 w/w % to about 22.4 w/w % of microcrystalline cellulose;
about 7.9 w/w % to about 8.1 w/w % of croscarmellose sodium;
about 1.4 w/w % to about 1.6 w/w % of magnesium stearate; and
an outer film coat.

In some embodiments, the tablet comprises:
about 20.46 w/w % of the compound of Formula Ia or Ib, or a pharmaceutically acceptable salt thereof;
about 0.0355 w/w % of the compound of Formula IIa or IIb, or a solvate thereof;
about 4.88 w/w % of copovidone;
about 1.33 w/w % of poloxamer 407;
about 41.40 w/w % of mannitol;
about 22.395 w/w % of microcrystalline cellulose;
about 8.0 w/w % of croscarmellose sodium;
about 1.5 w/w % of magnesium stearate; and
an outer film coat.

In some embodiments, the tablet comprises:
about 19 w/w % to about 21 w/w % of the sodium salt of the compound of Formula Ia or Ib;
about 0.03 w/w % to about 0.04 w/w % of the monohydrate of the compound of Formula IIa or IIb;
about 4 w/w % to about 6 w/w % of copovidone;
about 0.1 w/w % to about 2 w/w % of poloxamer 407;
about 40 w/w % to about 42 w/w % of mannitol;
about 22 w/w % to about 23 w/w % of microcrystalline cellulose;
about 7 w/w % to about 9 w/w % of croscarmellose sodium;
about 1.0 w/w % to about 2.0 w/w % of magnesium stearate; and
an outer film coat.

In some embodiments, the tablet comprises:

about 19.8 w/w % to about 20.8 w/w % of the sodium salt of the compound of Formula Ia or Ib;
about 0.03 w/w % to about 0.04 w/w % of the monohydrate of the compound of Formula IIa or IIb;
about 4 w/w % to about 5 w/w % of copovidone;
about 1 w/w % to about 2 w/w % of poloxamer 407;
about 41 w/w % to about 42 w/w % of mannitol;
about 22.3 w/w % to about 22.4 w/w % of microcrystalline cellulose;
about 7.9 w/w % to about 8.1 w/w % of croscarmellose sodium;
about 1.4 w/w % to about 1.6 w/w % of magnesium stearate; and
an outer film coat.

In some embodiments, the tablet comprises:
about 20.46 w/w % of the sodium salt of the compound of Formula Ia or Ib;
about 0.0355 w/w % of the monohydrate of the compound of Formula IIa or IIb;
about 4.88 w/w % of copovidone;
about 1.33 w/w % of poloxamer 407;
about 41.40 w/w % of mannitol;
about 22.395 w/w % of microcrystalline cellulose;
about 8.0 w/w % of croscarmellose sodium;
about 1.5 w/w % of magnesium stearate; and
an outer film coat.

In some embodiments, the tablet consists essentially of:
about 19 w/w % to about 21 w/w % of the compound of Formula Ia or Ib, or a pharmaceutically acceptable salt thereof;
about 0.03 w/w % to about 0.04 w/w % of the compound of Formula IIa or IIb, or a solvate thereof;
about 4 w/w % to about 6 w/w % of copovidone;
about 0.1 w/w % to about 2 w/w % of poloxamer 407;
about 40 w/w % to about 42 w/w % of mannitol;
about 22 w/w % to about 23 w/w % of microcrystalline cellulose;
about 7 w/w % to about 9 w/w % of croscarmellose sodium; and
about 1.0 w/w % to about 2.0 w/w % of magnesium stearate.

In some embodiments, the tablet consists essentially of:
about 19.8 w/w % to about 20.8 w/w % of the compound of Formula Ia or Ib, or a pharmaceutically acceptable salt thereof;
about 0.03 w/w % to about 0.04 w/w % of the compound of Formula IIa or IIb, or a solvate thereof;
about 4 w/w % to about 5 w/w % of copovidone;
about 1 w/w % to about 2 w/w % of poloxamer 407;
about 41 w/w % to about 42 w/w % of mannitol;
about 22.3 w/w % to about 22.4 w/w % of microcrystalline cellulose;
about 7.9 w/w % to about 8.1 w/w % of croscarmellose sodium; and
about 1.4 w/w % to about 1.6 w/w % of magnesium stearate.

In some embodiments, the tablet consists essentially of:
about 20.46 w/w % of the compound of Formula Ia or Ib, or a pharmaceutically acceptable salt thereof;
about 0.0355 w/w % of the compound of Formula IIa or IIb, or a solvate thereof;
about 4.88 w/w % of copovidone;
about 1.33 w/w % of poloxamer 407;
about 41.40 w/w % of mannitol;
about 22.395 w/w % of microcrystalline cellulose;
about 8.0 w/w % of croscarmellose sodium; and
about 1.5 w/w % of magnesium stearate.

In some embodiments, the tablet consists essentially of:
about 19 w/w % to about 21 w/w % of the sodium salt of the compound of Formula Ia or Ib;
about 0.03 w/w % to about 0.04 w/w % of the monohydrate of the compound of Formula IIa or IIb;
about 4 w/w % to about 6 w/w % of copovidone;
about 0.1 w/w % to about 2 w/w % of poloxamer 407;
about 40 w/w % to about 42 w/w % of mannitol;
about 22 w/w % to about 23 w/w % of microcrystalline cellulose;
about 7 w/w % to about 9 w/w % of croscarmellose sodium; and
about 1.0 w/w % to about 2.0 w/w % of magnesium stearate.

In some embodiments, the tablet consists essentially of:
about 19.8 w/w % to about 20.8 w/w % of the sodium salt of the compound of Formula Ia or Ib;
about 0.03 w/w % to about 0.04 w/w % of the monohydrate of the compound of Formula IIa or IIb;
about 4 w/w % to about 5 w/w % of copovidone;
about 1 w/w % to about 2 w/w % of poloxamer 407;
about 41 w/w % to about 42 w/w % of mannitol;
about 22.3 w/w % to about 22.4 w/w % of microcrystalline cellulose;
about 7.9 w/w % to about 8.1 w/w % of croscarmellose sodium; and
about 1.4 w/w % to about 1.6 w/w % of magnesium stearate.

In some embodiments, the tablet consists essentially of:
about 20.46 w/w % of the sodium salt of the compound of Formula Ia or Ib;
about 0.0355 w/w % of the monohydrate of the compound of Formula IIa or IIb;
about 4.88 w/w % of copovidone;
about 1.33 w/w % of poloxamer 407;
about 41.40 w/w % of mannitol;
about 22.395 w/w % of microcrystalline cellulose;
about 8.0 w/w % of croscarmellose sodium; and
about 1.5 w/w % of magnesium stearate.

In some embodiments, the tablet consists essentially of:
about 19 w/w % to about 21 w/w % of the compound of Formula Ia or Ib, or a pharmaceutically acceptable salt thereof;
about 0.03 w/w % to about 0.04 w/w % of the compound of Formula IIa or IIb, or a solvate thereof;
about 4 w/w % to about 6 w/w % of copovidone;
about 0.1 w/w % to about 2 w/w % of poloxamer 407;
about 40 w/w % to about 42 w/w % of mannitol;
about 22 w/w % to about 23 w/w % of microcrystalline cellulose;
about 7 w/w % to about 9 w/w % of croscarmellose sodium;
about 1.0 w/w % to about 2.0 w/w % of magnesium stearate; and
an outer film coat.

In some embodiments, the tablet consists essentially of:
about 19.8 w/w % to about 20.8 w/w % of the compound of Formula Ia or Ib, or a pharmaceutically acceptable salt thereof;
about 0.03 w/w % to about 0.04 w/w % of the compound of Formula IIa or IIb, or a solvate thereof;
about 4 w/w % to about 5 w/w % of copovidone;
about 1 w/w % to about 2 w/w % of poloxamer 407;
about 41 w/w % to about 42 w/w % of mannitol;
about 22.3 w/w % to about 22.4 w/w % of microcrystalline cellulose;
about 7.9 w/w % to about 8.1 w/w % of croscarmellose sodium;
about 1.4 w/w % to about 1.6 w/w % of magnesium stearate; and
an outer film coat.

In some embodiments, the tablet consists essentially of:
about 20.46 w/w % of the compound of Formula Ia or Ib, or a pharmaceutically acceptable salt thereof;
about 0.0355 w/w % of the compound of Formula IIa or IIb, or a solvate thereof;
about 4.88 w/w % of copovidone;
about 1.33 w/w % of poloxamer 407;
about 41.40 w/w % of mannitol;
about 22.395 w/w % of microcrystalline cellulose;
about 8.0 w/w % of croscarmellose sodium;
about 1.5 w/w % of magnesium stearate; and
an outer film coat.

In some embodiments, the tablet consists essentially of:
about 19 w/w % to about 21 w/w % of the sodium salt of the compound of Formula Ia or Ib;
about 0.03 w/w % to about 0.04 w/w % of the monohydrate of the compound of Formula IIa or IIb;
about 4 w/w % to about 6 w/w % of copovidone;
about 0.1 w/w % to about 2 w/w % of poloxamer 407;
about 40 w/w % to about 42 w/w % of mannitol;
about 22 w/w % to about 23 w/w % of microcrystalline cellulose;
about 7 w/w % to about 9 w/w % of croscarmellose sodium;
about 1.0 w/w % to about 2.0 w/w % of magnesium stearate; and
an outer film coat.

In some embodiments, the tablet consists essentially of:
about 19.8 w/w % to about 20.8 w/w % of the sodium salt of the compound of Formula Ia or Ib;
about 0.03 w/w % to about 0.04 w/w % of the monohydrate of the compound of Formula IIa or IIb;
about 4 w/w % to about 5 w/w % of copovidone;
about 1 w/w % to about 2 w/w % of poloxamer 407;
about 41 w/w % to about 42 w/w % of mannitol;
about 22.3 w/w % to about 22.4 w/w % of microcrystalline cellulose;
about 7.9 w/w % to about 8.1 w/w % of croscarmellose sodium;
about 1.4 w/w % to about 1.6 w/w % of magnesium stearate; and
an outer film coat.

In some embodiments, the tablet consists essentially of:
about 20.46 w/w % of the sodium salt of the compound of Formula Ia or Ib;
about 0.0355 w/w % of the monohydrate of the compound of Formula IIa or IIb;
about 4.88 w/w % of copovidone;
about 1.33 w/w % of poloxamer 407;
about 41.40 w/w % of mannitol;
about 22.395 w/w % of microcrystalline cellulose;
about 8.0 w/w % of croscarmellose sodium;
about 1.5 w/w % of magnesium stearate; and
an outer film coat.

In some embodiments, the tablet consists of:
about 19 w/w % to about 21 w/w % of the compound of Formula Ia or Ib, or a pharmaceutically acceptable salt thereof;
about 0.03 w/w % to about 0.04 w/w % of the compound of Formula IIa or IIb, or a solvate thereof;
about 4 w/w % to about 6 w/w % of copovidone;
about 0.1 w/w % to about 2 w/w % of poloxamer 407;
about 40 w/w % to about 42 w/w % of mannitol;
about 22 w/w % to about 23 w/w % of microcrystalline cellulose;
about 7 w/w % to about 9 w/w % of croscarmellose sodium; and
about 1.0 w/w % to about 2.0 w/w % of magnesium stearate.

In some embodiments, the tablet consists of:
about 19.8 w/w % to about 20.8 w/w % of the compound of Formula Ia or Ib, or a pharmaceutically acceptable salt thereof;
about 0.03 w/w % to about 0.04 w/w % of the compound of Formula IIa or IIb, or a solvate thereof;
about 4 w/w % to about 5 w/w % of copovidone;
about 1 w/w % to about 2 w/w % of poloxamer 407;
about 41 w/w % to about 42 w/w % of mannitol;
about 22.3 w/w % to about 22.4 w/w % of microcrystalline cellulose;
about 7.9 w/w % to about 8.1 w/w % of croscarmellose sodium; and
about 1.4 w/w % to about 1.6 w/w % of magnesium stearate.

In some embodiments, the tablet consists of:
about 20.46 w/w % of the compound of Formula Ia or Ib, or a pharmaceutically acceptable salt thereof;
about 0.0355 w/w % of the compound of Formula IIa or IIb, or a solvate thereof;
about 4.88 w/w % of copovidone;
about 1.33 w/w % of poloxamer 407;
about 41.40 w/w % of mannitol;
about 22.395 w/w % of microcrystalline cellulose;
about 8.0 w/w % of croscarmellose sodium; and
about 1.5 w/w % of magnesium stearate.

In some embodiments, the tablet consists of:
about 19 w/w % to about 21 w/w % of the sodium salt of the compound of Formula Ia or Ib;
about 0.03 w/w % to about 0.04 w/w % of the monohydrate of the compound of Formula IIa or IIb;
about 4 w/w % to about 6 w/w % of copovidone;
about 0.1 w/w % to about 2 w/w % of poloxamer 407;
about 40 w/w % to about 42 w/w % of mannitol;
about 22 w/w % to about 23 w/w % of microcrystalline cellulose;
about 7 w/w % to about 9 w/w % of croscarmellose sodium; and
about 1.0 w/w % to about 2.0 w/w % of magnesium stearate.

In some embodiments, the tablet consists of:
about 19.8 w/w % to about 20.8 w/w % of the sodium salt of the compound of Formula Ia or Ib;
about 0.03 w/w % to about 0.04 w/w % of the monohydrate of the compound of Formula IIa or IIb;
about 4 w/w % to about 5 w/w % of copovidone;
about 1 w/w % to about 2 w/w % of poloxamer 407;
about 41 w/w % to about 42 w/w % of mannitol;
about 22.3 w/w % to about 22.4 w/w % of microcrystalline cellulose;
about 7.9 w/w % to about 8.1 w/w % of croscarmellose sodium; and
about 1.4 w/w % to about 1.6 w/w % of magnesium stearate.

In some embodiments, the tablet consists of:
about 20.46 w/w % of the sodium salt of the compound of Formula Ia or Ib;
about 0.0355 w/w % of the monohydrate of the compound of Formula IIa or IIb;
about 4.88 w/w % of copovidone;
about 1.33 w/w % of poloxamer 407;
about 41.40 w/w % of mannitol;
about 22.395 w/w % of microcrystalline cellulose;
about 8.0 w/w % of croscarmellose sodium; and
about 1.5 w/w % of magnesium stearate.

In some embodiments, the tablet consists of:
about 19 w/w % to about 21 w/w % of the compound of Formula Ia or Ib, or a pharmaceutically acceptable salt thereof;
about 0.03 w/w % to about 0.04 w/w % of the compound of Formula IIa or IIb, or a solvate thereof;
about 4 w/w % to about 6 w/w % of copovidone;
about 0.1 w/w % to about 2 w/w % of poloxamer 407;
about 40 w/w % to about 42 w/w % of mannitol;
about 22 w/w % to about 23 w/w % of microcrystalline cellulose;
about 7 w/w % to about 9 w/w % of croscarmellose sodium;
about 1.0 w/w % to about 2.0 w/w % of magnesium stearate; and
an outer film coat.

In some embodiments, the tablet consists of:
about 19.8 w/w % to about 20.8 w/w % of the compound of Formula Ia or Ib, or a pharmaceutically acceptable salt thereof;
about 0.03 w/w % to about 0.04 w/w % of the compound of Formula IIa or IIb, or a solvate thereof;
about 4 w/w % to about 5 w/w % of copovidone;
about 1 w/w % to about 2 w/w % of poloxamer 407;
about 41 w/w % to about 42 w/w % of mannitol;
about 22.3 w/w % to about 22.4 w/w % of microcrystalline cellulose;
about 7.9 w/w % to about 8.1 w/w % of croscarmellose sodium;
about 1.4 w/w % to about 1.6 w/w % of magnesium stearate; and
an outer film coat.

In some embodiments, the tablet consists of:
about 20.46 w/w % of the compound of Formula Ia or Ib, or a pharmaceutically acceptable salt thereof;
about 0.0355 w/w % of the compound of Formula IIa or IIb, or a solvate thereof;
about 4.88 w/w % of copovidone;
about 1.33 w/w % of poloxamer 407;
about 41.40 w/w % of mannitol;
about 22.395 w/w % of microcrystalline cellulose;
about 8.0 w/w % of croscarmellose sodium;
about 1.5 w/w % of magnesium stearate; and
an outer film coat.

In some embodiments, the tablet consists of:
about 19 w/w % to about 21 w/w % of the sodium salt of the compound of Formula Ia or Ib;
about 0.03 w/w % to about 0.04 w/w % of the monohydrate of the compound of Formula IIa or IIb;
about 4 w/w % to about 6 w/w % of copovidone;
about 0.1 w/w % to about 2 w/w % of poloxamer 407;
about 40 w/w % to about 42 w/w % of mannitol;
about 22 w/w % to about 23 w/w % of microcrystalline cellulose;
about 7 w/w % to about 9 w/w % of croscarmellose sodium;
about 1.0 w/w % to about 2.0 w/w % of magnesium stearate; and
an outer film coat.

In some embodiments, the tablet consists of:
about 19.8 w/w % to about 20.8 w/w % of the sodium salt of the compound of Formula Ia or Ib;
about 0.03 w/w % to about 0.04 w/w % of the monohydrate of the compound of Formula IIa or IIb;
about 4 w/w % to about 5 w/w % of copovidone;
about 1 w/w % to about 2 w/w % of poloxamer 407;
about 41 w/w % to about 42 w/w % of mannitol;
about 22.3 w/w % to about 22.4 w/w % of microcrystalline cellulose;
about 7.9 w/w % to about 8.1 w/w % of croscarmellose sodium;
about 1.4 w/w % to about 1.6 w/w % of magnesium stearate; and
an outer film coat.

In some embodiments, the tablet consists of:
about 20.46 w/w % of the sodium salt of the compound of Formula Ia or Ib;
about 0.0355 w/w % of the monohydrate of the compound of Formula IIa or IIb;
about 4.88 w/w % of copovidone;
about 1.33 w/w % of poloxamer 407;
about 41.40 w/w % of mannitol;
about 22.395 w/w % of microcrystalline cellulose;
about 8.0 w/w % of croscarmellose sodium;
about 1.5 w/w % of magnesium stearate; and
an outer film coat.

The tablets disclosed herein may be uncoated or coated (in which case they include an outer film coat). Although uncoated tablets may be used, it is more usual to provide a coated tablet, in which case a conventional non-enteric coating may be used. Film coatings are known in the art and can be composed of hydrophilic polymer materials, but are not limited to, polysaccharide materials, such as hydroxypropylmethyl cellulose (HPMC), methylcellulose, hydroxyethyl cellulose (HEC), hydroxypropyl cellulose (HPC), poly(vinylalcohol-co-ethylene glycol) and other water soluble polymers. Though the water-soluble material included in the film coating of the tablets may include a single polymer material, it may also be formed using a mixture of more than one polymer. The coating may be white or colored. Suitable coatings include, but are not limited to, polymeric film coatings such as those comprising polyvinyl alcohol e.g. Opadry® QX Grey, Opadry® QX Pink, Opadry® Clear, Opadry® TF Pink, and Opadry® TF Grey. The amount of coating will generally be between about 1-8% of the uncoated tablet's weight.

In some embodiments, the tablet further comprises an outer film coat.

In some embodiments, the outer film coat is selected from the Opadry® QX Quick and Flexible Film Coating System 321A series. In some embodiments, the outer film coat is selected from the Opadry® II Complete Film Coating System 85F series. In some embodiments, the outer film coat is selected from the Opadry® TF High Opacity PVA Film Coating 273F series. In some embodiments, the outer film coat is selected from the Opadry® TF Titanium Free Formulated Film Coating 266U series.

In some embodiments, the outer film coat comprises Opadry® Clear.

In some embodiments, the outer film coat comprises Opadry® TF Pink.

In some embodiments, the outer film coat comprises Opadry® TF Grey.

In some embodiments, the outer film coat comprises about 0.5 w/w % to about 1.5 w/w % Opadry® Clear, for example, about 0.5 w/w %, about 0.6 w/w %, about 0.7 w/w %, about 0.8 w/w %, about 0.9 w/w %, about 1.0 w/w %, about 1.1 w/w %, about 1.2 w/w %, about 1.3 w/w %, about 1.4 w/w %, or about 1.5 w/w % Opadry® Clear. In some embodiments, the outer film coat comprises about 0.9 w/w % to about 1.1 w/w % Opadry® Clear. In some embodiments, the outer film coat comprises about 1 w/w % Opadry® Clear.

In some embodiments, the outer film coat comprises Opadry® Clear and further comprises Opadry® TF Pink or Opadry® TF Gray.

In some embodiments, the outer film coat comprises Opadry® Clear and Opadry® TF Pink. In some embodiments, the outer film coat further comprises about 3.5 w/w % to about 4.5 w/w % Opadry® TF Pink, for example, about 3.5 w/w %, about 3.6 w/w %, about 3.7 w/w %, about 3.8 w/w %, about 3.9 w/w %, about 4.0 w/w %, about 4.1 w/w %, about 4.2 w/w %, about 4.3 w/w %, about 4.4 w/w %, or about 4.5 w/w % Opadry® TF Pink. In some embodiments, the outer film coat comprises about 3.9 w/w % to about 4.1 w/w % Opadry® TF Pink. In some embodiments, the outer film coat comprises about 4 w/w % Opadry® TF Pink. In some embodiments, the outer film coat comprises about 1 w/w % Opadry® Clear and about 4 w/w % Opadry® TF Pink. In some embodiments, the outer film coat comprises about 0.9 w/w % to about 1.1 w/w % Opadry® Clear and about 3.9 w/w % to about 4.1 w/w % Opadry® TF Pink.

In some embodiments, the outer film coat comprises Opadry® Clear and Opadry® TF Gray. In some embodiments, the outer film coat comprises about 3.5 w/w % to about 4.5 w/w % Opadry® TF Gray, for example, about 3.5 w/w %, about 3.6 w/w %, about 3.7 w/w %, about 3.8 w/w %, about 3.9 w/w %, about 4.0 w/w %, about 4.1 w/w %, about 4.2 w/w %, about 4.3 w/w %, about 4.4 w/w %, or about 4.5 w/w % Opadry® TF Gray. In some embodiments, the outer film coat comprises about 3.9 w/w % to about 4.1 w/w % Opadry® TF Gray. In some embodiments, the outer film coat comprises about 4 w/w % Opadry® TF Gray. In some embodiments, the outer film coat comprises about 1 w/w % Opadry® Clear and about 4 w/w % Opadry® TF Gray. In some embodiments, the outer film coat comprises about 0.9 w/w % to about 1.1 w/w % Opadry® Clear and about 3.9 w/w % to about 4.1 w/w % Opadry® TF Gray.

In some embodiments, the tablet comprises about 100 mg to about 500 mg of the compound of Formula Ia or Ib, or a pharmaceutically acceptable salt, co-crystal, solvate, or combination thereof, for example, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, about 400 mg, about 405 mg, about 410 mg, about 415 mg, about 420 mg, about 425 mg, about 430 mg, about 435 mg, about 440 mg, about 445 mg, about 450 mg, about 455 mg, about 460 mg, about 465 mg, about 470 mg, about 475 mg, about 480 mg, about 485 mg, about 490 mg, about 495 mg, or about 500 mg of the compound of Formula Ia or Ib, or a pharmaceutically acceptable salt, co-crystal, solvate, or combination thereof.

In some embodiments, the tablet comprises about 200 mg to about 500 mg of the compound of Formula Ia or Ib, or a pharmaceutically acceptable salt, co-crystal, solvate, or combination thereof. In some embodiments, the tablet comprises about 250 mg to about 450 mg of the compound of Formula Ia or Ib, or a pharmaceutically acceptable salt, co-crystal, solvate, or combination thereof.

In some embodiments, the tablet comprises about 375 mg to about 425 mg of the compound of Formula Ia or Ib, or a pharmaceutically acceptable salt, co-crystal, solvate, or combination thereof. In some embodiments, the tablet comprises about 390 mg to about 410 mg of the compound of Formula Ia or Ib, or a pharmaceutically acceptable salt, co-crystal, solvate, or combination thereof. In some embodiments, the tablet comprises about 399 mg to about 401 mg of the compound of Formula Ia or Ib, or a pharmaceutically acceptable salt, co-crystal, solvate, or combination thereof. In some embodiments, the tablet comprises about 400 mg of the compound of Formula Ia or Ib, or a pharmaceutically acceptable salt, co-crystal, solvate, or combination thereof.

In some embodiments, the tablet comprises about 300 mg to about 500 mg of the compound of Formula Ia or Ib, or a pharmaceutically acceptable salt thereof. In some embodiments, the tablet comprises about 350 mg to about 450 mg of the compound of Formula Ia or Ib, or a pharmaceutically acceptable salt thereof. In some embodiments, the tablet comprises about 375 mg to about 425 mg of the compound of Formula Ia or Ib, or a pharmaceutically acceptable salt thereof. In some embodiments, the tablet comprises about 390 mg to about 410 mg of the compound of Formula Ia or Ib, or a pharmaceutically acceptable salt thereof. In some embodiments, the tablet comprises about 399 mg to about 401 mg of the compound of Formula Ia or Ib, or a pharmaceutically acceptable salt thereof. In some embodiments, the tablet comprises about 400 mg of the compound of Formula Ia or Ib, or a pharmaceutically acceptable salt thereof.

In some embodiments, the tablet comprises about 300 mg to about 500 mg of the sodium salt of the compound of Formula Ia or Ib. In some embodiments, the tablet comprises about 350 mg to about 450 mg of the sodium salt of the compound of Formula Ia or Ib. In some embodiments, the tablet comprises about 375 mg to about 425 mg of the sodium salt of the compound of Formula Ia or Ib. In some embodiments, the tablet comprises about 390 mg to about 410 mg of the sodium salt of the compound of Formula Ia or Ib. In some embodiments, the tablet comprises about 399 mg to about 401 mg of the sodium salt of the compound of Formula Ia or Ib. In some embodiments, the tablet comprises about 400 mg of the sodium salt of the compound of Formula Ia or Ib.

In some embodiments, the tablet comprises about 200 mg to about 400 mg of the compound of Formula Ia or Ib, or a pharmaceutically acceptable salt, co-crystal, solvate, or combination thereof, on a free acid basis. In some embodiments, the tablet comprises about 250 mg to about 450 mg of the compound of Formula Ia or Ib, or a pharmaceutically acceptable salt, co-crystal, solvate, or combination thereof, on a free acid basis.

In some embodiments, the tablet comprises about 275 mg to about 325 mg of the compound of Formula Ia or Ib, or a pharmaceutically acceptable salt, co-crystal, solvate, or combination thereof, on a free acid basis. In some embodiments, the tablet comprises about 290 mg to about 310 mg of the compound of Formula Ia or Ib, or a pharmaceutically acceptable salt, co-crystal, solvate, or combination thereof, on a free acid basis. In some embodiments, the tablet comprises about 299 mg to about 301 mg of the compound of Formula Ia or Ib, or a pharmaceutically acceptable salt, co-crystal, solvate, or combination thereof, on a free acid basis. In some embodiments, the tablet comprises about 300 mg of the compound of Formula Ia or Ib, or a pharmaceutically acceptable salt, co-crystal, solvate, or combination thereof, on a free acid basis.

In some embodiments, the tablet comprises about 200 mg to about 400 mg of the compound of Formula Ia or Ib, or a pharmaceutically acceptable salt thereof, on a free acid basis. In some embodiments, the tablet comprises about 250 mg to about 350 mg of the compound of Formula Ia or Ib, or a pharmaceutically acceptable salt thereof, on a free acid basis. In some embodiments, the tablet comprises about 275 mg to about 325 mg of the compound of Formula Ia or Ib, or a pharmaceutically acceptable salt thereof, on a free acid basis. In some embodiments, the tablet comprises about 290 mg to about 310 mg of the compound of Formula Ia or Ib, or a pharmaceutically acceptable salt thereof, on a free acid basis. In some embodiments, the tablet comprises about 299 mg to about 301 mg of the compound of Formula Ia or Ib, or a pharmaceutically acceptable salt thereof, on a free acid basis. In some embodiments, the tablet comprises about 300 mg of the compound of Formula Ia or Ib, or a pharmaceutically acceptable salt thereof, on a free acid basis.

In some embodiments, the tablet comprises about 0.1 mg to about 25 mg of the compound of Formula IIa or IIb, or a pharmaceutically acceptable salt, co-crystal, solvate, or combination thereof, for example, about 0.1 mg, about 0.2 mg, about 0.3 mg, about 0.4 mg, about 0.5 mg, about 0.6 mg, about 0.7 mg, about 0.8 mg, about 0.9 mg, about 1.0 mg, about 2.0 mg, about 3.0 mg, about 4.0 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, or about 25 mg of the compound of Formula IIa or IIb, or a pharmaceutically acceptable salt, co-crystal, solvate, or combination thereof.

In some embodiments, the tablet comprises about 0.4 mg to about 22 mg of the compound of Formula IIa or IIb, or a pharmaceutically acceptable salt, co-crystal, solvate, or combination thereof. In some embodiments, the tablet comprises about 0.1 mg to about 2.5 mg of the compound of Formula IIa or IIb, or a pharmaceutically acceptable salt, co-crystal, solvate, or combination thereof.

In some embodiments, the tablet comprises about 2.0 mg to about 2.2 mg of the compound of Formula IIa or IIb, or a pharmaceutically acceptable salt, co-crystal, solvate, or combination thereof. In some embodiments, the tablet comprises about 2.1 mg to about 2.2 mg of the compound of Formula IIa or IIb, or a pharmaceutically acceptable salt, co-crystal, solvate, or combination thereof. In some embodiments, the tablet comprises about 2.130 mg of the compound of Formula IIa or IIb, or a pharmaceutically acceptable salt, co-crystal, solvate, or combination thereof. In some embodiments, the tablet comprises about 2 mg of the compound of Formula IIa or IIb, or a pharmaceutically acceptable salt, co-crystal, solvate, or combination thereof, on a free base basis.

In some embodiments, the tablet comprises about 0.4 mg to about 0.6 mg of the compound of Formula IIa or IIb, or a pharmaceutically acceptable salt, co-crystal, solvate, or combination thereof. In some embodiments, the tablet comprises about 0.5 mg to about 0.6 mg of the compound of Formula IIa or IIb, or a pharmaceutically acceptable salt, co-crystal, solvate, or combination thereof. In some embodiments, the tablet comprises about 0.5325 mg of the compound of Formula IIa or IIb, or a pharmaceutically acceptable salt, co-crystal, solvate, or combination thereof. In some embodiments, the tablet comprises about 0.5 mg of the compound of Formula IIa or IIb, or a pharmaceutically acceptable salt, co-crystal, solvate, or combination thereof, on a free base basis.

In some embodiments, the tablet comprises about 0.4 mg to about 22 mg of the compound of Formula IIa or IIb, or a solvate thereof. In some embodiments, the tablet comprises about 0.1 mg to about 2.5 mg of the compound of Formula IIa or IIb, or a solvate thereof.

In some embodiments, the tablet comprises about 2.0 mg to about 2.2 mg of the compound of Formula IIa or IIb, or a solvate thereof. In some embodiments, the tablet comprises about 2.1 mg to about 2.2 mg of the compound of Formula IIa or IIb, or a solvate thereof. In some embodiments, the tablet comprises about 2.130 mg of the compound of Formula IIa or IIb, or a solvate thereof. In some embodiments, the tablet comprises about 2 mg of the compound of Formula IIa or IIb, or a solvate thereof, on a free base basis.

In some embodiments, the tablet comprises about 0.4 mg to about 0.6 mg of the compound of Formula IIa or IIb, or a solvate thereof. In some embodiments, the tablet comprises about 0.5 mg to about 0.6 mg of the compound of Formula IIa or IIb, or a solvate thereof. In some embodiments, the tablet comprises about 0.5325 mg of the compound of Formula IIa or IIb, or a solvate thereof. In some embodiments, the tablet comprises about 0.5 mg of the compound of Formula IIa or IIb, or a solvate thereof, on a free base basis.

In some embodiments, the tablet comprises about 0.4 mg to about 22 mg of the monohydrate of the compound of Formula IIa or IIb. In some embodiments, the tablet comprises about 0.1 mg to about 2.5 mg of the monohydrate of the compound of Formula IIa or IIb.

In some embodiments, the tablet comprises about 2.0 mg to about 2.2 mg of the monohydrate of the compound of Formula IIa or IIb. In some embodiments, the tablet comprises about 2.1 mg to about 2.2 mg of the monohydrate of the compound of Formula IIa or IIb. In some embodiments, the tablet comprises about 2.130 mg of the monohydrate of the compound of Formula IIa or IIb.

In some embodiments, the tablet comprises about 0.4 mg to about 0.6 mg of the monohydrate of the compound of Formula IIa or IIb. In some embodiments, the tablet comprises about 0.5 mg to about 0.6 mg of the monohydrate of the compound of Formula IIa or IIb. In some embodiments, the tablet comprises about 0.5 mg of the monohydrate of the compound of Formula IIa or IIb. In some embodiments, the tablet comprises about 0.5325 mg of the monohydrate of the compound of Formula IIa or IIb.

In some embodiments, the tablet comprises:
about 300 mg to about 500 mg of the compound of Formula Ia or Ib, or a pharmaceutically acceptable salt, co-crystal, solvate, or combination thereof, and
about 0.4 mg to about 22 mg of the compound of Formula IIa or IIb, or a pharmaceutically acceptable salt, co-crystal, solvate, or combination thereof.

In some embodiments, the tablet comprises:
about 400 mg of the compound of Formula Ia or Ib, or a pharmaceutically acceptable salt, co-crystal, solvate, or combination thereof; and
about 2.1 mg to about 2.2 mg of the compound of Formula IIa or IIb, or a pharmaceutically acceptable salt, co-crystal, solvate, or combination thereof.

In some embodiments, the tablet comprises:
about 400 mg of the compound of Formula Ia or Ib, or a pharmaceutically acceptable salt, co-crystal, solvate, or combination thereof; and
about 2.130 mg of the compound of Formula IIa or IIb, or a pharmaceutically acceptable salt, co-crystal, solvate, or combination thereof.

In some embodiments, the tablet comprises:
about 400 mg of the compound of Formula Ia or Ib, or a pharmaceutically acceptable salt, co-crystal, solvate, or combination thereof; and
about 2.0 mg of the compound of Formula IIa or IIb, or a pharmaceutically acceptable salt, co-crystal, solvate, or combination thereof, on a free base basis.

In some embodiments, the tablet comprises:
about 400 mg of the compound of Formula Ia or Ib, or a pharmaceutically acceptable salt, co-crystal, solvate, or combination thereof; and
about 0.5 mg to about 0.6 mg of the compound of Formula IIa or IIb, or a pharmaceutically acceptable salt, co-crystal, solvate, or combination thereof.

In some embodiments, the tablet comprises:
about 400 mg of the compound of Formula Ia or Ib, or a pharmaceutically acceptable salt, co-crystal, solvate, or combination thereof; and
about 0.5325 mg of the compound of Formula IIa or IIb, or a pharmaceutically acceptable salt, co-crystal, solvate, or combination thereof.

In some embodiments, the tablet comprises:
about 400 mg of the compound of Formula Ia or Ib, or a pharmaceutically acceptable salt, co-crystal, solvate, or combination thereof; and
about 0.5 mg of the compound of Formula IIa or IIb, or a pharmaceutically acceptable salt, co-crystal, solvate, or combination thereof, on a free base basis.

In some embodiments, the tablet comprises:
about 300 mg to about 500 mg of the compound of Formula Ia or Ib, or a pharmaceutically acceptable salt, co-crystal, solvate, or combination thereof;
about 0.4 mg to about 22 mg of the compound of Formula IIa or IIb, or a pharmaceutically acceptable salt, co-crystal, solvate, or combination thereof;
about 600 mg to about 700 mg of mannitol;
about 300 mg to about 350 mg of microcrystalline cellulose;
about 100 mg to about 150 mg croscarmellose sodium; and
about 20 mg to about 25 mg magnesium stearate.

In some embodiments, the tablet comprises:
about 400 mg of the compound of Formula Ia or Ib, or a pharmaceutically acceptable salt, co-crystal, solvate, or combination thereof;
about 2.1 mg to about 2.2 mg of the compound of Formula IIa or IIb, or a pharmaceutically acceptable salt, co-crystal, solvate, or combination thereof;
about 615 mg to about 625 mg of mannitol;
about 330 mg to about 340 mg of microcrystalline cellulose;
about 115 mg to about 125 mg croscarmellose sodium; and
about 20 mg to about 25 mg magnesium stearate.

In some embodiments, the tablet comprises:
about 400 mg of the compound of Formula Ia or Ib, or a pharmaceutically acceptable salt, co-crystal, solvate, or combination thereof;
about 2.130 mg of the compound of Formula IIa or IIb, or a pharmaceutically acceptable salt, co-crystal, solvate, or combination thereof;
about 621 mg of mannitol;
about 334.32 mg of microcrystalline cellulose;
about 120 mg croscarmellose sodium; and
about 22.5 mg magnesium stearate.

In some embodiments, the tablet comprises:
about 400 mg of the compound of Formula Ia or Ib, or a pharmaceutically acceptable salt, co-crystal, solvate, or combination thereof;
about 2.0 mg of the compound of Formula IIa or IIb, or a pharmaceutically acceptable salt, co-crystal, solvate, or combination thereof, on a free base basis;
about 621 mg of mannitol;
about 334.32 mg of microcrystalline cellulose;
about 120 mg croscarmellose sodium; and
about 22.5 mg magnesium stearate.

In some embodiments, the tablet comprises:
about 400 mg of the compound of Formula Ia or Ib, or a pharmaceutically acceptable salt, co-crystal, solvate, or combination thereof;
about 0.5 mg to about 0.6 mg of the compound of Formula IIa or IIb, or a pharmaceutically acceptable salt, co-crystal, solvate, or combination thereof;
about 615 mg to about 625 mg of mannitol;
about 330 mg to about 340 mg of microcrystalline cellulose;
about 115 mg to about 125 mg croscarmellose sodium; and
about 20 mg to about 25 mg magnesium stearate.

In some embodiments, the tablet comprises:
about 400 mg of the compound of Formula Ia or Ib, or a pharmaceutically acceptable salt, co-crystal, solvate, or combination thereof;
about 0.5325 mg of the compound of Formula IIa or IIb, or a pharmaceutically acceptable salt, co-crystal, solvate, or combination thereof;
about 621 mg of mannitol;
about 335.9175 mg of microcrystalline cellulose;
about 120 mg croscarmellose sodium; and
about 22.5 mg magnesium stearate.

In some embodiments, the tablet comprises:
about 400 mg of the compound of Formula Ia or Ib, or a pharmaceutically acceptable salt, co-crystal, solvate, or combination thereof;
about 0.5 mg of the compound of Formula IIa or IIb, or a pharmaceutically acceptable salt, co-crystal, solvate, or combination thereof, on a free base basis;
about 621 mg of mannitol;
about 335.9175 mg of microcrystalline cellulose;
about 120 mg croscarmellose sodium; and
about 22.5 mg magnesium stearate.

In some embodiments, the tablet comprises:
about 200 mg to about 400 mg of the compound of Formula Ia or Ib, or a pharmaceutically acceptable salt, co-crystal, solvate, or combination thereof, on a free acid basis; and
about 0.4 mg to about 22 mg of the compound of Formula IIa or IIb, or a pharmaceutically acceptable salt, co-crystal, solvate, or combination thereof.

In some embodiments, the tablet comprises:
about 300 mg of the compound of Formula Ia or Ib, or a pharmaceutically acceptable salt, co-crystal, solvate, or combination thereof, on a free acid basis; and
about 2.1 mg to about 2.2 mg of the compound of Formula IIa or IIb, or a pharmaceutically acceptable salt, co-crystal, solvate, or combination thereof.

In some embodiments, the tablet comprises:
about 300 mg of the compound of Formula Ia or Ib, or a pharmaceutically acceptable salt, co-crystal, solvate, or combination thereof, on a free acid basis; and
about 2.130 mg of the compound of Formula IIa or IIb, or a pharmaceutically acceptable salt, co-crystal, solvate, or combination thereof.

In some embodiments, the tablet comprises:
about 300 mg of the compound of Formula Ia or Ib, or a pharmaceutically acceptable salt, co-crystal, solvate, or combination thereof, on a free acid basis; and
about 2.0 mg of the compound of Formula IIa or IIb, or a pharmaceutically acceptable salt, co-crystal, solvate, or combination thereof, on a free base basis.

In some embodiments, the tablet comprises:
about 300 mg of the compound of Formula Ia or Ib, or a pharmaceutically acceptable salt, co-crystal, solvate, or combination thereof, on a free acid basis; and
about 0.5 mg to about 0.6 mg of the compound of Formula IIa or IIb, or a pharmaceutically acceptable salt, co-crystal, solvate, or combination thereof.

In some embodiments, the tablet comprises:
about 300 mg of the compound of Formula Ia or Ib, or a pharmaceutically acceptable salt, co-crystal, solvate, or combination thereof, on a free acid basis; and
about 0.5325 mg of the compound of Formula IIa or IIb, or a pharmaceutically acceptable salt, co-crystal, solvate, or combination thereof.

In some embodiments, the tablet comprises:
about 300 mg of the compound of Formula Ia or Ib, or a pharmaceutically acceptable salt, co-crystal, solvate, or combination thereof, on a free acid basis; and
about 0.5 mg of the compound of Formula IIa or IIb, or a pharmaceutically acceptable salt, co-crystal, solvate, or combination thereof, on a free base basis.

In some embodiments, the tablet comprises:
about 200 mg to about 400 mg of the compound of Formula Ia or Ib, or a pharmaceutically acceptable salt, co-crystal, solvate, or combination thereof, on a free acid basis;
about 0.4 mg to about 22 mg of the compound of Formula IIa or IIb, or a pharmaceutically acceptable salt, co-crystal, solvate, or combination thereof;
about 600 mg to about 700 mg of mannitol;
about 300 mg to about 350 mg of microcrystalline cellulose;
about 100 mg to about 150 mg croscarmellose sodium; and
about 20 mg to about 25 mg magnesium stearate.

In some embodiments, the tablet comprises:
about 300 mg of the compound of Formula Ia or Ib, or a pharmaceutically acceptable salt, co-crystal, solvate, or combination thereof, on a free acid basis;
about 2.1 mg to about 2.2 mg of the compound of Formula IIa or IIb, or a pharmaceutically acceptable salt, co-crystal, solvate, or combination thereof;
about 615 mg to about 625 mg of mannitol;
about 330 mg to about 340 mg of microcrystalline cellulose;
about 115 mg to about 125 mg croscarmellose sodium; and
about 20 mg to about 25 mg magnesium stearate.

In some embodiments, the tablet comprises:
about 300 mg of the compound of Formula Ia or Ib, or a pharmaceutically acceptable salt, co-crystal, solvate, or combination thereof, on a free acid basis;
about 2.130 mg of the compound of Formula IIa or IIb, or a pharmaceutically acceptable salt, co-crystal, solvate, or combination thereof;
about 621 mg of mannitol;
about 334.32 mg of microcrystalline cellulose;
about 120 mg croscarmellose sodium; and
about 22.5 mg magnesium stearate.

In some embodiments, the tablet comprises:
about 300 mg of the compound of Formula Ia or Ib, or a pharmaceutically acceptable salt, co-crystal, solvate, or combination thereof, on a free acid basis;
about 2.0 mg of the compound of Formula IIa or IIb, or a pharmaceutically acceptable salt, co-crystal, solvate, or combination thereof, on a free base basis;
about 621 mg of mannitol;
about 334.32 mg of microcrystalline cellulose;
about 120 mg croscarmellose sodium; and
about 22.5 mg magnesium stearate.

In some embodiments, the tablet comprises:
about 300 mg of the compound of Formula Ia or Ib, or a pharmaceutically acceptable salt, co-crystal, solvate, or combination thereof, on a free acid basis;
about 0.5 mg to about 0.6 mg of the compound of Formula IIa or IIb, or a pharmaceutically acceptable salt, co-crystal, solvate, or combination thereof;
about 615 mg to about 625 mg of mannitol;
about 330 mg to about 340 mg of microcrystalline cellulose;
about 115 mg to about 125 mg croscarmellose sodium; and
about 20 mg to about 25 mg magnesium stearate.

In some embodiments, the tablet comprises:
about 300 mg of the compound of Formula Ia or Ib, or a pharmaceutically acceptable salt, co-crystal, solvate, or combination thereof, on a free acid basis;
about 0.5325 mg of the compound of Formula IIa or IIb, or a pharmaceutically acceptable salt, co-crystal, solvate, or combination thereof;
about 621 mg of mannitol;
about 335.9175 mg of microcrystalline cellulose;
about 120 mg croscarmellose sodium; and
about 22.5 mg magnesium stearate.

In some embodiments, the tablet comprises:
about 300 mg of the compound of Formula Ia or Ib, or a pharmaceutically acceptable salt, co-crystal, solvate, or combination thereof, on a free acid basis;
about 0.5 mg of the compound of Formula IIa or IIb, or a pharmaceutically acceptable salt, co-crystal, solvate, or combination thereof, on a free base basis;
about 621 mg of mannitol;
about 335.9175 mg of microcrystalline cellulose;
about 120 mg croscarmellose sodium; and
about 22.5 mg magnesium stearate.

In some embodiments, the tablet is about 1000 mg to about 1600 mg, for example, about 1000 mg, about 1050 mg, about 1100 mg, about 1150 mg, about 1200 mg, about 1250 mg, about 1300 mg, about 1350 mg, about 1400 mg, about 1450 mg, about 1500 mg, about 1550 mg, or about 1600 mg. In some embodiments, the tablet is about 1400 mg to about 1600 mg. In some embodiments, the tablet is about 1450 mg to about 1550 mg. In some embodiments, the tablet is about 1500 mg.

In some embodiments, the tablet comprises:
about 200 mg to about 400 mg of the compound of Formula Ia or Ib, or a pharmaceutically acceptable salt thereof, on a free acid basis;
about 0.1 mg to about 2.5 mg of the compound of Formula IIa or IIb, or a solvate thereof, on a free base basis;
about 50 mg to about 100 mg of copovidone;
about 5 mg to about 30 mg of poloxamer 407;
about 600 mg to about 700 mg of mannitol;
about 300 mg to about 350 mg of microcrystalline cellulose;
about 100 mg to about 150 mg of croscarmellose sodium; and
about 20 mg to about 25 mg of magnesium stearate.

In some embodiments, the tablet comprises:
about 300 mg of the compound of Formula Ia or Ib, or a pharmaceutically acceptable salt thereof, on a free acid basis;
about 2 mg of the compound of Formula IIa or IIb, or a solvate thereof, on a free base basis;
about 65 mg to about 75 mg of copovidone;
about 15 mg to about 25 mg of poloxamer 407;
about 615 mg to about 625 mg of mannitol;
about 330 mg to about 340 mg of microcrystalline cellulose;
about 115 mg to about 125 mg of croscarmellose sodium; and
about 20 mg to about 25 mg of magnesium stearate.

In some embodiments, the tablet comprises:
about 300 mg of the compound of Formula Ia or Ib, or a pharmaceutically acceptable salt thereof, on a free acid basis;
about 2 mg of the compound of Formula IIa or IIb, or a solvate thereof, on a free base basis;
about 73.2 mg of copovidone;
about 19.95 mg of poloxamer 407;
about 621 mg of mannitol;
about 334.32 mg of microcrystalline cellulose;
about 120 mg of croscarmellose sodium; and
about 22.5 mg of magnesium stearate.

In some embodiments, the tablet comprises:
about 200 mg to about 400 mg of the sodium salt of the compound of Formula Ia or Ib;
about 0.1 mg to about 2.5 mg of the monohydrate of the compound of Formula IIa or IIb;
about 50 mg to about 100 mg of copovidone;
about 5 mg to about 30 mg of poloxamer 407;
about 600 mg to about 700 mg of mannitol;
about 300 mg to about 350 mg of microcrystalline cellulose;
about 100 mg to about 150 mg of croscarmellose sodium; and
about 20 mg to about 25 mg of magnesium stearate.

In some embodiments, the tablet comprises:
about 306.8 mg of the sodium salt of the compound of Formula Ia or Ib;
about 2.1 mg to about 2.2 mg of the monohydrate of the compound of Formula IIa or IIb;
about 65 mg to about 75 mg of copovidone;
about 15 mg to about 25 mg of poloxamer 407;
about 615 mg to about 625 mg of mannitol;
about 330 mg to about 340 mg of microcrystalline cellulose;
about 115 mg to about 125 mg of croscarmellose sodium; and
about 20 mg to about 25 mg of magnesium stearate.

In some embodiments, the tablet comprises:
about 306.8 mg of the sodium salt of the compound of Formula Ia or Ib;
about 2.130 mg of the monohydrate of the compound of Formula IIa or IIb;
about 65 mg to about 75 mg of copovidone;
about 15 mg to about 25 mg of poloxamer 407;
about 615 mg to about 625 mg of mannitol;
about 330 mg to about 340 mg of microcrystalline cellulose;
about 115 mg to about 125 mg of croscarmellose sodium; and
about 20 mg to about 25 mg of magnesium stearate.

In some embodiments, the tablet comprises:
about 306.8 mg of the sodium salt of the compound of Formula Ia or Ib;
about 2.130 mg of the monohydrate of the compound of Formula IIa or IIb;
about 73.2 mg of copovidone;
about 19.95 mg of poloxamer 407;
about 621 mg of mannitol;
about 334.32 mg of microcrystalline cellulose;
about 120 mg of croscarmellose sodium; and
about 22.5 mg of magnesium stearate.

In some embodiments, the tablet comprises:
about 200 mg to about 400 mg of the compound of Formula Ia or Ib, or a pharmaceutically acceptable salt thereof, on a free acid basis;
about 0.1 mg to about 2.5 mg of the compound of Formula IIa or IIb, or a solvate thereof, on a free base basis;
about 50 mg to about 100 mg of copovidone;
about 5 mg to about 30 mg of poloxamer 407;
about 600 mg to about 700 mg of mannitol;
about 300 mg to about 350 mg of microcrystalline cellulose;
about 100 mg to about 150 mg of croscarmellose sodium;
about 20 mg to about 25 mg of magnesium stearate; and
an outer film coat.

In some embodiments, the tablet comprises:
about 300 mg of the compound of Formula Ia or Ib, or a pharmaceutically acceptable salt thereof, on a free acid basis;
about 2 mg of the compound of Formula IIa or IIb, or a solvate thereof, on a free base basis;
about 65 mg to about 75 mg of copovidone;
about 15 mg to about 25 mg of poloxamer 407;
about 615 mg to about 625 mg of mannitol;
about 330 mg to about 340 mg of microcrystalline cellulose;
about 115 mg to about 125 mg of croscarmellose sodium;
about 20 mg to about 25 mg of magnesium stearate; and
an outer film coat.

In some embodiments, the tablet comprises:
about 300 mg of the compound of Formula Ia or Ib, or a pharmaceutically acceptable salt thereof, on a free acid basis;
about 2 mg of the compound of Formula IIa or IIb, or a solvate thereof, on a free base basis, on a free base basis;
about 73.2 mg of copovidone;
about 19.95 mg of poloxamer 407;
about 621 mg of mannitol;
about 334.32 mg of microcrystalline cellulose;
about 120 mg of croscarmellose sodium;
about 22.5 mg of magnesium stearate; and
an outer film coat.

In some embodiments, the tablet comprises:
about 200 mg to about 400 mg of the sodium salt of the compound of Formula Ia or Ib;
about 0.1 mg to about 2.5 mg of the monohydrate of the compound of Formula IIa or IIb;
about 50 mg to about 100 mg of copovidone;
about 5 mg to about 30 mg of poloxamer 407;
about 600 mg to about 700 mg of mannitol;
about 300 mg to about 350 mg of microcrystalline cellulose;
about 100 mg to about 150 mg of croscarmellose sodium;
about 20 mg to about 25 mg of magnesium stearate; and
an outer film coat.

In some embodiments, the tablet comprises:
about 306.8 mg of the sodium salt of the compound of Formula Ia or Ib;
about 2.1 mg to about 2.2 mg of the monohydrate of the compound of Formula IIa or IIb;
about 65 mg to about 75 mg of copovidone;
about 15 mg to about 25 mg of poloxamer 407;
about 615 mg to about 625 mg of mannitol;
about 330 mg to about 340 mg of microcrystalline cellulose;
about 115 mg to about 125 mg of croscarmellose sodium;
about 20 mg to about 25 mg of magnesium stearate; and
an outer film coat.

In some embodiments, the tablet comprises:
about 306.8 mg of the sodium salt of the compound of Formula Ia or Ib;
about 2.130 mg of the monohydrate of the compound of Formula IIa or IIb;
about 65 mg to about 75 mg of copovidone;
about 15 mg to about 25 mg of poloxamer 407;
about 615 mg to about 625 mg of mannitol;
about 330 mg to about 340 mg of microcrystalline cellulose;
about 115 mg to about 125 mg of croscarmellose sodium; and
about 20 mg to about 25 mg of magnesium stearate.

In some embodiments, the tablet comprises:
about 306.8 mg of the sodium salt of the compound of Formula Ia or Ib;
about 2.130 mg of the monohydrate of the compound of Formula IIa or IIb;
about 73.2 mg of copovidone;
about 19.95 mg of poloxamer 407;
about 621 mg of mannitol;
about 334.32 mg of microcrystalline cellulose;
about 120 mg of croscarmellose sodium;
about 22.5 mg of magnesium stearate; and
an outer film coat.

In some embodiments, the tablet consists essentially of:
about 200 mg to about 400 mg of the compound of Formula Ia or Ib, or a pharmaceutically acceptable salt thereof, on a free acid basis;
about 0.1 mg to about 2.5 mg of the compound of Formula IIa or IIb, or a solvate thereof, on a free base basis;
about 50 mg to about 100 mg of copovidone;
about 5 mg to about 30 mg of poloxamer 407;
about 600 mg to about 700 mg of mannitol;
about 300 mg to about 350 mg of microcrystalline cellulose;
about 100 mg to about 150 mg of croscarmellose sodium; and
about 20 mg to about 25 mg of magnesium stearate.

In some embodiments, the tablet consists essentially of:
about 300 mg of the compound of Formula Ia or Ib, or a pharmaceutically acceptable salt thereof, on a free acid basis;
about 2 mg of the compound of Formula IIa or IIb, or a solvate thereof, on a free base basis;
about 65 mg to about 75 mg of copovidone;
about 15 mg to about 25 mg of poloxamer 407;
about 615 mg to about 625 mg of mannitol;
about 330 mg to about 340 mg of microcrystalline cellulose;
about 115 mg to about 125 mg of croscarmellose sodium; and
about 20 mg to about 25 mg of magnesium stearate.

In some embodiments, the tablet consists essentially of:
about 300 mg of the compound of Formula Ia or Ib, or a pharmaceutically acceptable salt thereof, on a free acid basis;
about 2 mg of the compound of Formula IIa or IIb, or a solvate thereof, on a free base basis;
about 73.2 mg of copovidone;
about 19.95 mg of poloxamer 407;
about 621 mg of mannitol;
about 334.32 mg of microcrystalline cellulose;
about 120 mg of croscarmellose sodium; and
about 22.5 mg of magnesium stearate.

In some embodiments, the tablet consists essentially of:
about 200 mg to about 400 mg of the sodium salt of the compound of Formula Ia or Ib;
about 0.1 mg to about 2.5 mg of the monohydrate of the compound of Formula IIa or IIb;
about 50 mg to about 100 mg of copovidone;
about 5 mg to about 30 mg of poloxamer 407;
about 600 mg to about 700 mg of mannitol;
about 300 mg to about 350 mg of microcrystalline cellulose;
about 100 mg to about 150 mg of croscarmellose sodium; and
about 20 mg to about 25 mg of magnesium stearate.

In some embodiments, the tablet consists essentially of:
about 306.8 mg of the sodium salt of the compound of Formula Ia or Ib;
about 2.1 mg to about 2.2 mg of the monohydrate of the compound of Formula IIa or IIb;
about 65 mg to about 75 mg of copovidone;
about 15 mg to about 25 mg of poloxamer 407;
about 615 mg to about 625 mg of mannitol;
about 330 mg to about 340 mg of microcrystalline cellulose;
about 115 mg to about 125 mg of croscarmellose sodium; and
about 20 mg to about 25 mg of magnesium stearate.

In some embodiments, the tablet consists essentially of:
about 306.8 mg of the sodium salt of the compound of Formula Ia or Ib;
about 2.130 mg of the monohydrate of the compound of Formula IIa or IIb;
about 65 mg to about 75 mg of copovidone;
about 15 mg to about 25 mg of poloxamer 407;
about 615 mg to about 625 mg of mannitol;
about 330 mg to about 340 mg of microcrystalline cellulose;
about 115 mg to about 125 mg of croscarmellose sodium; and
about 20 mg to about 25 mg of magnesium stearate.

In some embodiments, the tablet consists essentially of:
about 306.8 mg of the sodium salt of the compound of Formula Ia or Ib;
about 2.130 mg of the monohydrate of the compound of Formula IIa or IIb;
about 73.2 mg of copovidone;
about 19.95 mg of poloxamer 407;
about 621 mg of mannitol;
about 334.32 mg of microcrystalline cellulose;
about 120 mg of croscarmellose sodium; and
about 22.5 mg of magnesium stearate.

In some embodiments, the tablet consists essentially of:
about 200 mg to about 400 mg of the compound of Formula Ia or Ib, or a pharmaceutically acceptable salt thereof, on a free acid basis;
about 0.1 mg to about 2.5 mg of the compound of Formula IIa or IIb, or a solvate thereof, on a free base basis;
about 50 mg to about 100 mg of copovidone;
about 5 mg to about 30 mg of poloxamer 407;
about 600 mg to about 700 mg of mannitol;
about 300 mg to about 350 mg of microcrystalline cellulose;
about 100 mg to about 150 mg of croscarmellose sodium;
about 20 mg to about 25 mg of magnesium stearate; and
an outer film coat.

In some embodiments, the tablet consists essentially of:
about 300 mg of the compound of Formula Ia or Ib, or a pharmaceutically acceptable salt thereof, on a free acid basis;
about 2 mg of the compound of Formula IIa or IIb, or a solvate thereof, on a free base basis;
about 65 mg to about 75 mg of copovidone;
about 15 mg to about 25 mg of poloxamer 407;
about 615 mg to about 625 mg of mannitol;
about 330 mg to about 340 mg of microcrystalline cellulose;
about 115 mg to about 125 mg of croscarmellose sodium;
about 20 mg to about 25 mg of magnesium stearate; and
an outer film coat.

In some embodiments, the tablet consists essentially of:
about 300 mg of the compound of Formula Ia or Ib, or a pharmaceutically acceptable salt thereof, on a free acid basis;
about 2 mg of the compound of Formula IIa or IIb, or a solvate thereof, on a free base basis, on a free base basis;
about 73.2 mg of copovidone;
about 19.95 mg of poloxamer 407;
about 621 mg of mannitol;
about 334.32 mg of microcrystalline cellulose;
about 120 mg of croscarmellose sodium;
about 22.5 mg of magnesium stearate; and
an outer film coat.

In some embodiments, the tablet consists essentially of:
about 200 mg to about 400 mg of the sodium salt of the compound of Formula Ia or Ib;
about 0.1 mg to about 2.5 mg of the monohydrate of the compound of Formula IIa or IIb;
about 50 mg to about 100 mg of copovidone;
about 5 mg to about 30 mg of poloxamer 407;
about 600 mg to about 700 mg of mannitol;
about 300 mg to about 350 mg of microcrystalline cellulose;
about 100 mg to about 150 mg of croscarmellose sodium;
about 20 mg to about 25 mg of magnesium stearate; and
an outer film coat.

In some embodiments, the tablet consists essentially of:
about 306.8 mg of the sodium salt of the compound of Formula Ia or Ib;
about 2.1 mg to about 2.2 mg of the monohydrate of the compound of Formula IIa or IIb;
about 65 mg to about 75 mg of copovidone;
about 15 mg to about 25 mg of poloxamer 407;
about 615 mg to about 625 mg of mannitol;
about 330 mg to about 340 mg of microcrystalline cellulose;
about 115 mg to about 125 mg of croscarmellose sodium;
about 20 mg to about 25 mg of magnesium stearate; and
an outer film coat.

In some embodiments, the tablet consists essentially of:
about 306.8 mg of the sodium salt of the compound of Formula Ia or Ib;
about 2.130 mg of the monohydrate of the compound of Formula IIa or IIb;
about 65 mg to about 75 mg of copovidone;
about 15 mg to about 25 mg of poloxamer 407;
about 615 mg to about 625 mg of mannitol;
about 330 mg to about 340 mg of microcrystalline cellulose;
about 115 mg to about 125 mg of croscarmellose sodium; and
about 20 mg to about 25 mg of magnesium stearate.

In some embodiments, the tablet consists essentially of:
about 306.8 mg of the sodium salt of the compound of Formula Ia or Ib;
about 2.130 mg of the monohydrate of the compound of Formula IIa or IIb;
about 73.2 mg of copovidone;
about 19.95 mg of poloxamer 407;
about 621 mg of mannitol;
about 334.32 mg of microcrystalline cellulose;
about 120 mg of croscarmellose sodium;
about 22.5 mg of magnesium stearate; and
an outer film coat.

In some embodiments, the tablet consists of:
about 200 mg to about 400 mg of the compound of Formula Ia or Ib, or a pharmaceutically acceptable salt thereof, on a free acid basis;
about 0.1 mg to about 2.5 mg of the compound of Formula IIa or IIb, or a solvate thereof, on a free base basis;
about 50 mg to about 100 mg of copovidone;
about 5 mg to about 30 mg of poloxamer 407;
about 600 mg to about 700 mg of mannitol;
about 300 mg to about 350 mg of microcrystalline cellulose;
about 100 mg to about 150 mg of croscarmellose sodium; and
about 20 mg to about 25 mg of magnesium stearate.

In some embodiments, the tablet consists of:
about 300 mg of the compound of Formula Ia or Ib, or a pharmaceutically acceptable salt thereof, on a free acid basis;
about 2 mg of the compound of Formula IIa or IIb, or a solvate thereof, on a free base basis;
about 65 mg to about 75 mg of copovidone;
about 15 mg to about 25 mg of poloxamer 407;
about 615 mg to about 625 mg of mannitol;
about 330 mg to about 340 mg of microcrystalline cellulose;
about 115 mg to about 125 mg of croscarmellose sodium; and
about 20 mg to about 25 mg of magnesium stearate.

In some embodiments, the tablet consists of:
about 300 mg of the compound of Formula Ia or Ib, or a pharmaceutically acceptable salt thereof, on a free acid basis;
about 2 mg of the compound of Formula IIa or IIb, or a solvate thereof, on a free base basis;
about 73.2 mg of copovidone;
about 19.95 mg of poloxamer 407;
about 621 mg of mannitol;
about 334.32 mg of microcrystalline cellulose;
about 120 mg of croscarmellose sodium; and
about 22.5 mg of magnesium stearate.

In some embodiments, the tablet consists of:
about 200 mg to about 400 mg of the sodium salt of the compound of Formula Ia or Ib;
about 0.1 mg to about 2.5 mg of the monohydrate of the compound of Formula IIa or IIb;
about 50 mg to about 100 mg of copovidone;
about 5 mg to about 30 mg of poloxamer 407;
about 600 mg to about 700 mg of mannitol;
about 300 mg to about 350 mg of microcrystalline cellulose;
about 100 mg to about 150 mg of croscarmellose sodium; and
about 20 mg to about 25 mg of magnesium stearate.

In some embodiments, the tablet consists of:
about 306.8 mg of the sodium salt of the compound of Formula Ia or Ib;
about 2.1 mg to about 2.2 mg of the monohydrate of the compound of Formula IIa or IIb;
about 65 mg to about 75 mg of copovidone;
about 15 mg to about 25 mg of poloxamer 407;
about 615 mg to about 625 mg of mannitol;
about 330 mg to about 340 mg of microcrystalline cellulose;
about 115 mg to about 125 mg of croscarmellose sodium; and
about 20 mg to about 25 mg of magnesium stearate.

In some embodiments, the tablet consists of:
about 306.8 mg of the sodium salt of the compound of Formula Ia or Ib;
about 2.130 mg of the monohydrate of the compound of Formula IIa or IIb;
about 65 mg to about 75 mg of copovidone;
about 15 mg to about 25 mg of poloxamer 407;
about 615 mg to about 625 mg of mannitol;
about 330 mg to about 340 mg of microcrystalline cellulose;
about 115 mg to about 125 mg of croscarmellose sodium; and
about 20 mg to about 25 mg of magnesium stearate.

In some embodiments, the tablet consists of:
about 306.8 mg of the sodium salt of the compound of Formula Ia or Ib;
about 2.130 mg of the monohydrate of the compound of Formula IIa or IIb;
about 73.2 mg of copovidone;
about 19.95 mg of poloxamer 407;
about 621 mg of mannitol;
about 334.32 mg of microcrystalline cellulose;
about 120 mg of croscarmellose sodium; and
about 22.5 mg of magnesium stearate.

In some embodiments, the tablet consists of:
about 200 mg to about 400 mg of the compound of Formula Ia or Ib, or a pharmaceutically acceptable salt thereof, on a free acid basis;
about 0.1 mg to about 2.5 mg of the compound of Formula IIa or IIb, or a solvate thereof, on a free base basis;
about 50 mg to about 100 mg of copovidone;
about 5 mg to about 30 mg of poloxamer 407;
about 600 mg to about 700 mg of mannitol;
about 300 mg to about 350 mg of microcrystalline cellulose;
about 100 mg to about 150 mg of croscarmellose sodium;
about 20 mg to about 25 mg of magnesium stearate; and
an outer film coat.

In some embodiments, the tablet consists of:
about 300 mg of the compound of Formula Ia or Ib, or a pharmaceutically acceptable salt thereof, on a free acid basis;
about 2 mg of the compound of Formula IIa or IIb, or a solvate thereof, on a free base basis;
about 65 mg to about 75 mg of copovidone;
about 15 mg to about 25 mg of poloxamer 407;
about 615 mg to about 625 mg of mannitol;
about 330 mg to about 340 mg of microcrystalline cellulose;
about 115 mg to about 125 mg of croscarmellose sodium;
about 20 mg to about 25 mg of magnesium stearate; and
an outer film coat.

In some embodiments, the tablet consists of:
about 300 mg of the compound of Formula Ia or Ib, or a pharmaceutically acceptable salt thereof, on a free acid basis;
about 2 mg of the compound of Formula IIa or IIb, or a solvate thereof, on a free base basis, on a free base basis;
about 73.2 mg of copovidone;
about 19.95 mg of poloxamer 407;
about 621 mg of mannitol;
about 334.32 mg of microcrystalline cellulose;
about 120 mg of croscarmellose sodium;
about 22.5 mg of magnesium stearate; and
an outer film coat.

In some embodiments, the tablet consists of:
about 200 mg to about 400 mg of the sodium salt of the compound of Formula Ia or Ib;
about 0.1 mg to about 2.5 mg of the monohydrate of the compound of Formula IIa or IIb;
about 50 mg to about 100 mg of copovidone;
about 5 mg to about 30 mg of poloxamer 407;
about 600 mg to about 700 mg of mannitol;
about 300 mg to about 350 mg of microcrystalline cellulose;
about 100 mg to about 150 mg of croscarmellose sodium;
about 20 mg to about 25 mg of magnesium stearate; and
an outer film coat.

In some embodiments, the tablet consists of:
about 306.8 mg of the sodium salt of the compound of Formula Ia or Ib;
about 2.1 mg to about 2.2 mg of the monohydrate of the compound of Formula IIa or IIb;
about 65 mg to about 75 mg of copovidone;
about 15 mg to about 25 mg of poloxamer 407;
about 615 mg to about 625 mg of mannitol;
about 330 mg to about 340 mg of microcrystalline cellulose;
about 115 mg to about 125 mg of croscarmellose sodium;
about 20 mg to about 25 mg of magnesium stearate; and
an outer film coat.

In some embodiments, the tablet consists of:
about 306.8 mg of the sodium salt of the compound of Formula Ia or Ib;
about 2.130 mg of the monohydrate of the compound of Formula IIa or IIb;
about 65 mg to about 75 mg of copovidone;
about 15 mg to about 25 mg of poloxamer 407;
about 615 mg to about 625 mg of mannitol;
about 330 mg to about 340 mg of microcrystalline cellulose;
about 115 mg to about 125 mg of croscarmellose sodium; and
about 20 mg to about 25 mg of magnesium stearate.

In some embodiments, the tablet consists of:
about 306.8 mg of the sodium salt of the compound of Formula Ia or Ib;
about 2.130 mg of the monohydrate of the compound of Formula IIa or IIb;
about 73.2 mg of copovidone;
about 19.95 mg of poloxamer 407;
about 621 mg of mannitol;
about 334.32 mg of microcrystalline cellulose;
about 120 mg of croscarmellose sodium;
about 22.5 mg of magnesium stearate; and
an outer film coat.

In some embodiments, the tablet comprises:
about 200 mg to about 400 mg of the compound of Formula Ia or Ib, or a pharmaceutically acceptable salt thereof, on a free acid basis;
about 0.4 mg to about 0.6 mg of the compound of Formula IIa or IIb, or a solvate thereof, on a free base basis;
about 50 mg to about 100 mg of copovidone;
about 5 mg to about 30 mg of poloxamer 407;
about 600 mg to about 700 mg of mannitol;
about 300 mg to about 350 mg of microcrystalline cellulose;
about 100 mg to about 150 mg of croscarmellose sodium; and
about 20 mg to about 25 mg of magnesium stearate.

In some embodiments, the tablet comprises:
about 300 mg of the compound of Formula Ia or Ib, or a pharmaceutically acceptable salt thereof, on a free acid basis;
about 0.5 mg of the compound of Formula IIa or IIb, or a solvate thereof, on a free base basis;
about 65 mg to about 75 mg of copovidone;
about 15 mg to about 25 mg of poloxamer 407;
about 615 mg to about 625 mg of mannitol;
about 330 mg to about 340 mg of microcrystalline cellulose;
about 115 mg to about 125 mg of croscarmellose sodium; and
about 20 mg to about 25 mg of magnesium stearate.

In some embodiments, the tablet comprises:
about 300 mg of the compound of Formula Ia or Ib, or a pharmaceutically acceptable salt thereof, on a free acid basis;
about 0.5 mg of the compound of Formula IIa or IIb, or a solvate thereof, on a free base basis;
about 73.2 mg of copovidone;
about 19.95 mg of poloxamer 407;
about 621 mg of mannitol;
about 335.92 mg of microcrystalline cellulose;
about 120 mg of croscarmellose sodium; and
about 22.5 mg of magnesium stearate.

In some embodiments, the tablet comprises:
about 200 mg to about 400 mg of the sodium salt of the compound of Formula Ia or Ib;
about 0.4 mg to about 0.6 mg of the monohydrate of the compound of Formula IIa or IIb;
about 50 mg to about 100 mg of copovidone;
about 5 mg to about 30 mg of poloxamer 407;
about 600 mg to about 700 mg of mannitol;
about 300 mg to about 350 mg of microcrystalline cellulose;
about 100 mg to about 150 mg of croscarmellose sodium; and
about 20 mg to about 25 mg of magnesium stearate.

In some embodiments, the tablet comprises:
about 306.8 mg of the sodium salt of the compound of Formula Ia or Ib;
about 0.5 mg to about 0.6 mg of the monohydrate of the compound of Formula IIa or IIb;
about 65 mg to about 75 mg of copovidone;
about 15 mg to about 25 mg of poloxamer 407;
about 615 mg to about 625 mg of mannitol;
about 330 mg to about 340 mg of microcrystalline cellulose;
about 115 mg to about 125 mg of croscarmellose sodium; and
about 20 mg to about 25 mg of magnesium stearate.

In some embodiments, the tablet comprises:
about 306.8 mg of the sodium salt of the compound of Formula Ia or Ib;
about 0.53 mg of the monohydrate of the compound of Formula IIa or IIb;
about 65 mg to about 75 mg of copovidone;
about 15 mg to about 25 mg of poloxamer 407;
about 615 mg to about 625 mg of mannitol;
about 330 mg to about 340 mg of microcrystalline cellulose;
about 115 mg to about 125 mg of croscarmellose sodium; and
about 20 mg to about 25 mg of magnesium stearate.

In some embodiments, the tablet comprises:
about 306.8 mg of the sodium salt of the compound of Formula Ia or Ib;
about 0.53 mg of the monohydrate of the compound of Formula IIa or IIb;
about 73.2 mg of copovidone;
about 19.95 mg of poloxamer 407;
about 621 mg of mannitol;
about 335.92 mg of microcrystalline cellulose;
about 120 mg of croscarmellose sodium; and
about 22.5 mg of magnesium stearate.

In some embodiments, the tablet comprises:
about 200 mg to about 400 mg of the compound of Formula Ia or Ib, or a pharmaceutically acceptable salt thereof, on a free acid basis;
about 0.4 mg to about 0.6 mg of the compound of Formula IIa or IIb, or a solvate thereof, on a free base basis;
about 50 mg to about 100 mg of copovidone;
about 5 mg to about 30 mg of poloxamer 407;
about 600 mg to about 700 mg of mannitol;
about 300 mg to about 350 mg of microcrystalline cellulose;
about 100 mg to about 150 mg of croscarmellose sodium;
about 20 mg to about 25 mg of magnesium stearate; and
an outer film coat.

In some embodiments, the tablet comprises:
about 300 mg of the compound of Formula Ia or Ib, or a pharmaceutically acceptable salt thereof, on a free acid basis;
about 0.5 mg of the compound of Formula IIa or IIb, or a solvate thereof, on a free base basis;
about 65 mg to about 75 mg of copovidone;
about 15 mg to about 25 mg of poloxamer 407;
about 615 mg to about 625 mg of mannitol;
about 330 mg to about 340 mg of microcrystalline cellulose;
about 115 mg to about 125 mg of croscarmellose sodium;
about 20 mg to about 25 mg of magnesium stearate; and
an outer film coat.

In some embodiments, the tablet comprises:
about 300 mg of the compound of Formula Ia or Ib, or a pharmaceutically acceptable salt thereof, on a free acid basis;
about 0.5 mg of the compound of Formula IIa or IIb, or a solvate thereof, on a free base basis, on a free base basis;
about 73.2 mg of copovidone;
about 19.95 mg of poloxamer 407;
about 621 mg of mannitol;
about 335.92 mg of microcrystalline cellulose;
about 120 mg of croscarmellose sodium;
about 22.5 mg of magnesium stearate; and
an outer film coat.

In some embodiments, the tablet comprises:
about 200 mg to about 400 mg of the sodium salt of the compound of Formula Ia or Ib;
about 0.4 mg to about 0.6 mg of the monohydrate of the compound of Formula IIa or IIb;
about 50 mg to about 100 mg of copovidone;
about 5 mg to about 30 mg of poloxamer 407;
about 600 mg to about 700 mg of mannitol;
about 300 mg to about 350 mg of microcrystalline cellulose;
about 100 mg to about 150 mg of croscarmellose sodium;
about 20 mg to about 25 mg of magnesium stearate; and
an outer film coat.

In some embodiments, the tablet comprises:
about 306.8 mg of the sodium salt of the compound of Formula Ia or Ib;
about 0.5 mg to about 0.6 mg of the monohydrate of the compound of Formula IIa or IIb;
about 65 mg to about 75 mg of copovidone;
about 15 mg to about 25 mg of poloxamer 407;
about 615 mg to about 625 mg of mannitol;
about 330 mg to about 340 mg of microcrystalline cellulose;
about 115 mg to about 125 mg of croscarmellose sodium;
about 20 mg to about 25 mg of magnesium stearate; and
an outer film coat.

In some embodiments, the tablet comprises:
about 306.8 mg of the sodium salt of the compound of Formula Ia or Ib;
about 0.53 mg of the monohydrate of the compound of Formula IIa or IIb;
about 65 mg to about 75 mg of copovidone;
about 15 mg to about 25 mg of poloxamer 407;
about 615 mg to about 625 mg of mannitol;
about 330 mg to about 340 mg of microcrystalline cellulose;
about 115 mg to about 125 mg of croscarmellose sodium; and
about 20 mg to about 25 mg of magnesium stearate.

In some embodiments, the tablet comprises:
about 306.8 mg of the sodium salt of the compound of Formula Ia or Ib;
about 0.53 mg of the monohydrate of the compound of Formula IIa or IIb;
about 73.2 mg of copovidone;
about 19.95 mg of poloxamer 407;
about 621 mg of mannitol;
about 335.92 mg of microcrystalline cellulose;
about 120 mg of croscarmellose sodium;
about 22.5 mg of magnesium stearate; and
an outer film coat.

In some embodiments, the tablet consists essentially of:
about 200 mg to about 400 mg of the compound of Formula Ia or Ib, or a pharmaceutically acceptable salt thereof, on a free acid basis;
about 0.4 mg to about 0.6 mg of the compound of Formula IIa or IIb, or a solvate thereof, on a free base basis;
about 50 mg to about 100 mg of copovidone;
about 5 mg to about 30 mg of poloxamer 407;
about 600 mg to about 700 mg of mannitol;
about 300 mg to about 350 mg of microcrystalline cellulose;
about 100 mg to about 150 mg of croscarmellose sodium; and
about 20 mg to about 25 mg of magnesium stearate.

In some embodiments, the tablet consists essentially of:
about 300 mg of the compound of Formula Ia or Ib, or a pharmaceutically acceptable salt thereof, on a free acid basis;
about 0.5 mg of the compound of Formula IIa or IIb, or a solvate thereof, on a free base basis;
about 65 mg to about 75 mg of copovidone;
about 15 mg to about 25 mg of poloxamer 407;
about 615 mg to about 625 mg of mannitol;
about 330 mg to about 340 mg of microcrystalline cellulose;
about 115 mg to about 125 mg of croscarmellose sodium; and
about 20 mg to about 25 mg of magnesium stearate.

In some embodiments, the tablet consists essentially of:
about 300 mg of the compound of Formula Ia or Ib, or a pharmaceutically acceptable salt thereof, on a free acid basis;
about 0.5 mg of the compound of Formula IIa or IIb, or a solvate thereof, on a free base basis;
about 73.2 mg of copovidone;
about 19.95 mg of poloxamer 407;
about 621 mg of mannitol;
about 335.92 mg of microcrystalline cellulose;
about 120 mg of croscarmellose sodium; and
about 22.5 mg of magnesium stearate.

In some embodiments, the tablet consists essentially of:
about 200 mg to about 400 mg of the sodium salt of the compound of Formula Ia or Ib;
about 0.4 mg to about 0.6 mg of the monohydrate of the compound of Formula IIa or IIb;
about 50 mg to about 100 mg of copovidone;
about 5 mg to about 30 mg of poloxamer 407;
about 600 mg to about 700 mg of mannitol;
about 300 mg to about 350 mg of microcrystalline cellulose;
about 100 mg to about 150 mg of croscarmellose sodium; and
about 20 mg to about 25 mg of magnesium stearate.

In some embodiments, the tablet consists essentially of:
about 306.8 mg of the sodium salt of the compound of Formula Ia or Ib;
about 0.5 mg to about 0.6 mg of the monohydrate of the compound of Formula IIa or IIb;
about 65 mg to about 75 mg of copovidone;
about 15 mg to about 25 mg of poloxamer 407;
about 615 mg to about 625 mg of mannitol;
about 330 mg to about 340 mg of microcrystalline cellulose;
about 115 mg to about 125 mg of croscarmellose sodium; and
about 20 mg to about 25 mg of magnesium stearate.

In some embodiments, the tablet consists essentially of:
about 306.8 mg of the sodium salt of the compound of Formula Ia or Ib;
about 0.53 mg of the monohydrate of the compound of Formula IIa or IIb;
about 65 mg to about 75 mg of copovidone;
about 15 mg to about 25 mg of poloxamer 407;
about 615 mg to about 625 mg of mannitol;
about 330 mg to about 340 mg of microcrystalline cellulose;
about 115 mg to about 125 mg of croscarmellose sodium; and
about 20 mg to about 25 mg of magnesium stearate.

In some embodiments, the tablet consists essentially of:
about 306.8 mg of the sodium salt of the compound of Formula Ia or Ib;
about 0.53 mg of the monohydrate of the compound of Formula IIa or IIb;
about 73.2 mg of copovidone;
about 19.95 mg of poloxamer 407;
about 621 mg of mannitol;
about 335.92 mg of microcrystalline cellulose;
about 120 mg of croscarmellose sodium; and
about 22.5 mg of magnesium stearate.

In some embodiments, the tablet consists essentially of:

about 200 mg to about 400 mg of the compound of Formula Ia or Ib, or a pharmaceutically acceptable salt thereof, on a free acid basis;
about 0.4 mg to about 0.6 mg of the compound of Formula IIa or IIb, or a solvate thereof, on a free base basis;
about 50 mg to about 100 mg of copovidone;
about 5 mg to about 30 mg of poloxamer 407;
about 600 mg to about 700 mg of mannitol;
about 300 mg to about 350 mg of microcrystalline cellulose;
about 100 mg to about 150 mg of croscarmellose sodium;
about 20 mg to about 25 mg of magnesium stearate; and
an outer film coat.

In some embodiments, the tablet consists essentially of:
about 300 mg of the compound of Formula Ia or Ib, or a pharmaceutically acceptable salt thereof, on a free acid basis;
about 0.5 mg of the compound of Formula IIa or IIb, or a solvate thereof, on a free base basis;
about 65 mg to about 75 mg of copovidone;
about 15 mg to about 25 mg of poloxamer 407;
about 615 mg to about 625 mg of mannitol;
about 330 mg to about 340 mg of microcrystalline cellulose;
about 115 mg to about 125 mg of croscarmellose sodium;
about 20 mg to about 25 mg of magnesium stearate; and
an outer film coat.

In some embodiments, the tablet consists essentially of:
about 300 mg of the compound of Formula Ia or Ib, or a pharmaceutically acceptable salt thereof, on a free acid basis;
about 0.5 mg of the compound of Formula IIa or IIb, or a solvate thereof, on a free base basis, on a free base basis;
about 73.2 mg of copovidone;
about 19.95 mg of poloxamer 407;
about 621 mg of mannitol;
about 335.92 mg of microcrystalline cellulose;
about 120 mg of croscarmellose sodium;
about 22.5 mg of magnesium stearate; and
an outer film coat.

In some embodiments, the tablet consists essentially of:
about 200 mg to about 400 mg of the sodium salt of the compound of Formula Ia or Ib;
about 0.4 mg to about 0.6 mg of the monohydrate of the compound of Formula IIa or IIb;
about 50 mg to about 100 mg of copovidone;
about 5 mg to about 30 mg of poloxamer 407;
about 600 mg to about 700 mg of mannitol;
about 300 mg to about 350 mg of microcrystalline cellulose;
about 100 mg to about 150 mg of croscarmellose sodium;
about 20 mg to about 25 mg of magnesium stearate; and
an outer film coat.

In some embodiments, the tablet consists essentially of:
about 306.8 mg of the sodium salt of the compound of Formula Ia or Ib;
about 0.5 mg to about 0.6 mg of the monohydrate of the compound of Formula IIa or IIb;
about 65 mg to about 75 mg of copovidone;
about 15 mg to about 25 mg of poloxamer 407;
about 615 mg to about 625 mg of mannitol;
about 330 mg to about 340 mg of microcrystalline cellulose;
about 115 mg to about 125 mg of croscarmellose sodium;
about 20 mg to about 25 mg of magnesium stearate; and
an outer film coat.

In some embodiments, the tablet consists essentially of:
about 306.8 mg of the sodium salt of the compound of Formula Ia or Ib;
about 0.53 mg of the monohydrate of the compound of Formula IIa or IIb;
about 65 mg to about 75 mg of copovidone;
about 15 mg to about 25 mg of poloxamer 407;
about 615 mg to about 625 mg of mannitol;
about 330 mg to about 340 mg of microcrystalline cellulose;
about 115 mg to about 125 mg of croscarmellose sodium; and
about 20 mg to about 25 mg of magnesium stearate.

In some embodiments, the tablet consists essentially of:
about 306.8 mg of the sodium salt of the compound of Formula Ia or Ib;
about 0.53 mg of the monohydrate of the compound of Formula IIa or IIb;
about 73.2 mg of copovidone;
about 19.95 mg of poloxamer 407;
about 621 mg of mannitol;
about 335.92 mg of microcrystalline cellulose;
about 120 mg of croscarmellose sodium;
about 22.5 mg of magnesium stearate; and
an outer film coat.

In some embodiments, the tablet consists of:
about 200 mg to about 400 mg of the compound of Formula Ia or Ib, or a pharmaceutically acceptable salt thereof, on a free acid basis;
about 0.4 mg to about 0.6 mg of the compound of Formula IIa or IIb, or a solvate thereof, on a free base basis;
about 50 mg to about 100 mg of copovidone;
about 5 mg to about 30 mg of poloxamer 407;
about 600 mg to about 700 mg of mannitol;
about 300 mg to about 350 mg of microcrystalline cellulose;
about 100 mg to about 150 mg of croscarmellose sodium; and
about 20 mg to about 25 mg of magnesium stearate.

In some embodiments, the tablet consists of:
about 300 mg of the compound of Formula Ia or Ib, or a pharmaceutically acceptable salt thereof, on a free acid basis;
about 0.5 mg of the compound of Formula IIa or IIb, or a solvate thereof, on a free base basis;
about 65 mg to about 75 mg of copovidone;
about 15 mg to about 25 mg of poloxamer 407;
about 615 mg to about 625 mg of mannitol;
about 330 mg to about 340 mg of microcrystalline cellulose;
about 115 mg to about 125 mg of croscarmellose sodium; and
about 20 mg to about 25 mg of magnesium stearate.

In some embodiments, the tablet consists of:
about 300 mg of the compound of Formula Ia or Ib, or a pharmaceutically acceptable salt thereof, on a free acid basis;
about 0.5 mg of the compound of Formula IIa or IIb, or a solvate thereof, on a free base basis;
about 73.2 mg of copovidone;
about 19.95 mg of poloxamer 407;
about 621 mg of mannitol;
about 335.92 mg of microcrystalline cellulose;
about 120 mg of croscarmellose sodium; and
about 22.5 mg of magnesium stearate.

In some embodiments, the tablet consists of:
about 200 mg to about 400 mg of the sodium salt of the compound of Formula Ia or Ib;
about 0.4 mg to about 0.6 mg of the monohydrate of the compound of Formula IIa or IIb;
about 50 mg to about 100 mg of copovidone;
about 5 mg to about 30 mg of poloxamer 407;
about 600 mg to about 700 mg of mannitol;
about 300 mg to about 350 mg of microcrystalline cellulose;
about 100 mg to about 150 mg of croscarmellose sodium; and
about 20 mg to about 25 mg of magnesium stearate.

In some embodiments, the tablet consists of:
about 306.8 mg of the sodium salt of the compound of Formula Ia or Ib;
about 0.5 mg to about 0.6 mg of the monohydrate of the compound of Formula IIa or IIb;
about 65 mg to about 75 mg of copovidone;
about 15 mg to about 25 mg of poloxamer 407;
about 615 mg to about 625 mg of mannitol;
about 330 mg to about 340 mg of microcrystalline cellulose;
about 115 mg to about 125 mg of croscarmellose sodium; and
about 20 mg to about 25 mg of magnesium stearate.

In some embodiments, the tablet consists of:
about 306.8 mg of the sodium salt of the compound of Formula Ia or Ib;
about 0.53 mg of the monohydrate of the compound of Formula IIa or IIb;
about 65 mg to about 75 mg of copovidone;
about 15 mg to about 25 mg of poloxamer 407;
about 615 mg to about 625 mg of mannitol;
about 330 mg to about 340 mg of microcrystalline cellulose;
about 115 mg to about 125 mg of croscarmellose sodium; and
about 20 mg to about 25 mg of magnesium stearate.

In some embodiments, the tablet consists of:
about 306.8 mg of the sodium salt of the compound of Formula Ia or Ib;
about 0.53 mg of the monohydrate of the compound of Formula IIa or IIb;
about 73.2 mg of copovidone;
about 19.95 mg of poloxamer 407;
about 621 mg of mannitol;
about 335.92 mg of microcrystalline cellulose;
about 120 mg of croscarmellose sodium; and
about 22.5 mg of magnesium stearate.

In some embodiments, the tablet consists of:
about 200 mg to about 400 mg of the compound of Formula Ia or Ib, or a pharmaceutically acceptable salt thereof, on a free acid basis;
about 0.4 mg to about 0.6 mg of the compound of Formula IIa or IIb, or a solvate thereof, on a free base basis;
about 50 mg to about 100 mg of copovidone;
about 5 mg to about 30 mg of poloxamer 407;
about 600 mg to about 700 mg of mannitol;
about 300 mg to about 350 mg of microcrystalline cellulose;
about 100 mg to about 150 mg of croscarmellose sodium;
about 20 mg to about 25 mg of magnesium stearate; and
an outer film coat.

In some embodiments, the tablet consists of:
about 300 mg of the compound of Formula Ia or Ib, or a pharmaceutically acceptable salt thereof, on a free acid basis;
about 0.5 mg of the compound of Formula IIa or IIb, or a solvate thereof, on a free base basis;
about 65 mg to about 75 mg of copovidone;
about 15 mg to about 25 mg of poloxamer 407;
about 615 mg to about 625 mg of mannitol;
about 330 mg to about 340 mg of microcrystalline cellulose;
about 115 mg to about 125 mg of croscarmellose sodium;
about 20 mg to about 25 mg of magnesium stearate; and
an outer film coat.

In some embodiments, the tablet consists of:
about 300 mg of the compound of Formula Ia or Ib, or a pharmaceutically acceptable salt thereof, on a free acid basis;
about 0.5 mg of the compound of Formula IIa or IIb, or a solvate thereof, on a free base basis, on a free base basis;
about 73.2 mg of copovidone;
about 19.95 mg of poloxamer 407;
about 621 mg of mannitol;
about 335.92 mg of microcrystalline cellulose;
about 120 mg of croscarmellose sodium;
about 22.5 mg of magnesium stearate; and
an outer film coat.

In some embodiments, the tablet consists of:
about 200 mg to about 400 mg of the sodium salt of the compound of Formula Ia or Ib;
about 0.4 mg to about 0.6 mg of the monohydrate of the compound of Formula IIa or IIb;
about 50 mg to about 100 mg of copovidone;
about 5 mg to about 30 mg of poloxamer 407;
about 600 mg to about 700 mg of mannitol;
about 300 mg to about 350 mg of microcrystalline cellulose;
about 100 mg to about 150 mg of croscarmellose sodium;
about 20 mg to about 25 mg of magnesium stearate; and
an outer film coat.

In some embodiments, the tablet consists of:
about 306.8 mg of the sodium salt of the compound of Formula Ia or Ib;
about 0.5 mg to about 0.6 mg of the monohydrate of the compound of Formula IIa or IIb;
about 65 mg to about 75 mg of copovidone;
about 15 mg to about 25 mg of poloxamer 407;
about 615 mg to about 625 mg of mannitol;
about 330 mg to about 340 mg of microcrystalline cellulose;
about 115 mg to about 125 mg of croscarmellose sodium;
about 20 mg to about 25 mg of magnesium stearate; and
an outer film coat.

In some embodiments, the tablet consists of:
about 306.8 mg of the sodium salt of the compound of Formula Ia or Ib;
about 0.53 mg of the monohydrate of the compound of Formula IIa or IIb;
about 65 mg to about 75 mg of copovidone;
about 15 mg to about 25 mg of poloxamer 407;
about 615 mg to about 625 mg of mannitol;
about 330 mg to about 340 mg of microcrystalline cellulose;
about 115 mg to about 125 mg of croscarmellose sodium; and
about 20 mg to about 25 mg of magnesium stearate.

In some embodiments, the tablet consists of:
about 306.8 mg of the sodium salt of the compound of Formula Ia or Ib;
about 0.53 mg of the monohydrate of the compound of Formula IIa or IIb;
about 73.2 mg of copovidone;
about 19.95 mg of poloxamer 407;
about 621 mg of mannitol;
about 335.92 mg of microcrystalline cellulose;
about 120 mg of croscarmellose sodium;
about 22.5 mg of magnesium stearate; and
an outer film coat.

In some embodiments, the tablet comprises:
about 200 mg to about 400 mg of the compound of Formula Ia or Ib, or a pharmaceutically acceptable salt thereof, on a free acid basis;
about 0.4 mg to about 0.6 mg of the compound of Formula IIa or IIb, or a solvate thereof, on a free base basis;
about 50 mg to about 100 mg of copovidone;
about 5 mg to about 30 mg of poloxamer 407;
about 600 mg to about 700 mg of mannitol;
about 300 mg to about 350 mg of microcrystalline cellulose;
about 100 mg to about 150 mg of croscarmellose sodium; and
about 20 mg to about 25 mg of magnesium stearate.

In some embodiments, the tablet comprises:
about 300 mg of the compound of Formula Ia or Ib, or a pharmaceutically acceptable salt thereof, on a free acid basis;
about 0.5 mg of the compound of Formula IIa or IIb, or a solvate thereof, on a free base basis;
about 65 mg to about 75 mg of copovidone;
about 15 mg to about 25 mg of poloxamer 407;
about 615 mg to about 625 mg of mannitol;
about 330 mg to about 340 mg of microcrystalline cellulose;
about 115 mg to about 125 mg of croscarmellose sodium; and
about 20 mg to about 25 mg of magnesium stearate.

In some embodiments, the tablet comprises:
about 300 mg of the compound of Formula Ia or Ib, or a pharmaceutically acceptable salt thereof, on a free acid basis;
about 0.5 mg of the compound of Formula IIa or IIb, or a solvate thereof, on a free base basis;
about 73.2 mg of copovidone;
about 19.95 mg of poloxamer 407;
about 621 mg of mannitol;
about 335.92 mg of microcrystalline cellulose;
about 120 mg of croscarmellose sodium; and
about 22.5 mg of magnesium stearate.

In some embodiments, the tablet comprises:
about 200 mg to about 400 mg of the sodium salt of the compound of Formula Ia or Ib;
about 0.4 mg to about 0.6 mg of the monohydrate of the compound of Formula IIa or IIb;
about 50 mg to about 100 mg of copovidone;
about 5 mg to about 30 mg of poloxamer 407;
about 600 mg to about 700 mg of mannitol;
about 300 mg to about 350 mg of microcrystalline cellulose;
about 100 mg to about 150 mg of croscarmellose sodium; and
about 20 mg to about 25 mg of magnesium stearate.

In some embodiments, the tablet comprises:
about 306.8 mg of the sodium salt of the compound of Formula Ia or Ib;
about 0.5 mg to about 0.6 mg of the monohydrate of the compound of Formula IIa or IIb;
about 65 mg to about 75 mg of copovidone;
about 15 mg to about 25 mg of poloxamer 407;
about 615 mg to about 625 mg of mannitol;
about 330 mg to about 340 mg of microcrystalline cellulose;
about 115 mg to about 125 mg of croscarmellose sodium; and
about 20 mg to about 25 mg of magnesium stearate.

In some embodiments, the tablet comprises:
about 306.8 mg of the sodium salt of the compound of Formula Ia or Ib;
about 0.53 mg of the monohydrate of the compound of Formula IIa or IIb;
about 65 mg to about 75 mg of copovidone;
about 15 mg to about 25 mg of poloxamer 407;
about 615 mg to about 625 mg of mannitol;
about 330 mg to about 340 mg of microcrystalline cellulose;
about 115 mg to about 125 mg of croscarmellose sodium; and
about 20 mg to about 25 mg of magnesium stearate.

In some embodiments, the tablet comprises:
about 306.8 mg of the sodium salt of the compound of Formula Ia or Ib;
about 0.53 mg of the monohydrate of the compound of Formula IIa or IIb;
about 73.2 mg of copovidone;
about 19.95 mg of poloxamer 407;
about 621 mg of mannitol;
about 335.92 mg of microcrystalline cellulose;
about 120 mg of croscarmellose sodium; and
about 22.5 mg of magnesium stearate.

In some embodiments, the tablet comprises:
about 200 mg to about 400 mg of the compound of Formula Ia or Ib, or a pharmaceutically acceptable salt thereof, on a free acid basis;
about 0.4 mg to about 0.6 mg of the compound of Formula IIa or IIb, or a solvate thereof, on a free base basis;
about 50 mg to about 100 mg of copovidone;
about 5 mg to about 30 mg of poloxamer 407;
about 600 mg to about 700 mg of mannitol;
about 300 mg to about 350 mg of microcrystalline cellulose;
about 100 mg to about 150 mg of croscarmellose sodium;
about 20 mg to about 25 mg of magnesium stearate; and
an outer film coat.

In some embodiments, the tablet comprises:
about 300 mg of the compound of Formula Ia or Ib, or a pharmaceutically acceptable salt thereof, on a free acid basis;
about 0.5 mg of the compound of Formula IIa or IIb, or a solvate thereof, on a free base basis;
about 65 mg to about 75 mg of copovidone;
about 15 mg to about 25 mg of poloxamer 407;
about 615 mg to about 625 mg of mannitol;
about 330 mg to about 340 mg of microcrystalline cellulose;
about 115 mg to about 125 mg of croscarmellose sodium;
about 20 mg to about 25 mg of magnesium stearate; and
an outer film coat.

In some embodiments, the tablet comprises:
about 300 mg of the compound of Formula Ia or Ib, or a pharmaceutically acceptable salt thereof, on a free acid basis;
about 0.5 mg of the compound of Formula IIa or IIb, or a solvate thereof, on a free base basis, on a free base basis;
about 73.2 mg of copovidone;
about 19.95 mg of poloxamer 407;
about 621 mg of mannitol;
about 335.92 mg of microcrystalline cellulose;
about 120 mg of croscarmellose sodium;
about 22.5 mg of magnesium stearate; and
an outer film coat.

In some embodiments, the tablet comprises:
about 200 mg to about 400 mg of the sodium salt of the compound of Formula Ia or Ib;
about 0.4 mg to about 0.6 mg of the monohydrate of the compound of Formula IIa or IIb;
about 50 mg to about 100 mg of copovidone;
about 5 mg to about 30 mg of poloxamer 407;
about 600 mg to about 700 mg of mannitol;
about 300 mg to about 350 mg of microcrystalline cellulose;
about 100 mg to about 150 mg of croscarmellose sodium;
about 20 mg to about 25 mg of magnesium stearate; and
an outer film coat.

In some embodiments, the tablet comprises:
about 306.8 mg of the sodium salt of the compound of Formula Ia or Ib;
about 0.5 mg to about 0.6 mg of the monohydrate of the compound of Formula IIa or IIb;
about 65 mg to about 75 mg of copovidone;
about 15 mg to about 25 mg of poloxamer 407;
about 615 mg to about 625 mg of mannitol;
about 330 mg to about 340 mg of microcrystalline cellulose;
about 115 mg to about 125 mg of croscarmellose sodium;
about 20 mg to about 25 mg of magnesium stearate; and
an outer film coat.

In some embodiments, the tablet comprises:
about 306.8 mg of the sodium salt of the compound of Formula Ia or Ib;
about 0.53 mg of the monohydrate of the compound of Formula IIa or IIb;
about 65 mg to about 75 mg of copovidone;
about 15 mg to about 25 mg of poloxamer 407;
about 615 mg to about 625 mg of mannitol;
about 330 mg to about 340 mg of microcrystalline cellulose;
about 115 mg to about 125 mg of croscarmellose sodium; and
about 20 mg to about 25 mg of magnesium stearate.

In some embodiments, the tablet comprises:
about 306.8 mg of the sodium salt of the compound of Formula Ia or Ib;
about 0.53 mg of the monohydrate of the compound of Formula IIa or IIb;
about 73.2 mg of copovidone;
about 19.95 mg of poloxamer 407;
about 621 mg of mannitol;
about 335.92 mg of microcrystalline cellulose;
about 120 mg of croscarmellose sodium;
about 22.5 mg of magnesium stearate; and
an outer film coat.

In some embodiments, the tablet consists essentially of:
about 200 mg to about 400 mg of the compound of Formula Ia or Ib, or a pharmaceutically acceptable salt thereof, on a free acid basis;
about 0.4 mg to about 0.6 mg of the compound of Formula IIa or IIb, or a solvate thereof, on a free base basis;
about 50 mg to about 100 mg of copovidone;
about 5 mg to about 30 mg of poloxamer 407;
about 600 mg to about 700 mg of mannitol;
about 300 mg to about 350 mg of microcrystalline cellulose;
about 100 mg to about 150 mg of croscarmellose sodium; and
about 20 mg to about 25 mg of magnesium stearate.

In some embodiments, the tablet consists essentially of:
about 300 mg of the compound of Formula Ia or Ib, or a pharmaceutically acceptable salt thereof, on a free acid basis;
about 0.5 mg of the compound of Formula IIa or IIb, or a solvate thereof, on a free base basis;
about 65 mg to about 75 mg of copovidone;
about 15 mg to about 25 mg of poloxamer 407;
about 615 mg to about 625 mg of mannitol;
about 330 mg to about 340 mg of microcrystalline cellulose;
about 115 mg to about 125 mg of croscarmellose sodium; and
about 20 mg to about 25 mg of magnesium stearate.

In some embodiments, the tablet consists essentially of:
about 300 mg of the compound of Formula Ia or Ib, or a pharmaceutically acceptable salt thereof, on a free acid basis;
about 0.5 mg of the compound of Formula IIa or IIb, or a solvate thereof, on a free base basis;
about 73.2 mg of copovidone;
about 19.95 mg of poloxamer 407;
about 621 mg of mannitol;
about 335.92 mg of microcrystalline cellulose;
about 120 mg of croscarmellose sodium; and
about 22.5 mg of magnesium stearate.

In some embodiments, the tablet consists essentially of:
about 200 mg to about 400 mg of the sodium salt of the compound of Formula Ia or Ib;
about 0.4 mg to about 0.6 mg of the monohydrate of the compound of Formula IIa or IIb;
about 50 mg to about 100 mg of copovidone;
about 5 mg to about 30 mg of poloxamer 407;
about 600 mg to about 700 mg of mannitol;
about 300 mg to about 350 mg of microcrystalline cellulose;
about 100 mg to about 150 mg of croscarmellose sodium; and
about 20 mg to about 25 mg of magnesium stearate.

In some embodiments, the tablet consists essentially of:
about 306.8 mg of the sodium salt of the compound of Formula Ia or Ib;
about 0.5 mg to about 0.6 mg of the monohydrate of the compound of Formula IIa or IIb;
about 65 mg to about 75 mg of copovidone;
about 15 mg to about 25 mg of poloxamer 407;
about 615 mg to about 625 mg of mannitol;
about 330 mg to about 340 mg of microcrystalline cellulose;
about 115 mg to about 125 mg of croscarmellose sodium; and
about 20 mg to about 25 mg of magnesium stearate.

In some embodiments, the tablet consists essentially of:
about 306.8 mg of the sodium salt of the compound of Formula Ia or Ib;
about 0.53 mg of the monohydrate of the compound of Formula IIa or IIb;
about 65 mg to about 75 mg of copovidone;
about 15 mg to about 25 mg of poloxamer 407;
about 615 mg to about 625 mg of mannitol;
about 330 mg to about 340 mg of microcrystalline cellulose;
about 115 mg to about 125 mg of croscarmellose sodium; and
about 20 mg to about 25 mg of magnesium stearate.

In some embodiments, the tablet consists essentially of:
about 306.8 mg of the sodium salt of the compound of Formula Ia or Ib;
about 0.53 mg of the monohydrate of the compound of Formula IIa or IIb;
about 73.2 mg of copovidone;
about 19.95 mg of poloxamer 407;
about 621 mg of mannitol;
about 335.92 mg of microcrystalline cellulose;
about 120 mg of croscarmellose sodium; and
about 22.5 mg of magnesium stearate.

In some embodiments, the tablet consists essentially of:
about 200 mg to about 400 mg of the compound of Formula Ia or Ib, or a pharmaceutically acceptable salt thereof, on a free acid basis;
about 0.4 mg to about 0.6 mg of the compound of Formula IIa or IIb, or a solvate thereof, on a free base basis;
about 50 mg to about 100 mg of copovidone;
about 5 mg to about 30 mg of poloxamer 407;
about 600 mg to about 700 mg of mannitol;
about 300 mg to about 350 mg of microcrystalline cellulose;
about 100 mg to about 150 mg of croscarmellose sodium;
about 20 mg to about 25 mg of magnesium stearate; and
an outer film coat.

In some embodiments, the tablet consists essentially of:
about 300 mg of the compound of Formula Ia or Ib, or a pharmaceutically acceptable salt thereof, on a free acid basis;
about 0.5 mg of the compound of Formula IIa or IIb, or a solvate thereof, on a free base basis;
about 65 mg to about 75 mg of copovidone;
about 15 mg to about 25 mg of poloxamer 407;
about 615 mg to about 625 mg of mannitol;
about 330 mg to about 340 mg of microcrystalline cellulose;
about 115 mg to about 125 mg of croscarmellose sodium;
about 20 mg to about 25 mg of magnesium stearate; and
an outer film coat.

In some embodiments, the tablet consists essentially of:
about 300 mg of the compound of Formula Ia or Ib, or a pharmaceutically acceptable salt thereof, on a free acid basis;
about 0.5 mg of the compound of Formula IIa or IIb, or a solvate thereof, on a free base basis, on a free base basis;
about 73.2 mg of copovidone;
about 19.95 mg of poloxamer 407;
about 621 mg of mannitol;
about 335.92 mg of microcrystalline cellulose;
about 120 mg of croscarmellose sodium;
about 22.5 mg of magnesium stearate; and
an outer film coat.

In some embodiments, the tablet consists essentially of:
about 200 mg to about 400 mg of the sodium salt of the compound of Formula Ia or Ib;
about 0.4 mg to about 0.6 mg of the monohydrate of the compound of Formula IIa or IIb;
about 50 mg to about 100 mg of copovidone;
about 5 mg to about 30 mg of poloxamer 407;
about 600 mg to about 700 mg of mannitol;
about 300 mg to about 350 mg of microcrystalline cellulose;
about 100 mg to about 150 mg of croscarmellose sodium;
about 20 mg to about 25 mg of magnesium stearate; and
an outer film coat.

In some embodiments, the tablet consists essentially of:
about 306.8 mg of the sodium salt of the compound of Formula Ia or Ib;
about 0.5 mg to about 0.6 mg of the monohydrate of the compound of Formula IIa or IIb;
about 65 mg to about 75 mg of copovidone;
about 15 mg to about 25 mg of poloxamer 407;
about 615 mg to about 625 mg of mannitol;
about 330 mg to about 340 mg of microcrystalline cellulose;
about 115 mg to about 125 mg of croscarmellose sodium;
about 20 mg to about 25 mg of magnesium stearate; and
an outer film coat.

In some embodiments, the tablet consists essentially of:
about 306.8 mg of the sodium salt of the compound of Formula Ia or Ib;
about 0.53 mg of the monohydrate of the compound of Formula IIa or IIb;
about 65 mg to about 75 mg of copovidone;
about 15 mg to about 25 mg of poloxamer 407;
about 615 mg to about 625 mg of mannitol;
about 330 mg to about 340 mg of microcrystalline cellulose;
about 115 mg to about 125 mg of croscarmellose sodium; and
about 20 mg to about 25 mg of magnesium stearate.

In some embodiments, the tablet consists essentially of:
about 306.8 mg of the sodium salt of the compound of Formula Ia or Ib;
about 0.53 mg of the monohydrate of the compound of Formula IIa or IIb;
about 73.2 mg of copovidone;
about 19.95 mg of poloxamer 407;
about 621 mg of mannitol;
about 335.92 mg of microcrystalline cellulose;
about 120 mg of croscarmellose sodium;
about 22.5 mg of magnesium stearate; and
an outer film coat.

In some embodiments, the tablet consists of:
about 200 mg to about 400 mg of the compound of Formula Ia or Ib, or a pharmaceutically acceptable salt thereof, on a free acid basis;
about 0.4 mg to about 0.6 mg of the compound of Formula IIa or IIb, or a solvate thereof, on a free base basis;
about 50 mg to about 100 mg of copovidone;
about 5 mg to about 30 mg of poloxamer 407;
about 600 mg to about 700 mg of mannitol;
about 300 mg to about 350 mg of microcrystalline cellulose;
about 100 mg to about 150 mg of croscarmellose sodium; and
about 20 mg to about 25 mg of magnesium stearate.

In some embodiments, the tablet consists of:
about 300 mg of the compound of Formula Ia or Ib, or a pharmaceutically acceptable salt thereof, on a free acid basis;
about 0.5 mg of the compound of Formula IIa or IIb, or a solvate thereof, on a free base basis;
about 65 mg to about 75 mg of copovidone;
about 15 mg to about 25 mg of poloxamer 407;
about 615 mg to about 625 mg of mannitol;
about 330 mg to about 340 mg of microcrystalline cellulose;
about 115 mg to about 125 mg of croscarmellose sodium; and
about 20 mg to about 25 mg of magnesium stearate.

In some embodiments, the tablet consists of:
about 300 mg of the compound of Formula Ia or Ib, or a pharmaceutically acceptable salt thereof, on a free acid basis;
about 0.5 mg of the compound of Formula IIa or IIb, or a solvate thereof, on a free base basis;
about 73.2 mg of copovidone;
about 19.95 mg of poloxamer 407;
about 621 mg of mannitol;
about 335.92 mg of microcrystalline cellulose;
about 120 mg of croscarmellose sodium; and
about 22.5 mg of magnesium stearate.

In some embodiments, the tablet consists of:
about 200 mg to about 400 mg of the sodium salt of the compound of Formula Ia or Ib;
about 0.4 mg to about 0.6 mg of the monohydrate of the compound of Formula IIa or IIb;
about 50 mg to about 100 mg of copovidone;
about 5 mg to about 30 mg of poloxamer 407;
about 600 mg to about 700 mg of mannitol;
about 300 mg to about 350 mg of microcrystalline cellulose;
about 100 mg to about 150 mg of croscarmellose sodium; and
about 20 mg to about 25 mg of magnesium stearate.

In some embodiments, the tablet consists of:
about 306.8 mg of the sodium salt of the compound of Formula Ia or Ib;
about 0.5 mg to about 0.6 mg of the monohydrate of the compound of Formula IIa or IIb;
about 65 mg to about 75 mg of copovidone;
about 15 mg to about 25 mg of poloxamer 407;
about 615 mg to about 625 mg of mannitol;
about 330 mg to about 340 mg of microcrystalline cellulose;
about 115 mg to about 125 mg of croscarmellose sodium; and
about 20 mg to about 25 mg of magnesium stearate.

In some embodiments, the tablet consists of:
about 306.8 mg of the sodium salt of the compound of Formula Ia or Ib;
about 0.53 mg of the monohydrate of the compound of Formula IIa or IIb;
about 65 mg to about 75 mg of copovidone;
about 15 mg to about 25 mg of poloxamer 407;
about 615 mg to about 625 mg of mannitol;
about 330 mg to about 340 mg of microcrystalline cellulose;
about 115 mg to about 125 mg of croscarmellose sodium; and
about 20 mg to about 25 mg of magnesium stearate.

In some embodiments, the tablet consists of:
about 306.8 mg of the sodium salt of the compound of Formula Ia or Ib;
about 0.53 mg of the monohydrate of the compound of Formula IIa or IIb;
about 73.2 mg of copovidone;
about 19.95 mg of poloxamer 407;
about 621 mg of mannitol;
about 335.92 mg of microcrystalline cellulose;
about 120 mg of croscarmellose sodium; and
about 22.5 mg of magnesium stearate.

In some embodiments, the tablet consists of:
about 200 mg to about 400 mg of the compound of Formula Ia or Ib, or a pharmaceutically acceptable salt thereof, on a free acid basis;
about 0.4 mg to about 0.6 mg of the compound of Formula IIa or IIb, or a solvate thereof, on a free base basis;
about 50 mg to about 100 mg of copovidone;
about 5 mg to about 30 mg of poloxamer 407;
about 600 mg to about 700 mg of mannitol;
about 300 mg to about 350 mg of microcrystalline cellulose;
about 100 mg to about 150 mg of croscarmellose sodium;
about 20 mg to about 25 mg of magnesium stearate; and
an outer film coat.

In some embodiments, the tablet consists of:
about 300 mg of the compound of Formula Ia or Ib, or a pharmaceutically acceptable salt thereof, on a free acid basis;
about 0.5 mg of the compound of Formula IIa or IIb, or a solvate thereof, on a free base basis;
about 65 mg to about 75 mg of copovidone;
about 15 mg to about 25 mg of poloxamer 407;
about 615 mg to about 625 mg of mannitol;
about 330 mg to about 340 mg of microcrystalline cellulose;
about 115 mg to about 125 mg of croscarmellose sodium;
about 20 mg to about 25 mg of magnesium stearate; and
an outer film coat.

In some embodiments, the tablet consists of:
about 300 mg of the compound of Formula Ia or Ib, or a pharmaceutically acceptable salt thereof, on a free acid basis;
about 0.5 mg of the compound of Formula IIa or IIb, or a solvate thereof, on a free base basis, on a free base basis;
about 73.2 mg of copovidone;
about 19.95 mg of poloxamer 407;
about 621 mg of mannitol;
about 335.92 mg of microcrystalline cellulose;
about 120 mg of croscarmellose sodium;
about 22.5 mg of magnesium stearate; and
an outer film coat.

In some embodiments, the tablet consists of:
about 200 mg to about 400 mg of the sodium salt of the compound of Formula Ia or Ib;
about 0.4 mg to about 0.6 mg of the monohydrate of the compound of Formula IIa or IIb;
about 50 mg to about 100 mg of copovidone;
about 5 mg to about 30 mg of poloxamer 407;
about 600 mg to about 700 mg of mannitol;
about 300 mg to about 350 mg of microcrystalline cellulose;
about 100 mg to about 150 mg of croscarmellose sodium;
about 20 mg to about 25 mg of magnesium stearate; and
an outer film coat.

In some embodiments, the tablet consists of:
about 306.8 mg of the sodium salt of the compound of Formula Ia or Ib;
about 0.5 mg to about 0.6 mg of the monohydrate of the compound of Formula IIa or IIb;
about 65 mg to about 75 mg of copovidone;
about 15 mg to about 25 mg of poloxamer 407;
about 615 mg to about 625 mg of mannitol;
about 330 mg to about 340 mg of microcrystalline cellulose;
about 115 mg to about 125 mg of croscarmellose sodium;
about 20 mg to about 25 mg of magnesium stearate; and
an outer film coat.

In some embodiments, the tablet consists of:
about 306.8 mg of the sodium salt of the compound of Formula Ia or Ib;
about 0.53 mg of the monohydrate of the compound of Formula IIa or IIb;
about 65 mg to about 75 mg of copovidone;
about 15 mg to about 25 mg of poloxamer 407;
about 615 mg to about 625 mg of mannitol;
about 330 mg to about 340 mg of microcrystalline cellulose;
about 115 mg to about 125 mg of croscarmellose sodium; and
about 20 mg to about 25 mg of magnesium stearate.

In some embodiments, the tablet consists of:
about 306.8 mg of the sodium salt of the compound of Formula Ia or Ib;
about 0.53 mg of the monohydrate of the compound of Formula IIa or IIb;
about 73.2 mg of copovidone;
about 19.95 mg of poloxamer 407;
about 621 mg of mannitol;
about 335.92 mg of microcrystalline cellulose;
about 120 mg of croscarmellose sodium;
about 22.5 mg of magnesium stearate; and
an outer film coat.

The tablets disclosed herein can be prepared by methodologies known in the pharmaceutical art. The terms “effective amount” or “therapeutically effective amount” refer to an amount of the compound of Formula Ia or Ib, or a pharmaceutically acceptable salt, co-crystal, solvate, or combination thereof, and the compound of Formula IIa or IIb, or a pharmaceutically acceptable salt, co-crystal, solvate, or combination thereof, which when administered to a patient in need thereof, is sufficient to effect treating an HIV infection as described herein. Such an amount would be sufficient to elicit the biological or medical response of a tissue system, or patient that is sought by a researcher or clinician. The amount of the compound of Formula Ia or Ib, or a pharmaceutically acceptable salt, co-crystal, solvate, or combination thereof, and the compound of Formula IIa or IIb, or a pharmaceutically acceptable salt, co-crystal, solvate, or combination thereof, which constitutes a therapeutically effective amount will vary depending on such factors as the compound, salt, free acid, or composition used for administration, the time of administration, the route of administration, the rate of excretion of the compound, the duration of the treatment, the type of disease-state or disorder being treated and its severity, and the age, body weight, general health, sex and diet of the patient. Such a therapeutically effective amount can be determined routinely by one of ordinary skill in the art having regard to their own knowledge, the state of the art, and this disclosure.

### Methods of Use

In some embodiments, the methods provided herein comprise treating a human immunodeficiency virus (HIV) infection in the patient. In some embodiments, the methods provided herein comprise treating a HIV infection in the patient. In some embodiments, the HIV is HIV-1. In some embodiments, the HIV is HIV-2. In some embodiments, the HIV is HIV-1 and HIV-2.

As used herein, “HIV” or “Human Immunodeficiency Virus” refers to HIV-1 and/or to HIV-2.

The term “patient” is meant to refer to a human who is in need of therapeutic treatment for a viral infection, such as HIV infection.

Administration of the compound of Formula Ia or Ib, or a pharmaceutically acceptable salt, co-crystal, solvate, or combination thereof, and compound of Formula IIa or IIb, or a pharmaceutically acceptable salt, co-crystal, solvate, or combination thereof, can be carried out via any of the accepted modes of administration of agents for serving similar utilities. In some embodiments, the administration is oral administration.

In some embodiments, the methods provided herein further comprise administering one or more initiation (i.e., loading) doses of the tablet provided herein, for a first period of time.

In some embodiments, the first period of time is about one day to about 10 days. In some embodiments, the first period of time is one day. In some embodiments, the first period of time is two days.

In some embodiments, the initiation dosage provided herein comprises one or more oral administrations of a tablet provided herein.

In some embodiments, the initiation dosage is administered orally as one or two tablets provided herein (e.g., one or two tablets on day 1; one or two tablets on day 2; one or two tablets on day 1 and one or two tablets on day 2; and the like). In some embodiments, the oral administrations are administered as two tablets provided herein.

In some embodiments, the initiation dosage is administered orally as two tablets provided herein on day 1; and as two tablets provided herein on day 2.

In some embodiments, each tablet of the initiation dosage comprises:
about 26.6 w/w % to about 26.7 w/w % of the spray dried dispersion of the compound of Formula Ia or Ib, or a pharmaceutically acceptable salt, co-crystal, solvate, or combination thereof;
about 0.03 w/w % to about 0.04 w/w % of the compound of Formula IIa or IIb, or a pharmaceutically acceptable salt, co-crystal, solvate, or combination thereof; and
one or more pharmaceutically acceptable excipients.

In some embodiments, each tablet of the initiation dosage comprises:
about 26.67 w/w % of the spray dried dispersion of the compound of Formula Ia or Ib, or a pharmaceutically acceptable salt, co-crystal, solvate, or combination thereof;
about 0.0355 w/w % of the compound of Formula IIa or IIb, or a pharmaceutically acceptable salt, co-crystal, solvate, or combination thereof; and
one or more pharmaceutically acceptable excipients.

In some embodiments, each tablet of the initiation dosage comprises:
about 26.6 w/w % to about 26.7 w/w % of the spray dried dispersion of the compound of Formula Ia or Ib, or a pharmaceutically acceptable salt, co-crystal, solvate, or combination thereof;
about 0.03 w/w % to about 0.04 w/w % of the compound of Formula IIa or IIb, or a solvate thereof, and
one or more pharmaceutically acceptable excipients.

In some embodiments, each tablet of the initiation dosage comprises:
about 26.67 w/w % of the spray dried dispersion of the compound of Formula Ia or Ib, or a pharmaceutically acceptable salt, co-crystal, solvate, or combination thereof;
about 0.0355 w/w % of the compound of Formula IIa or IIb, or a solvate thereof, and
one or more pharmaceutically acceptable excipients.

In some embodiments, each tablet of the initiation dosage comprises:
about 26.6 w/w % to about 26.7 w/w % of the spray dried dispersion of the compound of Formula Ia or Ib, or a pharmaceutically acceptable salt, co-crystal, solvate, or combination thereof;
about 0.03 w/w % to about 0.04 w/w % of the monohydrate of the compound of Formula IIa or IIb; and
one or more pharmaceutically acceptable excipients.

In some embodiments, each tablet of the initiation dosage comprises:
about 26.67 w/w % of the spray dried dispersion of the compound of Formula Ia or Ib, or a pharmaceutically acceptable salt, co-crystal, solvate, or combination thereof;
about 0.0355 w/w % of the monohydrate of the compound of Formula IIa or IIb; and
one or more pharmaceutically acceptable excipients.

In some embodiments, each tablet of the initiation dosage comprises:
about 26.6 w/w % to about 26.7 w/w % of the spray dried dispersion of the compound of Formula Ia or Ib, or a pharmaceutically acceptable salt, co-crystal, solvate, or combination thereof;
about 0.03 w/w % to about 0.04 w/w % of the compound of Formula IIa or IIb, or a pharmaceutically acceptable salt, co-crystal, solvate, or combination thereof;
about 41 w/w % to about 42 w/w % of mannitol;
about 22.3 w/w % to about 22.4 w/w % of microcrystalline cellulose;
about 7.9 w/w % to about 8.1 w/w % of croscarmellose sodium; and
about 1.4 w/w % to about 1.6 w/w % of magnesium stearate.

In some embodiments, each tablet of the initiation dosage comprises:
about 26.67 w/w % of the compound of the spray dried dispersion of the compound of Formula Ia or Ib, or a pharmaceutically acceptable salt, co-crystal, solvate, or combination thereof;
about 0.0355 w/w % of the compound of Formula IIa or IIb, or a pharmaceutically acceptable salt, co-crystal, solvate, or combination thereof;
about 41.4 w/w % of mannitol;
about 22.3945 w/w % of microcrystalline cellulose;
about 8.0 w/w % of croscarmellose sodium; and
about 1.5 w/w % of magnesium stearate.

In some embodiments, each tablet of the initiation dosage comprises:
about 19 w/w % to about 21 w/w % of the compound of Formula Ia or Ib, or a pharmaceutically acceptable salt thereof;
about 0.03 w/w % to about 0.04 w/w % of the compound of Formula IIa or IIb, or a solvate thereof;
about 4 w/w % to about 6 w/w % of copovidone;
about 0.1 w/w % to about 2 w/w % of poloxamer 407;
about 40 w/w % to about 42 w/w % of mannitol;
about 22 w/w % to about 23 w/w % of microcrystalline cellulose;
about 7 w/w % to about 9 w/w % of croscarmellose sodium; and
about 1.0 w/w % to about 2.0 w/w % of magnesium stearate.

In some embodiments, each tablet of the initiation dosage comprises:
about 19.8 w/w % to about 20.8 w/w % of the compound of Formula Ia or Ib, or a pharmaceutically acceptable salt thereof;
about 0.03 w/w % to about 0.04 w/w % of the compound of Formula IIa or IIb, or a solvate thereof;
about 4 w/w % to about 5 w/w % of copovidone;
about 1 w/w % to about 2 w/w % of poloxamer 407;
about 41 w/w % to about 42 w/w % of mannitol;
about 22.3 w/w % to about 22.4 w/w % of microcrystalline cellulose;
about 7.9 w/w % to about 8.1 w/w % of croscarmellose sodium; and
about 1.4 w/w % to about 1.6 w/w % of magnesium stearate.

In some embodiments, each tablet of the initiation dosage comprises:
about 20.46 w/w % of the compound of Formula Ia or Ib, or a pharmaceutically acceptable salt thereof;
about 0.0355 w/w % of the compound of Formula IIa or IIb, or a solvate thereof;
about 4.88 w/w % of copovidone;
about 1.33 w/w % of poloxamer 407;
about 41.40 w/w % of mannitol;
about 22.395 w/w % of microcrystalline cellulose;
about 8.0 w/w % of croscarmellose sodium; and
about 1.5 w/w % of magnesium stearate.

In some embodiments, each tablet of the initiation dosage comprises:
about 19 w/w % to about 21 w/w % of the sodium salt of the compound of Formula Ia or Ib;
about 0.03 w/w % to about 0.04 w/w % of the monohydrate of the compound of Formula Ila or IIb;
about 4 w/w % to about 6 w/w % of copovidone;
about 0.1 w/w % to about 2 w/w % of poloxamer 407;
about 40 w/w % to about 42 w/w % of mannitol;
about 22 w/w % to about 23 w/w % of microcrystalline cellulose;
about 7 w/w % to about 9 w/w % of croscarmellose sodium; and
about 1.0 w/w % to about 2.0 w/w % of magnesium stearate.

In some embodiments, each tablet of the initiation dosage comprises:
about 19.8 w/w % to about 20.8 w/w % of the sodium salt of the compound of Formula Ia or Ib;
about 0.03 w/w % to about 0.04 w/w % of the monohydrate of the compound of Formula IIa or IIb;
about 4 w/w % to about 5 w/w % of copovidone;
about 1 w/w % to about 2 w/w % of poloxamer 407;
about 41 w/w % to about 42 w/w % of mannitol;
about 22.3 w/w % to about 22.4 w/w % of microcrystalline cellulose;
about 7.9 w/w % to about 8.1 w/w % of croscarmellose sodium; and
about 1.4 w/w % to about 1.6 w/w % of magnesium stearate.

In some embodiments, each tablet of the initiation dosage comprises:
about 20.46 w/w % of the sodium salt of the compound of Formula Ia or Ib;
about 0.0355 w/w % of the monohydrate of the compound of Formula IIa or IIb;
about 4.88 w/w % of copovidone;
about 1.33 w/w % of poloxamer 407;
about 41.40 w/w % of mannitol;
about 22.395 w/w % of microcrystalline cellulose;
about 8.0 w/w % of croscarmellose sodium; and
about 1.5 w/w % of magnesium stearate.

In some embodiments, each tablet of the initiation dosage comprises:
about 19 w/w % to about 21 w/w % of the compound of Formula Ia or Ib, or a pharmaceutically acceptable salt thereof;
about 0.03 w/w % to about 0.04 w/w % of the compound of Formula IIa or IIb, or a solvate thereof;
about 4 w/w % to about 6 w/w % of copovidone;
about 0.1 w/w % to about 2 w/w % of poloxamer 407;
about 40 w/w % to about 42 w/w % of mannitol;
about 22 w/w % to about 23 w/w % of microcrystalline cellulose;
about 7 w/w % to about 9 w/w % of croscarmellose sodium;
about 1.0 w/w % to about 2.0 w/w % of magnesium stearate; and
an outer film coat.

In some embodiments, each tablet of the initiation dosage comprises:
about 19.8 w/w % to about 20.8 w/w % of the compound of Formula Ia or Ib, or a pharmaceutically acceptable salt thereof;
about 0.03 w/w % to about 0.04 w/w % of the compound of Formula IIa or IIb, or a solvate thereof;
about 4 w/w % to about 5 w/w % of copovidone;
about 1 w/w % to about 2 w/w % of poloxamer 407;
about 41 w/w % to about 42 w/w % of mannitol;
about 22.3 w/w % to about 22.4 w/w % of microcrystalline cellulose;
about 7.9 w/w % to about 8.1 w/w % of croscarmellose sodium;
about 1.4 w/w % to about 1.6 w/w % of magnesium stearate; and
an outer film coat.

In some embodiments, each tablet of the initiation dosage comprises:
about 20.46 w/w % of the compound of Formula Ia or Ib, or a pharmaceutically acceptable salt thereof;
about 0.0355 w/w % of the compound of Formula IIa or IIb, or a solvate thereof;
about 4.88 w/w % of copovidone;
about 1.33 w/w % of poloxamer 407;
about 41.40 w/w % of mannitol;
about 22.395 w/w % of microcrystalline cellulose;
about 8.0 w/w % of croscarmellose sodium;
about 1.5 w/w % of magnesium stearate; and
an outer film coat.

In some embodiments, each tablet of the initiation dosage comprises:
about 19 w/w % to about 21 w/w % of the sodium salt of the compound of Formula Ia or Ib;
about 0.03 w/w % to about 0.04 w/w % of the monohydrate of the compound of Formula IIa or IIb;
about 4 w/w % to about 6 w/w % of copovidone;
about 0.1 w/w % to about 2 w/w % of poloxamer 407;
about 40 w/w % to about 42 w/w % of mannitol;
about 22 w/w % to about 23 w/w % of microcrystalline cellulose;
about 7 w/w % to about 9 w/w % of croscarmellose sodium;
about 1.0 w/w % to about 2.0 w/w % of magnesium stearate; and
an outer film coat.

In some embodiments, each tablet of the initiation dosage comprises:
about 19.8 w/w % to about 20.8 w/w % of the sodium salt of the compound of Formula Ia or Ib;
about 0.03 w/w % to about 0.04 w/w % of the monohydrate of the compound of Formula IIa or IIb;
about 4 w/w % to about 5 w/w % of copovidone;
about 1 w/w % to about 2 w/w % of poloxamer 407;
about 41 w/w % to about 42 w/w % of mannitol;
about 22.3 w/w % to about 22.4 w/w % of microcrystalline cellulose;
about 7.9 w/w % to about 8.1 w/w % of croscarmellose sodium;
about 1.4 w/w % to about 1.6 w/w % of magnesium stearate; and
an outer film coat.

In some embodiments, each tablet of the initiation dosage comprises:
about 20.46 w/w % of the sodium salt of the compound of Formula Ia or Ib;
about 0.0355 w/w % of the monohydrate of the compound of Formula IIa or IIb;
about 4.88 w/w % of copovidone;
about 1.33 w/w % of poloxamer 407;
about 41.40 w/w % of mannitol;
about 22.395 w/w % of microcrystalline cellulose;
about 8.0 w/w % of croscarmellose sodium;
about 1.5 w/w % of magnesium stearate; and
an outer film coat.

In some embodiments, each tablet of the initiation dosage consists essentially of:
about 19 w/w % to about 21 w/w % of the compound of Formula Ia or Ib, or a pharmaceutically acceptable salt thereof;
about 0.03 w/w % to about 0.04 w/w % of the compound of Formula IIa or IIb, or a solvate thereof;
about 4 w/w % to about 6 w/w % of copovidone;
about 0.1 w/w % to about 2 w/w % of poloxamer 407;
about 40 w/w % to about 42 w/w % of mannitol;
about 22 w/w % to about 23 w/w % of microcrystalline cellulose;
about 7 w/w % to about 9 w/w % of croscarmellose sodium; and
about 1.0 w/w % to about 2.0 w/w % of magnesium stearate.

In some embodiments, each tablet of the initiation dosage consists essentially of:
about 19.8 w/w % to about 20.8 w/w % of the compound of Formula Ia or Ib, or a pharmaceutically acceptable salt thereof;
about 0.03 w/w % to about 0.04 w/w % of the compound of Formula IIa or IIb, or a solvate thereof;
about 4 w/w % to about 5 w/w % of copovidone;
about 1 w/w % to about 2 w/w % of poloxamer 407;
about 41 w/w % to about 42 w/w % of mannitol;
about 22.3 w/w % to about 22.4 w/w % of microcrystalline cellulose;
about 7.9 w/w % to about 8.1 w/w % of croscarmellose sodium; and
about 1.4 w/w % to about 1.6 w/w % of magnesium stearate.

In some embodiments, each tablet of the initiation dosage consists essentially of:
about 20.46 w/w % of the compound of Formula Ia or Ib, or a pharmaceutically acceptable salt thereof;
about 0.0355 w/w % of the compound of Formula IIa or IIb, or a solvate thereof;
about 4.88 w/w % of copovidone;
about 1.33 w/w % of poloxamer 407;
about 41.40 w/w % of mannitol;
about 22.395 w/w % of microcrystalline cellulose;
about 8.0 w/w % of croscarmellose sodium; and
about 1.5 w/w % of magnesium stearate.

In some embodiments, each tablet of the initiation dosage consists essentially of:
about 19 w/w % to about 21 w/w % of the sodium salt of the compound of Formula Ia or Ib;
about 0.03 w/w % to about 0.04 w/w % of the monohydrate of the compound of Formula IIa or IIb;
about 4 w/w % to about 6 w/w % of copovidone;
about 0.1 w/w % to about 2 w/w % of poloxamer 407;
about 40 w/w % to about 42 w/w % of mannitol;
about 22 w/w % to about 23 w/w % of microcrystalline cellulose;
about 7 w/w % to about 9 w/w % of croscarmellose sodium; and
about 1.0 w/w % to about 2.0 w/w % of magnesium stearate.

In some embodiments, each tablet of the initiation dosage consists essentially of:
about 19.8 w/w % to about 20.8 w/w % of the sodium salt of the compound of Formula Ia or Ib;
about 0.03 w/w % to about 0.04 w/w % of the monohydrate of the compound of Formula IIa or IIb;
about 4 w/w % to about 5 w/w % of copovidone;
about 1 w/w % to about 2 w/w % of poloxamer 407;
about 41 w/w % to about 42 w/w % of mannitol;
about 22.3 w/w % to about 22.4 w/w % of microcrystalline cellulose;
about 7.9 w/w % to about 8.1 w/w % of croscarmellose sodium; and
about 1.4 w/w % to about 1.6 w/w % of magnesium stearate.

In some embodiments, each tablet of the initiation dosage consists essentially of:
about 20.46 w/w % of the sodium salt of the compound of Formula Ia or Ib;
about 0.0355 w/w % of the monohydrate of the compound of Formula IIa or IIb;
about 4.88 w/w % of copovidone;
about 1.33 w/w % of poloxamer 407;
about 41.40 w/w % of mannitol;
about 22.395 w/w % of microcrystalline cellulose;
about 8.0 w/w % of croscarmellose sodium; and
about 1.5 w/w % of magnesium stearate.

In some embodiments, each tablet of the initiation dosage consists essentially of:
about 19 w/w % to about 21 w/w % of the compound of Formula Ia or Ib, or a pharmaceutically acceptable salt thereof;
about 0.03 w/w % to about 0.04 w/w % of the compound of Formula IIa or IIb, or a solvate thereof;
about 4 w/w % to about 6 w/w % of copovidone;
about 0.1 w/w % to about 2 w/w % of poloxamer 407;
about 40 w/w % to about 42 w/w % of mannitol;
about 22 w/w % to about 23 w/w % of microcrystalline cellulose;
about 7 w/w % to about 9 w/w % of croscarmellose sodium;
about 1.0 w/w % to about 2.0 w/w % of magnesium stearate; and
an outer film coat.

In some embodiments, each tablet of the initiation dosage consists essentially of:
about 19.8 w/w % to about 20.8 w/w % of the compound of Formula Ia or Ib, or a pharmaceutically acceptable salt thereof;
about 0.03 w/w % to about 0.04 w/w % of the compound of Formula IIa or IIb, or a solvate thereof;
about 4 w/w % to about 5 w/w % of copovidone;
about 1 w/w % to about 2 w/w % of poloxamer 407;
about 41 w/w % to about 42 w/w % of mannitol;
about 22.3 w/w % to about 22.4 w/w % of microcrystalline cellulose;
about 7.9 w/w % to about 8.1 w/w % of croscarmellose sodium;
about 1.4 w/w % to about 1.6 w/w % of magnesium stearate; and
an outer film coat.

In some embodiments, each tablet of the initiation dosage consists essentially of:
about 20.46 w/w % of the compound of Formula Ia or Ib, or a pharmaceutically acceptable salt thereof;
about 0.0355 w/w % of the compound of Formula IIa or IIb, or a solvate thereof;
about 4.88 w/w % of copovidone;
about 1.33 w/w % of poloxamer 407;
about 41.40 w/w % of mannitol;
about 22.395 w/w % of microcrystalline cellulose;
about 8.0 w/w % of croscarmellose sodium;
about 1.5 w/w % of magnesium stearate; and
an outer film coat.

In some embodiments, each tablet of the initiation dosage consists essentially of:
about 19 w/w % to about 21 w/w % of the sodium salt of the compound of Formula Ia or Ib;
about 0.03 w/w % to about 0.04 w/w % of the monohydrate of the compound of Formula IIa or IIb;
about 4 w/w % to about 6 w/w % of copovidone;
about 0.1 w/w % to about 2 w/w % of poloxamer 407;
about 40 w/w % to about 42 w/w % of mannitol;
about 22 w/w % to about 23 w/w % of microcrystalline cellulose;
about 7 w/w % to about 9 w/w % of croscarmellose sodium;
about 1.0 w/w % to about 2.0 w/w % of magnesium stearate; and
an outer film coat.

In some embodiments, each tablet of the initiation dosage consists essentially of:
about 19.8 w/w % to about 20.8 w/w % of the sodium salt of the compound of Formula Ia or Ib;
about 0.03 w/w % to about 0.04 w/w % of the monohydrate of the compound of Formula IIa or IIb;
about 4 w/w % to about 5 w/w % of copovidone;
about 1 w/w % to about 2 w/w % of poloxamer 407;
about 41 w/w % to about 42 w/w % of mannitol;
about 22.3 w/w % to about 22.4 w/w % of microcrystalline cellulose;
about 7.9 w/w % to about 8.1 w/w % of croscarmellose sodium;
about 1.4 w/w % to about 1.6 w/w % of magnesium stearate; and
an outer film coat.

In some embodiments, each tablet of the initiation dosage consists essentially of:
about 20.46 w/w % of the sodium salt of the compound of Formula Ia or Ib;
about 0.0355 w/w % of the monohydrate of the compound of Formula IIa or IIb;
about 4.88 w/w % of copovidone;
about 1.33 w/w % of poloxamer 407;
about 41.40 w/w % of mannitol;
about 22.395 w/w % of microcrystalline cellulose;
about 8.0 w/w % of croscarmellose sodium;
about 1.5 w/w % of magnesium stearate; and
an outer film coat.

In some embodiments, each tablet of the initiation dosage consists of:
about 19 w/w % to about 21 w/w % of the compound of Formula Ia or Ib, or a pharmaceutically acceptable salt thereof;
about 0.03 w/w % to about 0.04 w/w % of the compound of Formula IIa or IIb, or a solvate thereof;
about 4 w/w % to about 6 w/w % of copovidone;
about 0.1 w/w % to about 2 w/w % of poloxamer 407;
about 40 w/w % to about 42 w/w % of mannitol;
about 22 w/w % to about 23 w/w % of microcrystalline cellulose;
about 7 w/w % to about 9 w/w % of croscarmellose sodium; and
about 1.0 w/w % to about 2.0 w/w % of magnesium stearate.

In some embodiments, each tablet of the initiation dosage consists of:
about 19.8 w/w % to about 20.8 w/w % of the compound of Formula Ia or Ib, or a pharmaceutically acceptable salt thereof;
about 0.03 w/w % to about 0.04 w/w % of the compound of Formula IIa or IIb, or a solvate thereof;
about 4 w/w % to about 5 w/w % of copovidone;
about 1 w/w % to about 2 w/w % of poloxamer 407;
about 41 w/w % to about 42 w/w % of mannitol;
about 22.3 w/w % to about 22.4 w/w % of microcrystalline cellulose;
about 7.9 w/w % to about 8.1 w/w % of croscarmellose sodium; and
about 1.4 w/w % to about 1.6 w/w % of magnesium stearate.

In some embodiments, each tablet of the initiation dosage consists of:
about 20.46 w/w % of the compound of Formula Ia or Ib, or a pharmaceutically acceptable salt thereof;
about 0.0355 w/w % of the compound of Formula IIa or IIb, or a solvate thereof;
about 4.88 w/w % of copovidone;
about 1.33 w/w % of poloxamer 407;
about 41.40 w/w % of mannitol;
about 22.395 w/w % of microcrystalline cellulose;
about 8.0 w/w % of croscarmellose sodium; and
about 1.5 w/w % of magnesium stearate.

In some embodiments, each tablet of the initiation dosage consists of:
about 19 w/w % to about 21 w/w % of the sodium salt of the compound of Formula Ia or Ib;
about 0.03 w/w % to about 0.04 w/w % of the monohydrate of the compound of Formula IIa or IIb;
about 4 w/w % to about 6 w/w % of copovidone;
about 0.1 w/w % to about 2 w/w % of poloxamer 407;
about 40 w/w % to about 42 w/w % of mannitol;
about 22 w/w % to about 23 w/w % of microcrystalline cellulose;
about 7 w/w % to about 9 w/w % of croscarmellose sodium; and
about 1.0 w/w % to about 2.0 w/w % of magnesium stearate.

In some embodiments, each tablet of the initiation dosage consists of:
about 19.8 w/w % to about 20.8 w/w % of the sodium salt of the compound of Formula Ia or Ib;
about 0.03 w/w % to about 0.04 w/w % of the monohydrate of the compound of Formula IIa or IIb;
about 4 w/w % to about 5 w/w % of copovidone;
about 1 w/w % to about 2 w/w % of poloxamer 407;
about 41 w/w % to about 42 w/w % of mannitol;
about 22.3 w/w % to about 22.4 w/w % of microcrystalline cellulose;
about 7.9 w/w % to about 8.1 w/w % of croscarmellose sodium; and
about 1.4 w/w % to about 1.6 w/w % of magnesium stearate.

In some embodiments, each tablet of the initiation dosage consists of:
about 20.46 w/w % of the sodium salt of the compound of Formula Ia or Ib;
about 0.0355 w/w % of the monohydrate of the compound of Formula IIa or IIb;
about 4.88 w/w % of copovidone;
about 1.33 w/w % of poloxamer 407;
about 41.40 w/w % of mannitol;
about 22.395 w/w % of microcrystalline cellulose;
about 8.0 w/w % of croscarmellose sodium; and
about 1.5 w/w % of magnesium stearate.

In some embodiments, each tablet of the initiation dosage consists of:
about 19 w/w % to about 21 w/w % of the compound of Formula Ia or Ib, or a pharmaceutically acceptable salt thereof;
about 0.03 w/w % to about 0.04 w/w % of the compound of Formula IIa or IIb, or a solvate thereof;
about 4 w/w % to about 6 w/w % of copovidone;
about 0.1 w/w % to about 2 w/w % of poloxamer 407;
about 40 w/w % to about 42 w/w % of mannitol;
about 22 w/w % to about 23 w/w % of microcrystalline cellulose;
about 7 w/w % to about 9 w/w % of croscarmellose sodium;
about 1.0 w/w % to about 2.0 w/w % of magnesium stearate; and
an outer film coat.

In some embodiments, each tablet of the initiation dosage consists of:
about 19.8 w/w % to about 20.8 w/w % of the compound of Formula Ia or Ib, or a pharmaceutically acceptable salt thereof;
about 0.03 w/w % to about 0.04 w/w % of the compound of Formula IIa or IIb, or a solvate thereof;
about 4 w/w % to about 5 w/w % of copovidone;
about 1 w/w % to about 2 w/w % of poloxamer 407;
about 41 w/w % to about 42 w/w % of mannitol;
about 22.3 w/w % to about 22.4 w/w % of microcrystalline cellulose;
about 7.9 w/w % to about 8.1 w/w % of croscarmellose sodium;
about 1.4 w/w % to about 1.6 w/w % of magnesium stearate; and
an outer film coat.

In some embodiments, each tablet of the initiation dosage consists of:
about 20.46 w/w % of the compound of Formula Ia or Ib, or a pharmaceutically acceptable salt thereof;
about 0.0355 w/w % of the compound of Formula IIa or IIb, or a solvate thereof;
about 4.88 w/w % of copovidone;
about 1.33 w/w % of poloxamer 407;
about 41.40 w/w % of mannitol;
about 22.395 w/w % of microcrystalline cellulose;
about 8.0 w/w % of croscarmellose sodium;
about 1.5 w/w % of magnesium stearate; and
an outer film coat.

In some embodiments, each tablet of the initiation dosage consists of:
about 19 w/w % to about 21 w/w % of the sodium salt of the compound of Formula Ia or Ib;
about 0.03 w/w % to about 0.04 w/w % of the monohydrate of the compound of Formula IIa or IIb;
about 4 w/w % to about 6 w/w % of copovidone;
about 0.1 w/w % to about 2 w/w % of poloxamer 407;
about 40 w/w % to about 42 w/w % of mannitol;
about 22 w/w % to about 23 w/w % of microcrystalline cellulose;
about 7 w/w % to about 9 w/w % of croscarmellose sodium;
about 1.0 w/w % to about 2.0 w/w % of magnesium stearate; and
an outer film coat.

In some embodiments, each tablet of the initiation dosage consists of:
about 19.8 w/w % to about 20.8 w/w % of the sodium salt of the compound of Formula Ia or Ib;
about 0.03 w/w % to about 0.04 w/w % of the monohydrate of the compound of Formula IIa or IIb;
about 4 w/w % to about 5 w/w % of copovidone;
about 1 w/w % to about 2 w/w % of poloxamer 407;
about 41 w/w % to about 42 w/w % of mannitol;
about 22.3 w/w % to about 22.4 w/w % of microcrystalline cellulose;
about 7.9 w/w % to about 8.1 w/w % of croscarmellose sodium;
about 1.4 w/w % to about 1.6 w/w % of magnesium stearate; and
an outer film coat.

In some embodiments, each tablet of the initiation dosage consists of:
about 20.46 w/w % of the sodium salt of the compound of Formula Ia or Ib;
about 0.0355 w/w % of the monohydrate of the compound of Formula IIa or IIb;
about 4.88 w/w % of copovidone;
about 1.33 w/w % of poloxamer 407;
about 41.40 w/w % of mannitol;
about 22.395 w/w % of microcrystalline cellulose;
about 8.0 w/w % of croscarmellose sodium;
about 1.5 w/w % of magnesium stearate; and
an outer film coat.

In some embodiments, each tablet of the initiation dosage comprises:
about 400 mg of the spray dried dispersion of the compound of Formula Ia or Ib, or a pharmaceutically acceptable salt, co-crystal, solvate, or combination thereof; and
about 0.5 mg to about 0.6 mg of the compound of Formula IIa or IIb, or a pharmaceutically acceptable salt, co-crystal, solvate, or combination thereof.

In some embodiments, each tablet of the initiation dosage comprises:
about 400 mg of the spray dried dispersion of the compound of Formula Ia or Ib, or a pharmaceutically acceptable salt, co-crystal, solvate, or combination thereof; and
about 0.5325 mg of the compound of Formula IIa or IIb, or a pharmaceutically acceptable salt, co-crystal, solvate, or combination thereof.

In some embodiments, each tablet of the initiation dosage comprises:
about 400 mg of the spray dried dispersion of the compound of Formula Ia or Ib, or a pharmaceutically acceptable salt, co-crystal, solvate, or combination thereof; and
about 0.5 mg of the compound of Formula IIa or IIb, or a pharmaceutically acceptable salt, co-crystal, solvate, or combination thereof, on a free base basis.

In some embodiments, each tablet of the initiation dosage comprises:
about 400 mg of the spray dried dispersion of the compound of Formula Ia or Ib, or a pharmaceutically acceptable salt, co-crystal, solvate, or combination thereof;
about 0.5 mg to about 0.6 mg of the compound of Formula IIa or IIb, or a pharmaceutically acceptable salt, co-crystal, solvate, or combination thereof;
about 615 mg to about 625 mg of mannitol;
about 330 mg to about 340 mg of microcrystalline cellulose;
about 115 mg to about 125 mg croscarmellose sodium; and
about 20 mg to about 25 mg magnesium stearate.

In some embodiments, each tablet of the initiation dosage comprises:

about 400 mg of the spray dried dispersion of the compound of Formula Ia or Ib, or a pharmaceutically acceptable salt, co-crystal, solvate, or combination thereof;
about 0.5325 mg of the compound of Formula IIa or IIb, or a pharmaceutically acceptable salt, co-crystal, solvate, or combination thereof;
about 621 mg of mannitol;
about 335.9175 mg of microcrystalline cellulose;
about 120 mg croscarmellose sodium; and
about 22.5 mg magnesium stearate.

In some embodiments, each tablet of the initiation dosage comprises:
about 400 mg of the spray dried dispersion of the compound of Formula Ia or Ib, or a pharmaceutically acceptable salt, co-crystal, solvate, or combination thereof;
about 0.5 mg of the compound of Formula IIa or IIb, or a pharmaceutically acceptable salt, co-crystal, solvate, or combination thereof, on a free base basis;
about 621 mg of mannitol;
about 335.9175 mg of microcrystalline cellulose;
about 120 mg croscarmellose sodium; and
about 22.5 mg magnesium stearate.

In some embodiments, each tablet of the initiation dosage comprises:
about 26.6 w/w % to about 26.7 w/w % of the spray dried dispersion of the compound of Formula Ia or Ib, or a pharmaceutically acceptable salt thereof;
about 0.03 w/w % to about 0.04 w/w % of the compound of Formula IIa or IIb, or a pharmaceutically acceptable salt, co-crystal, solvate, or combination thereof; and
one or more pharmaceutically acceptable excipients.

In some embodiments, each tablet of the initiation dosage comprises:
about 26.67 w/w % of the spray dried dispersion of the spray dried dispersion of the compound of Formula Ia or Ib, or a pharmaceutically acceptable salt thereof;
about 0.0355 w/w % of the compound of Formula IIa or IIb, or a pharmaceutically acceptable salt, co-crystal, solvate, or combination thereof; and
one or more pharmaceutically acceptable excipients.

In some embodiments, each tablet of the initiation dosage comprises:
about 26.6 w/w % to about 26.7 w/w % of the spray dried dispersion of the compound of Formula Ia or Ib, or a pharmaceutically acceptable salt thereof;
about 0.03 w/w % to about 0.04 w/w % of the compound of Formula IIa or IIb, or a solvate thereof, and
one or more pharmaceutically acceptable excipients.

In some embodiments, each tablet of the initiation dosage comprises:
about 26.67 w/w % of the spray dried dispersion of the compound of Formula Ia or Ib, or a pharmaceutically acceptable salt thereof;
about 0.0355 w/w % of the compound of Formula IIa or IIb, or a solvate thereof, and
one or more pharmaceutically acceptable excipients.

In some embodiments, each tablet of the initiation dosage comprises:
about 26.6 w/w % to about 26.7 w/w % of the spray dried dispersion of the compound of Formula Ia or Ib, or a pharmaceutically acceptable salt thereof;
about 0.03 w/w % to about 0.04 w/w % of the monohydrate of the compound of Formula IIa or IIb; and
one or more pharmaceutically acceptable excipients.

In some embodiments, each tablet of the initiation dosage comprises:
about 26.67 w/w % of the spray dried dispersion of the compound of Formula Ia or Ib, or a pharmaceutically acceptable salt thereof;
about 0.0355 w/w % of the monohydrate of the compound of Formula IIa or IIb; and
one or more pharmaceutically acceptable excipients.

In some embodiments, each tablet of the initiation dosage comprises:
about 26.6 w/w % to about 26.7 w/w % of the spray dried dispersion of the compound of Formula Ia or Ib, or a pharmaceutically acceptable salt thereof;
about 0.03 w/w % to about 0.04 w/w % of the compound of Formula IIa or IIb, or a pharmaceutically acceptable salt, co-crystal, solvate, or combination thereof;
about 41 w/w % to about 42 w/w % of mannitol;
about 22.3 w/w % to about 22.4 w/w % of microcrystalline cellulose;
about 7.9 w/w % to about 8.1 w/w % of croscarmellose sodium; and
about 1.4 w/w % to about 1.6 w/w % of magnesium stearate.

In some embodiments, each tablet of the initiation dosage comprises:
about 26.67 w/w % of the spray dried dispersion of the compound of Formula Ia or Ib, or a pharmaceutically acceptable salt thereof;
about 0.0355 w/w % of the compound of Formula IIa or IIb, or a pharmaceutically acceptable salt, co-crystal, solvate, or combination thereof;
about 41.4 w/w % of mannitol;
about 22.3945 w/w % of microcrystalline cellulose;
about 8.0 w/w % of croscarmellose sodium; and
about 1.5 w/w % of magnesium stearate.

In some embodiments, each tablet of the initiation dosage comprises:
about 400 mg of the spray dried dispersion of the compound of Formula Ia or Ib, or a pharmaceutically acceptable salt thereof, and
about 0.5 mg to about 0.6 mg of the compound of Formula IIa or IIb, or a pharmaceutically acceptable salt, co-crystal, solvate, or combination thereof.

In some embodiments, each tablet of the initiation dosage comprises:
about 400 mg of the spray dried dispersion of the compound of Formula Ia or Ib, or a pharmaceutically acceptable salt thereof, and
about 0.5325 mg of the compound of Formula IIa or IIb, or a pharmaceutically acceptable salt, co-crystal, solvate, or combination thereof.

In some embodiments, each tablet of the initiation dosage comprises:
about 400 mg of the spray dried dispersion of the compound of Formula Ia or Ib, or a pharmaceutically acceptable salt thereof, and
about 0.5 mg of the compound of Formula IIa or IIb, or a pharmaceutically acceptable salt, co-crystal, solvate, or combination thereof, on a free base basis.

In some embodiments, each tablet of the initiation dosage comprises:
about 400 mg of the spray dried dispersion of the compound of Formula Ia or Ib, or a pharmaceutically acceptable salt thereof;
about 0.5 mg to about 0.6 mg of the compound of Formula IIa or IIb, or a pharmaceutically acceptable salt, co-crystal, solvate, or combination thereof;
about 615 mg to about 625 mg of mannitol;
about 330 mg to about 340 mg of microcrystalline cellulose;
about 115 mg to about 125 mg croscarmellose sodium; and
about 20 mg to about 25 mg magnesium stearate.

In some embodiments, each tablet of the initiation dosage comprises:
about 400 mg of the spray dried dispersion of the compound of Formula Ia or Ib, or a pharmaceutically acceptable salt thereof;
about 0.5325 mg of the compound of Formula IIa or IIb, or a pharmaceutically acceptable salt, co-crystal, solvate, or combination thereof;
about 621 mg of mannitol;
about 335.9175 mg of microcrystalline cellulose;
about 120 mg croscarmellose sodium; and
about 22.5 mg magnesium stearate.

In some embodiments, each tablet of the initiation dosage comprises:
about 400 mg of the spray dried dispersion of the compound of Formula Ia or Ib, or a pharmaceutically acceptable salt thereof;
about 0.5 mg of the compound of Formula IIa or IIb, or a pharmaceutically acceptable salt, co-crystal, solvate, or combination thereof, on a free base basis;
about 621 mg of mannitol;
about 335.9175 mg of microcrystalline cellulose;
about 120 mg croscarmellose sodium; and
about 22.5 mg magnesium stearate.

In some embodiments, each tablet of the initiation dosage comprises:
about 26.6 w/w % to about 26.7 w/w % of the spray dried dispersion of the sodium salt of the compound of Formula Ia or Ib;
about 0.03 w/w % to about 0.04 w/w % of the compound of Formula IIa or IIb, or a pharmaceutically acceptable salt, co-crystal, solvate, or combination thereof; and
one or more pharmaceutically acceptable excipients.

In some embodiments, each tablet of the initiation dosage comprises:
about 26.67 w/w % of the spray dried dispersion of the sodium salt of the compound of Formula Ia or Ib;
about 0.0355 w/w % of the compound of Formula IIa or IIb, or a pharmaceutically acceptable salt, co-crystal, solvate, or combination thereof; and
one or more pharmaceutically acceptable excipients.

In some embodiments, each tablet of the initiation dosage comprises:
about 26.6 w/w % to about 26.7 w/w % of the spray dried dispersion of the sodium salt of the compound of Formula Ia or Ib;
about 0.03 w/w % to about 0.04 w/w % of the compound of Formula IIa or IIb, or a solvate thereof, and
one or more pharmaceutically acceptable excipients.

In some embodiments, each tablet of the initiation dosage comprises:
about 26.67 w/w % of the spray dried dispersion of the sodium salt of the compound of Formula Ia or Ib;
about 0.0355 w/w % of the compound of Formula IIa or IIb, or a solvate thereof, and
one or more pharmaceutically acceptable excipients.

In some embodiments, each tablet of the initiation dosage comprises:
about 26.6 w/w % to about 26.7 w/w % of the spray dried dispersion of the sodium salt of the compound of Formula Ia or Ib;
about 0.03 w/w % to about 0.04 w/w % of the monohydrate of the compound of Formula IIa or IIb; and
one or more pharmaceutically acceptable excipients.

In some embodiments, each tablet of the initiation dosage comprises:
about 26.67 w/w % of the spray dried dispersion of the sodium salt of the compound of Formula Ia or Ib;
about 0.0355 w/w % of the monohydrate of the compound of Formula IIa or IIb; and
one or more pharmaceutically acceptable excipients.

In some embodiments, each tablet of the initiation dosage comprises:
about 26.6 w/w % to about 26.7 w/w % of the spray dried dispersion of the sodium salt of the compound of Formula Ia or Ib;
about 0.03 w/w % to about 0.04 w/w % of the compound of Formula IIa or IIb, or a pharmaceutically acceptable salt, co-crystal, solvate, or combination thereof;
about 41 w/w % to about 42 w/w % of mannitol;
about 22.3 w/w % to about 22.4 w/w % of microcrystalline cellulose;
about 7.9 w/w % to about 8.1 w/w % of croscarmellose sodium; and
about 1.4 w/w % to about 1.6 w/w % of magnesium stearate.

In some embodiments, each tablet of the initiation dosage comprises:
about 26.67 w/w % of the spray dried dispersion of the sodium salt of the compound of Formula Ia or Ib;
about 0.0355 w/w % of the compound of Formula IIa or IIb, or a pharmaceutically acceptable salt, co-crystal, solvate, or combination thereof;
about 41.4 w/w % of mannitol;
about 22.3945 w/w % of microcrystalline cellulose;
about 8.0 w/w % of croscarmellose sodium; and
about 1.5 w/w % of magnesium stearate.

In some embodiments, each tablet of the initiation dosage comprises:
about 400 mg of the spray dried dispersion of the sodium salt of the compound of Formula Ia or Ib; and
about 0.5 mg to about 0.6 mg of the compound of Formula IIa or IIb, or a pharmaceutically acceptable salt, co-crystal, solvate, or combination thereof.

In some embodiments, each tablet of the initiation dosage comprises:
about 400 mg of the spray dried dispersion of the sodium salt of the compound of Formula Ia or Ib; and
about 0.5325 mg of the compound of Formula IIa or IIb, or a pharmaceutically acceptable salt, co-crystal, solvate, or combination thereof.

In some embodiments, each tablet of the initiation dosage comprises:
about 400 mg of the spray dried dispersion of the sodium salt of the compound of Formula Ia or Ib; and
about 0.5 mg of the compound of Formula IIa or IIb, or a pharmaceutically acceptable salt, co-crystal, solvate, or combination thereof, on a free base basis.

In some embodiments, each tablet of the initiation dosage comprises:
about 400 mg of the spray dried dispersion of the sodium salt of the compound of Formula Ia or Ib;
about 0.5 mg to about 0.6 mg of the compound of Formula IIa or IIb, or a pharmaceutically acceptable salt, co-crystal, solvate, or combination thereof;
about 615 mg to about 625 mg of mannitol;
about 330 mg to about 340 mg of microcrystalline cellulose;
about 115 mg to about 125 mg croscarmellose sodium; and
about 20 mg to about 25 mg magnesium stearate.

In some embodiments, each tablet of the initiation dosage comprises:
about 400 mg of the spray dried dispersion of the sodium salt of the compound of Formula Ia or Ib;
about 0.5325 mg of the compound of Formula IIa or IIb, or a pharmaceutically acceptable salt, co-crystal, solvate, or combination thereof;
about 621 mg of mannitol;
about 335.9175 mg of microcrystalline cellulose;
about 120 mg croscarmellose sodium; and
about 22.5 mg magnesium stearate.

In some embodiments, each tablet of the initiation dosage comprises:
about 400 mg of the spray dried dispersion of the sodium salt of the compound of Formula Ia or Ib;
about 0.5 mg of the compound of Formula IIa or IIb, or a pharmaceutically acceptable salt, co-crystal, solvate, or combination thereof, on a free base basis;
about 621 mg of mannitol;
about 335.9175 mg of microcrystalline cellulose;
about 120 mg croscarmellose sodium; and
about 22.5 mg magnesium stearate.

In some embodiments, each tablet of the initiation dosage comprises:
about 26.6 w/w % to about 26.7 w/w % of the spray dried dispersion of the sodium salt of the compound of Formula Ia;
about 0.03 w/w % to about 0.04 w/w % of the compound of Formula IIa or IIb, or a pharmaceutically acceptable salt, co-crystal, solvate, or combination thereof; and
one or more pharmaceutically acceptable excipients.

In some embodiments, each tablet of the initiation dosage comprises:
about 26.67 w/w % of the spray dried dispersion of the sodium salt of the compound of Formula Ia;
about 0.0355 w/w % of the compound of Formula IIa or IIb, or a pharmaceutically acceptable salt, co-crystal, solvate, or combination thereof; and
one or more pharmaceutically acceptable excipients.

In some embodiments, each tablet of the initiation dosage comprises:
about 26.6 w/w % to about 26.7 w/w % of the spray dried dispersion of the sodium salt of the compound of Formula Ia;
about 0.03 w/w % to about 0.04 w/w % of the compound of Formula IIa or IIb, or a solvate thereof, and
one or more pharmaceutically acceptable excipients.

In some embodiments, each tablet of the initiation dosage comprises:
about 26.67 w/w % of the spray dried dispersion of the sodium salt of the compound of Formula Ia;
about 0.0355 w/w % of the compound of Formula IIa or IIb, or a solvate thereof, and
one or more pharmaceutically acceptable excipients.

In some embodiments, each tablet of the initiation dosage comprises:
about 26.6 w/w % to about 26.7 w/w % of the spray dried dispersion of the sodium salt of the compound of Formula Ia;
about 0.03 w/w % to about 0.04 w/w % of the monohydrate of the compound of Formula IIa or IIb; and
one or more pharmaceutically acceptable excipients.

In some embodiments, each tablet of the initiation dosage comprises:
about 26.67 w/w % of the spray dried dispersion of the sodium salt of the compound of Formula Ia;
about 0.0355 w/w % of the monohydrate of the compound of Formula IIa or IIb; and
one or more pharmaceutically acceptable excipients.

In some embodiments, each tablet of the initiation dosage comprises:
about 26.6 w/w % to about 26.7 w/w % of the spray dried dispersion of the sodium salt of the compound of Formula Ia;
about 0.03 w/w % to about 0.04 w/w % of the compound of Formula IIa or IIb, or a pharmaceutically acceptable salt, co-crystal, solvate, or combination thereof;
about 41 w/w % to about 42 w/w % of mannitol;
about 22.3 w/w % to about 22.4 w/w % of microcrystalline cellulose;
about 7.9 w/w % to about 8.1 w/w % of croscarmellose sodium; and
about 1.4 w/w % to about 1.6 w/w % of magnesium stearate.

In some embodiments, each tablet of the initiation dosage comprises:
about 26.67 w/w % of the spray dried dispersion of the sodium salt of the compound of Formula Ia;
about 0.0355 w/w % of the compound of Formula IIa or IIb, or a pharmaceutically acceptable salt, co-crystal, solvate, or combination thereof;
about 41.4 w/w % of mannitol;
about 22.3945 w/w % of microcrystalline cellulose;
about 8.0 w/w % of croscarmellose sodium; and
about 1.5 w/w % of magnesium stearate.

In some embodiments, each tablet of the initiation dosage comprises:
about 400 mg of the spray dried dispersion of the sodium salt of the compound of Formula Ia; and
about 0.5 mg to about 0.6 mg of the compound of Formula IIa or IIb, or a pharmaceutically acceptable salt, co-crystal, solvate, or combination thereof.

In some embodiments, each tablet of the initiation dosage comprises:
about 400 mg of the spray dried dispersion of the sodium salt of the compound of Formula Ia; and
about 0.5325 mg of the compound of Formula IIa or IIb, or a pharmaceutically acceptable salt, co-crystal, solvate, or combination thereof.

In some embodiments, each tablet of the initiation dosage comprises:
about 400 mg of the spray dried dispersion of the sodium salt of the compound of Formula Ia; and
about 0.5 mg of the compound of Formula IIa or IIb, or a pharmaceutically acceptable salt, co-crystal, solvate, or combination thereof, on a free base basis.

In some embodiments, each tablet of the initiation dosage comprises:
about 400 mg of the spray dried dispersion of the sodium salt of the compound of Formula Ia;
about 0.5 mg to about 0.6 mg of the compound of Formula IIa or IIb, or a pharmaceutically acceptable salt, co-crystal, solvate, or combination thereof;
about 615 mg to about 625 mg of mannitol;
about 330 mg to about 340 mg of microcrystalline cellulose;
about 115 mg to about 125 mg croscarmellose sodium; and
about 20 mg to about 25 mg magnesium stearate.

In some embodiments, each tablet of the initiation dosage comprises:
about 400 mg of the spray dried dispersion of the sodium salt of the compound of Formula Ia;
about 0.5325 mg of the compound of Formula IIa or IIb, or a pharmaceutically acceptable salt, co-crystal, solvate, or combination thereof;
about 621 mg of mannitol;
about 335.9175 mg of microcrystalline cellulose;
about 120 mg croscarmellose sodium; and
about 22.5 mg magnesium stearate.

In some embodiments, each tablet of the initiation dosage comprises:
about 400 mg of the spray dried dispersion of the sodium salt of the compound of Formula Ia;
about 0.5 mg of the compound of Formula IIa or IIb, or a pharmaceutically acceptable salt, co-crystal, solvate, or combination thereof, on a free base basis;
about 621 mg of mannitol;
about 335.9175 mg of microcrystalline cellulose;
about 120 mg croscarmellose sodium; and
about 22.5 mg magnesium stearate.

In some embodiments, each tablet of the initiation dosage comprises:
about 26.6 w/w % to about 26.7 w/w % of the lenacapavir spray dried dispersion;
about 0.03 w/w % to about 0.04 w/w % of the compound of Formula IIa or IIb, or a pharmaceutically acceptable salt, co-crystal, solvate, or combination thereof; and
one or more pharmaceutically acceptable excipients.

In some embodiments, each tablet of the initiation dosage comprises:
about 26.67 w/w % of the lenacapavir spray dried dispersion;
about 0.0355 w/w % of the compound of Formula IIa or IIb, or a pharmaceutically acceptable salt, co-crystal, solvate, or combination thereof; and
one or more pharmaceutically acceptable excipients.

In some embodiments, each tablet of the initiation dosage comprises:
about 26.6 w/w % to about 26.7 w/w % of the lenacapavir spray dried dispersion;
about 0.03 w/w % to about 0.04 w/w % of the compound of Formula IIa or IIb, or a solvate thereof, and
one or more pharmaceutically acceptable excipients.

In some embodiments, each tablet of the initiation dosage comprises:
about 26.67 w/w % of the lenacapavir spray dried dispersion;
about 0.0355 w/w % of the compound of Formula IIa or IIb, or a solvate thereof, and
one or more pharmaceutically acceptable excipients.

In some embodiments, each tablet of the initiation dosage comprises:
about 26.6 w/w % to about 26.7 w/w % of the lenacapavir spray dried dispersion;
about 0.03 w/w % to about 0.04 w/w % of the monohydrate of the compound of Formula IIa or IIb; and
one or more pharmaceutically acceptable excipients.

In some embodiments, each tablet of the initiation dosage comprises:
about 26.67 w/w % of the lenacapavir spray dried dispersion;
about 0.0355 w/w % of the monohydrate of the compound of Formula IIa or IIb; and
one or more pharmaceutically acceptable excipients.

In some embodiments, each tablet of the initiation dosage comprises:
about 26.6 w/w % to about 26.7 w/w % of the lenacapavir spray dried dispersion;
about 0.03 w/w % to about 0.04 w/w % of the compound of Formula IIa or IIb, or a pharmaceutically acceptable salt, co-crystal, solvate, or combination thereof;
about 41 w/w % to about 42 w/w % of mannitol;
about 22.3 w/w % to about 22.4 w/w % of microcrystalline cellulose;
about 7.9 w/w % to about 8.1 w/w % of croscarmellose sodium; and
about 1.4 w/w % to about 1.6 w/w % of magnesium stearate.

In some embodiments, each tablet of the initiation dosage comprises:
about 26.67 w/w % of the compound of the lenacapavir spray dried dispersion;
about 0.0355 w/w % of the compound of Formula IIa or IIb, or a pharmaceutically acceptable salt, co-crystal, solvate, or combination thereof;
about 41.4 w/w % of mannitol;
about 22.3945 w/w % of microcrystalline cellulose;
about 8.0 w/w % of croscarmellose sodium; and
about 1.5 w/w % of magnesium stearate.

In some embodiments, each tablet of the initiation dosage comprises:
about 400 mg of the lenacapavir spray dried dispersion; and
about 0.5 mg to about 0.6 mg of the compound of Formula IIa or IIb, or a pharmaceutically acceptable salt, co-crystal, solvate, or combination thereof.

In some embodiments, each tablet of the initiation dosage comprises:
about 400 mg of the lenacapavir spray dried dispersion; and
about 0.5325 mg of the compound of Formula IIa or IIb, or a pharmaceutically acceptable salt, co-crystal, solvate, or combination thereof.

In some embodiments, each tablet of the initiation dosage comprises:
about 400 mg of the lenacapavir spray dried dispersion; and
about 0.5 mg of the compound of Formula IIa or IIb, or a pharmaceutically acceptable salt, co-crystal, solvate, or combination thereof, on a free base basis.

In some embodiments, each tablet of the initiation dosage comprises:
about 400 mg of the lenacapavir spray dried dispersion;
about 0.5 mg to about 0.6 mg of the compound of Formula IIa or IIb, or a pharmaceutically acceptable salt, co-crystal, solvate, or combination thereof;
about 615 mg to about 625 mg of mannitol;
about 330 mg to about 340 mg of microcrystalline cellulose;
about 115 mg to about 125 mg croscarmellose sodium; and
about 20 mg to about 25 mg magnesium stearate.

In some embodiments, each tablet of the initiation dosage comprises:
about 400 mg of the lenacapavir spray dried dispersion;
about 0.5325 mg of the compound of Formula IIa or IIb, or a pharmaceutically acceptable salt, co-crystal, solvate, or combination thereof;
about 621 mg of mannitol;
about 335.9175 mg of microcrystalline cellulose;
about 120 mg croscarmellose sodium; and
about 22.5 mg magnesium stearate.

In some embodiments, each tablet of the initiation dosage comprises:
about 400 mg of the lenacapavir spray dried dispersion;
about 0.5 mg of the compound of Formula IIa or IIb, or a pharmaceutically acceptable salt, co-crystal, solvate, or combination thereof, on a free base basis;
about 621 mg of mannitol;
about 335.9175 mg of microcrystalline cellulose;
about 120 mg croscarmellose sodium; and
about 22.5 mg magnesium stearate.

In some embodiments, each tablet of the initiation dosage comprises:
about 300 mg of the compound of Formula Ia or Ib, or a pharmaceutically acceptable salt, co-crystal, solvate, or combination thereof, on a free acid basis; and
about 0.5 mg to about 0.6 mg of the compound of Formula IIa or IIb, or a pharmaceutically acceptable salt, co-crystal, solvate, or combination thereof.

In some embodiments, each tablet of the initiation dosage comprises:
about 300 mg of the compound of Formula Ia or Ib, or a pharmaceutically acceptable salt, co-crystal, solvate, or combination thereof, on a free acid basis; and
about 0.5325 mg of the compound of Formula IIa or IIb, or a pharmaceutically acceptable salt, co-crystal, solvate, or combination thereof.

In some embodiments, each tablet of the initiation dosage comprises:
about 300 mg of the compound of Formula Ia or Ib, or a pharmaceutically acceptable salt, co-crystal, solvate, or combination thereof, on a free acid basis; and
about 0.5 mg of the compound of Formula IIa or IIb, or a pharmaceutically acceptable salt, co-crystal, solvate, or combination thereof, on a free base basis.

In some embodiments, each tablet of the initiation dosage comprises:
about 300 mg of the compound of Formula Ia or Ib, or a pharmaceutically acceptable salt, co-crystal, solvate, or combination thereof, on a free acid basis;
about 0.5 mg to about 0.6 mg of the compound of Formula IIa or IIb, or a pharmaceutically acceptable salt, co-crystal, solvate, or combination thereof;
about 615 mg to about 625 mg of mannitol;
about 330 mg to about 340 mg of microcrystalline cellulose;
about 115 mg to about 125 mg croscarmellose sodium; and
about 20 mg to about 25 mg magnesium stearate.

In some embodiments, each tablet of the initiation dosage comprises:
about 300 mg of the compound of Formula Ia or Ib, or a pharmaceutically acceptable salt, co-crystal, solvate, or combination thereof, on a free acid basis;
about 0.5325 mg of the compound of Formula IIa or IIb, or a pharmaceutically acceptable salt, co-crystal, solvate, or combination thereof;
about 621 mg of mannitol;
about 335.9175 mg of microcrystalline cellulose;
about 120 mg croscarmellose sodium; and
about 22.5 mg magnesium stearate.

In some embodiments, each tablet of the initiation dosage comprises:
about 300 mg of the compound of Formula Ia or Ib, or a pharmaceutically acceptable salt, co-crystal, solvate, or combination thereof, on a free acid basis;
about 0.5 mg of the compound of Formula IIa or IIb, or a pharmaceutically acceptable salt, co-crystal, solvate, or combination thereof, on a free base basis;
about 621 mg of mannitol;
about 335.9175 mg of microcrystalline cellulose;
about 120 mg croscarmellose sodium; and
about 22.5 mg magnesium stearate.

In some embodiments, each tablet of the initiation dosage comprises:
about 200 mg to about 400 mg of the compound of Formula Ia or Ib, or a pharmaceutically acceptable salt thereof, on a free acid basis;
about 0.4 mg to about 0.6 mg of the compound of Formula IIa or IIb, or a solvate thereof, on a free base basis;
about 50 mg to about 100 mg of copovidone;
about 5 mg to about 30 mg of poloxamer 407;
about 600 mg to about 700 mg of mannitol;
about 300 mg to about 350 mg of microcrystalline cellulose;
about 100 mg to about 150 mg of croscarmellose sodium; and
about 20 mg to about 25 mg of magnesium stearate.

In some embodiments, each tablet of the initiation dosage comprises:
about 300 mg of the compound of Formula Ia or Ib, or a pharmaceutically acceptable salt thereof, on a free acid basis;
about 0.5 mg of the compound of Formula IIa or IIb, or a solvate thereof, on a free base basis;
about 65 mg to about 75 mg of copovidone;
about 15 mg to about 25 mg of poloxamer 407;
about 615 mg to about 625 mg of mannitol;
about 330 mg to about 340 mg of microcrystalline cellulose;
about 115 mg to about 125 mg of croscarmellose sodium; and
about 20 mg to about 25 mg of magnesium stearate.

In some embodiments, each tablet of the initiation dosage comprises:
about 300 mg of the compound of Formula Ia or Ib, or a pharmaceutically acceptable salt thereof, on a free acid basis;
about 0.5 mg of the compound of Formula IIa or IIb, or a solvate thereof, on a free base basis;
about 73.2 mg of copovidone;
about 19.95 mg of poloxamer 407;
about 621 mg of mannitol;
about 335.92 mg of microcrystalline cellulose;
about 120 mg of croscarmellose sodium; and
about 22.5 mg of magnesium stearate.

In some embodiments, each tablet of the initiation dosage comprises:
about 200 mg to about 400 mg of the sodium salt of the compound of Formula Ia or Ib;
about 0.4 mg to about 0.6 mg of the monohydrate of the compound of Formula IIa or IIb;
about 50 mg to about 100 mg of copovidone;
about 5 mg to about 30 mg of poloxamer 407;
about 600 mg to about 700 mg of mannitol;
about 300 mg to about 350 mg of microcrystalline cellulose;
about 100 mg to about 150 mg of croscarmellose sodium; and
about 20 mg to about 25 mg of magnesium stearate.

In some embodiments, each tablet of the initiation dosage comprises:
about 306.8 mg of the sodium salt of the compound of Formula Ia or Ib;
about 0.5 mg to about 0.6 mg of the monohydrate of the compound of Formula IIa or IIb;
about 65 mg to about 75 mg of copovidone;
about 15 mg to about 25 mg of poloxamer 407;
about 615 mg to about 625 mg of mannitol;
about 330 mg to about 340 mg of microcrystalline cellulose;
about 115 mg to about 125 mg of croscarmellose sodium; and
about 20 mg to about 25 mg of magnesium stearate.

In some embodiments, each tablet of the initiation dosage comprises:
about 306.8 mg of the sodium salt of the compound of Formula Ia or Ib;
about 0.53 mg of the monohydrate of the compound of Formula IIa or IIb;
about 65 mg to about 75 mg of copovidone;
about 15 mg to about 25 mg of poloxamer 407;
about 615 mg to about 625 mg of mannitol;
about 330 mg to about 340 mg of microcrystalline cellulose;
about 115 mg to about 125 mg of croscarmellose sodium; and
about 20 mg to about 25 mg of magnesium stearate.

In some embodiments, each tablet of the initiation dosage comprises:
about 306.8 mg of the sodium salt of the compound of Formula Ia or Ib;
about 0.53 mg of the monohydrate of the compound of Formula IIa or IIb;
about 73.2 mg of copovidone;
about 19.95 mg of poloxamer 407;
about 621 mg of mannitol;
about 335.92 mg of microcrystalline cellulose;
about 120 mg of croscarmellose sodium; and
about 22.5 mg of magnesium stearate.

In some embodiments, each tablet of the initiation dosage comprises:
about 200 mg to about 400 mg of the compound of Formula Ia or Ib, or a pharmaceutically acceptable salt thereof, on a free acid basis;
about 0.4 mg to about 0.6 mg of the compound of Formula IIa or IIb, or a solvate thereof, on a free base basis;
about 50 mg to about 100 mg of copovidone;
about 5 mg to about 30 mg of poloxamer 407;
about 600 mg to about 700 mg of mannitol;
about 300 mg to about 350 mg of microcrystalline cellulose;
about 100 mg to about 150 mg of croscarmellose sodium;
about 20 mg to about 25 mg of magnesium stearate; and
an outer film coat.

In some embodiments, each tablet of the initiation dosage comprises:
about 300 mg of the compound of Formula Ia or Ib, or a pharmaceutically acceptable salt thereof, on a free acid basis;
about 0.5 mg of the compound of Formula IIa or IIb, or a solvate thereof, on a free base basis;
about 65 mg to about 75 mg of copovidone;
about 15 mg to about 25 mg of poloxamer 407;
about 615 mg to about 625 mg of mannitol;
about 330 mg to about 340 mg of microcrystalline cellulose;
about 115 mg to about 125 mg of croscarmellose sodium;
about 20 mg to about 25 mg of magnesium stearate; and
an outer film coat.

In some embodiments, each tablet of the initiation dosage comprises:
about 300 mg of the compound of Formula Ia or Ib, or a pharmaceutically acceptable salt thereof, on a free acid basis;
about 0.5 mg of the compound of Formula IIa or IIb, or a solvate thereof, on a free base basis, on a free base basis;
about 73.2 mg of copovidone;
about 19.95 mg of poloxamer 407;
about 621 mg of mannitol;
about 335.92 mg of microcrystalline cellulose;
about 120 mg of croscarmellose sodium;
about 22.5 mg of magnesium stearate; and
an outer film coat.

In some embodiments, each tablet of the initiation dosage comprises:
about 200 mg to about 400 mg of the sodium salt of the compound of Formula Ia or Ib;
about 0.4 mg to about 0.6 mg of the monohydrate of the compound of Formula IIa or IIb;
about 50 mg to about 100 mg of copovidone;
about 5 mg to about 30 mg of poloxamer 407;
about 600 mg to about 700 mg of mannitol;
about 300 mg to about 350 mg of microcrystalline cellulose;
about 100 mg to about 150 mg of croscarmellose sodium;
about 20 mg to about 25 mg of magnesium stearate; and
an outer film coat.

In some embodiments, each tablet of the initiation dosage comprises:
about 306.8 mg of the sodium salt of the compound of Formula Ia or Ib;
about 0.5 mg to about 0.6 mg of the monohydrate of the compound of Formula IIa or IIb;
about 65 mg to about 75 mg of copovidone;
about 15 mg to about 25 mg of poloxamer 407;
about 615 mg to about 625 mg of mannitol;
about 330 mg to about 340 mg of microcrystalline cellulose;
about 115 mg to about 125 mg of croscarmellose sodium;
about 20 mg to about 25 mg of magnesium stearate; and
an outer film coat.

In some embodiments, each tablet of the initiation dosage comprises:
about 306.8 mg of the sodium salt of the compound of Formula Ia or Ib;
about 0.53 mg of the monohydrate of the compound of Formula IIa or IIb;
about 65 mg to about 75 mg of copovidone;
about 15 mg to about 25 mg of poloxamer 407;
about 615 mg to about 625 mg of mannitol;
about 330 mg to about 340 mg of microcrystalline cellulose;
about 115 mg to about 125 mg of croscarmellose sodium; and
about 20 mg to about 25 mg of magnesium stearate.

In some embodiments, each tablet of the initiation dosage comprises:
about 306.8 mg of the sodium salt of the compound of Formula Ia or Ib;
about 0.53 mg of the monohydrate of the compound of Formula IIa or IIb;
about 73.2 mg of copovidone;
about 19.95 mg of poloxamer 407;
about 621 mg of mannitol;
about 335.92 mg of microcrystalline cellulose;
about 120 mg of croscarmellose sodium;
about 22.5 mg of magnesium stearate; and
an outer film coat.

In some embodiments, each tablet of the initiation dosage consists essentially of:
about 200 mg to about 400 mg of the compound of Formula Ia or Ib, or a pharmaceutically acceptable salt thereof, on a free acid basis;
about 0.4 mg to about 0.6 mg of the compound of Formula IIa or IIb, or a solvate thereof, on a free base basis;
about 50 mg to about 100 mg of copovidone;
about 5 mg to about 30 mg of poloxamer 407;
about 600 mg to about 700 mg of mannitol;
about 300 mg to about 350 mg of microcrystalline cellulose;
about 100 mg to about 150 mg of croscarmellose sodium; and
about 20 mg to about 25 mg of magnesium stearate.

In some embodiments, each tablet of the initiation dosage consists essentially of:
about 300 mg of the compound of Formula Ia or Ib, or a pharmaceutically acceptable salt thereof, on a free acid basis;
about 0.5 mg of the compound of Formula IIa or IIb, or a solvate thereof, on a free base basis;
about 65 mg to about 75 mg of copovidone;
about 15 mg to about 25 mg of poloxamer 407;
about 615 mg to about 625 mg of mannitol;
about 330 mg to about 340 mg of microcrystalline cellulose;
about 115 mg to about 125 mg of croscarmellose sodium; and
about 20 mg to about 25 mg of magnesium stearate.

In some embodiments, each tablet of the initiation dosage consists essentially of:
about 300 mg of the compound of Formula Ia or Ib, or a pharmaceutically acceptable salt thereof, on a free acid basis;
about 0.5 mg of the compound of Formula IIa or IIb, or a solvate thereof, on a free base basis;
about 73.2 mg of copovidone;
about 19.95 mg of poloxamer 407;
about 621 mg of mannitol;
about 335.92 mg of microcrystalline cellulose;
about 120 mg of croscarmellose sodium; and
about 22.5 mg of magnesium stearate.

In some embodiments, each tablet of the initiation dosage consists essentially of:
about 200 mg to about 400 mg of the sodium salt of the compound of Formula Ia or Ib;
about 0.4 mg to about 0.6 mg of the monohydrate of the compound of Formula IIa or IIb;
about 50 mg to about 100 mg of copovidone;
about 5 mg to about 30 mg of poloxamer 407;
about 600 mg to about 700 mg of mannitol;
about 300 mg to about 350 mg of microcrystalline cellulose;
about 100 mg to about 150 mg of croscarmellose sodium; and
about 20 mg to about 25 mg of magnesium stearate.

In some embodiments, each tablet of the initiation dosage consists essentially of:
about 306.8 mg of the sodium salt of the compound of Formula Ia or Ib;
about 0.5 mg to about 0.6 mg of the monohydrate of the compound of Formula IIa or IIb;
about 65 mg to about 75 mg of copovidone;
about 15 mg to about 25 mg of poloxamer 407;
about 615 mg to about 625 mg of mannitol;
about 330 mg to about 340 mg of microcrystalline cellulose;
about 115 mg to about 125 mg of croscarmellose sodium; and
about 20 mg to about 25 mg of magnesium stearate.

In some embodiments, each tablet of the initiation dosage consists essentially of:
about 306.8 mg of the sodium salt of the compound of Formula Ia or Ib;
about 0.53 mg of the monohydrate of the compound of Formula IIa or IIb;
about 65 mg to about 75 mg of copovidone;
about 15 mg to about 25 mg of poloxamer 407;
about 615 mg to about 625 mg of mannitol;
about 330 mg to about 340 mg of microcrystalline cellulose;
about 115 mg to about 125 mg of croscarmellose sodium; and
about 20 mg to about 25 mg of magnesium stearate.

In some embodiments, each tablet of the initiation dosage consists essentially of:
about 306.8 mg of the sodium salt of the compound of Formula Ia or Ib;
about 0.53 mg of the monohydrate of the compound of Formula IIa or IIb;
about 73.2 mg of copovidone;
about 19.95 mg of poloxamer 407;
about 621 mg of mannitol;
about 335.92 mg of microcrystalline cellulose;
about 120 mg of croscarmellose sodium; and
about 22.5 mg of magnesium stearate.

In some embodiments, each tablet of the initiation dosage consists essentially of:
about 200 mg to about 400 mg of the compound of Formula Ia or Ib, or a pharmaceutically acceptable salt thereof, on a free acid basis;
about 0.4 mg to about 0.6 mg of the compound of Formula IIa or IIb, or a solvate thereof, on a free base basis;
about 50 mg to about 100 mg of copovidone;
about 5 mg to about 30 mg of poloxamer 407;
about 600 mg to about 700 mg of mannitol;
about 300 mg to about 350 mg of microcrystalline cellulose;
about 100 mg to about 150 mg of croscarmellose sodium;
about 20 mg to about 25 mg of magnesium stearate; and
an outer film coat.

In some embodiments, each tablet of the initiation dosage consists essentially of:
about 300 mg of the compound of Formula Ia or Ib, or a pharmaceutically acceptable salt thereof, on a free acid basis;
about 0.5 mg of the compound of Formula IIa or IIb, or a solvate thereof, on a free base basis;
about 65 mg to about 75 mg of copovidone;
about 15 mg to about 25 mg of poloxamer 407;
about 615 mg to about 625 mg of mannitol;
about 330 mg to about 340 mg of microcrystalline cellulose;
about 115 mg to about 125 mg of croscarmellose sodium;
about 20 mg to about 25 mg of magnesium stearate; and
an outer film coat.

In some embodiments, each tablet of the initiation dosage consists essentially of:
about 300 mg of the compound of Formula Ia or Ib, or a pharmaceutically acceptable salt thereof, on a free acid basis;
about 0.5 mg of the compound of Formula IIa or IIb, or a solvate thereof, on a free base basis, on a free base basis;
about 73.2 mg of copovidone;
about 19.95 mg of poloxamer 407;
about 621 mg of mannitol;
about 335.92 mg of microcrystalline cellulose;
about 120 mg of croscarmellose sodium;
about 22.5 mg of magnesium stearate; and
an outer film coat.

In some embodiments, each tablet of the initiation dosage consists essentially of:
about 200 mg to about 400 mg of the sodium salt of the compound of Formula Ia or Ib;
about 0.4 mg to about 0.6 mg of the monohydrate of the compound of Formula IIa or IIb;
about 50 mg to about 100 mg of copovidone;
about 5 mg to about 30 mg of poloxamer 407;
about 600 mg to about 700 mg of mannitol;
about 300 mg to about 350 mg of microcrystalline cellulose;
about 100 mg to about 150 mg of croscarmellose sodium;
about 20 mg to about 25 mg of magnesium stearate; and
an outer film coat.

In some embodiments, each tablet of the initiation dosage consists essentially of:
about 306.8 mg of the sodium salt of the compound of Formula Ia or Ib;
about 0.5 mg to about 0.6 mg of the monohydrate of the compound of Formula IIa or IIb;
about 65 mg to about 75 mg of copovidone;
about 15 mg to about 25 mg of poloxamer 407;
about 615 mg to about 625 mg of mannitol;
about 330 mg to about 340 mg of microcrystalline cellulose;
about 115 mg to about 125 mg of croscarmellose sodium;
about 20 mg to about 25 mg of magnesium stearate; and
an outer film coat.

In some embodiments, each tablet of the initiation dosage consists essentially of:
about 306.8 mg of the sodium salt of the compound of Formula Ia or Ib;
about 0.53 mg of the monohydrate of the compound of Formula IIa or IIb;
about 65 mg to about 75 mg of copovidone;
about 15 mg to about 25 mg of poloxamer 407;
about 615 mg to about 625 mg of mannitol;
about 330 mg to about 340 mg of microcrystalline cellulose;
about 115 mg to about 125 mg of croscarmellose sodium; and
about 20 mg to about 25 mg of magnesium stearate.

In some embodiments, each tablet of the initiation dosage consists essentially of:
about 306.8 mg of the sodium salt of the compound of Formula Ia or Ib;
about 0.53 mg of the monohydrate of the compound of Formula IIa or IIb;
about 73.2 mg of copovidone;
about 19.95 mg of poloxamer 407;
about 621 mg of mannitol;
about 335.92 mg of microcrystalline cellulose;
about 120 mg of croscarmellose sodium;
about 22.5 mg of magnesium stearate; and
an outer film coat.

In some embodiments, each tablet of the initiation dosage consists of:
about 200 mg to about 400 mg of the compound of Formula Ia or Ib, or a pharmaceutically acceptable salt thereof, on a free acid basis;
about 0.4 mg to about 0.6 mg of the compound of Formula IIa or IIb, or a solvate thereof, on a free base basis;
about 50 mg to about 100 mg of copovidone;
about 5 mg to about 30 mg of poloxamer 407;
about 600 mg to about 700 mg of mannitol;
about 300 mg to about 350 mg of microcrystalline cellulose;
about 100 mg to about 150 mg of croscarmellose sodium; and
about 20 mg to about 25 mg of magnesium stearate.

In some embodiments, each tablet of the initiation dosage consists of:
about 300 mg of the compound of Formula Ia or Ib, or a pharmaceutically acceptable salt thereof, on a free acid basis;
about 0.5 mg of the compound of Formula IIa or IIb, or a solvate thereof, on a free base basis;
about 65 mg to about 75 mg of copovidone;
about 15 mg to about 25 mg of poloxamer 407;
about 615 mg to about 625 mg of mannitol;
about 330 mg to about 340 mg of microcrystalline cellulose;
about 115 mg to about 125 mg of croscarmellose sodium; and
about 20 mg to about 25 mg of magnesium stearate.

In some embodiments, each tablet of the initiation dosage consists of:
about 300 mg of the compound of Formula Ia or Ib, or a pharmaceutically acceptable salt thereof, on a free acid basis;
about 0.5 mg of the compound of Formula IIa or IIb, or a solvate thereof, on a free base basis;
about 73.2 mg of copovidone;
about 19.95 mg of poloxamer 407;
about 621 mg of mannitol;
about 335.92 mg of microcrystalline cellulose;
about 120 mg of croscarmellose sodium; and
about 22.5 mg of magnesium stearate.

In some embodiments, each tablet of the initiation dosage consists of:
about 200 mg to about 400 mg of the sodium salt of the compound of Formula Ia or Ib;
about 0.4 mg to about 0.6 mg of the monohydrate of the compound of Formula IIa or IIb;
about 50 mg to about 100 mg of copovidone;
about 5 mg to about 30 mg of poloxamer 407;
about 600 mg to about 700 mg of mannitol;
about 300 mg to about 350 mg of microcrystalline cellulose;
about 100 mg to about 150 mg of croscarmellose sodium; and
about 20 mg to about 25 mg of magnesium stearate.

In some embodiments, each tablet of the initiation dosage consists of:
about 306.8 mg of the sodium salt of the compound of Formula Ia or Ib;
about 0.5 mg to about 0.6 mg of the monohydrate of the compound of Formula IIa or IIb;
about 65 mg to about 75 mg of copovidone;
about 15 mg to about 25 mg of poloxamer 407;
about 615 mg to about 625 mg of mannitol;
about 330 mg to about 340 mg of microcrystalline cellulose;
about 115 mg to about 125 mg of croscarmellose sodium; and
about 20 mg to about 25 mg of magnesium stearate.

In some embodiments, each tablet of the initiation dosage consists of:
about 306.8 mg of the sodium salt of the compound of Formula Ia or Ib;
about 0.53 mg of the monohydrate of the compound of Formula IIa or IIb;
about 65 mg to about 75 mg of copovidone;
about 15 mg to about 25 mg of poloxamer 407;
about 615 mg to about 625 mg of mannitol;
about 330 mg to about 340 mg of microcrystalline cellulose;
about 115 mg to about 125 mg of croscarmellose sodium; and
about 20 mg to about 25 mg of magnesium stearate.

In some embodiments, each tablet of the initiation dosage consists of:
about 306.8 mg of the sodium salt of the compound of Formula Ia or Ib;
about 0.53 mg of the monohydrate of the compound of Formula IIa or IIb;
about 73.2 mg of copovidone;
about 19.95 mg of poloxamer 407;
about 621 mg of mannitol;
about 335.92 mg of microcrystalline cellulose;
about 120 mg of croscarmellose sodium; and
about 22.5 mg of magnesium stearate.

In some embodiments, each tablet of the initiation dosage consists of:
about 200 mg to about 400 mg of the compound of Formula Ia or Ib, or a pharmaceutically acceptable salt thereof, on a free acid basis;
about 0.4 mg to about 0.6 mg of the compound of Formula IIa or IIb, or a solvate thereof, on a free base basis;
about 50 mg to about 100 mg of copovidone;
about 5 mg to about 30 mg of poloxamer 407;
about 600 mg to about 700 mg of mannitol;
about 300 mg to about 350 mg of microcrystalline cellulose;
about 100 mg to about 150 mg of croscarmellose sodium;
about 20 mg to about 25 mg of magnesium stearate; and
an outer film coat.

In some embodiments, each tablet of the initiation dosage consists of:
about 300 mg of the compound of Formula Ia or Ib, or a pharmaceutically acceptable salt thereof, on a free acid basis;
about 0.5 mg of the compound of Formula IIa or IIb, or a solvate thereof, on a free base basis;
about 65 mg to about 75 mg of copovidone;
about 15 mg to about 25 mg of poloxamer 407;
about 615 mg to about 625 mg of mannitol;
about 330 mg to about 340 mg of microcrystalline cellulose;
about 115 mg to about 125 mg of croscarmellose sodium;
about 20 mg to about 25 mg of magnesium stearate; and
an outer film coat.

In some embodiments, each tablet of the initiation dosage consists of:
about 300 mg of the compound of Formula Ia or Ib, or a pharmaceutically acceptable salt thereof, on a free acid basis;
about 0.5 mg of the compound of Formula IIa or IIb, or a solvate thereof, on a free base basis, on a free base basis;
about 73.2 mg of copovidone;
about 19.95 mg of poloxamer 407;
about 621 mg of mannitol;
about 335.92 mg of microcrystalline cellulose;
about 120 mg of croscarmellose sodium;
about 22.5 mg of magnesium stearate; and
an outer film coat.

In some embodiments, each tablet of the initiation dosage consists of:
about 200 mg to about 400 mg of the sodium salt of the compound of Formula Ia or Ib;
about 0.4 mg to about 0.6 mg of the monohydrate of the compound of Formula IIa or IIb;
about 50 mg to about 100 mg of copovidone;
about 5 mg to about 30 mg of poloxamer 407;
about 600 mg to about 700 mg of mannitol;
about 300 mg to about 350 mg of microcrystalline cellulose;
about 100 mg to about 150 mg of croscarmellose sodium;
about 20 mg to about 25 mg of magnesium stearate; and
an outer film coat.

In some embodiments, each tablet of the initiation dosage consists of:
about 306.8 mg of the sodium salt of the compound of Formula Ia or Ib;
about 0.5 mg to about 0.6 mg of the monohydrate of the compound of Formula IIa or IIb;
about 65 mg to about 75 mg of copovidone;
about 15 mg to about 25 mg of poloxamer 407;
about 615 mg to about 625 mg of mannitol;
about 330 mg to about 340 mg of microcrystalline cellulose;
about 115 mg to about 125 mg of croscarmellose sodium;
about 20 mg to about 25 mg of magnesium stearate; and
an outer film coat.

In some embodiments, each tablet of the initiation dosage consists of:
about 306.8 mg of the sodium salt of the compound of Formula Ia or Ib;
about 0.53 mg of the monohydrate of the compound of Formula IIa or IIb;
about 65 mg to about 75 mg of copovidone;
about 15 mg to about 25 mg of poloxamer 407;
about 615 mg to about 625 mg of mannitol;
about 330 mg to about 340 mg of microcrystalline cellulose;
about 115 mg to about 125 mg of croscarmellose sodium; and
about 20 mg to about 25 mg of magnesium stearate.

In some embodiments, each tablet of the initiation dosage consists of:
about 306.8 mg of the sodium salt of the compound of Formula Ia or Ib;
about 0.53 mg of the monohydrate of the compound of Formula IIa or IIb;
about 73.2 mg of copovidone;
about 19.95 mg of poloxamer 407;
about 621 mg of mannitol;
about 335.92 mg of microcrystalline cellulose;
about 120 mg of croscarmellose sodium;
about 22.5 mg of magnesium stearate; and
an outer film coat.

In some embodiments, the method comprises administering to the patient an initiation dose comprising two tablets on day 1, and two tablets on day 2, wherein each tablet of the initiation dose comprises:
about 26.6 w/w % to about 26.7 w/w % of the spray dried dispersion of the compound of Formula Ia or Ib, or a pharmaceutically acceptable salt, co-crystal, solvate, or combination thereof;
about 0.03 w/w % to about 0.04 w/w % of the compound of Formula IIa or IIb, or a pharmaceutically acceptable salt, co-crystal, solvate, or combination thereof; and
one or more pharmaceutically acceptable excipients.

In some embodiments, the method comprises administering to the patient an initiation dose comprising two tablets on day 1, and two tablets on day 2, wherein each tablet of the initiation dose comprises:
about 26.67 w/w % of the spray dried dispersion of the compound of Formula Ia or Ib, or a pharmaceutically acceptable salt, co-crystal, solvate, or combination thereof;
about 0.0355 w/w % of the compound of Formula IIa or IIb, or a pharmaceutically acceptable salt, co-crystal, solvate, or combination thereof; and
one or more pharmaceutically acceptable excipients.

In some embodiments, the method comprises administering to the patient an initiation dose comprising two tablets on day 1, and two tablets on day 2, wherein each tablet of the initiation dose comprises:
about 26.6 w/w % to about 26.7 w/w % of the spray dried dispersion of the compound of Formula Ia or Ib, or a pharmaceutically acceptable salt, co-crystal, solvate, or combination thereof;
about 0.03 w/w % to about 0.04 w/w % of the compound of Formula IIa or IIb, or a solvate thereof, and
one or more pharmaceutically acceptable excipients.

In some embodiments, the method comprises administering to the patient an initiation dose comprising two tablets on day 1, and two tablets on day 2, wherein each tablet of the initiation dose comprises:
about 26.67 w/w % of the spray dried dispersion of the compound of Formula Ia or Ib, or a pharmaceutically acceptable salt, co-crystal, solvate, or combination thereof;
about 0.0355 w/w % of the compound of Formula IIa or IIb, or a solvate thereof, and
one or more pharmaceutically acceptable excipients.

In some embodiments, the method comprises administering to the patient an initiation dose comprising two tablets on day 1, and two tablets on day 2, wherein each tablet of the initiation dose comprises:
about 26.6 w/w % to about 26.7 w/w % of the spray dried dispersion of the compound of Formula Ia or Ib, or a pharmaceutically acceptable salt, co-crystal, solvate, or combination thereof;
about 0.03 w/w % to about 0.04 w/w % of the monohydrate of the compound of Formula Ila or IIb; and
one or more pharmaceutically acceptable excipients.

In some embodiments, the method comprises administering to the patient an initiation dose comprising two tablets on day 1, and two tablets on day 2, wherein each tablet of the initiation dose comprises:
about 26.67 w/w % of the spray dried dispersion of the compound of Formula Ia or Ib, or a pharmaceutically acceptable salt, co-crystal, solvate, or combination thereof;
about 0.0355 w/w % of the monohydrate of the compound of Formula IIa or IIb; and
one or more pharmaceutically acceptable excipients.

In some embodiments, the method comprises administering to the patient an initiation dose comprising two tablets on day 1, and two tablets on day 2, wherein each tablet of the initiation dose comprises:
about 26.6 w/w % to about 26.7 w/w % of the spray dried dispersion of the compound of Formula Ia or Ib, or a pharmaceutically acceptable salt, co-crystal, solvate, or combination thereof;
about 0.03 w/w % to about 0.04 w/w % of the compound of Formula IIa or IIb, or a pharmaceutically acceptable salt, co-crystal, solvate, or combination thereof;
about 41 w/w % to about 42 w/w % of mannitol;
about 22.3 w/w % to about 22.4 w/w % of microcrystalline cellulose;
about 7.9 w/w % to about 8.1 w/w % of croscarmellose sodium; and
about 1.4 w/w % to about 1.6 w/w % of magnesium stearate.

In some embodiments, the method comprises administering to the patient an initiation dose comprising two tablets on day 1, and two tablets on day 2, wherein each tablet of the initiation dose comprises:
about 26.67 w/w % of the spray dried dispersion of the compound of Formula Ia or Ib, or a pharmaceutically acceptable salt, co-crystal, solvate, or combination thereof;
about 0.0355 w/w % of the compound of Formula IIa or IIb, or a pharmaceutically acceptable salt, co-crystal, solvate, or combination thereof;
about 41.4 w/w % of mannitol;
about 22.3945 w/w % of microcrystalline cellulose;
about 8.0 w/w % of croscarmellose sodium; and
about 1.5 w/w % of magnesium stearate.

In some embodiments, the method comprises administering to the patient an initiation dose comprising two tablets on day 1, and two tablets on day 2, wherein each tablet of the initiation dose comprises:
about 400 mg of the spray dried dispersion of the compound of Formula Ia or Ib, or a pharmaceutically acceptable salt, co-crystal, solvate, or combination thereof; and
about 0.5 mg to about 0.6 mg of the compound of Formula IIa or IIb, or a pharmaceutically acceptable salt, co-crystal, solvate, or combination thereof.

In some embodiments, the method comprises administering to the patient an initiation dose comprising two tablets on day 1, and two tablets on day 2, wherein each tablet of the initiation dose comprises:
about 400 mg of the spray dried dispersion of the compound of Formula Ia or Ib, or a pharmaceutically acceptable salt, co-crystal, solvate, or combination thereof; and
about 0.5325 mg of the compound of Formula IIa or IIb, or a pharmaceutically acceptable salt, co-crystal, solvate, or combination thereof.

In some embodiments, the method comprises administering to the patient an initiation dose comprising two tablets on day 1, and two tablets on day 2, wherein each tablet of the initiation dose comprises:
about 400 mg of the spray dried dispersion of the compound of Formula Ia or Ib, or a pharmaceutically acceptable salt, co-crystal, solvate, or combination thereof; and
about 0.5 mg of the compound of Formula IIa or IIb, or a pharmaceutically acceptable salt, co-crystal, solvate, or combination thereof, on a free base basis.

In some embodiments, the method comprises administering to the patient an initiation dose comprising two tablets on day 1, and two tablets on day 2, wherein each tablet of the initiation dose comprises:
about 400 mg of the spray dried dispersion of the compound of Formula Ia or Ib, or a pharmaceutically acceptable salt, co-crystal, solvate, or combination thereof;
about 0.5 mg to about 0.6 mg of the compound of Formula IIa or IIb, or a pharmaceutically acceptable salt, co-crystal, solvate, or combination thereof;
about 615 mg to about 625 mg of mannitol;
about 330 mg to about 340 mg of microcrystalline cellulose;
about 115 mg to about 125 mg croscarmellose sodium; and
about 20 mg to about 25 mg magnesium stearate.

In some embodiments, the method comprises administering to the patient an initiation dose comprising two tablets on day 1, and two tablets on day 2, wherein each tablet of the initiation dose comprises:
about 400 mg of the spray dried dispersion of the compound of Formula Ia or Ib, or a pharmaceutically acceptable salt, co-crystal, solvate, or combination thereof;
about 0.5325 mg of the compound of Formula IIa or IIb, or a pharmaceutically acceptable salt, co-crystal, solvate, or combination thereof;
about 621 mg of mannitol;
about 335.9175 mg of microcrystalline cellulose;
about 120 mg croscarmellose sodium; and
about 22.5 mg magnesium stearate.

In some embodiments, the method comprises administering to the patient an initiation dose comprising two tablets on day 1, and two tablets on day 2, wherein each tablet of the initiation dose comprises:
about 400 mg of the spray dried dispersion of the compound of Formula Ia or Ib, or a pharmaceutically acceptable salt, co-crystal, solvate, or combination thereof;
about 0.5 mg of the compound of Formula IIa or IIb, or a pharmaceutically acceptable salt, co-crystal, solvate, or combination thereof, on a free base basis;
about 621 mg of mannitol;
about 335.9175 mg of microcrystalline cellulose;
about 120 mg croscarmellose sodium; and
about 22.5 mg magnesium stearate.

In some embodiments, the method comprises administering to the patient an initiation dose comprising two tablets on day 1, and two tablets on day 2, wherein each tablet of the initiation dose comprises:
about 26.6 w/w % to about 26.7 w/w % of the spray dried dispersion of the compound of Formula Ia or Ib, or a pharmaceutically acceptable salt thereof;
about 0.03 w/w % to about 0.04 w/w % of the compound of Formula IIa or IIb, or a pharmaceutically acceptable salt, co-crystal, solvate, or combination thereof; and
one or more pharmaceutically acceptable excipients.

In some embodiments, the method comprises administering to the patient an initiation dose comprising two tablets on day 1, and two tablets on day 2, wherein each tablet of the initiation dose comprises:
about 26.67 w/w % of the spray dried dispersion of the compound of Formula Ia or Ib, or a pharmaceutically acceptable salt thereof;
about 0.0355 w/w % of the compound of Formula IIa or IIb, or a pharmaceutically acceptable salt, co-crystal, solvate, or combination thereof; and
one or more pharmaceutically acceptable excipients.

In some embodiments, the method comprises administering to the patient an initiation dose comprising two tablets on day 1, and two tablets on day 2, wherein each tablet of the initiation dose comprises:
about 26.6 w/w % to about 26.7 w/w % of the spray dried dispersion of the compound of Formula Ia or Ib, or a pharmaceutically acceptable salt thereof;
about 0.03 w/w % to about 0.04 w/w % of the compound of Formula IIa or IIb, or a solvate thereof, and
one or more pharmaceutically acceptable excipients.

In some embodiments, the method comprises administering to the patient an initiation dose comprising two tablets on day 1, and two tablets on day 2, wherein each tablet of the initiation dose comprises:
about 26.67 w/w % of the spray dried dispersion of the compound of Formula Ia or Ib, or a pharmaceutically acceptable salt thereof;
about 0.0355 w/w % of the compound of Formula IIa or IIb, or a solvate thereof, and
one or more pharmaceutically acceptable excipients.

In some embodiments, the method comprises administering to the patient an initiation dose comprising two tablets on day 1, and two tablets on day 2, wherein each tablet of the initiation dose comprises:
about 26.6 w/w % to about 26.7 w/w % of the spray dried dispersion of the compound of Formula Ia or Ib, or a pharmaceutically acceptable salt thereof;
about 0.03 w/w % to about 0.04 w/w % of the monohydrate of the compound of Formula IIa or IIb; and
one or more pharmaceutically acceptable excipients.

In some embodiments, the method comprises administering to the patient an initiation dose comprising two tablets on day 1, and two tablets on day 2, wherein each tablet of the initiation dose comprises:
about 26.67 w/w % of the spray dried dispersion of the compound of Formula Ia or Ib, or a pharmaceutically acceptable salt thereof;
about 0.0355 w/w % of the monohydrate of the compound of Formula IIa or IIb; and
one or more pharmaceutically acceptable excipients.

In some embodiments, the method comprises administering to the patient an initiation dose comprising two tablets on day 1, and two tablets on day 2, wherein each tablet of the initiation dose comprises:
about 26.6 w/w % to about 26.7 w/w % of the spray dried dispersion of the compound of Formula Ia or Ib, or a pharmaceutically acceptable salt thereof;
about 0.03 w/w % to about 0.04 w/w % of the compound of Formula IIa or IIb, or a pharmaceutically acceptable salt, co-crystal, solvate, or combination thereof;
about 41 w/w % to about 42 w/w % of mannitol;
about 22.3 w/w % to about 22.4 w/w % of microcrystalline cellulose;
about 7.9 w/w % to about 8.1 w/w % of croscarmellose sodium; and
about 1.4 w/w % to about 1.6 w/w % of magnesium stearate.

In some embodiments, the method comprises administering to the patient an initiation dose comprising two tablets on day 1, and two tablets on day 2, wherein each tablet of the initiation dose comprises:
about 26.67 w/w % of the spray dried dispersion of the compound of Formula Ia or Ib, or a pharmaceutically acceptable salt thereof;
about 0.0355 w/w % of the compound of Formula IIa or IIb, or a pharmaceutically acceptable salt, co-crystal, solvate, or combination thereof;
about 41.4 w/w % of mannitol;
about 22.3945 w/w % of microcrystalline cellulose;
about 8.0 w/w % of croscarmellose sodium; and
about 1.5 w/w % of magnesium stearate.

In some embodiments, the method comprises administering to the patient an initiation dose comprising two tablets on day 1, and two tablets on day 2, wherein each tablet of the initiation dose comprises:
about 400 mg of the spray dried dispersion of the compound of Formula Ia or Ib, or a pharmaceutically acceptable salt thereof, and
about 0.5 mg to about 0.6 mg of the compound of Formula IIa or IIb, or a pharmaceutically acceptable salt, co-crystal, solvate, or combination thereof.

In some embodiments, the method comprises administering to the patient an initiation dose comprising two tablets on day 1, and two tablets on day 2, wherein each tablet of the initiation dose comprises:
about 400 mg of the spray dried dispersion of the compound of Formula Ia or Ib, or a pharmaceutically acceptable salt thereof, and
about 0.5325 mg of the compound of Formula IIa or IIb, or a pharmaceutically acceptable salt, co-crystal, solvate, or combination thereof.

In some embodiments, the method comprises administering to the patient an initiation dose comprising two tablets on day 1, and two tablets on day 2, wherein each tablet of the initiation dose comprises:
about 400 mg of the spray dried dispersion of the compound of Formula Ia or Ib, or a pharmaceutically acceptable salt thereof, and
about 0.5 mg of the compound of Formula IIa or IIb, or a pharmaceutically acceptable salt, co-crystal, solvate, or combination thereof, on a free base basis.

In some embodiments, the method comprises administering to the patient an initiation dose comprising two tablets on day 1, and two tablets on day 2, wherein each tablet of the initiation dose comprises:
about 400 mg of the spray dried dispersion of the compound of Formula Ia or Ib, or a pharmaceutically acceptable salt thereof;
about 0.5 mg to about 0.6 mg of the compound of Formula IIa or IIb, or a pharmaceutically acceptable salt, co-crystal, solvate, or combination thereof;
about 615 mg to about 625 mg of mannitol;
about 330 mg to about 340 mg of microcrystalline cellulose;
about 115 mg to about 125 mg croscarmellose sodium; and
about 20 mg to about 25 mg magnesium stearate.

In some embodiments, the method comprises administering to the patient an initiation dose comprising two tablets on day 1, and two tablets on day 2, wherein each tablet of the initiation dose comprises:
about 400 mg of the spray dried dispersion of the compound of Formula Ia or Ib, or a pharmaceutically acceptable salt thereof;
about 0.5325 mg of the compound of Formula IIa or IIb, or a pharmaceutically acceptable salt, co-crystal, solvate, or combination thereof;
about 621 mg of mannitol;
about 335.9175 mg of microcrystalline cellulose;
about 120 mg croscarmellose sodium; and
about 22.5 mg magnesium stearate.

In some embodiments, the method comprises administering to the patient an initiation dose comprising two tablets on day 1, and two tablets on day 2, wherein each tablet of the initiation dose comprises:
about 400 mg of the spray dried dispersion of the compound of Formula Ia or Ib, or a pharmaceutically acceptable salt thereof;
about 0.5 mg of the compound of Formula IIa or IIb, or a pharmaceutically acceptable salt, co-crystal, solvate, or combination thereof, on a free base basis;
about 621 mg of mannitol;
about 335.9175 mg of microcrystalline cellulose;
about 120 mg croscarmellose sodium; and
about 22.5 mg magnesium stearate.

In some embodiments, the method comprises administering to the patient an initiation dose comprising two tablets on day 1, and two tablets on day 2, wherein each tablet of the initiation dose comprises:
about 26.6 w/w % to about 26.7 w/w % of the spray dried dispersion of the sodium salt of the compound of Formula Ia or Ib;
about 0.03 w/w % to about 0.04 w/w % of the compound of Formula IIa or IIb, or a pharmaceutically acceptable salt, co-crystal, solvate, or combination thereof; and
one or more pharmaceutically acceptable excipients; and

In some embodiments, the method comprises administering to the patient an initiation dose comprising two tablets on day 1, and two tablets on day 2, wherein each tablet of the initiation dose comprises:
about 26.67 w/w % of the spray dried dispersion of the sodium salt of the compound of Formula Ia or Ib;
about 0.0355 w/w % of the compound of Formula IIa or IIb, or a pharmaceutically acceptable salt, co-crystal, solvate, or combination thereof; and
one or more pharmaceutically acceptable excipients.

In some embodiments, the method comprises administering to the patient an initiation dose comprising two tablets on day 1, and two tablets on day 2, wherein each tablet of the initiation dose comprises:
about 26.6 w/w % to about 26.7 w/w % of the spray dried dispersion of the sodium salt of the compound of Formula Ia or Ib;
about 0.03 w/w % to about 0.04 w/w % of the compound of Formula IIa or IIb, or a solvate thereof, and
one or more pharmaceutically acceptable excipients.

In some embodiments, the method comprises administering to the patient an initiation dose comprising two tablets on day 1, and two tablets on day 2, wherein each tablet of the initiation dose comprises:
about 26.67 w/w % of the spray dried dispersion of the sodium salt of the compound of Formula Ia or Ib;
about 0.0355 w/w % of the compound of Formula IIa or IIb, or a solvate thereof, and
one or more pharmaceutically acceptable excipients.

In some embodiments, the method comprises administering to the patient an initiation dose comprising two tablets on day 1, and two tablets on day 2, wherein each tablet of the initiation dose comprises:
about 26.6 w/w % to about 26.7 w/w % of the spray dried dispersion of the sodium salt of the compound of Formula Ia or Ib;
about 0.03 w/w % to about 0.04 w/w % of the monohydrate of the compound of Formula IIa or IIb; and
one or more pharmaceutically acceptable excipients.

In some embodiments, the method comprises administering to the patient an initiation dose comprising two tablets on day 1, and two tablets on day 2, wherein each tablet of the initiation dose comprises:
about 26.67 w/w % of the spray dried dispersion of the sodium salt of the compound of Formula Ia or Ib;
about 0.0355 w/w % of the monohydrate of the compound of Formula IIa or IIb; and
one or more pharmaceutically acceptable excipients.

In some embodiments, the method comprises administering to the patient an initiation dose comprising two tablets on day 1, and two tablets on day 2, wherein each tablet of the initiation dose comprises:
about 26.6 w/w % to about 26.7 w/w % of the spray dried dispersion of the sodium salt of the compound of Formula Ia or Ib;
about 0.03 w/w % to about 0.04 w/w % of the compound of Formula IIa or IIb, or a pharmaceutically acceptable salt, co-crystal, solvate, or combination thereof;
about 41 w/w % to about 42 w/w % of mannitol;
about 22.3 w/w % to about 22.4 w/w % of microcrystalline cellulose;
about 7.9 w/w % to about 8.1 w/w % of croscarmellose sodium; and
about 1.4 w/w % to about 1.6 w/w % of magnesium stearate.

In some embodiments, the method comprises administering to the patient an initiation dose comprising two tablets on day 1, and two tablets on day 2, wherein each tablet of the initiation dose comprises:
about 26.67 w/w % of the spray dried dispersion of the sodium salt of the compound of Formula Ia or Ib;
about 0.0355 w/w % of the compound of Formula IIa or IIb, or a pharmaceutically acceptable salt, co-crystal, solvate, or combination thereof;
about 41.4 w/w % of mannitol;
about 22.3945 w/w % of microcrystalline cellulose;
about 8.0 w/w % of croscarmellose sodium; and
about 1.5 w/w % of magnesium stearate.

In some embodiments, the method comprises administering to the patient an initiation dose comprising two tablets on day 1, and two tablets on day 2, wherein each tablet of the initiation dose comprises:
about 400 mg of the spray dried dispersion of the sodium salt of the compound of Formula Ia or Ib; and
about 0.5 mg to about 0.6 mg of the compound of Formula IIa or IIb, or a pharmaceutically acceptable salt, co-crystal, solvate, or combination thereof.

In some embodiments, the method comprises administering to the patient an initiation dose comprising two tablets on day 1, and two tablets on day 2, wherein each tablet of the initiation dose comprises:
about 400 mg of the spray dried dispersion of the sodium salt of the compound of Formula Ia or Ib; and
about 0.5325 mg of the compound of Formula IIa or IIb, or a pharmaceutically acceptable salt, co-crystal, solvate, or combination thereof.

In some embodiments, the method comprises administering to the patient an initiation dose comprising two tablets on day 1, and two tablets on day 2, wherein each tablet of the initiation dose comprises:
about 400 mg of the spray dried dispersion of the sodium salt of the compound of Formula Ia or Ib; and
about 0.5 mg of the compound of Formula IIa or IIb, or a pharmaceutically acceptable salt, co-crystal, solvate, or combination thereof, on a free base basis.

In some embodiments, the method comprises administering to the patient an initiation dose comprising two tablets on day 1, and two tablets on day 2, wherein each tablet of the initiation dose comprises:
about 400 mg of the spray dried dispersion of the sodium salt of the compound of Formula Ia or Ib;
about 0.5 mg to about 0.6 mg of the compound of Formula IIa or IIb, or a pharmaceutically acceptable salt, co-crystal, solvate, or combination thereof;
about 615 mg to about 625 mg of mannitol;
about 330 mg to about 340 mg of microcrystalline cellulose;
about 115 mg to about 125 mg croscarmellose sodium; and
about 20 mg to about 25 mg magnesium stearate.

In some embodiments, the method comprises administering to the patient an initiation dose comprising two tablets on day 1, and two tablets on day 2, wherein each tablet of the initiation dose comprises:
about 400 mg of the spray dried dispersion of the sodium salt of the compound of Formula Ia or Ib;
about 0.5325 mg of the compound of Formula IIa or IIb, or a pharmaceutically acceptable salt, co-crystal, solvate, or combination thereof;
about 621 mg of mannitol;
about 335.9175 mg of microcrystalline cellulose;
about 120 mg croscarmellose sodium; and
about 22.5 mg magnesium stearate.

In some embodiments, the method comprises administering to the patient an initiation dose comprising two tablets on day 1, and two tablets on day 2, wherein each tablet of the initiation dose comprises:
about 400 mg of the spray dried dispersion of the sodium salt of the compound of Formula Ia or Ib;
about 0.5 mg of the compound of Formula IIa or IIb, or a pharmaceutically acceptable salt, co-crystal, solvate, or combination thereof, on a free base basis;
about 621 mg of mannitol;
about 335.9175 mg of microcrystalline cellulose;
about 120 mg croscarmellose sodium; and
about 22.5 mg magnesium stearate.

In some embodiments, the method comprises administering to the patient an initiation dose comprising two tablets on day 1, and two tablets on day 2, wherein each tablet of the initiation dose comprises:
about 26.6 w/w % to about 26.7 w/w % of the spray dried dispersion of the sodium salt of the compound of Formula Ia;
about 0.03 w/w % to about 0.04 w/w % of the compound of Formula IIa or IIb, or a pharmaceutically acceptable salt, co-crystal, solvate, or combination thereof; and
one or more pharmaceutically acceptable excipients; and

In some embodiments, the method comprises administering to the patient an initiation dose comprising two tablets on day 1, and two tablets on day 2, wherein each tablet of the initiation dose comprises:
about 26.67 w/w % of the spray dried dispersion of the spray dried dispersion of the sodium salt of the compound of Formula Ia;
about 0.0355 w/w % of the compound of Formula IIa or IIb, or a pharmaceutically acceptable salt, co-crystal, solvate, or combination thereof; and
one or more pharmaceutically acceptable excipients.

In some embodiments, the method comprises administering to the patient an initiation dose comprising two tablets on day 1, and two tablets on day 2, wherein each tablet of the initiation dose comprises:
about 26.6 w/w % to about 26.7 w/w % of the spray dried dispersion of the sodium salt of the compound of Formula Ia;
about 0.03 w/w % to about 0.04 w/w % of the compound of Formula IIa or IIb, or a solvate thereof, and
one or more pharmaceutically acceptable excipients.

In some embodiments, the method comprises administering to the patient an initiation dose comprising two tablets on day 1, and two tablets on day 2, wherein each tablet of the initiation dose comprises:
about 26.67 w/w % of the spray dried dispersion of the sodium salt of the compound of Formula Ia;
about 0.0355 w/w % of the compound of Formula IIa or IIb, or a solvate thereof, and
one or more pharmaceutically acceptable excipients.

In some embodiments, the method comprises administering to the patient an initiation dose comprising two tablets on day 1, and two tablets on day 2, wherein each tablet of the initiation dose comprises:
about 26.6 w/w % to about 26.7 w/w % of the spray dried dispersion of the sodium salt of the compound of Formula Ia;
about 0.03 w/w % to about 0.04 w/w % of the monohydrate of the compound of Formula IIa or IIb; and
one or more pharmaceutically acceptable excipients.

In some embodiments, the method comprises administering to the patient an initiation dose comprising two tablets on day 1, and two tablets on day 2, wherein each tablet of the initiation dose comprises:
about 26.67 w/w % of the spray dried dispersion of the sodium salt of the compound of Formula Ia;
about 0.0355 w/w % of the monohydrate of the compound of Formula IIa or IIb; and
one or more pharmaceutically acceptable excipients.

In some embodiments, the method comprises administering to the patient an initiation dose comprising two tablets on day 1, and two tablets on day 2, wherein each tablet of the initiation dose comprises:
about 26.6 w/w % to about 26.7 w/w % of the spray dried dispersion of the sodium salt of the compound of Formula Ia;
about 0.03 w/w % to about 0.04 w/w % of the compound of Formula IIa or IIb, or a pharmaceutically acceptable salt, co-crystal, solvate, or combination thereof;
about 41 w/w % to about 42 w/w % of mannitol;
about 22.3 w/w % to about 22.4 w/w % of microcrystalline cellulose;
about 7.9 w/w % to about 8.1 w/w % of croscarmellose sodium; and
about 1.4 w/w % to about 1.6 w/w % of magnesium stearate.

In some embodiments, the method comprises administering to the patient an initiation dose comprising two tablets on day 1, and two tablets on day 2, wherein each tablet of the initiation dose comprises:
about 26.67 w/w % of the spray dried dispersion of the sodium salt of the compound of Formula Ia;
about 0.0355 w/w % of the compound of Formula IIa or IIb, or a pharmaceutically acceptable salt, co-crystal, solvate, or combination thereof;
about 41.4 w/w % of mannitol;
about 22.3945 w/w % of microcrystalline cellulose;
about 8.0 w/w % of croscarmellose sodium; and
about 1.5 w/w % of magnesium stearate.

In some embodiments, the method comprises administering to the patient an initiation dose comprising two tablets on day 1, and two tablets on day 2, wherein each tablet of the initiation dose comprises:
about 400 mg of the spray dried dispersion of the sodium salt of the compound of Formula Ia; and
about 0.5 mg to about 0.6 mg of the compound of Formula IIa or IIb, or a pharmaceutically acceptable salt, co-crystal, solvate, or combination thereof.

In some embodiments, the method comprises administering to the patient an initiation dose comprising two tablets on day 1, and two tablets on day 2, wherein each tablet of the initiation dose comprises:
about 400 mg of the spray dried dispersion of the sodium salt of the compound of Formula Ia; and
about 0.5325 mg of the compound of Formula IIa or IIb, or a pharmaceutically acceptable salt, co-crystal, solvate, or combination thereof.

In some embodiments, the method comprises administering to the patient an initiation dose comprising two tablets on day 1, and two tablets on day 2, wherein each tablet of the initiation dose comprises:
about 400 mg of the spray dried dispersion of the sodium salt of the compound of Formula Ia; and
about 0.5 mg of the compound of Formula IIa or IIb, or a pharmaceutically acceptable salt, co-crystal, solvate, or combination thereof, on a free base basis.

In some embodiments, the method comprises administering to the patient an initiation dose comprising two tablets on day 1, and two tablets on day 2, wherein each tablet of the initiation dose comprises:
about 400 mg of the spray dried dispersion of the sodium salt of the compound of Formula Ia;
about 0.5 mg to about 0.6 mg of the compound of Formula IIa or IIb, or a pharmaceutically acceptable salt, co-crystal, solvate, or combination thereof;
about 615 mg to about 625 mg of mannitol;
about 330 mg to about 340 mg of microcrystalline cellulose;
about 115 mg to about 125 mg croscarmellose sodium; and
about 20 mg to about 25 mg magnesium stearate.

In some embodiments, the method comprises administering to the patient an initiation dose comprising two tablets on day 1, and two tablets on day 2, wherein each tablet of the initiation dose comprises:
about 400 mg of the spray dried dispersion of the sodium salt of the compound of Formula Ia;
about 0.5325 mg of the compound of Formula IIa or IIb, or a pharmaceutically acceptable salt, co-crystal, solvate, or combination thereof;
about 621 mg of mannitol;
about 335.9175 mg of microcrystalline cellulose;
about 120 mg croscarmellose sodium; and
about 22.5 mg magnesium stearate.

In some embodiments, the method comprises administering to the patient an initiation dose comprising two tablets on day 1, and two tablets on day 2, wherein each tablet of the initiation dose comprises:
about 400 mg of the spray dried dispersion of the sodium salt of the compound of Formula Ia;
about 0.5 mg of the compound of Formula IIa or IIb, or a pharmaceutically acceptable salt, co-crystal, solvate, or combination thereof, on a free base basis;
about 621 mg of mannitol;
about 335.9175 mg of microcrystalline cellulose;
about 120 mg croscarmellose sodium; and
about 22.5 mg magnesium stearate.

In some embodiments, the method comprises administering to the patient an initiation dose comprising two tablets on day 1, and two tablets on day 2, wherein each tablet of the initiation dose comprises:
about 26.6 w/w % to about 26.7 w/w % of the lenacapavir spray dried dispersion;
about 0.03 w/w % to about 0.04 w/w % of the compound of Formula IIa or IIb, or a pharmaceutically acceptable salt, co-crystal, solvate, or combination thereof; and
one or more pharmaceutically acceptable excipients; and

In some embodiments, the method comprises administering to the patient an initiation dose comprising two tablets on day 1, and two tablets on day 2, wherein each tablet of the initiation dose comprises:
about 26.67 w/w % of the lenacapavir spray dried dispersion;
about 0.0355 w/w % of the compound of Formula IIa or IIb, or a pharmaceutically acceptable salt, co-crystal, solvate, or combination thereof; and
one or more pharmaceutically acceptable excipients.

In some embodiments, the method comprises administering to the patient an initiation dose comprising two tablets on day 1, and two tablets on day 2, wherein each tablet of the initiation dose comprises:
about 26.6 w/w % to about 26.7 w/w % of the lenacapavir spray dried dispersion;
about 0.03 w/w % to about 0.04 w/w % of the compound of Formula IIa or IIb, or a solvate thereof, and
one or more pharmaceutically acceptable excipients.

In some embodiments, the method comprises administering to the patient an initiation dose comprising two tablets on day 1, and two tablets on day 2, wherein each tablet of the initiation dose comprises:
about 26.67 w/w % of the lenacapavir spray dried dispersion;
about 0.0355 w/w % of the compound of Formula IIa or IIb, or a solvate thereof, and
one or more pharmaceutically acceptable excipients.

In some embodiments, the method comprises administering to the patient an initiation dose comprising two tablets on day 1, and two tablets on day 2, wherein each tablet of the initiation dose comprises:

about 26.6 w/w % to about 26.7 w/w % of the lenacapavir spray dried dispersion;
about 0.03 w/w % to about 0.04 w/w % of the monohydrate of the compound of Formula IIa or IIb; and
one or more pharmaceutically acceptable excipients.

In some embodiments, the method comprises administering to the patient an initiation dose comprising two tablets on day 1, and two tablets on day 2, wherein each tablet of the initiation dose comprises:
about 26.67 w/w % of the lenacapavir spray dried dispersion;
about 0.0355 w/w % of the monohydrate of the compound of Formula IIa or IIb; and
one or more pharmaceutically acceptable excipients.

In some embodiments, the method comprises administering to the patient an initiation dose comprising two tablets on day 1, and two tablets on day 2, wherein each tablet of the initiation dose comprises:
about 26.6 w/w % to about 26.7 w/w % of the lenacapavir spray dried dispersion;
about 0.03 w/w % to about 0.04 w/w % of the compound of Formula IIa or IIb, or a pharmaceutically acceptable salt, co-crystal, solvate, or combination thereof;
about 41 w/w % to about 42 w/w % of mannitol;
about 22.3 w/w % to about 22.4 w/w % of microcrystalline cellulose;
about 7.9 w/w % to about 8.1 w/w % of croscarmellose sodium; and
about 1.4 w/w % to about 1.6 w/w % of magnesium stearate.

In some embodiments, the method comprises administering to the patient an initiation dose comprising two tablets on day 1, and two tablets on day 2, wherein each tablet of the initiation dose comprises:
about 26.67 w/w % of the compound of the lenacapavir spray dried dispersion;
about 0.0355 w/w % of the compound of Formula IIa or IIb, or a pharmaceutically acceptable salt, co-crystal, solvate, or combination thereof;
about 41.4 w/w % of mannitol;
about 22.3945 w/w % of microcrystalline cellulose;
about 8.0 w/w % of croscarmellose sodium; and
about 1.5 w/w % of magnesium stearate.

In some embodiments, the method comprises administering to the patient an initiation dose comprising two tablets on day 1, and two tablets on day 2, wherein each tablet of the initiation dose comprises:
about 400 mg of the lenacapavir spray dried dispersion; and
about 0.5 mg to about 0.6 mg of the compound of Formula IIa or IIb, or a pharmaceutically acceptable salt, co-crystal, solvate, or combination thereof.

In some embodiments, the method comprises administering to the patient an initiation dose comprising two tablets on day 1, and two tablets on day 2, wherein each tablet of the initiation dose comprises:
about 400 mg of the lenacapavir spray dried dispersion; and
about 0.5325 mg of the compound of Formula IIa or IIb, or a pharmaceutically acceptable salt, co-crystal, solvate, or combination thereof.

In some embodiments, the method comprises administering to the patient an initiation dose comprising two tablets on day 1, and two tablets on day 2, wherein each tablet of the initiation dose comprises:
about 400 mg of the lenacapavir spray dried dispersion; and
about 0.5 mg of the compound of Formula IIa or IIb, or a pharmaceutically acceptable salt, co-crystal, solvate, or combination thereof, on a free base basis.

In some embodiments, the method comprises administering to the patient an initiation dose comprising two tablets on day 1, and two tablets on day 2, wherein each tablet of the initiation dose comprises:
about 400 mg of the lenacapavir spray dried dispersion;
about 0.5 mg to about 0.6 mg of the compound of Formula IIa or IIb, or a pharmaceutically acceptable salt, co-crystal, solvate, or combination thereof;
about 615 mg to about 625 mg of mannitol;
about 330 mg to about 340 mg of microcrystalline cellulose;
about 115 mg to about 125 mg croscarmellose sodium; and
about 20 mg to about 25 mg magnesium stearate.

In some embodiments, the method comprises administering to the patient an initiation dose comprising two tablets on day 1, and two tablets on day 2, wherein each tablet of the initiation dose comprises:
about 400 mg of the lenacapavir spray dried dispersion;
about 0.5325 mg of the compound of Formula IIa or IIb, or a pharmaceutically acceptable salt, co-crystal, solvate, or combination thereof;
about 621 mg of mannitol;
about 335.9175 mg of microcrystalline cellulose;
about 120 mg croscarmellose sodium; and
about 22.5 mg magnesium stearate.

In some embodiments, the method comprises administering to the patient an initiation dose comprising two tablets on day 1, and two tablets on day 2, wherein each tablet of the initiation dose comprises:
about 400 mg of the lenacapavir spray dried dispersion;
about 0.5 mg of the compound of Formula IIa or IIb, or a pharmaceutically acceptable salt, co-crystal, solvate, or combination thereof, on a free base basis;
about 621 mg of mannitol;
about 335.9175 mg of microcrystalline cellulose;
about 120 mg croscarmellose sodium; and
about 22.5 mg magnesium stearate.

In some embodiments, the method comprises administering to the patient an initiation dose comprising two tablets on day 1, and two tablets on day 2, wherein each tablet of the initiation dose comprises:
about 300 mg of the compound of Formula Ia or Ib, or a pharmaceutically acceptable salt, co-crystal, solvate, or combination thereof, on a free acid basis; and
about 0.5 mg to about 0.6 mg of the compound of Formula IIa or IIb, or a pharmaceutically acceptable salt, co-crystal, solvate, or combination thereof.

In some embodiments, the method comprises administering to the patient an initiation dose comprising two tablets on day 1, and two tablets on day 2, wherein each tablet of the initiation dose comprises:
about 300 mg of the compound of Formula Ia or Ib, or a pharmaceutically acceptable salt, co-crystal, solvate, or combination thereof, on a free acid basis; and
about 0.5325 mg of the compound of Formula IIa or IIb, or a pharmaceutically acceptable salt, co-crystal, solvate, or combination thereof.

In some embodiments, the method comprises administering to the patient an initiation dose comprising two tablets on day 1, and two tablets on day 2, wherein each tablet of the initiation dose comprises:
about 300 mg of the compound of Formula Ia or Ib, or a pharmaceutically acceptable salt, co-crystal, solvate, or combination thereof, on a free acid basis;
about 0.5 mg of the compound of Formula IIa or IIb, or a pharmaceutically acceptable salt, co-crystal, solvate, or combination thereof, on a free base basis.

In some embodiments, the method comprises administering to the patient an initiation dose comprising two tablets on day 1, and two tablets on day 2, wherein each tablet of the initiation dose comprises:
about 300 mg of the compound of Formula Ia or Ib, or a pharmaceutically acceptable salt, co-crystal, solvate, or combination thereof, on a free acid basis;
about 0.5 mg to about 0.6 mg of the compound of Formula IIa or IIb, or a pharmaceutically acceptable salt, co-crystal, solvate, or combination thereof;
about 615 mg to about 625 mg of mannitol;
about 330 mg to about 340 mg of microcrystalline cellulose;
about 115 mg to about 125 mg croscarmellose sodium; and
about 20 mg to about 25 mg magnesium stearate.

In some embodiments, the method comprises administering to the patient an initiation dose comprising two tablets on day 1, and two tablets on day 2, wherein each tablet of the initiation dose comprises:
about 300 mg of the compound of Formula Ia or Ib, or a pharmaceutically acceptable salt, co-crystal, solvate, or combination thereof, on a free acid basis;
about 0.5325 mg of the compound of Formula IIa or IIb, or a pharmaceutically acceptable salt, co-crystal, solvate, or combination thereof;
about 621 mg of mannitol;
about 335.9175 mg of microcrystalline cellulose;
about 120 mg croscarmellose sodium; and
about 22.5 mg magnesium stearate.

In some embodiments, the method comprises administering to the patient an initiation dose comprising two tablets on day 1, and two tablets on day 2, wherein each tablet of the initiation dose comprises:
about 300 mg of the compound of Formula Ia or Ib, or a pharmaceutically acceptable salt, co-crystal, solvate, or combination thereof, on a free acid basis;
about 0.5 mg of the compound of Formula IIa or IIb, or a pharmaceutically acceptable salt, co-crystal, solvate, or combination thereof, on a free base basis;
about 621 mg of mannitol;
about 335.9175 mg of microcrystalline cellulose;
about 120 mg croscarmellose sodium; and
about 22.5 mg magnesium stearate.

In some embodiments, the second period of time begins about one week after the beginning of the first period of time (e.g., where the first period of time begins on day 1, the second period of time begins on about on day 8).

In some embodiments, each maintenance dosage comprises orally administering to the patient one tablet provided herein, once weekly.

In some embodiments, each tablet of the maintenance dosage comprises:
about 26.6 w/w % to about 26.7 w/w % of the spray dried dispersion of the compound of Formula Ia or Ib, or a pharmaceutically acceptable salt, co-crystal, solvate, or combination thereof;
about 0.14 w/w % to about 0.15 w/w % of the compound of Formula IIa or IIb, or a pharmaceutically acceptable salt, co-crystal, solvate, or combination thereof; and
one or more pharmaceutically acceptable excipients.

In some embodiments, each tablet of the maintenance dosage comprises:
about 26.67 w/w % of the spray dried dispersion of the compound of Formula Ia or Ib, or a pharmaceutically acceptable salt, co-crystal, solvate, or combination thereof;
about 0.142 w/w % of the compound of Formula IIa or IIb, or a pharmaceutically acceptable salt, co-crystal, solvate, or combination thereof; and
one or more pharmaceutically acceptable excipients.

In some embodiments, each tablet of the maintenance dosage comprises:
about 26.6 w/w % to about 26.7 w/w % of the spray dried dispersion of the compound of Formula Ia or Ib, or a pharmaceutically acceptable salt, co-crystal, solvate, or combination thereof;
about 0.14 w/w % to about 0.15 w/w % of the compound of Formula IIa or IIb, or a solvate thereof, and
one or more pharmaceutically acceptable excipients.

In some embodiments, each tablet of the maintenance dosage comprises:
about 26.67 w/w % of the spray dried dispersion of the compound of Formula Ia or Ib, or a pharmaceutically acceptable salt, co-crystal, solvate, or combination thereof;
about 0.142 w/w % of the compound of Formula IIa or IIb, or a solvate thereof; and
one or more pharmaceutically acceptable excipients.

In some embodiments, each tablet of the maintenance dosage comprises:
about 26.6 w/w % to about 26.7 w/w % of the spray dried dispersion of the compound of Formula Ia or Ib, or a pharmaceutically acceptable salt, co-crystal, solvate, or combination thereof;
about 0.14 w/w % to about 0.15 w/w % of the monohydrate of the compound of Formula IIa or IIb; and
one or more pharmaceutically acceptable excipients.

In some embodiments, each tablet of the maintenance dosage comprises:
about 26.67 w/w % of the spray dried dispersion of the compound of Formula Ia or Ib, or a pharmaceutically acceptable salt, co-crystal, solvate, or combination thereof;
about 0.142 w/w % of the monohydrate of the compound of Formula IIa or IIb; and
one or more pharmaceutically acceptable excipients.

In some embodiments, each tablet of the maintenance dosage comprises:
about 26.6 w/w % to about 26.7 w/w % of the spray dried dispersion of the compound of Formula Ia or Ib, or a pharmaceutically acceptable salt, co-crystal, solvate, or combination thereof;
about 0.14 w/w % to about 0.15 w/w % of the compound of Formula IIa or IIb, or a pharmaceutically acceptable salt, co-crystal, solvate, or combination thereof;
about 41 w/w % to about 42 w/w % of mannitol;
about 22.2 w/w % to about 22.3 w/w % of microcrystalline cellulose;
about 7.9 w/w % to about 8.1 w/w % of croscarmellose sodium; and
about 1.4 w/w % to about 1.6 w/w % of magnesium stearate.

In some embodiments, each tablet of the maintenance dosage comprises:
about 26.67 w/w % of the spray dried dispersion of the compound of Formula Ia or Ib, or a pharmaceutically acceptable salt, co-crystal, solvate, or combination thereof;
about 0.142 w/w % of the compound of Formula IIa or IIb, or a pharmaceutically acceptable salt, co-crystal, solvate, or combination thereof;
about 41.40 w/w % of mannitol;
about 22.288 w/w % microcrystalline cellulose;
about 8.0 w/w % of croscarmellose sodium; and
about 1.5 w/w % of magnesium stearate.

In some embodiments, each tablet of the maintenance dosage comprises:
about 19 w/w % to about 21 w/w % of the compound of Formula Ia or Ib, or a pharmaceutically acceptable salt thereof;
about 0.14 w/w % to about 0.15 w/w % of the compound of Formula IIa or IIb, or a solvate thereof;
about 4 w/w % to about 6 w/w % of copovidone;
about 0.1 w/w % to about 2 w/w % of poloxamer 407;
about 40 w/w % to about 42 w/w % of mannitol;
about 22 w/w % to about 23 w/w % of microcrystalline cellulose;
about 7 w/w % to about 9 w/w % of croscarmellose sodium; and
about 1.0 w/w % to about 2.0 w/w % of magnesium stearate.

In some embodiments, each tablet of the maintenance dosage comprises:
about 19.8 w/w % to about 20.8 w/w % of the compound of Formula Ia or Ib, or a pharmaceutically acceptable salt thereof;
about 0.14 w/w % to about 0.15 w/w % of the compound of Formula IIa or IIb, or a solvate thereof;
about 4 w/w % to about 5 w/w % of copovidone;
about 1 w/w % to about 2 w/w % of poloxamer 407;
about 41 w/w % to about 42 w/w % of mannitol;
about 22.2 w/w % to about 22.3 w/w % of microcrystalline cellulose;
about 7.9 w/w % to about 8.1 w/w % of croscarmellose sodium; and
about 1.4 w/w % to about 1.6 w/w % of magnesium stearate.

In some embodiments, each tablet of the maintenance dosage comprises:
about 20.46 w/w % of the compound of Formula Ia or Ib, or a pharmaceutically acceptable salt thereof;
about 0.142 w/w % of the compound of Formula IIa or IIb, or a solvate thereof;
about 4.88 w/w % of copovidone;
about 1.33 w/w % of poloxamer 407;
about 41.40 w/w % of mannitol;
about 22.288 w/w % of microcrystalline cellulose;
about 8.0 w/w % of croscarmellose sodium; and
about 1.5 w/w % of magnesium stearate.

In some embodiments, each tablet of the maintenance dosage comprises:
about 19 w/w % to about 21 w/w % of the sodium salt of the compound of Formula Ia or Ib;
about 0.14 w/w % to about 0.15 w/w % of the monohydrate of the compound of Formula IIa or IIb;
about 4 w/w % to about 6 w/w % of copovidone;
about 0.1 w/w % to about 2 w/w % of poloxamer 407;
about 40 w/w % to about 42 w/w % of mannitol;
about 22 w/w % to about 23 w/w % of microcrystalline cellulose;
about 7 w/w % to about 9 w/w % of croscarmellose sodium; and
about 1.0 w/w % to about 2.0 w/w % of magnesium stearate.

In some embodiments, each tablet of the maintenance dosage comprises:
about 19.8 w/w % to about 20.8 w/w % of the sodium salt of the compound of Formula Ia or Ib;
about 0.14 w/w % to about 0.15 w/w % of the monohydrate of the compound of Formula IIa or IIb;
about 4 w/w % to about 5 w/w % of copovidone;
about 1 w/w % to about 2 w/w % of poloxamer 407;
about 41 w/w % to about 42 w/w % of mannitol;
about 22.2 w/w % to about 22.3 w/w % of microcrystalline cellulose;
about 7.9 w/w % to about 8.1 w/w % of croscarmellose sodium; and
about 1.4 w/w % to about 1.6 w/w % of magnesium stearate.

In some embodiments, each tablet of the maintenance dosage comprises:
about 20.46 w/w % of the sodium salt of the compound of Formula Ia or Ib;
about 0.142 w/w % of the monohydrate of the compound of Formula IIa or IIb;
about 4.88 w/w % of copovidone;
about 1.33 w/w % of poloxamer 407;
about 41.40 w/w % of mannitol;
about 22.288 w/w % of microcrystalline cellulose;
about 8.0 w/w % of croscarmellose sodium; and
about 1.5 w/w % of magnesium stearate.

In some embodiments, each tablet of the maintenance dosage comprises:
about 19 w/w % to about 21 w/w % of the compound of Formula Ia or Ib, or a pharmaceutically acceptable salt thereof;
about 0.14 w/w % to about 0.15 w/w % of the compound of Formula IIa or IIb, or a solvate thereof;
about 4 w/w % to about 6 w/w % of copovidone;
about 0.1 w/w % to about 2 w/w % of poloxamer 407;
about 40 w/w % to about 42 w/w % of mannitol;
about 22 w/w % to about 23 w/w % of microcrystalline cellulose;
about 7 w/w % to about 9 w/w % of croscarmellose sodium;
about 1.0 w/w % to about 2.0 w/w % of magnesium stearate; and
an outer film coat.

In some embodiments, each tablet of the maintenance dosage comprises:
about 19.8 w/w % to about 20.8 w/w % of the compound of Formula Ia or Ib, or a pharmaceutically acceptable salt thereof;
about 0.14 w/w % to about 0.15 w/w % of the compound of Formula IIa or IIb, or a solvate thereof;
about 4 w/w % to about 5 w/w % of copovidone;
about 1 w/w % to about 2 w/w % of poloxamer 407;
about 41 w/w % to about 42 w/w % of mannitol;
about 22.2 w/w % to about 22.3 w/w % of microcrystalline cellulose;
about 7.9 w/w % to about 8.1 w/w % of croscarmellose sodium;
about 1.4 w/w % to about 1.6 w/w % of magnesium stearate; and
an outer film coat.

In some embodiments, each tablet of the maintenance dosage comprises:
about 20.46 w/w % of the compound of Formula Ia or Ib, or a pharmaceutically acceptable salt thereof;
about 0.142 w/w % of the compound of Formula IIa or IIb, or a solvate thereof;
about 4.88 w/w % of copovidone;
about 1.33 w/w % of poloxamer 407;
about 41.40 w/w % of mannitol;
about 22.288 w/w % of microcrystalline cellulose;
about 8.0 w/w % of croscarmellose sodium;
about 1.5 w/w % of magnesium stearate; and
an outer film coat.

In some embodiments, each tablet of the maintenance dosage comprises:
about 19 w/w % to about 21 w/w % of the sodium salt of the compound of Formula Ia or Ib;
about 0.14 w/w % to about 0.15 w/w % of the monohydrate of the compound of Formula IIa or IIb;
about 4 w/w % to about 6 w/w % of copovidone;
about 0.1 w/w % to about 2 w/w % of poloxamer 407;
about 40 w/w % to about 42 w/w % of mannitol;
about 22 w/w % to about 23 w/w % of microcrystalline cellulose;
about 7 w/w % to about 9 w/w % of croscarmellose sodium;
about 1.0 w/w % to about 2.0 w/w % of magnesium stearate; and
an outer film coat.

In some embodiments, each tablet of the maintenance dosage comprises:
about 19.8 w/w % to about 20.8 w/w % of the sodium salt of the compound of Formula Ia or Ib;
about 0.14 w/w % to about 0.15 w/w % of the monohydrate of the compound of Formula IIa or IIb;
about 4 w/w % to about 5 w/w % of copovidone;
about 1 w/w % to about 2 w/w % of poloxamer 407;
about 41 w/w % to about 42 w/w % of mannitol;
about 22.2 w/w % to about 22.3 w/w % of microcrystalline cellulose;
about 7.9 w/w % to about 8.1 w/w % of croscarmellose sodium;
about 1.4 w/w % to about 1.6 w/w % of magnesium stearate; and
an outer film coat.

In some embodiments, each tablet of the maintenance dosage comprises:
about 20.46 w/w % of the sodium salt of the compound of Formula Ia or Ib;
about 0.142 w/w % of the monohydrate of the compound of Formula IIa or IIb;
about 4.88 w/w % of copovidone;
about 1.33 w/w % of poloxamer 407;
about 41.40 w/w % of mannitol;
about 22.288 w/w % of microcrystalline cellulose;
about 8.0 w/w % of croscarmellose sodium;
about 1.5 w/w % of magnesium stearate; and
an outer film coat.

In some embodiments, each tablet of the maintenance dosage consists essentially of:
about 19 w/w % to about 21 w/w % of the compound of Formula Ia or Ib, or a pharmaceutically acceptable salt thereof;
about 0.14 w/w % to about 0.15 w/w % of the compound of Formula IIa or IIb, or a solvate thereof;
about 4 w/w % to about 6 w/w % of copovidone;
about 0.1 w/w % to about 2 w/w % of poloxamer 407;
about 40 w/w % to about 42 w/w % of mannitol;
about 22 w/w % to about 23 w/w % of microcrystalline cellulose;
about 7 w/w % to about 9 w/w % of croscarmellose sodium; and
about 1.0 w/w % to about 2.0 w/w % of magnesium stearate.

In some embodiments, each tablet of the maintenance dosage consists essentially of:
about 19.8 w/w % to about 20.8 w/w % of the compound of Formula Ia or Ib, or a pharmaceutically acceptable salt thereof;
about 0.14 w/w % to about 0.15 w/w % of the compound of Formula IIa or IIb, or a solvate thereof;
about 4 w/w % to about 5 w/w % of copovidone;
about 1 w/w % to about 2 w/w % of poloxamer 407;
about 41 w/w % to about 42 w/w % of mannitol;
about 22.2 w/w % to about 22.3 w/w % of microcrystalline cellulose;
about 7.9 w/w % to about 8.1 w/w % of croscarmellose sodium; and
about 1.4 w/w % to about 1.6 w/w % of magnesium stearate.

In some embodiments, each tablet of the maintenance dosage consists essentially of:
about 20.46 w/w % of the compound of Formula Ia or Ib, or a pharmaceutically acceptable salt thereof;
about 0.142 w/w % of the compound of Formula IIa or IIb, or a solvate thereof;
about 4.88 w/w % of copovidone;
about 1.33 w/w % of poloxamer 407;
about 41.40 w/w % of mannitol;
about 22.288 w/w % of microcrystalline cellulose;
about 8.0 w/w % of croscarmellose sodium; and
about 1.5 w/w % of magnesium stearate.

In some embodiments, each tablet of the maintenance dosage consists essentially of:
about 19 w/w % to about 21 w/w % of the sodium salt of the compound of Formula Ia or Ib;
about 0.14 w/w % to about 0.15 w/w % of the monohydrate of the compound of Formula IIa or IIb;
about 4 w/w % to about 6 w/w % of copovidone;
about 0.1 w/w % to about 2 w/w % of poloxamer 407;
about 40 w/w % to about 42 w/w % of mannitol;
about 22 w/w % to about 23 w/w % of microcrystalline cellulose;
about 7 w/w % to about 9 w/w % of croscarmellose sodium; and
about 1.0 w/w % to about 2.0 w/w % of magnesium stearate.

In some embodiments, each tablet of the maintenance dosage consists essentially of:
about 19.8 w/w % to about 20.8 w/w % of the sodium salt of the compound of Formula Ia or Ib;
about 0.14 w/w % to about 0.15 w/w % of the monohydrate of the compound of Formula IIa or IIb;
about 4 w/w % to about 5 w/w % of copovidone;
about 1 w/w % to about 2 w/w % of poloxamer 407;
about 41 w/w % to about 42 w/w % of mannitol;
about 22.2 w/w % to about 22.3 w/w % of microcrystalline cellulose;
about 7.9 w/w % to about 8.1 w/w % of croscarmellose sodium; and
about 1.4 w/w % to about 1.6 w/w % of magnesium stearate.

In some embodiments, each tablet of the maintenance dosage consists essentially of:
about 20.46 w/w % of the sodium salt of the compound of Formula Ia or Ib;
about 0.142 w/w % of the monohydrate of the compound of Formula IIa or IIb;
about 4.88 w/w % of copovidone;
about 1.33 w/w % of poloxamer 407;
about 41.40 w/w % of mannitol;
about 22.288 w/w % of microcrystalline cellulose;
about 8.0 w/w % of croscarmellose sodium; and
about 1.5 w/w % of magnesium stearate.

In some embodiments, each tablet of the maintenance dosage consists essentially of:
about 19 w/w % to about 21 w/w % of the compound of Formula Ia or Ib, or a pharmaceutically acceptable salt thereof;
about 0.14 w/w % to about 0.15 w/w % of the compound of Formula IIa or IIb, or a solvate thereof;
about 4 w/w % to about 6 w/w % of copovidone;
about 0.1 w/w % to about 2 w/w % of poloxamer 407;
about 40 w/w % to about 42 w/w % of mannitol;
about 22 w/w % to about 23 w/w % of microcrystalline cellulose;
about 7 w/w % to about 9 w/w % of croscarmellose sodium;
about 1.0 w/w % to about 2.0 w/w % of magnesium stearate; and
an outer film coat.

In some embodiments, each tablet of the maintenance dosage consists essentially of:
about 19.8 w/w % to about 20.8 w/w % of the compound of Formula Ia or Ib, or a pharmaceutically acceptable salt thereof;
about 0.14 w/w % to about 0.15 w/w % of the compound of Formula IIa or IIb, or a solvate thereof;
about 4 w/w % to about 5 w/w % of copovidone;
about 1 w/w % to about 2 w/w % of poloxamer 407;
about 41 w/w % to about 42 w/w % of mannitol;
about 22.2 w/w % to about 22.3 w/w % of microcrystalline cellulose;
about 7.9 w/w % to about 8.1 w/w % of croscarmellose sodium;
about 1.4 w/w % to about 1.6 w/w % of magnesium stearate; and
an outer film coat.

In some embodiments, each tablet of the maintenance dosage consists essentially of:
about 20.46 w/w % of the compound of Formula Ia or Ib, or a pharmaceutically acceptable salt thereof;
about 0.142 w/w % of the compound of Formula IIa or IIb, or a solvate thereof;
about 4.88 w/w % of copovidone;
about 1.33 w/w % of poloxamer 407;
about 41.40 w/w % of mannitol;
about 22.288 w/w % of microcrystalline cellulose;
about 8.0 w/w % of croscarmellose sodium;
about 1.5 w/w % of magnesium stearate; and
an outer film coat.

In some embodiments, each tablet of the maintenance dosage consists essentially of:
about 19 w/w % to about 21 w/w % of the sodium salt of the compound of Formula Ia or Ib;
about 0.14 w/w % to about 0.15 w/w % of the monohydrate of the compound of Formula IIa or IIb;
about 4 w/w % to about 6 w/w % of copovidone;
about 0.1 w/w % to about 2 w/w % of poloxamer 407;
about 40 w/w % to about 42 w/w % of mannitol;
about 22 w/w % to about 23 w/w % of microcrystalline cellulose;
about 7 w/w % to about 9 w/w % of croscarmellose sodium;
about 1.0 w/w % to about 2.0 w/w % of magnesium stearate; and
an outer film coat.

In some embodiments, each tablet of the maintenance dosage consists essentially of:
about 19.8 w/w % to about 20.8 w/w % of the sodium salt of the compound of Formula Ia or Ib;
about 0.14 w/w % to about 0.15 w/w % of the monohydrate of the compound of Formula IIa or IIb;
about 4 w/w % to about 5 w/w % of copovidone;
about 1 w/w % to about 2 w/w % of poloxamer 407;
about 41 w/w % to about 42 w/w % of mannitol;
about 22.2 w/w % to about 22.3 w/w % of microcrystalline cellulose;
about 7.9 w/w % to about 8.1 w/w % of croscarmellose sodium;
about 1.4 w/w % to about 1.6 w/w % of magnesium stearate; and
an outer film coat.

In some embodiments, each tablet of the maintenance dosage consists essentially of:
about 20.46 w/w % of the sodium salt of the compound of Formula Ia or Ib;
about 0.142 w/w % of the monohydrate of the compound of Formula IIa or IIb;
about 4.88 w/w % of copovidone;
about 1.33 w/w % of poloxamer 407;
about 41.40 w/w % of mannitol;
about 22.288 w/w % of microcrystalline cellulose;
about 8.0 w/w % of croscarmellose sodium;
about 1.5 w/w % of magnesium stearate; and
an outer film coat.

In some embodiments, each tablet of the maintenance dosage consists of:
about 19 w/w % to about 21 w/w % of the compound of Formula Ia or Ib, or a pharmaceutically acceptable salt thereof;
about 0.14 w/w % to about 0.15 w/w % of the compound of Formula IIa or IIb, or a solvate thereof;
about 4 w/w % to about 6 w/w % of copovidone;
about 0.1 w/w % to about 2 w/w % of poloxamer 407;
about 40 w/w % to about 42 w/w % of mannitol;
about 22 w/w % to about 23 w/w % of microcrystalline cellulose;
about 7 w/w % to about 9 w/w % of croscarmellose sodium; and
about 1.0 w/w % to about 2.0 w/w % of magnesium stearate.

In some embodiments, each tablet of the maintenance dosage consists of:
about 19.8 w/w % to about 20.8 w/w % of the compound of Formula Ia or Ib, or a pharmaceutically acceptable salt thereof;
about 0.14 w/w % to about 0.15 w/w % of the compound of Formula IIa or IIb, or a solvate thereof;
about 4 w/w % to about 5 w/w % of copovidone;
about 1 w/w % to about 2 w/w % of poloxamer 407;
about 41 w/w % to about 42 w/w % of mannitol;
about 22.2 w/w % to about 22.3 w/w % of microcrystalline cellulose;
about 7.9 w/w % to about 8.1 w/w % of croscarmellose sodium; and
about 1.4 w/w % to about 1.6 w/w % of magnesium stearate.

In some embodiments, each tablet of the maintenance dosage consists of:
about 20.46 w/w % of the compound of Formula Ia or Ib, or a pharmaceutically acceptable salt thereof;
about 0.142 w/w % of the compound of Formula IIa or IIb, or a solvate thereof;
about 4.88 w/w % of copovidone;
about 1.33 w/w % of poloxamer 407;
about 41.40 w/w % of mannitol;
about 22.288 w/w % of microcrystalline cellulose;
about 8.0 w/w % of croscarmellose sodium; and
about 1.5 w/w % of magnesium stearate.

In some embodiments, each tablet of the maintenance dosage consists of:
about 19 w/w % to about 21 w/w % of the sodium salt of the compound of Formula Ia or Ib;
about 0.14 w/w % to about 0.15 w/w % of the monohydrate of the compound of Formula IIa or IIb;
about 4 w/w % to about 6 w/w % of copovidone;
about 0.1 w/w % to about 2 w/w % of poloxamer 407;
about 40 w/w % to about 42 w/w % of mannitol;
about 22 w/w % to about 23 w/w % of microcrystalline cellulose;
about 7 w/w % to about 9 w/w % of croscarmellose sodium; and
about 1.0 w/w % to about 2.0 w/w % of magnesium stearate.

In some embodiments, each tablet of the maintenance dosage consists of:
about 19.8 w/w % to about 20.8 w/w % of the sodium salt of the compound of Formula Ia or Ib;
about 0.14 w/w % to about 0.15 w/w % of the monohydrate of the compound of Formula Ila or IIb;
about 4 w/w % to about 5 w/w % of copovidone;
about 1 w/w % to about 2 w/w % of poloxamer 407;
about 41 w/w % to about 42 w/w % of mannitol;
about 22.2 w/w % to about 22.3 w/w % of microcrystalline cellulose;
about 7.9 w/w % to about 8.1 w/w % of croscarmellose sodium; and
about 1.4 w/w % to about 1.6 w/w % of magnesium stearate.

In some embodiments, each tablet of the maintenance dosage consists of:
about 20.46 w/w % of the sodium salt of the compound of Formula Ia or Ib;
about 0.142 w/w % of the monohydrate of the compound of Formula IIa or IIb;
about 4.88 w/w % of copovidone;
about 1.33 w/w % of poloxamer 407;
about 41.40 w/w % of mannitol;
about 22.288 w/w % of microcrystalline cellulose;
about 8.0 w/w % of croscarmellose sodium; and
about 1.5 w/w % of magnesium stearate.

In some embodiments, each tablet of the maintenance dosage consists of:
about 19 w/w % to about 21 w/w % of the compound of Formula Ia or Ib, or a pharmaceutically acceptable salt thereof;
about 0.14 w/w % to about 0.15 w/w % of the compound of Formula IIa or IIb, or a solvate thereof;
about 4 w/w % to about 6 w/w % of copovidone;
about 0.1 w/w % to about 2 w/w % of poloxamer 407;
about 40 w/w % to about 42 w/w % of mannitol;
about 22 w/w % to about 23 w/w % of microcrystalline cellulose;
about 7 w/w % to about 9 w/w % of croscarmellose sodium;
about 1.0 w/w % to about 2.0 w/w % of magnesium stearate; and
an outer film coat.

In some embodiments, each tablet of the maintenance dosage consists of:
about 19.8 w/w % to about 20.8 w/w % of the compound of Formula Ia or Ib, or a pharmaceutically acceptable salt thereof;
about 0.14 w/w % to about 0.15 w/w % of the compound of Formula IIa or IIb, or a solvate thereof;
about 4 w/w % to about 5 w/w % of copovidone;
about 1 w/w % to about 2 w/w % of poloxamer 407;
about 41 w/w % to about 42 w/w % of mannitol;
about 22.2 w/w % to about 22.3 w/w % of microcrystalline cellulose;
about 7.9 w/w % to about 8.1 w/w % of croscarmellose sodium;
about 1.4 w/w % to about 1.6 w/w % of magnesium stearate; and
an outer film coat.

In some embodiments, each tablet of the maintenance dosage consists of:
about 20.46 w/w % of the compound of Formula Ia or Ib, or a pharmaceutically acceptable salt thereof;
about 0.142 w/w % of the compound of Formula IIa or IIb, or a solvate thereof;
about 4.88 w/w % of copovidone;
about 1.33 w/w % of poloxamer 407;
about 41.40 w/w % of mannitol;
about 22.288 w/w % of microcrystalline cellulose;
about 8.0 w/w % of croscarmellose sodium;
about 1.5 w/w % of magnesium stearate; and
an outer film coat.

In some embodiments, each tablet of the maintenance dosage consists of:
about 19 w/w % to about 21 w/w % of the sodium salt of the compound of Formula Ia or Ib;
about 0.14 w/w % to about 0.15 w/w % of the monohydrate of the compound of Formula IIa or IIb;
about 4 w/w % to about 6 w/w % of copovidone;
about 0.1 w/w % to about 2 w/w % of poloxamer 407;
about 40 w/w % to about 42 w/w % of mannitol;
about 22 w/w % to about 23 w/w % of microcrystalline cellulose;
about 7 w/w % to about 9 w/w % of croscarmellose sodium;
about 1.0 w/w % to about 2.0 w/w % of magnesium stearate; and
an outer film coat.

In some embodiments, each tablet of the maintenance dosage consists of:
about 19.8 w/w % to about 20.8 w/w % of the sodium salt of the compound of Formula Ia or Ib;
about 0.14 w/w % to about 0.15 w/w % of the monohydrate of the compound of Formula IIa or IIb;
about 4 w/w % to about 5 w/w % of copovidone;
about 1 w/w % to about 2 w/w % of poloxamer 407;
about 41 w/w % to about 42 w/w % of mannitol;
about 22.2 w/w % to about 22.3 w/w % of microcrystalline cellulose;
about 7.9 w/w % to about 8.1 w/w % of croscarmellose sodium;
about 1.4 w/w % to about 1.6 w/w % of magnesium stearate; and
an outer film coat.

In some embodiments, each tablet of the maintenance dosage consists of:
about 20.46 w/w % of the sodium salt of the compound of Formula Ia or Ib;
about 0.142 w/w % of the monohydrate of the compound of Formula IIa or IIb;
about 4.88 w/w % of copovidone;
about 1.33 w/w % of poloxamer 407;
about 41.40 w/w % of mannitol;
about 22.288 w/w % of microcrystalline cellulose;
about 8.0 w/w % of croscarmellose sodium;
about 1.5 w/w % of magnesium stearate; and
an outer film coat.

In some embodiments, each tablet of the maintenance dosage comprises:
about 400 mg of the spray dried dispersion of the compound of Formula Ia or Ib, or a pharmaceutically acceptable salt, co-crystal, solvate, or combination thereof; and
about 2.1 mg to about 2.2 mg of the compound of Formula IIa or IIb, or a pharmaceutically acceptable salt, co-crystal, solvate, or combination thereof.

In some embodiments, each tablet of the maintenance dosage comprises:
about 400 mg of the spray dried dispersion of the compound of Formula Ia or Ib, or a pharmaceutically acceptable salt, co-crystal, solvate, or combination thereof; and
about 2.130 mg of the compound of Formula IIa or IIb, or a pharmaceutically acceptable salt, co-crystal, solvate, or combination thereof.

In some embodiments, each tablet of the maintenance dosage comprises:
about 400 mg of the spray dried dispersion of the compound of Formula Ia or Ib, or a pharmaceutically acceptable salt, co-crystal, solvate, or combination thereof; and
about 2.0 mg of the compound of Formula IIa or IIb, or a pharmaceutically acceptable salt, co-crystal, solvate, or combination thereof, on a free base basis.

In some embodiments, each tablet of the maintenance dosage comprises:
about 400 mg of the spray dried dispersion of the compound of Formula Ia or Ib, or a pharmaceutically acceptable salt, co-crystal, solvate, or combination thereof;
about 2.1 mg to about 2.2 mg of the compound of Formula IIa or IIb, or a pharmaceutically acceptable salt, co-crystal, solvate, or combination thereof;
about 615 mg to about 625 mg of mannitol;
about 330 mg to about 340 mg of microcrystalline cellulose;
about 115 mg to about 125 mg croscarmellose sodium; and
about 20 mg to about 25 mg magnesium stearate.

In some embodiments, each tablet of the maintenance dosage comprises:
about 400 mg of the spray dried dispersion of the compound of Formula Ia or Ib, or a pharmaceutically acceptable salt, co-crystal, solvate, or combination thereof;
about 2.130 mg of the compound of Formula IIa or IIb, or a pharmaceutically acceptable salt, co-crystal, solvate, or combination thereof;
about 621 mg of mannitol;
about 334.32 mg of microcrystalline cellulose;
about 120 mg croscarmellose sodium; and
about 22.5 mg magnesium stearate.

In some embodiments, each tablet of the maintenance dosage comprises:
about 400 mg of the spray dried dispersion of the compound of Formula Ia or Ib, or a pharmaceutically acceptable salt, co-crystal, solvate, or combination thereof;
about 2.0 mg of the compound of Formula IIa or IIb, or a pharmaceutically acceptable salt, co-crystal, solvate, or combination thereof, on a free base basis;
about 621 mg of mannitol;
about 334.32 mg of microcrystalline cellulose;
about 120 mg croscarmellose sodium; and
about 22.5 mg magnesium stearate.

In some embodiments, each tablet of the maintenance dosage comprises:
about 26.6 w/w % to about 26.7 w/w % of the spray dried dispersion of the compound of Formula Ia or Ib, or a pharmaceutically acceptable salt thereof;
about 0.14 w/w % to about 0.15 w/w % of the compound of Formula IIa or IIb, or a pharmaceutically acceptable salt, co-crystal, solvate, or combination thereof; and
one or more pharmaceutically acceptable excipients.

In some embodiments, each tablet of the maintenance dosage comprises:
about 26.67 w/w % of the spray dried dispersion of the compound of Formula Ia or Ib, or a pharmaceutically acceptable salt thereof;
about 0.142 w/w % of the compound of Formula IIa or IIb, or a pharmaceutically acceptable salt, co-crystal, solvate, or combination thereof; and
one or more pharmaceutically acceptable excipients.

In some embodiments, each tablet of the maintenance dosage comprises:
about 26.6 w/w % to about 26.7 w/w % of the spray dried dispersion of the compound of Formula Ia or Ib, or a pharmaceutically acceptable salt thereof;
about 0.14 w/w % to about 0.15 w/w % of the compound of Formula IIa or IIb, or a solvate thereof, and
one or more pharmaceutically acceptable excipients.

In some embodiments, each tablet of the maintenance dosage comprises:
about 26.67 w/w % of the spray dried dispersion of the compound of Formula Ia or Ib, or a pharmaceutically acceptable salt thereof;
about 0.142 w/w % of the compound of Formula IIa or IIb, or a solvate thereof; and
one or more pharmaceutically acceptable excipients.

In some embodiments, each tablet of the maintenance dosage comprises:
about 26.6 w/w % to about 26.7 w/w % of the spray dried dispersion of the compound of Formula Ia or Ib, or a pharmaceutically acceptable salt thereof;
about 0.14 w/w % to about 0.15 w/w % of the monohydrate of the compound of Formula IIa or IIb; and
one or more pharmaceutically acceptable excipients.

In some embodiments, each tablet of the maintenance dosage comprises:
about 26.67 w/w % of the spray dried dispersion of the compound of Formula Ia or Ib, or a pharmaceutically acceptable salt thereof;
about 0.142 w/w % of the monohydrate of the compound of Formula IIa or IIb; and
one or more pharmaceutically acceptable excipients.

In some embodiments, each tablet of the maintenance dosage comprises:
about 26.6 w/w % to about 26.7 w/w % of the spray dried dispersion of the compound of Formula Ia or Ib, or a pharmaceutically acceptable salt thereof;
about 0.14 w/w % to about 0.15 w/w % of the compound of Formula IIa or IIb, or a pharmaceutically acceptable salt, co-crystal, solvate, or combination thereof;
about 41 w/w % to about 42 w/w % of mannitol;
about 22.2 w/w % to about 22.3 w/w % of microcrystalline cellulose;
about 7.9 w/w % to about 8.1 w/w % of croscarmellose sodium; and
about 1.4 w/w % to about 1.6 w/w % of magnesium stearate.

In some embodiments, each tablet of the maintenance dosage comprises:
about 26.67 w/w % of the spray dried dispersion of the compound of Formula Ia or Ib, or a pharmaceutically acceptable salt thereof;
about 0.142 w/w % of the compound of Formula IIa or IIb, or a pharmaceutically acceptable salt, co-crystal, solvate, or combination thereof;
about 41.40 w/w % of mannitol;
about 22.288 w/w % microcrystalline cellulose;
about 8.0 w/w % of croscarmellose sodium; and
about 1.5 w/w % of magnesium stearate.

In some embodiments, each tablet of the maintenance dosage comprises:
about 400 mg of the spray dried dispersion of the compound of Formula Ia or Ib, or a pharmaceutically acceptable salt thereof; and
about 2.1 mg to about 2.2 mg of the compound of Formula IIa or IIb, or a pharmaceutically acceptable salt, co-crystal, solvate, or combination thereof.

In some embodiments, each tablet of the maintenance dosage comprises:
about 400 mg of the spray dried dispersion of the compound of Formula Ia or Ib, or a pharmaceutically acceptable salt thereof; and
about 2.130 mg of the compound of Formula IIa or IIb, or a pharmaceutically acceptable salt, co-crystal, solvate, or combination thereof.

In some embodiments, each tablet of the maintenance dosage comprises:
about 400 mg of the spray dried dispersion of the compound of Formula Ia or Ib, or a pharmaceutically acceptable salt thereof; and
about 2.0 mg of the compound of Formula IIa or IIb, or a pharmaceutically acceptable salt, co-crystal, solvate, or combination thereof, on a free base basis.

In some embodiments, each tablet of the maintenance dosage comprises:
about 400 mg of the spray dried dispersion of the compound of Formula Ia or Ib, or a pharmaceutically acceptable salt thereof;
about 2.1 mg to about 2.2 mg of the compound of Formula IIa or IIb, or a pharmaceutically acceptable salt, co-crystal, solvate, or combination thereof;
about 615 mg to about 625 mg of mannitol;
about 330 mg to about 340 mg of microcrystalline cellulose;
about 115 mg to about 125 mg croscarmellose sodium; and
about 20 mg to about 25 mg magnesium stearate.

In some embodiments, each tablet of the maintenance dosage comprises:
about 400 mg of the spray dried dispersion of the compound of Formula Ia or Ib, or a pharmaceutically acceptable salt thereof;
about 2.130 mg of the compound of Formula IIa or IIb, or a pharmaceutically acceptable salt, co-crystal, solvate, or combination thereof;
about 621 mg of mannitol;
about 334.32 mg of microcrystalline cellulose;
about 120 mg croscarmellose sodium; and
about 22.5 mg magnesium stearate.

In some embodiments, each tablet of the maintenance dosage comprises:
about 400 mg of the spray dried dispersion of the compound of Formula Ia or Ib, or a pharmaceutically acceptable salt thereof;
about 2.0 mg of the compound of Formula IIa or IIb, or a pharmaceutically acceptable salt, co-crystal, solvate, or combination thereof, on a free base basis;
about 621 mg of mannitol;
about 334.32 mg of microcrystalline cellulose;
about 120 mg croscarmellose sodium; and
about 22.5 mg magnesium stearate.

In some embodiments, each tablet of the maintenance dosage comprises:
about 26.6 w/w % to about 26.7 w/w % of the spray dried dispersion of the sodium salt of the compound of Formula Ia or Ib;
about 0.14 w/w % to about 0.15 w/w % of the compound of Formula IIa or IIb, or a pharmaceutically acceptable salt, co-crystal, solvate, or combination thereof; and
one or more pharmaceutically acceptable excipients.

In some embodiments, each tablet of the maintenance dosage comprises:
about 26.67 w/w % of the spray dried dispersion of the sodium salt of the compound of Formula Ia or Ib;
about 0.142 w/w % of the compound of Formula IIa or IIb, or a pharmaceutically acceptable salt, co-crystal, solvate, or combination thereof; and
one or more pharmaceutically acceptable excipients.

In some embodiments, each tablet of the maintenance dosage comprises:
about 26.6 w/w % to about 26.7 w/w % of the spray dried dispersion of the sodium salt of the compound of Formula Ia or Ib;
about 0.14 w/w % to about 0.15 w/w % of the compound of Formula IIa or IIb, or a solvate thereof, and
one or more pharmaceutically acceptable excipients.

In some embodiments, each tablet of the maintenance dosage comprises:
about 26.67 w/w % of the spray dried dispersion of the sodium salt of the compound of Formula Ia or Ib;
about 0.142 w/w % of the compound of Formula IIa or IIb, or a solvate thereof; and
one or more pharmaceutically acceptable excipients.

In some embodiments, each tablet of the maintenance dosage comprises:
about 26.6 w/w % to about 26.7 w/w % of the spray dried dispersion of the sodium salt of the compound of Formula Ia or Ib;
about 0.14 w/w % to about 0.15 w/w % of the monohydrate of the compound of Formula IIa or IIb; and
one or more pharmaceutically acceptable excipients.

In some embodiments, each tablet of the maintenance dosage comprises:
about 26.67 w/w % of the spray dried dispersion of the sodium salt of the compound of Formula Ia or Ib;
about 0.142 w/w % of the monohydrate of the compound of Formula IIa or IIb; and
one or more pharmaceutically acceptable excipients.

In some embodiments, each tablet of the maintenance dosage comprises:
about 26.6 w/w % to about 26.7 w/w % of the spray dried dispersion of the sodium salt of the compound of Formula Ia or Ib;
about 0.14 w/w % to about 0.15 w/w % of the compound of Formula IIa or IIb, or a pharmaceutically acceptable salt, co-crystal, solvate, or combination thereof;
about 41 w/w % to about 42 w/w % of mannitol;
about 22.2 w/w % to about 22.3 w/w % of microcrystalline cellulose;
about 7.9 w/w % to about 8.1 w/w % of croscarmellose sodium; and
about 1.4 w/w % to about 1.6 w/w % of magnesium stearate.

In some embodiments, each tablet of the maintenance dosage comprises:
about 26.67 w/w % of the spray dried dispersion of the sodium salt of the compound of Formula Ia or Ib;
about 0.142 w/w % of the compound of Formula IIa or IIb, or a pharmaceutically acceptable salt, co-crystal, solvate, or combination thereof;
about 41.40 w/w % of mannitol;
about 22.288 w/w % microcrystalline cellulose;
about 8.0 w/w % of croscarmellose sodium; and
about 1.5 w/w % of magnesium stearate.

In some embodiments, each tablet of the maintenance dosage comprises:
about 400 mg of the spray dried dispersion of the sodium salt of the compound of Formula Ia or Ib; and
about 2.1 mg to about 2.2 mg of the compound of Formula IIa or IIb, or a pharmaceutically acceptable salt, co-crystal, solvate, or combination thereof.

In some embodiments, each tablet of the maintenance dosage comprises:
about 400 mg of the spray dried dispersion of the sodium salt of the compound of Formula Ia or Ib; and
about 2.130 mg of the compound of Formula IIa or IIb, or a pharmaceutically acceptable salt, co-crystal, solvate, or combination thereof.

In some embodiments, each tablet of the maintenance dosage comprises:
about 400 mg of the spray dried dispersion of the sodium salt of the compound of Formula Ia or Ib; and
about 2.0 mg of the compound of Formula IIa or IIb, or a pharmaceutically acceptable salt, co-crystal, solvate, or combination thereof, on a free base basis.

In some embodiments, each tablet of the maintenance dosage comprises:
about 400 mg of the spray dried dispersion of the sodium salt of the compound of Formula Ia or Ib;
about 2.1 mg to about 2.2 mg of the compound of Formula IIa or IIb, or a pharmaceutically acceptable salt, co-crystal, solvate, or combination thereof;
about 615 mg to about 625 mg of mannitol;
about 330 mg to about 340 mg of microcrystalline cellulose;
about 115 mg to about 125 mg croscarmellose sodium; and
about 20 mg to about 25 mg magnesium stearate.

In some embodiments, each tablet of the maintenance dosage comprises:
about 400 mg of the spray dried dispersion of the sodium salt of the compound of Formula Ia or Ib;
about 2.130 mg of the compound of Formula IIa or IIb, or a pharmaceutically acceptable salt, co-crystal, solvate, or combination thereof;
about 621 mg of mannitol;
about 334.32 mg of microcrystalline cellulose;
about 120 mg croscarmellose sodium; and
about 22.5 mg magnesium stearate.

In some embodiments, each tablet of the maintenance dosage comprises:
about 400 mg of the spray dried dispersion of the sodium salt of the compound of Formula Ia or Ib;
about 2.0 mg of the compound of Formula IIa or IIb, or a pharmaceutically acceptable salt, co-crystal, solvate, or combination thereof, on a free base basis;
about 621 mg of mannitol;
about 334.32 mg of microcrystalline cellulose;
about 120 mg croscarmellose sodium; and
about 22.5 mg magnesium stearate.

In some embodiments, each tablet of the maintenance dosage comprises:
about 26.6 w/w % to about 26.7 w/w % of the spray dried dispersion of the sodium salt of the compound of Formula Ia;
about 0.14 w/w % to about 0.15 w/w % of the compound of Formula IIa or IIb, or a pharmaceutically acceptable salt, co-crystal, solvate, or combination thereof; and
one or more pharmaceutically acceptable excipients.

In some embodiments, each tablet of the maintenance dosage comprises:
about 26.67 w/w % of the spray dried dispersion of the sodium salt of the compound of Formula Ia;
about 0.142 w/w % of the compound of Formula IIa or IIb, or a pharmaceutically acceptable salt, co-crystal, solvate, or combination thereof; and
one or more pharmaceutically acceptable excipients.

In some embodiments, each tablet of the maintenance dosage comprises:
about 26.6 w/w % to about 26.7 w/w % of the spray dried dispersion of the sodium salt of the compound of Formula Ia;
about 0.14 w/w % to about 0.15 w/w % of the compound of Formula IIa or IIb, or a solvate thereof, and
one or more pharmaceutically acceptable excipients.

In some embodiments, each tablet of the maintenance dosage comprises:
about 26.67 w/w % of the spray dried dispersion of the sodium salt of the compound of Formula Ia;
about 0.142 w/w % of the compound of Formula IIa or IIb, or a solvate thereof; and
one or more pharmaceutically acceptable excipients.

In some embodiments, each tablet of the maintenance dosage comprises:
about 26.6 w/w % to about 26.7 w/w % of the spray dried dispersion of the sodium salt of the compound of Formula Ia;
about 0.14 w/w % to about 0.15 w/w % of the monohydrate of the compound of Formula IIa or IIb; and
one or more pharmaceutically acceptable excipients.

In some embodiments, each tablet of the maintenance dosage comprises:
about 26.67 w/w % of the spray dried dispersion of the sodium salt of the compound of Formula Ia;
about 0.142 w/w % of the monohydrate of the compound of Formula IIa or IIb; and
one or more pharmaceutically acceptable excipients.

In some embodiments, each tablet of the maintenance dosage comprises:
about 26.6 w/w % to about 26.7 w/w % of the spray dried dispersion of the sodium salt of the compound of Formula Ia;
about 0.14 w/w % to about 0.15 w/w % of the compound of Formula IIa or IIb, or a pharmaceutically acceptable salt, co-crystal, solvate, or combination thereof;
about 41 w/w % to about 42 w/w % of mannitol;
about 22.2 w/w % to about 22.3 w/w % of microcrystalline cellulose;
about 7.9 w/w % to about 8.1 w/w % of croscarmellose sodium; and
about 1.4 w/w % to about 1.6 w/w % of magnesium stearate.

In some embodiments, each tablet of the maintenance dosage comprises:
about 26.67 w/w % of the spray dried dispersion of the sodium salt of the compound of Formula Ia;
about 0.142 w/w % of the compound of Formula IIa or IIb, or a pharmaceutically acceptable salt, co-crystal, solvate, or combination thereof;
about 41.40 w/w % of mannitol;
about 22.288 w/w % microcrystalline cellulose;
about 8.0 w/w % of croscarmellose sodium; and
about 1.5 w/w % of magnesium stearate.

In some embodiments, each tablet of the maintenance dosage comprises:
about 400 mg of the spray dried dispersion of the sodium salt of the compound of Formula Ia; and
about 2.1 mg to about 2.2 mg of the compound of Formula IIa or IIb, or a pharmaceutically acceptable salt, co-crystal, solvate, or combination thereof.

In some embodiments, each tablet of the maintenance dosage comprises:
about 400 mg of the spray dried dispersion of the sodium salt of the compound of Formula Ia; and
about 2.130 mg of the compound of Formula IIa or IIb, or a pharmaceutically acceptable salt, co-crystal, solvate, or combination thereof.

In some embodiments, each tablet of the maintenance dosage comprises:
about 400 mg of the spray dried dispersion of the sodium salt of the compound of Formula Ia; and
about 2.0 mg of the compound of Formula IIa or IIb, or a pharmaceutically acceptable salt, co-crystal, solvate, or combination thereof, on a free base basis.

In some embodiments, each tablet of the maintenance dosage comprises:
about 400 mg of the spray dried dispersion of the sodium salt of the compound of Formula Ia;
about 2.1 mg to about 2.2 mg of the compound of Formula IIa or IIb, or a pharmaceutically acceptable salt, co-crystal, solvate, or combination thereof;
about 615 mg to about 625 mg of mannitol;
about 330 mg to about 340 mg of microcrystalline cellulose;
about 115 mg to about 125 mg croscarmellose sodium; and
about 20 mg to about 25 mg magnesium stearate.

In some embodiments, each tablet of the maintenance dosage comprises:
about 400 mg of the spray dried dispersion of the sodium salt of the compound of Formula Ia;
about 2.130 mg of the compound of Formula IIa or IIb, or a pharmaceutically acceptable salt, co-crystal, solvate, or combination thereof;
about 621 mg of mannitol;
about 334.32 mg of microcrystalline cellulose;
about 120 mg croscarmellose sodium; and
about 22.5 mg magnesium stearate.

In some embodiments, each tablet of the maintenance dosage comprises:
about 400 mg of the spray dried dispersion of the sodium salt of the compound of Formula Ia;
about 2.0 mg of the compound of Formula IIa or IIb, or a pharmaceutically acceptable salt, co-crystal, solvate, or combination thereof, on a free base basis;
about 621 mg of mannitol;
about 334.32 mg of microcrystalline cellulose;
about 120 mg croscarmellose sodium; and
about 22.5 mg magnesium stearate.

In some embodiments, each tablet of the maintenance dosage comprises:
about 26.6 w/w % to about 26.7 w/w % of the lenacapavir spray dried dispersion;
about 0.14 w/w % to about 0.15 w/w % of the compound of Formula IIa or IIb, or a pharmaceutically acceptable salt, co-crystal, solvate, or combination thereof; and
one or more pharmaceutically acceptable excipients.

In some embodiments, each tablet of the maintenance dosage comprises:
about 26.67 w/w % of the lenacapavir spray dried dispersion;
about 0.142 w/w % of the compound of Formula IIa or IIb, or a pharmaceutically acceptable salt, co-crystal, solvate, or combination thereof; and
one or more pharmaceutically acceptable excipients.

In some embodiments, each tablet of the maintenance dosage comprises:
about 26.6 w/w % to about 26.7 w/w % of the lenacapavir spray dried dispersion;
about 0.14 w/w % to about 0.15 w/w % of the compound of Formula IIa or IIb, or a solvate thereof, and
one or more pharmaceutically acceptable excipients.

In some embodiments, each tablet of the maintenance dosage comprises:
about 26.67 w/w % of the lenacapavir spray dried dispersion;
about 0.142 w/w % of the compound of Formula IIa or IIb, or a solvate thereof; and
one or more pharmaceutically acceptable excipients.

In some embodiments, each tablet of the maintenance dosage comprises:
about 26.6 w/w % to about 26.7 w/w % of the lenacapavir spray dried dispersion;
about 0.14 w/w % to about 0.15 w/w % of the monohydrate of the compound of Formula IIa or IIb; and
one or more pharmaceutically acceptable excipients.

In some embodiments, each tablet of the maintenance dosage comprises:
about 26.67 w/w % of the lenacapavir spray dried dispersion;
about 0.142 w/w % of the monohydrate of the compound of Formula IIa or IIb; and
one or more pharmaceutically acceptable excipients.

In some embodiments, each tablet of the maintenance dosage comprises:
about 26.6 w/w % to about 26.7 w/w % of the lenacapavir spray dried dispersion;
about 0.14 w/w % to about 0.15 w/w % of the compound of Formula IIa or IIb, or a pharmaceutically acceptable salt, co-crystal, solvate, or combination thereof;
about 41 w/w % to about 42 w/w % of mannitol;
about 22.2 w/w % to about 22.3 w/w % of microcrystalline cellulose;
about 7.9 w/w % to about 8.1 w/w % of croscarmellose sodium; and
about 1.4 w/w % to about 1.6 w/w % of magnesium stearate.

In some embodiments, each tablet of the maintenance dosage comprises:
about 26.67 w/w % of the lenacapavir spray dried dispersion;
about 0.142 w/w % of the compound of Formula IIa or IIb, or a pharmaceutically acceptable salt, co-crystal, solvate, or combination thereof;
about 41.40 w/w % of mannitol;
about 22.288 w/w % microcrystalline cellulose;
about 8.0 w/w % of croscarmellose sodium; and
about 1.5 w/w % of magnesium stearate.

In some embodiments, each tablet of the maintenance dosage comprises:
about 400 mg of the lenacapavir spray dried dispersion; and
about 2.1 mg to about 2.2 mg of the compound of Formula IIa or IIb, or a pharmaceutically acceptable salt, co-crystal, solvate, or combination thereof.

In some embodiments, each tablet of the maintenance dosage comprises:
about 400 mg of the lenacapavir spray dried dispersion; and
about 2.130 mg of the compound of Formula IIa or IIb, or a pharmaceutically acceptable salt, co-crystal, solvate, or combination thereof.

In some embodiments, each tablet of the maintenance dosage comprises:
about 400 mg of the lenacapavir spray dried dispersion; and
about 2.0 mg of the compound of Formula IIa or IIb, or a pharmaceutically acceptable salt, co-crystal, solvate, or combination thereof, on a free base basis.

In some embodiments, each tablet of the maintenance dosage comprises:
about 400 mg of the lenacapavir spray dried dispersion;
about 2.1 mg to about 2.2 mg of the compound of Formula IIa or IIb, or a pharmaceutically acceptable salt, co-crystal, solvate, or combination thereof;
about 615 mg to about 625 mg of mannitol;
about 330 mg to about 340 mg of microcrystalline cellulose;
about 115 mg to about 125 mg croscarmellose sodium; and
about 20 mg to about 25 mg magnesium stearate.

In some embodiments, each tablet of the maintenance dosage comprises:
about 400 mg of the lenacapavir spray dried dispersion;
about 2.130 mg of the compound of Formula IIa or IIb, or a pharmaceutically acceptable salt, co-crystal, solvate, or combination thereof;
about 621 mg of mannitol;
about 334.32 mg of microcrystalline cellulose;
about 120 mg croscarmellose sodium; and
about 22.5 mg magnesium stearate.

In some embodiments, each tablet of the maintenance dosage comprises:
about 400 mg of the lenacapavir spray dried dispersion;
about 2.0 mg of the compound of Formula IIa or IIb, or a pharmaceutically acceptable salt, co-crystal, solvate, or combination thereof, on a free base basis;
about 621 mg of mannitol;
about 334.32 mg of microcrystalline cellulose;
about 120 mg croscarmellose sodium; and
about 22.5 mg magnesium stearate.

In some embodiments, each tablet of the maintenance dosage comprises:
about 300 mg of the compound of Formula Ia or Ib, or a pharmaceutically acceptable salt, co-crystal, solvate, or combination thereof, on a free acid basis; and
about 2.1 mg to about 2.2 mg of the compound of Formula IIa or IIb, or a pharmaceutically acceptable salt, co-crystal, solvate, or combination thereof.

In some embodiments, each tablet of the maintenance dosage comprises:
about 300 mg of the compound of Formula Ia or Ib, or a pharmaceutically acceptable salt, co-crystal, solvate, or combination thereof, on a free acid basis; and
about 2.130 mg of the compound of Formula IIa or IIb, or a pharmaceutically acceptable salt, co-crystal, solvate, or combination thereof.

In some embodiments, each tablet of the maintenance dosage comprises:
about 300 mg of the compound of Formula Ia or Ib, or a pharmaceutically acceptable salt, co-crystal, solvate, or combination thereof, on a free acid basis; and
about 2.0 mg of the compound of Formula IIa or IIb, or a pharmaceutically acceptable salt, co-crystal, solvate, or combination thereof, on a free base basis.

In some embodiments, each tablet of the maintenance dosage comprises:
about 300 mg of the compound of Formula Ia or Ib, or a pharmaceutically acceptable salt, co-crystal, solvate, or combination thereof, on a free acid basis;
about 2.1 mg to about 2.2 mg of the compound of Formula IIa or IIb, or a pharmaceutically acceptable salt, co-crystal, solvate, or combination thereof;
about 615 mg to about 625 mg of mannitol;
about 330 mg to about 340 mg of microcrystalline cellulose;
about 115 mg to about 125 mg croscarmellose sodium; and
about 20 mg to about 25 mg magnesium stearate.

In some embodiments, each tablet of the maintenance dosage comprises:
about 300 mg of the compound of Formula Ia or Ib, or a pharmaceutically acceptable salt, co-crystal, solvate, or combination thereof, on a free acid basis;
about 2.130 mg of the compound of Formula IIa or IIb, or a pharmaceutically acceptable salt, co-crystal, solvate, or combination thereof;
about 621 mg of mannitol;
about 334.32 mg of microcrystalline cellulose;
about 120 mg croscarmellose sodium; and
about 22.5 mg magnesium stearate.

In some embodiments, each tablet of the maintenance dosage comprises:
about 300 mg of the compound of Formula Ia or Ib, or a pharmaceutically acceptable salt, co-crystal, solvate, or combination thereof, on a free acid basis;
about 2.0 mg of the compound of Formula IIa or IIb, or a pharmaceutically acceptable salt, co-crystal, solvate, or combination thereof, on a free base basis;
about 621 mg of mannitol;
about 334.32 mg of microcrystalline cellulose;
about 120 mg croscarmellose sodium; and
about 22.5 mg magnesium stearate.

In some embodiments, each tablet of the maintenance dosage comprises:
about 200 mg to about 400 mg of the compound of Formula Ia or Ib, or a pharmaceutically acceptable salt thereof, on a free acid basis;
about 0.1 mg to about 2.5 mg of the compound of Formula IIa or IIb, or a solvate thereof, on a free base basis;
about 50 mg to about 100 mg of copovidone;
about 5 mg to about 30 mg of poloxamer 407;
about 600 mg to about 700 mg of mannitol;
about 300 mg to about 350 mg of microcrystalline cellulose;
about 100 mg to about 150 mg of croscarmellose sodium; and
about 20 mg to about 25 mg of magnesium stearate.

In some embodiments, each tablet of the maintenance dosage comprises:
about 300 mg of the compound of Formula Ia or Ib, or a pharmaceutically acceptable salt thereof, on a free acid basis;
about 2 mg of the compound of Formula IIa or IIb, or a solvate thereof, on a free base basis;
about 65 mg to about 75 mg of copovidone;
about 15 mg to about 25 mg of poloxamer 407;
about 615 mg to about 625 mg of mannitol;
about 330 mg to about 340 mg of microcrystalline cellulose;
about 115 mg to about 125 mg of croscarmellose sodium; and
about 20 mg to about 25 mg of magnesium stearate.

In some embodiments, each tablet of the maintenance dosage comprises:
about 300 mg of the compound of Formula Ia or Ib, or a pharmaceutically acceptable salt thereof, on a free acid basis;
about 2 mg of the compound of Formula IIa or IIb, or a solvate thereof, on a free base basis;
about 73.2 mg of copovidone;
about 19.95 mg of poloxamer 407;
about 621 mg of mannitol;
about 334.32 mg of microcrystalline cellulose;
about 120 mg of croscarmellose sodium; and
about 22.5 mg of magnesium stearate.

In some embodiments, each tablet of the maintenance dosage comprises:
about 200 mg to about 400 mg of the sodium salt of the compound of Formula Ia or Ib;
about 0.1 mg to about 2.5 mg of the monohydrate of the compound of Formula IIa or IIb;
about 50 mg to about 100 mg of copovidone;
about 5 mg to about 30 mg of poloxamer 407;
about 600 mg to about 700 mg of mannitol;
about 300 mg to about 350 mg of microcrystalline cellulose;
about 100 mg to about 150 mg of croscarmellose sodium; and
about 20 mg to about 25 mg of magnesium stearate.

In some embodiments, each tablet of the maintenance dosage comprises:
about 306.8 mg of the sodium salt of the compound of Formula Ia or Ib;
about 2.1 mg to about 2.2 mg of the monohydrate of the compound of Formula IIa or IIb;
about 65 mg to about 75 mg of copovidone;
about 15 mg to about 25 mg of poloxamer 407;
about 615 mg to about 625 mg of mannitol;
about 330 mg to about 340 mg of microcrystalline cellulose;
about 115 mg to about 125 mg of croscarmellose sodium; and
about 20 mg to about 25 mg of magnesium stearate.

In some embodiments, each tablet of the maintenance dosage comprises:
about 306.8 mg of the sodium salt of the compound of Formula Ia or Ib;
about 2.130 mg of the monohydrate of the compound of Formula IIa or IIb;
about 65 mg to about 75 mg of copovidone;
about 15 mg to about 25 mg of poloxamer 407;
about 615 mg to about 625 mg of mannitol;
about 330 mg to about 340 mg of microcrystalline cellulose;
about 115 mg to about 125 mg of croscarmellose sodium; and
about 20 mg to about 25 mg of magnesium stearate.

In some embodiments, each tablet of the maintenance dosage comprises:
about 306.8 mg of the sodium salt of the compound of Formula Ia or Ib;
about 2.130 mg of the monohydrate of the compound of Formula IIa or IIb;
about 73.2 mg of copovidone;
about 19.95 mg of poloxamer 407;
about 621 mg of mannitol;
about 334.32 mg of microcrystalline cellulose;
about 120 mg of croscarmellose sodium; and
about 22.5 mg of magnesium stearate.

In some embodiments, each tablet of the maintenance dosage comprises:
about 200 mg to about 400 mg of the compound of Formula Ia or Ib, or a pharmaceutically acceptable salt thereof, on a free acid basis;
about 0.1 mg to about 2.5 mg of the compound of Formula IIa or IIb, or a solvate thereof, on a free base basis;
about 50 mg to about 100 mg of copovidone;
about 5 mg to about 30 mg of poloxamer 407;
about 600 mg to about 700 mg of mannitol;
about 300 mg to about 350 mg of microcrystalline cellulose;
about 100 mg to about 150 mg of croscarmellose sodium;
about 20 mg to about 25 mg of magnesium stearate; and
an outer film coat.

In some embodiments, each tablet of the maintenance dosage comprises:
about 300 mg of the compound of Formula Ia or Ib, or a pharmaceutically acceptable salt thereof, on a free acid basis;
about 2 mg of the compound of Formula IIa or IIb, or a solvate thereof, on a free base basis;
about 65 mg to about 75 mg of copovidone;
about 15 mg to about 25 mg of poloxamer 407;
about 615 mg to about 625 mg of mannitol;
about 330 mg to about 340 mg of microcrystalline cellulose;
about 115 mg to about 125 mg of croscarmellose sodium;
about 20 mg to about 25 mg of magnesium stearate; and
an outer film coat.

In some embodiments, each tablet of the maintenance dosage comprises:
about 300 mg of the compound of Formula Ia or Ib, or a pharmaceutically acceptable salt thereof, on a free acid basis;
about 2 mg of the compound of Formula IIa or IIb, or a solvate thereof, on a free base basis, on a free base basis;
about 73.2 mg of copovidone;
about 19.95 mg of poloxamer 407;
about 621 mg of mannitol;
about 334.32 mg of microcrystalline cellulose;
about 120 mg of croscarmellose sodium;
about 22.5 mg of magnesium stearate; and
an outer film coat.

In some embodiments, each tablet of the maintenance dosage comprises:
about 200 mg to about 400 mg of the sodium salt of the compound of Formula Ia or Ib;
about 0.1 mg to about 2.5 mg of the monohydrate of the compound of Formula IIa or IIb;
about 50 mg to about 100 mg of copovidone;
about 5 mg to about 30 mg of poloxamer 407;
about 600 mg to about 700 mg of mannitol;
about 300 mg to about 350 mg of microcrystalline cellulose;
about 100 mg to about 150 mg of croscarmellose sodium;
about 20 mg to about 25 mg of magnesium stearate; and
an outer film coat.

In some embodiments, each tablet of the maintenance dosage comprises:
about 306.8 mg of the sodium salt of the compound of Formula Ia or Ib;
about 2.1 mg to about 2.2 mg of the monohydrate of the compound of Formula IIa or IIb;
about 65 mg to about 75 mg of copovidone;
about 15 mg to about 25 mg of poloxamer 407;
about 615 mg to about 625 mg of mannitol;
about 330 mg to about 340 mg of microcrystalline cellulose;
about 115 mg to about 125 mg of croscarmellose sodium;
about 20 mg to about 25 mg of magnesium stearate; and
an outer film coat.

In some embodiments, each tablet of the maintenance dosage comprises:
about 306.8 mg of the sodium salt of the compound of Formula Ia or Ib;
about 2.130 mg of the monohydrate of the compound of Formula IIa or IIb;
about 65 mg to about 75 mg of copovidone;
about 15 mg to about 25 mg of poloxamer 407;
about 615 mg to about 625 mg of mannitol;
about 330 mg to about 340 mg of microcrystalline cellulose;
about 115 mg to about 125 mg of croscarmellose sodium; and
about 20 mg to about 25 mg of magnesium stearate.

In some embodiments, each tablet of the maintenance dosage comprises:
about 306.8 mg of the sodium salt of the compound of Formula Ia or Ib;
about 2.130 mg of the monohydrate of the compound of Formula IIa or IIb;
about 73.2 mg of copovidone;
about 19.95 mg of poloxamer 407;
about 621 mg of mannitol;
about 334.32 mg of microcrystalline cellulose;
about 120 mg of croscarmellose sodium;
about 22.5 mg of magnesium stearate; and
an outer film coat.

In some embodiments, each tablet of the maintenance dosage consists essentially of:
about 200 mg to about 400 mg of the compound of Formula Ia or Ib, or a pharmaceutically acceptable salt thereof, on a free acid basis;
about 0.1 mg to about 2.5 mg of the compound of Formula IIa or IIb, or a solvate thereof, on a free base basis;
about 50 mg to about 100 mg of copovidone;
about 5 mg to about 30 mg of poloxamer 407;
about 600 mg to about 700 mg of mannitol;
about 300 mg to about 350 mg of microcrystalline cellulose;
about 100 mg to about 150 mg of croscarmellose sodium; and
about 20 mg to about 25 mg of magnesium stearate.

In some embodiments, each tablet of the maintenance dosage consists essentially of:
about 300 mg of the compound of Formula Ia or Ib, or a pharmaceutically acceptable salt thereof, on a free acid basis;
about 2 mg of the compound of Formula IIa or IIb, or a solvate thereof, on a free base basis;
about 65 mg to about 75 mg of copovidone;
about 15 mg to about 25 mg of poloxamer 407;
about 615 mg to about 625 mg of mannitol;
about 330 mg to about 340 mg of microcrystalline cellulose;
about 115 mg to about 125 mg of croscarmellose sodium; and
about 20 mg to about 25 mg of magnesium stearate.

In some embodiments, each tablet of the maintenance dosage consists essentially of:
about 300 mg of the compound of Formula Ia or Ib, or a pharmaceutically acceptable salt thereof, on a free acid basis;
about 2 mg of the compound of Formula IIa or IIb, or a solvate thereof, on a free base basis;
about 73.2 mg of copovidone;
about 19.95 mg of poloxamer 407;
about 621 mg of mannitol;
about 334.32 mg of microcrystalline cellulose;
about 120 mg of croscarmellose sodium; and
about 22.5 mg of magnesium stearate.

In some embodiments, each tablet of the maintenance dosage consists essentially of:
about 200 mg to about 400 mg of the sodium salt of the compound of Formula Ia or Ib;
about 0.1 mg to about 2.5 mg of the monohydrate of the compound of Formula IIa or IIb;
about 50 mg to about 100 mg of copovidone;
about 5 mg to about 30 mg of poloxamer 407;
about 600 mg to about 700 mg of mannitol;
about 300 mg to about 350 mg of microcrystalline cellulose;
about 100 mg to about 150 mg of croscarmellose sodium; and
about 20 mg to about 25 mg of magnesium stearate.

In some embodiments, each tablet of the maintenance dosage consists essentially of:
about 306.8 mg of the sodium salt of the compound of Formula Ia or Ib;
about 2.1 mg to about 2.2 mg of the monohydrate of the compound of Formula IIa or IIb;
about 65 mg to about 75 mg of copovidone;
about 15 mg to about 25 mg of poloxamer 407;
about 615 mg to about 625 mg of mannitol;
about 330 mg to about 340 mg of microcrystalline cellulose;
about 115 mg to about 125 mg of croscarmellose sodium; and
about 20 mg to about 25 mg of magnesium stearate.

In some embodiments, each tablet of the maintenance dosage consists essentially of:
about 306.8 mg of the sodium salt of the compound of Formula Ia or Ib;
about 2.130 mg of the monohydrate of the compound of Formula IIa or IIb;
about 65 mg to about 75 mg of copovidone;
about 15 mg to about 25 mg of poloxamer 407;
about 615 mg to about 625 mg of mannitol;
about 330 mg to about 340 mg of microcrystalline cellulose;
about 115 mg to about 125 mg of croscarmellose sodium; and
about 20 mg to about 25 mg of magnesium stearate.

In some embodiments, each tablet of the maintenance dosage consists essentially of:
about 306.8 mg of the sodium salt of the compound of Formula Ia or Ib;
about 2.130 mg of the monohydrate of the compound of Formula IIa or IIb;
about 73.2 mg of copovidone;
about 19.95 mg of poloxamer 407;
about 621 mg of mannitol;
about 334.32 mg of microcrystalline cellulose;
about 120 mg of croscarmellose sodium; and
about 22.5 mg of magnesium stearate.

In some embodiments, each tablet of the maintenance dosage consists essentially of:
about 200 mg to about 400 mg of the compound of Formula Ia or Ib, or a pharmaceutically acceptable salt thereof, on a free acid basis;
about 0.1 mg to about 2.5 mg of the compound of Formula IIa or IIb, or a solvate thereof, on a free base basis;
about 50 mg to about 100 mg of copovidone;
about 5 mg to about 30 mg of poloxamer 407;
about 600 mg to about 700 mg of mannitol;
about 300 mg to about 350 mg of microcrystalline cellulose;
about 100 mg to about 150 mg of croscarmellose sodium;
about 20 mg to about 25 mg of magnesium stearate; and
an outer film coat.

In some embodiments, each tablet of the maintenance dosage consists essentially of:

about 300 mg of the compound of Formula Ia or Ib, or a pharmaceutically acceptable salt thereof, on a free acid basis;
about 2 mg of the compound of Formula IIa or IIb, or a solvate thereof, on a free base basis;
about 65 mg to about 75 mg of copovidone;
about 15 mg to about 25 mg of poloxamer 407;
about 615 mg to about 625 mg of mannitol;
about 330 mg to about 340 mg of microcrystalline cellulose;
about 115 mg to about 125 mg of croscarmellose sodium;
about 20 mg to about 25 mg of magnesium stearate; and
an outer film coat.

In some embodiments, each tablet of the maintenance dosage consists essentially of:
about 300 mg of the compound of Formula Ia or Ib, or a pharmaceutically acceptable salt thereof, on a free acid basis;
about 2 mg of the compound of Formula IIa or IIb, or a solvate thereof, on a free base basis, on a free base basis;
about 73.2 mg of copovidone;
about 19.95 mg of poloxamer 407;
about 621 mg of mannitol;
about 334.32 mg of microcrystalline cellulose;
about 120 mg of croscarmellose sodium;
about 22.5 mg of magnesium stearate; and
an outer film coat.

In some embodiments, each tablet of the maintenance dosage consists essentially of:
about 200 mg to about 400 mg of the sodium salt of the compound of Formula Ia or Ib;
about 0.1 mg to about 2.5 mg of the monohydrate of the compound of Formula IIa or IIb;
about 50 mg to about 100 mg of copovidone;
about 5 mg to about 30 mg of poloxamer 407;
about 600 mg to about 700 mg of mannitol;
about 300 mg to about 350 mg of microcrystalline cellulose;
about 100 mg to about 150 mg of croscarmellose sodium;
about 20 mg to about 25 mg of magnesium stearate; and
an outer film coat.

In some embodiments, each tablet of the maintenance dosage consists essentially of:
about 306.8 mg of the sodium salt of the compound of Formula Ia or Ib;
about 2.1 mg to about 2.2 mg of the monohydrate of the compound of Formula IIa or IIb;
about 65 mg to about 75 mg of copovidone;
about 15 mg to about 25 mg of poloxamer 407;
about 615 mg to about 625 mg of mannitol;
about 330 mg to about 340 mg of microcrystalline cellulose;
about 115 mg to about 125 mg of croscarmellose sodium;
about 20 mg to about 25 mg of magnesium stearate; and
an outer film coat.

In some embodiments, each tablet of the maintenance dosage consists essentially of:
about 306.8 mg of the sodium salt of the compound of Formula Ia or Ib;
about 2.130 mg of the monohydrate of the compound of Formula IIa or IIb;
about 65 mg to about 75 mg of copovidone;
about 15 mg to about 25 mg of poloxamer 407;
about 615 mg to about 625 mg of mannitol;
about 330 mg to about 340 mg of microcrystalline cellulose;
about 115 mg to about 125 mg of croscarmellose sodium; and
about 20 mg to about 25 mg of magnesium stearate.

In some embodiments, each tablet of the maintenance dosage consists essentially of:
about 306.8 mg of the sodium salt of the compound of Formula Ia or Ib;
about 2.130 mg of the monohydrate of the compound of Formula IIa or IIb;
about 73.2 mg of copovidone;
about 19.95 mg of poloxamer 407;
about 621 mg of mannitol;
about 334.32 mg of microcrystalline cellulose;
about 120 mg of croscarmellose sodium;
about 22.5 mg of magnesium stearate; and
an outer film coat.

In some embodiments, each tablet of the maintenance dosage consists of:
about 200 mg to about 400 mg of the compound of Formula Ia or Ib, or a pharmaceutically acceptable salt thereof, on a free acid basis;
about 0.1 mg to about 2.5 mg of the compound of Formula IIa or IIb, or a solvate thereof, on a free base basis;
about 50 mg to about 100 mg of copovidone;
about 5 mg to about 30 mg of poloxamer 407;
about 600 mg to about 700 mg of mannitol;
about 300 mg to about 350 mg of microcrystalline cellulose;
about 100 mg to about 150 mg of croscarmellose sodium; and
about 20 mg to about 25 mg of magnesium stearate.

In some embodiments, each tablet of the maintenance dosage consists of:
about 300 mg of the compound of Formula Ia or Ib, or a pharmaceutically acceptable salt thereof, on a free acid basis;
about 2 mg of the compound of Formula IIa or IIb, or a solvate thereof, on a free base basis;
about 65 mg to about 75 mg of copovidone;
about 15 mg to about 25 mg of poloxamer 407;
about 615 mg to about 625 mg of mannitol;
about 330 mg to about 340 mg of microcrystalline cellulose;
about 115 mg to about 125 mg of croscarmellose sodium; and
about 20 mg to about 25 mg of magnesium stearate.

In some embodiments, each tablet of the maintenance dosage consists of:
about 300 mg of the compound of Formula Ia or Ib, or a pharmaceutically acceptable salt thereof, on a free acid basis;
about 2 mg of the compound of Formula IIa or IIb, or a solvate thereof, on a free base basis;
about 73.2 mg of copovidone;
about 19.95 mg of poloxamer 407;
about 621 mg of mannitol;
about 334.32 mg of microcrystalline cellulose;
about 120 mg of croscarmellose sodium; and
about 22.5 mg of magnesium stearate.

In some embodiments, each tablet of the maintenance dosage consists of:
about 200 mg to about 400 mg of the sodium salt of the compound of Formula Ia or Ib;
about 0.1 mg to about 2.5 mg of the monohydrate of the compound of Formula IIa or IIb;
about 50 mg to about 100 mg of copovidone;
about 5 mg to about 30 mg of poloxamer 407;
about 600 mg to about 700 mg of mannitol;
about 300 mg to about 350 mg of microcrystalline cellulose;
about 100 mg to about 150 mg of croscarmellose sodium; and
about 20 mg to about 25 mg of magnesium stearate.

In some embodiments, each tablet of the maintenance dosage consists of:
about 306.8 mg of the sodium salt of the compound of Formula Ia or Ib;
about 2.1 mg to about 2.2 mg of the monohydrate of the compound of Formula IIa or IIb;
about 65 mg to about 75 mg of copovidone;
about 15 mg to about 25 mg of poloxamer 407;
about 615 mg to about 625 mg of mannitol;
about 330 mg to about 340 mg of microcrystalline cellulose;
about 115 mg to about 125 mg of croscarmellose sodium; and
about 20 mg to about 25 mg of magnesium stearate.

In some embodiments, each tablet of the maintenance dosage consists of:
about 306.8 mg of the sodium salt of the compound of Formula Ia or Ib;
about 2.130 mg of the monohydrate of the compound of Formula IIa or IIb;
about 65 mg to about 75 mg of copovidone;
about 15 mg to about 25 mg of poloxamer 407;
about 615 mg to about 625 mg of mannitol;
about 330 mg to about 340 mg of microcrystalline cellulose;
about 115 mg to about 125 mg of croscarmellose sodium; and
about 20 mg to about 25 mg of magnesium stearate.

In some embodiments, each tablet of the maintenance dosage consists of:
about 306.8 mg of the sodium salt of the compound of Formula Ia or Ib;
about 2.130 mg of the monohydrate of the compound of Formula IIa or IIb;
about 73.2 mg of copovidone;
about 19.95 mg of poloxamer 407;
about 621 mg of mannitol;
about 334.32 mg of microcrystalline cellulose;
about 120 mg of croscarmellose sodium; and
about 22.5 mg of magnesium stearate.

In some embodiments, each tablet of the maintenance dosage consists of:
about 200 mg to about 400 mg of the compound of Formula Ia or Ib, or a pharmaceutically acceptable salt thereof, on a free acid basis;
about 0.1 mg to about 2.5 mg of the compound of Formula IIa or IIb, or a solvate thereof, on a free base basis;
about 50 mg to about 100 mg of copovidone;
about 5 mg to about 30 mg of poloxamer 407;
about 600 mg to about 700 mg of mannitol;
about 300 mg to about 350 mg of microcrystalline cellulose;
about 100 mg to about 150 mg of croscarmellose sodium;
about 20 mg to about 25 mg of magnesium stearate; and
an outer film coat.

In some embodiments, each tablet of the maintenance dosage consists of:
about 300 mg of the compound of Formula Ia or Ib, or a pharmaceutically acceptable salt thereof, on a free acid basis;
about 2 mg of the compound of Formula IIa or IIb, or a solvate thereof, on a free base basis;
about 65 mg to about 75 mg of copovidone;
about 15 mg to about 25 mg of poloxamer 407;
about 615 mg to about 625 mg of mannitol;
about 330 mg to about 340 mg of microcrystalline cellulose;
about 115 mg to about 125 mg of croscarmellose sodium;
about 20 mg to about 25 mg of magnesium stearate; and
an outer film coat.

In some embodiments, each tablet of the maintenance dosage consists of:
about 300 mg of the compound of Formula Ia or Ib, or a pharmaceutically acceptable salt thereof, on a free acid basis;
about 2 mg of the compound of Formula IIa or IIb, or a solvate thereof, on a free base basis, on a free base basis;
about 73.2 mg of copovidone;
about 19.95 mg of poloxamer 407;
about 621 mg of mannitol;
about 334.32 mg of microcrystalline cellulose;
about 120 mg of croscarmellose sodium;
about 22.5 mg of magnesium stearate; and
an outer film coat.

In some embodiments, each tablet of the maintenance dosage consists of:
about 200 mg to about 400 mg of the sodium salt of the compound of Formula Ia or Ib;
about 0.1 mg to about 2.5 mg of the monohydrate of the compound of Formula IIa or IIb;
about 50 mg to about 100 mg of copovidone;
about 5 mg to about 30 mg of poloxamer 407;
about 600 mg to about 700 mg of mannitol;
about 300 mg to about 350 mg of microcrystalline cellulose;
about 100 mg to about 150 mg of croscarmellose sodium;
about 20 mg to about 25 mg of magnesium stearate; and
an outer film coat.

In some embodiments, each tablet of the maintenance dosage consists of:
about 306.8 mg of the sodium salt of the compound of Formula Ia or Ib;
about 2.1 mg to about 2.2 mg of the monohydrate of the compound of Formula IIa or IIb;
about 65 mg to about 75 mg of copovidone;
about 15 mg to about 25 mg of poloxamer 407;
about 615 mg to about 625 mg of mannitol;
about 330 mg to about 340 mg of microcrystalline cellulose;
about 115 mg to about 125 mg of croscarmellose sodium;
about 20 mg to about 25 mg of magnesium stearate; and
an outer film coat.

In some embodiments, each tablet of the maintenance dosage consists of:
about 306.8 mg of the sodium salt of the compound of Formula Ia or Ib;
about 2.130 mg of the monohydrate of the compound of Formula IIa or IIb;
about 65 mg to about 75 mg of copovidone;
about 15 mg to about 25 mg of poloxamer 407;
about 615 mg to about 625 mg of mannitol;
about 330 mg to about 340 mg of microcrystalline cellulose;
about 115 mg to about 125 mg of croscarmellose sodium; and
about 20 mg to about 25 mg of magnesium stearate.

In some embodiments, each tablet of the maintenance dosage consists of:
about 306.8 mg of the sodium salt of the compound of Formula Ia or Ib;
about 2.130 mg of the monohydrate of the compound of Formula IIa or IIb;
about 73.2 mg of copovidone;
about 19.95 mg of poloxamer 407;
about 621 mg of mannitol;
about 334.32 mg of microcrystalline cellulose;
about 120 mg of croscarmellose sodium;
about 22.5 mg of magnesium stearate; and
an outer film coat.

In some embodiments, the method comprises administering to the patient a maintenance dose comprising one tablet, once weekly, wherein the tablet comprises:
about 26.6 w/w % to about 26.7 w/w % of the spray dried dispersion of the compound of Formula Ia or Ib, or a pharmaceutically acceptable salt, co-crystal, solvate, or combination thereof;
about 0.14 w/w % to about 0.15 w/w % of the compound of Formula IIa or IIb, or a pharmaceutically acceptable salt, co-crystal, solvate, or combination thereof; and
one or more pharmaceutically acceptable excipients.

In some embodiments, the method comprises administering to the patient a maintenance dose comprising one tablet, once weekly, wherein the tablet comprises:
about 26.67 w/w % of the spray dried dispersion of the compound of Formula Ia or Ib, or a pharmaceutically acceptable salt, co-crystal, solvate, or combination thereof;
about 0.142 w/w % of the compound of Formula IIa or IIb, or a pharmaceutically acceptable salt, co-crystal, solvate, or combination thereof; and
one or more pharmaceutically acceptable excipients.

In some embodiments, the method comprises administering to the patient a maintenance dose comprising one tablet, once weekly, wherein the tablet comprises:
about 26.6 w/w % to about 26.7 w/w % of the spray dried dispersion of the compound of Formula Ia or Ib, or a pharmaceutically acceptable salt, co-crystal, solvate, or combination thereof;
about 0.14 w/w % to about 0.15 w/w % of the compound of Formula IIa or IIb, or a solvate thereof, and
one or more pharmaceutically acceptable excipients.

In some embodiments, the method comprises administering to the patient a maintenance dose comprising one tablet, once weekly, wherein the tablet comprises:
about 26.67 w/w % of the spray dried dispersion of the compound of Formula Ia or Ib, or a pharmaceutically acceptable salt, co-crystal, solvate, or combination thereof;
about 0.142 w/w % of the compound of Formula IIa or IIb, or a solvate thereof; and
one or more pharmaceutically acceptable excipients.

In some embodiments, the method comprises administering to the patient a maintenance dose comprising one tablet, once weekly, wherein the tablet comprises:
about 26.6 w/w % to about 26.7 w/w % of the spray dried dispersion of the compound of Formula Ia or Ib, or a pharmaceutically acceptable salt, co-crystal, solvate, or combination thereof;
about 0.14 w/w % to about 0.15 w/w % of the monohydrate of the compound of Formula IIa or IIb; and
one or more pharmaceutically acceptable excipients.

In some embodiments, the method comprises administering to the patient a maintenance dose comprising one tablet, once weekly, wherein the tablet comprises:
about 26.67 w/w % of the spray dried dispersion of the compound of Formula Ia or Ib, or a pharmaceutically acceptable salt, co-crystal, solvate, or combination thereof;
about 0.142 w/w % of the monohydrate of the compound of Formula IIa or IIb; and
one or more pharmaceutically acceptable excipients.

In some embodiments, the method comprises administering to the patient a maintenance dose comprising one tablet, once weekly, wherein the tablet comprises:
about 26.6 w/w % to about 26.7 w/w % of the spray dried dispersion of the compound of Formula Ia or Ib, or a pharmaceutically acceptable salt, co-crystal, solvate, or combination thereof;
about 0.14 w/w % to about 0.15 w/w % of the compound of Formula IIa or IIb, or a pharmaceutically acceptable salt, co-crystal, solvate, or combination thereof;
about 41 w/w % to about 42 w/w % of mannitol;
about 22.2 w/w % to about 22.3 w/w % of microcrystalline cellulose;
about 7.9 w/w % to about 8.1 w/w % of croscarmellose sodium; and
about 1.4 w/w % to about 1.6 w/w % of magnesium stearate.

In some embodiments, the method comprises administering to the patient a maintenance dose comprising one tablet, once weekly, wherein the tablet comprises:
about 26.67 w/w % of the spray dried dispersion of the compound of Formula Ia or Ib, or a pharmaceutically acceptable salt, co-crystal, solvate, or combination thereof;
about 0.142 w/w % of the compound of Formula IIa or IIb, or a pharmaceutically acceptable salt, co-crystal, solvate, or combination thereof;
about 41.40 w/w % of mannitol;
about 22.288 w/w % microcrystalline cellulose;
about 8.0 w/w % of croscarmellose sodium; and
about 1.5 w/w % of magnesium stearate.

In some embodiments, the method comprises administering to the patient a maintenance dose comprising one tablet, once weekly, wherein the tablet comprises:
about 400 mg of the spray dried dispersion of the compound of Formula Ia or Ib, or a pharmaceutically acceptable salt, co-crystal, solvate, or combination thereof; and
about 2.1 mg to about 2.2 mg of the compound of Formula IIa or IIb, or a pharmaceutically acceptable salt, co-crystal, solvate, or combination thereof.

In some embodiments, the method comprises administering to the patient a maintenance dose comprising one tablet, once weekly, wherein the tablet comprises:
about 400 mg of the spray dried dispersion of the compound of Formula Ia or Ib, or a pharmaceutically acceptable salt, co-crystal, solvate, or combination thereof; and
about 2.130 mg of the compound of Formula IIa or IIb, or a pharmaceutically acceptable salt, co-crystal, solvate, or combination thereof.

In some embodiments, the method comprises administering to the patient a maintenance dose comprising one tablet, once weekly, wherein the tablet comprises:
about 400 mg of the spray dried dispersion of the compound of Formula Ia or Ib, or a pharmaceutically acceptable salt, co-crystal, solvate, or combination thereof; and
about 2.0 mg of the compound of Formula IIa or IIb, or a pharmaceutically acceptable salt, co-crystal, solvate, or combination thereof, on a free base basis.

In some embodiments, the method comprises administering to the patient a maintenance dose comprising one tablet, once weekly, wherein the tablet comprises:
about 400 mg of the spray dried dispersion of the compound of Formula Ia or Ib, or a pharmaceutically acceptable salt, co-crystal, solvate, or combination thereof;
about 2.1 mg to about 2.2 mg of the compound of Formula IIa or IIb, or a pharmaceutically acceptable salt, co-crystal, solvate, or combination thereof;
about 615 mg to about 625 mg of mannitol;
about 330 mg to about 340 mg of microcrystalline cellulose;
about 115 mg to about 125 mg croscarmellose sodium; and
about 20 mg to about 25 mg magnesium stearate.

In some embodiments, the method comprises administering to the patient a maintenance dose comprising one tablet, once weekly, wherein the tablet comprises:
about 400 mg of the spray dried dispersion of the compound of Formula Ia or Ib, or a pharmaceutically acceptable salt, co-crystal, solvate, or combination thereof;
about 2.130 mg of the compound of Formula IIa or IIb, or a pharmaceutically acceptable salt, co-crystal, solvate, or combination thereof;
about 621 mg of mannitol;
about 334.32 mg of microcrystalline cellulose;
about 120 mg croscarmellose sodium; and
about 22.5 mg magnesium stearate.

In some embodiments, the method comprises administering to the patient a maintenance dose comprising one tablet, once weekly, wherein the tablet comprises:
about 400 mg of the spray dried dispersion of the compound of Formula Ia or Ib, or a pharmaceutically acceptable salt, co-crystal, solvate, or combination thereof;
about 2.0 mg of the compound of Formula IIa or IIb, or a pharmaceutically acceptable salt, co-crystal, solvate, or combination thereof, on a free base basis;
about 621 mg of mannitol;
about 334.32 mg of microcrystalline cellulose;
about 120 mg croscarmellose sodium; and
about 22.5 mg magnesium stearate.

In some embodiments, the method comprises administering to the patient a maintenance dose comprising one tablet, once weekly, wherein the tablet comprises:
about 26.6 w/w % to about 26.7 w/w % of the spray dried dispersion of the compound of Formula Ia or Ib, or a pharmaceutically acceptable salt thereof;
about 0.14 w/w % to about 0.15 w/w % of the compound of Formula IIa or IIb, or a pharmaceutically acceptable salt, co-crystal, solvate, or combination thereof; and
one or more pharmaceutically acceptable excipients.

In some embodiments, the method comprises administering to the patient a maintenance dose comprising one tablet, once weekly, wherein the tablet comprises:
about 26.67 w/w % of the spray dried dispersion of the compound of Formula Ia or Ib, or a pharmaceutically acceptable salt thereof;
about 0.142 w/w % of the compound of Formula IIa or IIb, or a pharmaceutically acceptable salt, co-crystal, solvate, or combination thereof; and
one or more pharmaceutically acceptable excipients.

In some embodiments, the method comprises administering to the patient a maintenance dose comprising one tablet, once weekly, wherein the tablet comprises:
about 26.6 w/w % to about 26.7 w/w % of the spray dried dispersion of the compound of Formula Ia or Ib, or a pharmaceutically acceptable salt thereof;
about 0.14 w/w % to about 0.15 w/w % of the compound of Formula IIa or IIb, or a solvate thereof, and
one or more pharmaceutically acceptable excipients.

In some embodiments, the method comprises administering to the patient a maintenance dose comprising one tablet, once weekly, wherein the tablet comprises:
about 26.67 w/w % of the spray dried dispersion of the compound of Formula Ia or Ib, or a pharmaceutically acceptable salt thereof;
about 0.142 w/w % of the compound of Formula IIa or IIb, or a solvate thereof; and
one or more pharmaceutically acceptable excipients.

In some embodiments, the method comprises administering to the patient a maintenance dose comprising one tablet, once weekly, wherein the tablet comprises:
about 26.6 w/w % to about 26.7 w/w % of the spray dried dispersion of the compound of Formula Ia or Ib, or a pharmaceutically acceptable salt thereof;
about 0.14 w/w % to about 0.15 w/w % of the monohydrate of the compound of Formula IIa or IIb; and
one or more pharmaceutically acceptable excipients.

In some embodiments, the method comprises administering to the patient a maintenance dose comprising one tablet, once weekly, wherein the tablet comprises:
about 26.67 w/w % of the spray dried dispersion of the compound of Formula Ia or Ib, or a pharmaceutically acceptable salt thereof;
about 0.142 w/w % of the monohydrate of the compound of Formula IIa or IIb; and
one or more pharmaceutically acceptable excipients.

In some embodiments, the method comprises administering to the patient a maintenance dose comprising one tablet, once weekly, wherein the tablet comprises:
about 26.6 w/w % to about 26.7 w/w % of the spray dried dispersion of the compound of Formula Ia or Ib, or a pharmaceutically acceptable salt thereof;
about 0.14 w/w % to about 0.15 w/w % of the compound of Formula IIa or IIb, or a pharmaceutically acceptable salt, co-crystal, solvate, or combination thereof;
about 41 w/w % to about 42 w/w % of mannitol;
about 22.2 w/w % to about 22.3 w/w % of microcrystalline cellulose;
about 7.9 w/w % to about 8.1 w/w % of croscarmellose sodium; and
about 1.4 w/w % to about 1.6 w/w % of magnesium stearate.

In some embodiments, the method comprises administering to the patient a maintenance dose comprising one tablet, once weekly, wherein the tablet comprises:
about 26.67 w/w % of the spray dried dispersion of the compound of Formula Ia or Ib, or a pharmaceutically acceptable salt thereof;
about 0.142 w/w % of the compound of Formula IIa or IIb, or a pharmaceutically acceptable salt, co-crystal, solvate, or combination thereof;
about 41.40 w/w % of mannitol;
about 22.288 w/w % microcrystalline cellulose;
about 8.0 w/w % of croscarmellose sodium; and
about 1.5 w/w % of magnesium stearate.

In some embodiments, the method comprises administering to the patient a maintenance dose comprising one tablet, once weekly, wherein the tablet comprises:
about 400 mg of the spray dried dispersion of the compound of Formula Ia or Ib, or a pharmaceutically acceptable salt thereof, and
about 2.1 mg to about 2.2 mg of the compound of Formula IIa or IIb, or a pharmaceutically acceptable salt, co-crystal, solvate, or combination thereof.

In some embodiments, the method comprises administering to the patient a maintenance dose comprising one tablet, once weekly, wherein the tablet comprises:
about 400 mg of the spray dried dispersion of the compound of Formula Ia or Ib, or a pharmaceutically acceptable salt thereof, and
about 2.130 mg of the compound of Formula IIa or IIb, or a pharmaceutically acceptable salt, co-crystal, solvate, or combination thereof.

In some embodiments, the method comprises administering to the patient a maintenance dose comprising one tablet, once weekly, wherein the tablet comprises:
about 400 mg of the spray dried dispersion of the compound of Formula Ia or Ib, or a pharmaceutically acceptable salt thereof, and
about 2.0 mg of the compound of Formula IIa or IIb, or a pharmaceutically acceptable salt, co-crystal, solvate, or combination thereof, on a free base basis.

In some embodiments, the method comprises administering to the patient a maintenance dose comprising one tablet, once weekly, wherein the tablet comprises:
about 400 mg of the spray dried dispersion of the compound of Formula Ia or Ib, or a pharmaceutically acceptable salt thereof;
about 2.1 mg to about 2.2 mg of the compound of Formula IIa or IIb, or a pharmaceutically acceptable salt, co-crystal, solvate, or combination thereof;
about 615 mg to about 625 mg of mannitol;
about 330 mg to about 340 mg of microcrystalline cellulose;
about 115 mg to about 125 mg croscarmellose sodium; and
about 20 mg to about 25 mg magnesium stearate.

In some embodiments, the method comprises administering to the patient a maintenance dose comprising one tablet, once weekly, wherein the tablet comprises:
about 400 mg of the spray dried dispersion of the compound of Formula Ia or Ib, or a pharmaceutically acceptable salt thereof;
about 2.130 mg of the compound of Formula IIa or IIb, or a pharmaceutically acceptable salt, co-crystal, solvate, or combination thereof;
about 621 mg of mannitol;
about 334.32 mg of microcrystalline cellulose;
about 120 mg croscarmellose sodium; and
about 22.5 mg magnesium stearate.

In some embodiments, the method comprises administering to the patient a maintenance dose comprising one tablet, once weekly, wherein the tablet comprises:
about 400 mg of the spray dried dispersion of the compound of Formula Ia or Ib, or a pharmaceutically acceptable salt thereof;
about 2.0 mg of the compound of Formula IIa or IIb, or a pharmaceutically acceptable salt, co-crystal, solvate, or combination thereof, on a free base basis;
about 621 mg of mannitol;
about 334.32 mg of microcrystalline cellulose;
about 120 mg croscarmellose sodium; and
about 22.5 mg magnesium stearate.

In some embodiments, the method comprises administering to the patient a maintenance dose comprising one tablet, once weekly, wherein the tablet comprises:
about 26.6 w/w % to about 26.7 w/w % of the spray dried dispersion of the sodium salt of the compound of Formula Ia or Ib;
about 0.14 w/w % to about 0.15 w/w % of the compound of Formula IIa or IIb, or a pharmaceutically acceptable salt, co-crystal, solvate, or combination thereof; and
one or more pharmaceutically acceptable excipients.

In some embodiments, the method comprises administering to the patient a maintenance dose comprising one tablet, once weekly, wherein the tablet comprises:
about 26.67 w/w % of the spray dried dispersion of the sodium salt of the compound of Formula Ia or Ib;
about 0.142 w/w % of the compound of Formula IIa or IIb, or a pharmaceutically acceptable salt, co-crystal, solvate, or combination thereof; and
one or more pharmaceutically acceptable excipients.

In some embodiments, the method comprises administering to the patient a maintenance dose comprising one tablet, once weekly, wherein the tablet comprises:
about 26.6 w/w % to about 26.7 w/w % of the spray dried dispersion of the sodium salt of the compound of Formula Ia or Ib;
about 0.14 w/w % to about 0.15 w/w % of the compound of Formula IIa or IIb, or a solvate thereof, and
one or more pharmaceutically acceptable excipients.

In some embodiments, the method comprises administering to the patient a maintenance dose comprising one tablet, once weekly, wherein the tablet comprises:
about 26.67 w/w % of the spray dried dispersion of the sodium salt of the compound of Formula Ia or Ib;
about 0.142 w/w % of the compound of Formula IIa or IIb, or a solvate thereof; and
one or more pharmaceutically acceptable excipients.

In some embodiments, the method comprises administering to the patient a maintenance dose comprising one tablet, once weekly, wherein the tablet comprises:
about 26.6 w/w % to about 26.7 w/w % of the spray dried dispersion of the sodium salt of the compound of Formula Ia or Ib;
about 0.14 w/w % to about 0.15 w/w % of the monohydrate of the compound of Formula IIa or IIb; and
one or more pharmaceutically acceptable excipients.

In some embodiments, the method comprises administering to the patient a maintenance dose comprising one tablet, once weekly, wherein the tablet comprises:
about 26.67 w/w % of the spray dried dispersion of the sodium salt of the compound of Formula Ia or Ib;
about 0.142 w/w % of the monohydrate of the compound of Formula IIa or IIb; and
one or more pharmaceutically acceptable excipients.

In some embodiments, the method comprises administering to the patient a maintenance dose comprising one tablet, once weekly, wherein the tablet comprises:
about 26.6 w/w % to about 26.7 w/w % of the spray dried dispersion of the sodium salt of the compound of Formula Ia or Ib;
about 0.14 w/w % to about 0.15 w/w % of the compound of Formula IIa or IIb, or a pharmaceutically acceptable salt, co-crystal, solvate, or combination thereof;
about 41 w/w % to about 42 w/w % of mannitol;
about 22.2 w/w % to about 22.3 w/w % of microcrystalline cellulose;
about 7.9 w/w % to about 8.1 w/w % of croscarmellose sodium; and
about 1.4 w/w % to about 1.6 w/w % of magnesium stearate.

In some embodiments, the method comprises administering to the patient a maintenance dose comprising one tablet, once weekly, wherein the tablet comprises:
about 26.67 w/w % of the spray dried dispersion of the sodium salt of the compound of Formula Ia or Ib;
about 0.142 w/w % of the compound of Formula IIa or IIb, or a pharmaceutically acceptable salt, co-crystal, solvate, or combination thereof;
about 41.40 w/w % of mannitol;
about 22.288 w/w % microcrystalline cellulose;
about 8.0 w/w % of croscarmellose sodium; and
about 1.5 w/w % of magnesium stearate.

In some embodiments, the method comprises administering to the patient a maintenance dose comprising one tablet, once weekly, wherein the tablet comprises:
about 400 mg of the spray dried dispersion of the sodium salt of the compound of Formula Ia or Ib; and
about 2.1 mg to about 2.2 mg of the compound of Formula IIa or IIb, or a pharmaceutically acceptable salt, co-crystal, solvate, or combination thereof.

In some embodiments, the method comprises administering to the patient a maintenance dose comprising one tablet, once weekly, wherein the tablet comprises:
about 400 mg of the spray dried dispersion of the sodium salt of the compound of Formula Ia or Ib; and
about 2.130 mg of the compound of Formula IIa or IIb, or a pharmaceutically acceptable salt, co-crystal, solvate, or combination thereof.

In some embodiments, the method comprises administering to the patient a maintenance dose comprising one tablet, once weekly, wherein the tablet comprises:
about 400 mg of the spray dried dispersion of the sodium salt of the compound of Formula Ia or Ib; and
about 2.0 mg of the compound of Formula IIa or IIb, or a pharmaceutically acceptable salt, co-crystal, solvate, or combination thereof, on a free base basis.

In some embodiments, the method comprises administering to the patient a maintenance dose comprising one tablet, once weekly, wherein the tablet comprises:
about 400 mg of the spray dried dispersion of the sodium salt of the compound of Formula Ia or Ib;
about 2.1 mg to about 2.2 mg of the compound of Formula IIa or IIb, or a pharmaceutically acceptable salt, co-crystal, solvate, or combination thereof;
about 615 mg to about 625 mg of mannitol;
about 330 mg to about 340 mg of microcrystalline cellulose;
about 115 mg to about 125 mg croscarmellose sodium; and
about 20 mg to about 25 mg magnesium stearate.

In some embodiments, the method comprises administering to the patient a maintenance dose comprising one tablet, once weekly, wherein the tablet comprises:
about 400 mg of the spray dried dispersion of the sodium salt of the compound of Formula Ia or Ib;
about 2.130 mg of the compound of Formula IIa or IIb, or a pharmaceutically acceptable salt, co-crystal, solvate, or combination thereof;
about 621 mg of mannitol;
about 334.32 mg of microcrystalline cellulose;
about 120 mg croscarmellose sodium; and
about 22.5 mg magnesium stearate.

In some embodiments, the method comprises administering to the patient a maintenance dose comprising one tablet, once weekly, wherein the tablet comprises:
about 400 mg of the spray dried dispersion of the sodium salt of the compound of Formula Ia or Ib;
about 2.0 mg of the compound of Formula IIa or IIb, or a pharmaceutically acceptable salt, co-crystal, solvate, or combination thereof, on a free base basis;
about 621 mg of mannitol;
about 334.32 mg of microcrystalline cellulose;
about 120 mg croscarmellose sodium; and
about 22.5 mg magnesium stearate.

In some embodiments, the method comprises administering to the patient a maintenance dose comprising one tablet, once weekly, wherein the tablet comprises:
about 26.6 w/w % to about 26.7 w/w % of the spray dried dispersion of the sodium salt of the compound of Formula Ia;
about 0.14 w/w % to about 0.15 w/w % of the compound of Formula IIa or IIb, or a pharmaceutically acceptable salt, co-crystal, solvate, or combination thereof; and
one or more pharmaceutically acceptable excipients.

In some embodiments, the method comprises administering to the patient a maintenance dose comprising one tablet, once weekly, wherein the tablet comprises:
about 26.67 w/w % of the spray dried dispersion of the sodium salt of the compound of Formula Ia;
about 0.142 w/w % of the compound of Formula IIa or IIb, or a pharmaceutically acceptable salt, co-crystal, solvate, or combination thereof; and
one or more pharmaceutically acceptable excipients.

In some embodiments, the method comprises administering to the patient a maintenance dose comprising one tablet, once weekly, wherein the tablet comprises:
about 26.6 w/w % to about 26.7 w/w % of the spray dried dispersion of the sodium salt of the compound of Formula Ia;
about 0.14 w/w % to about 0.15 w/w % of the compound of Formula IIa or IIb, or a solvate thereof, and
one or more pharmaceutically acceptable excipients.

In some embodiments, the method comprises administering to the patient a maintenance dose comprising one tablet, once weekly, wherein the tablet comprises:
about 26.67 w/w % of the spray dried dispersion of the sodium salt of the compound of Formula Ia;
about 0.142 w/w % of the compound of Formula IIa or IIb, or a solvate thereof; and
one or more pharmaceutically acceptable excipients.

In some embodiments, the method comprises administering to the patient a maintenance dose comprising one tablet, once weekly, wherein the tablet comprises:
about 26.6 w/w % to about 26.7 w/w % of the spray dried dispersion of the sodium salt of the compound of Formula Ia;
about 0.14 w/w % to about 0.15 w/w % of the monohydrate of the compound of Formula IIa or IIb; and
one or more pharmaceutically acceptable excipients.

In some embodiments, the method comprises administering to the patient a maintenance dose comprising one tablet, once weekly, wherein the tablet comprises:
about 26.67 w/w % of the spray dried dispersion of the sodium salt of the compound of Formula Ia;
about 0.142 w/w % of the monohydrate of the compound of Formula IIa or IIb; and
one or more pharmaceutically acceptable excipients.

In some embodiments, the method comprises administering to the patient a maintenance dose comprising one tablet, once weekly, wherein the tablet comprises:
about 26.6 w/w % to about 26.7 w/w % of the spray dried dispersion of the sodium salt of the compound of Formula Ia;
about 0.14 w/w % to about 0.15 w/w % of the compound of Formula IIa or IIb, or a pharmaceutically acceptable salt, co-crystal, solvate, or combination thereof;
about 41 w/w % to about 42 w/w % of mannitol;
about 22.2 w/w % to about 22.3 w/w % of microcrystalline cellulose;
about 7.9 w/w % to about 8.1 w/w % of croscarmellose sodium; and
about 1.4 w/w % to about 1.6 w/w % of magnesium stearate.

In some embodiments, the method comprises administering to the patient a maintenance dose comprising one tablet, once weekly, wherein the tablet comprises:
about 26.67 w/w % of the spray dried dispersion of the sodium salt of the compound of Formula Ia;
about 0.142 w/w % of the compound of Formula IIa or IIb, or a pharmaceutically acceptable salt, co-crystal, solvate, or combination thereof;
about 41.40 w/w % of mannitol;
about 22.288 w/w % microcrystalline cellulose;
about 8.0 w/w % of croscarmellose sodium; and
about 1.5 w/w % of magnesium stearate.

In some embodiments, the method comprises administering to the patient a maintenance dose comprising one tablet, once weekly, wherein the tablet comprises:
about 400 mg of the spray dried dispersion of the sodium salt of the compound of Formula Ia; and
about 2.1 mg to about 2.2 mg of the compound of Formula IIa or IIb, or a pharmaceutically acceptable salt, co-crystal, solvate, or combination thereof.

In some embodiments, the method comprises administering to the patient a maintenance dose comprising one tablet, once weekly, wherein the tablet comprises:
about 400 mg of the spray dried dispersion of the sodium salt of the compound of Formula Ia; and
about 2.130 mg of the compound of Formula IIa or IIb, or a pharmaceutically acceptable salt, co-crystal, solvate, or combination thereof.

In some embodiments, the method comprises administering to the patient a maintenance dose comprising one tablet, once weekly, wherein the tablet comprises:
about 400 mg of the spray dried dispersion of the sodium salt of the compound of Formula Ia; and
about 2.0 mg of the compound of Formula IIa or IIb, or a pharmaceutically acceptable salt, co-crystal, solvate, or combination thereof, on a free base basis.

In some embodiments, the method comprises administering to the patient a maintenance dose comprising one tablet, once weekly, wherein the tablet comprises:
about 400 mg of the spray dried dispersion of the sodium salt of the compound of Formula Ia;
about 2.1 mg to about 2.2 mg of the compound of Formula IIa or IIb, or a pharmaceutically acceptable salt, co-crystal, solvate, or combination thereof;
about 615 mg to about 625 mg of mannitol;
about 330 mg to about 340 mg of microcrystalline cellulose;
about 115 mg to about 125 mg croscarmellose sodium; and
about 20 mg to about 25 mg magnesium stearate.

In some embodiments, the method comprises administering to the patient a maintenance dose comprising one tablet, once weekly, wherein the tablet comprises:
about 400 mg of the spray dried dispersion of the sodium salt of the compound of Formula Ia;
about 2.130 mg of the compound of Formula IIa or IIb, or a pharmaceutically acceptable salt, co-crystal, solvate, or combination thereof;
about 621 mg of mannitol;
about 334.32 mg of microcrystalline cellulose;
about 120 mg croscarmellose sodium; and
about 22.5 mg magnesium stearate.

In some embodiments, the method comprises administering to the patient a maintenance dose comprising one tablet, once weekly, wherein the tablet comprises:
about 400 mg of the spray dried dispersion of the sodium salt of the compound of Formula Ia;
about 2.0 mg of the compound of Formula IIa or IIb, or a pharmaceutically acceptable salt, co-crystal, solvate, or combination thereof, on a free base basis;
about 621 mg of mannitol;
about 334.32 mg of microcrystalline cellulose;
about 120 mg croscarmellose sodium; and
about 22.5 mg magnesium stearate.

In some embodiments, the method comprises administering to the patient a maintenance dose comprising one tablet, once weekly, wherein the tablet comprises:
about 26.6 w/w % to about 26.7 w/w % of the lenacapavir spray dried dispersion;
about 0.14 w/w % to about 0.15 w/w % of the compound of Formula IIa or IIb, or a pharmaceutically acceptable salt, co-crystal, solvate, or combination thereof; and
one or more pharmaceutically acceptable excipients.

In some embodiments, the method comprises administering to the patient a maintenance dose comprising one tablet, once weekly, wherein the tablet comprises:
about 26.67 w/w % of the lenacapavir spray dried dispersion;
about 0.142 w/w % of the compound of Formula IIa or IIb, or a pharmaceutically acceptable salt, co-crystal, solvate, or combination thereof; and
one or more pharmaceutically acceptable excipients.

In some embodiments, the method comprises administering to the patient a maintenance dose comprising one tablet, once weekly, wherein the tablet comprises:
about 26.6 w/w % to about 26.7 w/w % of the lenacapavir spray dried dispersion;
about 0.14 w/w % to about 0.15 w/w % of the compound of Formula IIa or IIb, or a solvate thereof, and
one or more pharmaceutically acceptable excipients.

In some embodiments, the method comprises administering to the patient a maintenance dose comprising one tablet, once weekly, wherein the tablet comprises:
about 26.67 w/w % of the lenacapavir spray dried dispersion;
about 0.142 w/w % of the compound of Formula IIa or IIb, or a solvate thereof; and
one or more pharmaceutically acceptable excipients.

In some embodiments, the method comprises administering to the patient a maintenance dose comprising one tablet, once weekly, wherein the tablet comprises:
about 26.6 w/w % to about 26.7 w/w % of the lenacapavir spray dried dispersion;
about 0.14 w/w % to about 0.15 w/w % of the monohydrate of the compound of Formula IIa or IIb; and
one or more pharmaceutically acceptable excipients.

In some embodiments, the method comprises administering to the patient a maintenance dose comprising one tablet, once weekly, wherein the tablet comprises:
about 26.67 w/w % of the lenacapavir spray dried dispersion;
about 0.142 w/w % of the monohydrate of the compound of Formula IIa or IIb; and
one or more pharmaceutically acceptable excipients.

In some embodiments, the method comprises administering to the patient a maintenance dose comprising one tablet, once weekly, wherein the tablet comprises:
about 26.6 w/w % to about 26.7 w/w % of the lenacapavir spray dried dispersion;
about 0.14 w/w % to about 0.15 w/w % of the compound of Formula IIa or IIb, or a pharmaceutically acceptable salt, co-crystal, solvate, or combination thereof;
about 41 w/w % to about 42 w/w % of mannitol;
about 22.2 w/w % to about 22.3 w/w % of microcrystalline cellulose;
about 7.9 w/w % to about 8.1 w/w % of croscarmellose sodium; and
about 1.4 w/w % to about 1.6 w/w % of magnesium stearate.

In some embodiments, the method comprises administering to the patient a maintenance dose comprising one tablet, once weekly, wherein the tablet comprises:
about 26.67 w/w % of the lenacapavir spray dried dispersion;
about 0.142 w/w % of the compound of Formula IIa or IIb, or a pharmaceutically acceptable salt, co-crystal, solvate, or combination thereof;
about 41.40 w/w % of mannitol;
about 22.288 w/w % microcrystalline cellulose;
about 8.0 w/w % of croscarmellose sodium; and
about 1.5 w/w % of magnesium stearate.

In some embodiments, the method comprises administering to the patient a maintenance dose comprising one tablet, once weekly, wherein the tablet comprises:
about 400 mg of the lenacapavir spray dried dispersion; and
about 2.1 mg to about 2.2 mg of the compound of Formula IIa or IIb, or a pharmaceutically acceptable salt, co-crystal, solvate, or combination thereof.

In some embodiments, the method comprises administering to the patient a maintenance dose comprising one tablet, once weekly, wherein the tablet comprises:
about 400 mg of the lenacapavir spray dried dispersion; and
about 2.130 mg of the compound of Formula IIa or IIb, or a pharmaceutically acceptable salt, co-crystal, solvate, or combination thereof.

In some embodiments, the method comprises administering to the patient a maintenance dose comprising one tablet, once weekly, wherein the tablet comprises:
about 400 mg of the lenacapavir spray dried dispersion; and
about 2.0 mg of the compound of Formula IIa or IIb, or a pharmaceutically acceptable salt, co-crystal, solvate, or combination thereof, on a free base basis.

In some embodiments, the method comprises administering to the patient a maintenance dose comprising one tablet, once weekly, wherein the tablet comprises:
about 400 mg of the lenacapavir spray dried dispersion;
about 2.1 mg to about 2.2 mg of the compound of Formula IIa or IIb, or a pharmaceutically acceptable salt, co-crystal, solvate, or combination thereof;
about 615 mg to about 625 mg of mannitol;
about 330 mg to about 340 mg of microcrystalline cellulose;
about 115 mg to about 125 mg croscarmellose sodium; and
about 20 mg to about 25 mg magnesium stearate.

In some embodiments, the method comprises administering to the patient a maintenance dose comprising one tablet, once weekly, wherein the tablet comprises:
about 400 mg of the lenacapavir spray dried dispersion;
about 2.130 mg of the compound of Formula IIa or IIb, or a pharmaceutically acceptable salt, co-crystal, solvate, or combination thereof;
about 621 mg of mannitol;
about 334.32 mg of microcrystalline cellulose;
about 120 mg croscarmellose sodium; and
about 22.5 mg magnesium stearate.

In some embodiments, the method comprises administering to the patient a maintenance dose comprising one tablet, once weekly, wherein the tablet comprises:
about 400 mg of the lenacapavir spray dried dispersion;
about 2.0 mg of the compound of Formula IIa or IIb, or a pharmaceutically acceptable salt, co-crystal, solvate, or combination thereof, on a free base basis;
about 621 mg of mannitol;
about 334.32 mg of microcrystalline cellulose;
about 120 mg croscarmellose sodium; and
about 22.5 mg magnesium stearate.

In some embodiments, the method comprises administering to the patient a maintenance dose comprising one tablet, once weekly, wherein the tablet comprises:
about 300 mg of the compound of Formula Ia or Ib, or a pharmaceutically acceptable salt, co-crystal, solvate, or combination thereof, on a free acid basis; and
about 2.1 mg to about 2.2 mg of the compound of Formula IIa or IIb, or a pharmaceutically acceptable salt, co-crystal, solvate, or combination thereof.

In some embodiments, the method comprises administering to the patient a maintenance dose comprising one tablet, once weekly, wherein the tablet comprises:
about 300 mg of the compound of Formula Ia or Ib, or a pharmaceutically acceptable salt, co-crystal, solvate, or combination thereof, on a free acid basis; and
about 2.130 mg of the compound of Formula IIa or IIb, or a pharmaceutically acceptable salt, co-crystal, solvate, or combination thereof.

In some embodiments, the method comprises administering to the patient a maintenance dose comprising one tablet, once weekly, wherein the tablet comprises:
about 300 mg of the compound of Formula Ia or Ib, or a pharmaceutically acceptable salt, co-crystal, solvate, or combination thereof, on a free acid basis; and
about 2.0 mg of the compound of Formula IIa or IIb, or a pharmaceutically acceptable salt, co-crystal, solvate, or combination thereof, on a free base basis.

In some embodiments, the method comprises administering to the patient a maintenance dose comprising one tablet, once weekly, wherein the tablet comprises:
about 300 mg of the compound of Formula Ia or Ib, or a pharmaceutically acceptable salt, co-crystal, solvate, or combination thereof, on a free acid basis;
about 2.1 mg to about 2.2 mg of the compound of Formula IIa or IIb, or a pharmaceutically acceptable salt, co-crystal, solvate, or combination thereof;
about 615 mg to about 625 mg of mannitol;
about 330 mg to about 340 mg of microcrystalline cellulose;
about 115 mg to about 125 mg croscarmellose sodium; and
about 20 mg to about 25 mg magnesium stearate.

In some embodiments, the method comprises administering to the patient a maintenance dose comprising one tablet, once weekly, wherein the tablet comprises:
about 300 mg of the compound of Formula Ia or Ib, or a pharmaceutically acceptable salt, co-crystal, solvate, or combination thereof, on a free acid basis;
about 2.130 mg of the compound of Formula IIa or IIb, or a pharmaceutically acceptable salt, co-crystal, solvate, or combination thereof;
about 621 mg of mannitol;
about 334.32 mg of microcrystalline cellulose;
about 120 mg croscarmellose sodium; and
about 22.5 mg magnesium stearate.

In some embodiments, the method comprises administering to the patient a maintenance dose comprising one tablet, once weekly, wherein the tablet comprises:
about 300 mg of the compound of Formula Ia or Ib, or a pharmaceutically acceptable salt, co-crystal, solvate, or combination thereof, on a free acid basis;
about 2.0 mg of the compound of Formula IIa or IIb, or a pharmaceutically acceptable salt, co-crystal, solvate, or combination thereof, on a free base basis;
about 621 mg of mannitol;
about 334.32 mg of microcrystalline cellulose;
about 120 mg croscarmellose sodium; and
about 22.5 mg magnesium stearate.

In some embodiments, the method comprises:
i) administering to the patient an initiation dose comprising two tablets on day 1, and two tablets on day 2, wherein each tablet of the initiation dose comprises:
   about 26.6 w/w % to about 26.7 w/w % of the spray dried dispersion of the compound of Formula Ia or Ib, or a pharmaceutically acceptable salt, co-crystal, solvate, or combination thereof;
   about 0.03 w/w % to about 0.04 w/w % of the compound of Formula IIa or IIb, or a pharmaceutically acceptable salt, co-crystal, solvate, or combination thereof; and
   one or more pharmaceutically acceptable excipients; and
ii) administering to the patient a maintenance dose comprising one tablet, once weekly, beginning on day 8, wherein the tablet comprises:
   about 26.6 w/w % to about 26.7 w/w % of the spray dried dispersion of the compound of Formula Ia or Ib, or a pharmaceutically acceptable salt, co-crystal, solvate, or combination thereof;
   about 0.14 w/w % to about 0.15 w/w % of the compound of Formula IIa or IIb, or a pharmaceutically acceptable salt, co-crystal, solvate, or combination thereof, and
   one or more pharmaceutically acceptable excipients.

In some embodiments, the method comprises:
i) administering to the patient an initiation dose comprising two tablets on day 1, and two tablets on day 2, wherein each tablet of the initiation dose comprises:
   about 26.67 w/w % of the spray dried dispersion of the compound of Formula Ia or Ib, or a pharmaceutically acceptable salt, co-crystal, solvate, or combination thereof;
   about 0.0355 w/w % of the compound of Formula IIa or IIb, or a pharmaceutically acceptable salt, co-crystal, solvate, or combination thereof; and
   one or more pharmaceutically acceptable excipients; and
ii) administering to the patient a maintenance dose comprising one tablet, once weekly, beginning on day 8, wherein the tablet comprises:
   about 26.67 w/w % of the spray dried dispersion of the compound of Formula Ia or Ib, or a pharmaceutically acceptable salt, co-crystal, solvate, or combination thereof;
   about 0.142 w/w % of the compound of Formula IIa or IIb, or a pharmaceutically acceptable salt, co-crystal, solvate, or combination thereof; and
   one or more pharmaceutically acceptable excipients.

In some embodiments, the method comprises:
i) administering to the patient an initiation dose comprising two tablets on day 1, and two tablets on day 2, wherein each tablet of the initiation dose comprises:
   about 26.6 w/w % to about 26.7 w/w % of the spray dried dispersion of the compound of Formula Ia or Ib, or a pharmaceutically acceptable salt, co-crystal, solvate, or combination thereof;
   about 0.03 w/w % to about 0.04 w/w % of the compound of Formula IIa or IIb, or a solvate thereof; and
   one or more pharmaceutically acceptable excipients; and
ii) administering to the patient a maintenance dose comprising one tablet, once weekly, beginning on day 8, wherein the tablet comprises:
   about 26.6 w/w % to about 26.7 w/w % of the spray dried dispersion of the compound of Formula Ia or Ib, or a pharmaceutically acceptable salt, co-crystal, solvate, or combination thereof;
   about 0.14 w/w % to about 0.15 w/w % of the compound of Formula IIa or IIb, or a solvate thereof; and
   one or more pharmaceutically acceptable excipients.

In some embodiments, the method comprises:
i) administering to the patient an initiation dose comprising two tablets on day 1, and two tablets on day 2, wherein each tablet of the initiation dose comprises:
   about 26.67 w/w % of the spray dried dispersion of the compound of Formula Ia or Ib, or a pharmaceutically acceptable salt, co-crystal, solvate, or combination thereof;
   about 0.0355 w/w % of the compound of Formula IIa or IIb, or a solvate thereof, and
   one or more pharmaceutically acceptable excipients; and
ii) administering to the patient a maintenance dose comprising one tablet, once weekly, beginning on day 8, wherein the tablet comprises:
   about 26.67 w/w % of the spray dried dispersion of the compound of Formula Ia or Ib, or a pharmaceutically acceptable salt, co-crystal, solvate, or combination thereof;
   about 0.142 w/w % of the compound of Formula IIa or IIb, or a solvate thereof, and
   one or more pharmaceutically acceptable excipients.

In some embodiments, the method comprises:
i) administering to the patient an initiation dose comprising two tablets on day 1, and two tablets on day 2, wherein each tablet of the initiation dose comprises:
   about 26.6 w/w % to about 26.7 w/w % of the spray dried dispersion of the compound of Formula Ia or Ib, or a pharmaceutically acceptable salt, co-crystal, solvate, or combination thereof;
   about 0.03 w/w % to about 0.04 w/w % of the monohydrate of the compound of Formula IIa or IIb; and
   one or more pharmaceutically acceptable excipients; and
ii) administering to the patient a maintenance dose comprising one tablet, once weekly, beginning on day 8, wherein the tablet comprises:
   about 26.6 w/w % to about 26.7 w/w % of the spray dried dispersion of the compound of Formula Ia or Ib, or a pharmaceutically acceptable salt, co-crystal, solvate, or combination thereof;
   about 0.14 w/w % to about 0.15 w/w % of the monohydrate of the compound of Formula IIa or IIb; and
   one or more pharmaceutically acceptable excipients.

In some embodiments, the method comprises:
i) administering to the patient an initiation dose comprising two tablets on day 1, and two tablets on day 2, wherein each tablet of the initiation dose comprises:
   about 26.67 w/w % of the spray dried dispersion of the compound of Formula Ia or Ib, or a pharmaceutically acceptable salt, co-crystal, solvate, or combination thereof;
   about 0.0355 w/w % of the monohydrate of the compound of Formula IIa or IIb; and
   one or more pharmaceutically acceptable excipients; and
ii) administering to the patient a maintenance dose comprising one tablet, once weekly, beginning on day 8, wherein the tablet comprises:
   about 26.67 w/w % of the spray dried dispersion of the compound of Formula Ia or Ib, or a pharmaceutically acceptable salt, co-crystal, solvate, or combination thereof;
   about 0.142 w/w % of the monohydrate of the compound of Formula IIa or IIb; and
   one or more pharmaceutically acceptable excipients.

In some embodiments, the method comprises:
i) administering to the patient an initiation dose comprising two tablets on day 1, and two tablets on day 2, wherein each tablet of the initiation dose comprises:
   about 26.6 w/w % to about 26.7 w/w % of the spray dried dispersion of the compound of Formula Ia or Ib, or a pharmaceutically acceptable salt, co-crystal, solvate, or combination thereof;
   about 0.03 w/w % to about 0.04 w/w % of the compound of Formula IIa or IIb, or a pharmaceutically acceptable salt, co-crystal, solvate, or combination thereof;
   about 41 w/w % to about 42 w/w % of mannitol;
   about 22.3 w/w % to about 22.4 w/w % of microcrystalline cellulose;
   about 7.9 w/w % to about 8.1 w/w % of croscarmellose sodium; and
   about 1.4 w/w % to about 1.6 w/w % of magnesium stearate; and
ii) administering to the patient a maintenance dose comprising one tablet, once weekly, beginning on day 8, wherein the tablet comprises:
   about 26.6 w/w % to about 26.7 w/w % of the spray dried dispersion of the compound of Formula Ia or Ib, or a pharmaceutically acceptable salt, co-crystal, solvate, or combination thereof;
   about 0.14 w/w % to about 0.15 w/w % of the compound of Formula IIa or IIb, or a pharmaceutically acceptable salt, co-crystal, solvate, or combination thereof;
   about 41 w/w % to about 42 w/w % of mannitol;
   about 22.2 w/w % to about 22.3 w/w % of microcrystalline cellulose;
   about 7.9 w/w % to about 8.1 w/w % of croscarmellose sodium; and
   about 1.4 w/w % to about 1.6 w/w % of magnesium stearate.

In some embodiments, the method comprises:
i) administering to the patient an initiation dose comprising two tablets on day 1, and two tablets on day 2, wherein each tablet of the initiation dose comprises:
   about 26.67 w/w % of the spray dried dispersion of the compound of Formula Ia or Ib, or a pharmaceutically acceptable salt, co-crystal, solvate, or combination thereof;
   about 0.0355 w/w % of the compound of Formula IIa or IIb, or a pharmaceutically acceptable salt, co-crystal, solvate, or combination thereof;
   about 41.40 w/w % of mannitol;
   about 22.3945 w/w % of microcrystalline cellulose;
   about 8.0 w/w % of croscarmellose sodium; and
   about 1.5 w/w % of magnesium stearate; and
ii) administering to the patient a maintenance dose comprising one tablet, once weekly, beginning on day 8, wherein the tablet comprises:
   about 26.67 w/w % of the spray dried dispersion of the compound of Formula Ia or Ib, or a pharmaceutically acceptable salt, co-crystal, solvate, or combination thereof;
   about 0.142 w/w % of the compound of Formula IIa or IIb, or a pharmaceutically acceptable salt, co-crystal, solvate, or combination thereof;
   about 41.40 w/w % of mannitol;
   about 22.288 w/w % microcrystalline cellulose;
   about 8.0 w/w % of croscarmellose sodium; and
   about 1.5 w/w % of magnesium stearate.

In some embodiments, the method comprises:
i) administering to the patient an initiation dose comprising two tablets on day 1, and two tablets on day 2, wherein each tablet of the initiation dose comprises:
   about 400 mg of the spray dried dispersion of the compound of Formula Ia or Ib, or a pharmaceutically acceptable salt, co-crystal, solvate, or combination thereof, and
   about 0.5 mg to about 0.6 mg of the compound of Formula IIa or IIb, or a pharmaceutically acceptable salt, co-crystal, solvate, or combination thereof, and
ii) administering to the patient a maintenance dose comprising one tablet, once weekly, beginning on day 8, wherein the tablet comprises:
   about 400 mg of the spray dried dispersion of the compound of Formula Ia or Ib, or a pharmaceutically acceptable salt, co-crystal, solvate, or combination thereof, and
   about 2.1 mg to about 2.2 mg of the compound of Formula IIa or IIb, or a pharmaceutically acceptable salt, co-crystal, solvate, or combination thereof.

In some embodiments, the method comprises:
i) administering to the patient an initiation dose comprising two tablets on day 1, and two tablets on day 2, wherein each tablet of the initiation dose comprises:
   about 400 mg of the spray dried dispersion of the compound of Formula Ia or Ib, or a pharmaceutically acceptable salt, co-crystal, solvate, or combination thereof, and
   about 0.5325 mg of the compound of Formula IIa or IIb, or a pharmaceutically acceptable salt, co-crystal, solvate, or combination thereof; and
ii) administering to the patient a maintenance dose comprising one tablet, once weekly, beginning on day 8, wherein the tablet comprises:
   about 400 mg of the spray dried dispersion of the compound of Formula Ia or Ib, or a pharmaceutically acceptable salt, co-crystal, solvate, or combination thereof, and
   about 2.130 mg of the compound of Formula IIa or IIb, or a pharmaceutically acceptable salt, co-crystal, solvate, or combination thereof.

In some embodiments, the method comprises:
i) administering to the patient an initiation dose comprising two tablets on day 1, and two tablets on day 2, wherein each tablet of the initiation dose comprises:
   about 400 mg of the spray dried dispersion of the compound of Formula Ia or Ib, or a pharmaceutically acceptable salt, co-crystal, solvate, or combination thereof, and
   about 0.5 mg of the compound of Formula IIa or IIb, or a pharmaceutically acceptable salt, co-crystal, solvate, or combination thereof,
on a free base basis; and
ii) administering to the patient a maintenance dose comprising one tablet, once weekly, beginning on day 8, wherein the tablet comprises:
   about 400 mg of the spray dried dispersion of the compound of Formula Ia or Ib, or a pharmaceutically acceptable salt, co-crystal, solvate, or combination thereof, and
   about 2.0 mg of the compound of Formula IIa or IIb, or a pharmaceutically acceptable salt, co-crystal, solvate, or combination thereof,
on a free base basis.

In some embodiments, the method comprises:
i) administering to the patient an initiation dose comprising two tablets on day 1, and two tablets on day 2, wherein each tablet of the initiation dose comprises:
   about 400 mg of the spray dried dispersion of the compound of Formula Ia or Ib, or a pharmaceutically acceptable salt, co-crystal, solvate, or combination thereof;
   about 0.5 mg to about 0.6 mg of the compound of Formula IIa or IIb, or a pharmaceutically acceptable salt, co-crystal, solvate, or combination thereof;
   about 615 mg to about 625 mg of mannitol;
   about 330 mg to about 340 mg of microcrystalline cellulose;
   about 115 mg to about 125 mg croscarmellose sodium; and
   about 20 mg to about 25 mg magnesium stearate; and
ii) administering to the patient a maintenance dose comprising one tablet, once weekly, beginning on day 8, wherein the tablet comprises:
   about 400 mg of the spray dried dispersion of the compound of Formula Ia or Ib, or a pharmaceutically acceptable salt, co-crystal, solvate, or combination thereof;
   about 2.1 mg to about 2.2 mg of the compound of Formula IIa or IIb, or a pharmaceutically acceptable salt, co-crystal, solvate, or combination thereof;
about 615 mg to about 625 mg of mannitol;
   about 330 mg to about 340 mg of microcrystalline cellulose;
   about 115 mg to about 125 mg croscarmellose sodium; and
   about 20 mg to about 25 mg magnesium stearate.

In some embodiments, the method comprises:
i) administering to the patient an initiation dose comprising two tablets on day 1, and two tablets on day 2, wherein each tablet of the initiation dose comprises:
   about 400 mg of the spray dried dispersion of the compound of Formula Ia or Ib, or a pharmaceutically acceptable salt, co-crystal, solvate, or combination thereof;
   about 0.5325 mg of the compound of Formula IIa or IIb, or a pharmaceutically acceptable salt, co-crystal, solvate, or combination thereof;
   about 621 mg of mannitol;
   about 335.9175 mg of microcrystalline cellulose;
   about 120 mg croscarmellose sodium; and
   about 22.5 mg magnesium stearate; and
ii) administering to the patient a maintenance dose comprising one tablet, once weekly, beginning on day 8, wherein the tablet comprises:
   about 400 mg of the spray dried dispersion of the compound of Formula Ia or Ib, or a pharmaceutically acceptable salt, co-crystal, solvate, or combination thereof;
   about 2.130 mg of the compound of Formula IIa or IIb, or a pharmaceutically acceptable salt, co-crystal, solvate, or combination thereof;
   about 621 mg of mannitol;
   about 334.32 mg of microcrystalline cellulose;
   about 120 mg croscarmellose sodium; and
   about 22.5 mg magnesium stearate.

In some embodiments, the method comprises:
i) administering to the patient an initiation dose comprising two tablets on day 1, and two tablets on day 2, wherein each tablet of the initiation dose comprises:
   about 400 mg of the spray dried dispersion of the compound of Formula Ia or Ib, or a pharmaceutically acceptable salt, co-crystal, solvate, or combination thereof;
   about 0.5 mg of the compound of Formula IIa or IIb, or a pharmaceutically acceptable salt, co-crystal, solvate, or combination thereof,
on a free base basis;
   about 621 mg of mannitol;
   about 335.9175 mg of microcrystalline cellulose;
   about 120 mg croscarmellose sodium; and
   about 22.5 mg magnesium stearate; and
ii) administering to the patient a maintenance dose comprising one tablet, once weekly, beginning on day 8, wherein the tablet comprises:
   about 400 mg of the spray dried dispersion of the compound of Formula Ia or Ib, or a pharmaceutically acceptable salt, co-crystal, solvate, or combination thereof;
   about 2.0 mg of the compound of Formula IIa or IIb, or a pharmaceutically acceptable salt, co-crystal, solvate, or combination thereof,
on a free base basis;
   about 621 mg of mannitol;
   about 334.32 mg of microcrystalline cellulose;
   about 120 mg croscarmellose sodium; and
about 22.5 mg magnesium stearate.

In some embodiments, the method comprises:
i) administering to the patient an initiation dose comprising two tablets on day 1, and two tablets on day 2, wherein each tablet of the initiation dose comprises:
   about 26.6 w/w % to about 26.7 w/w % of the spray dried dispersion of the compound of Formula Ia or Ib, or a pharmaceutically acceptable salt thereof;
   about 0.03 w/w % to about 0.04 w/w % of the compound of Formula IIa or IIb, or a pharmaceutically acceptable salt, co-crystal, solvate, or combination thereof, and
   one or more pharmaceutically acceptable excipients; and
ii) administering to the patient a maintenance dose comprising one tablet, once weekly, beginning on day 8, wherein the tablet comprises:
   about 26.6 w/w % to about 26.7 w/w % of the spray dried dispersion of the compound of Formula Ia or Ib, or a pharmaceutically acceptable salt thereof;
   about 0.14 w/w % to about 0.15 w/w % of the compound of Formula IIa or IIb, or a pharmaceutically acceptable salt, co-crystal, solvate, or combination thereof, and
   one or more pharmaceutically acceptable excipients.

In some embodiments, the method comprises:
i) administering to the patient an initiation dose comprising two tablets on day 1, and two tablets on day 2, wherein each tablet of the initiation dose comprises:
   about 26.67 w/w % of the spray dried dispersion of the compound of Formula Ia or Ib, or a pharmaceutically acceptable salt thereof;
   about 0.0355 w/w % of the compound of Formula IIa or IIb, or a pharmaceutically acceptable salt, co-crystal, solvate, or combination thereof; and
   one or more pharmaceutically acceptable excipients; and
ii) administering to the patient a maintenance dose comprising one tablet, once weekly, beginning on day 8, wherein the tablet comprises:
   about 26.67 w/w % of the spray dried dispersion of the compound of Formula Ia or Ib, or a pharmaceutically acceptable salt thereof;
   about 0.142 w/w % of the compound of Formula IIa or IIb, or a pharmaceutically acceptable salt, co-crystal, solvate, or combination thereof; and
   one or more pharmaceutically acceptable excipients.

In some embodiments, the method comprises:
i) administering to the patient an initiation dose comprising two tablets on day 1, and two tablets on day 2, wherein each tablet of the initiation dose comprises:
   about 26.6 w/w % to about 26.7 w/w % of the spray dried dispersion of the compound of Formula Ia or Ib, or a pharmaceutically acceptable salt thereof;
   about 0.03 w/w % to about 0.04 w/w % of the compound of Formula IIa or IIb, or a solvate thereof; and
   one or more pharmaceutically acceptable excipients; and
ii) administering to the patient a maintenance dose comprising one tablet, once weekly, beginning on day 8, wherein the tablet comprises:
   about 26.6 w/w % to about 26.7 w/w % of the spray dried dispersion of the compound of Formula Ia or Ib, or a pharmaceutically acceptable salt thereof;
   about 0.14 w/w % to about 0.15 w/w % of the compound of Formula IIa or IIb, or a solvate thereof; and
   one or more pharmaceutically acceptable excipients.

In some embodiments, the method comprises:
i) administering to the patient an initiation dose comprising two tablets on day 1, and two tablets on day 2, wherein each tablet of the initiation dose comprises:
   about 26.67 w/w % of the spray dried dispersion of the compound of Formula Ia or Ib, or a pharmaceutically acceptable salt thereof;
   about 0.0355 w/w % of the compound of Formula IIa or IIb, or a solvate thereof, and
   one or more pharmaceutically acceptable excipients; and
ii) administering to the patient a maintenance dose comprising one tablet, once weekly, beginning on day 8, wherein the tablet comprises:
   about 26.67 w/w % of the spray dried dispersion of the compound of Formula Ia or Ib, or a pharmaceutically acceptable salt thereof;
   about 0.142 w/w % of the compound of Formula IIa or IIb, or a solvate thereof, and
   one or more pharmaceutically acceptable excipients.

In some embodiments, the method comprises:
i) administering to the patient an initiation dose comprising two tablets on day 1, and two tablets on day 2, wherein each tablet of the initiation dose comprises:
   about 26.6 w/w % to about 26.7 w/w % of the spray dried dispersion of the compound of Formula Ia or Ib, or a pharmaceutically acceptable salt thereof;
   about 0.03 w/w % to about 0.04 w/w % of the monohydrate of the compound of Formula IIa or IIb; and
   one or more pharmaceutically acceptable excipients; and
ii) administering to the patient a maintenance dose comprising one tablet, once weekly, beginning on day 8, wherein the tablet comprises:
   about 26.6 w/w % to about 26.7 w/w % of the spray dried dispersion of the compound of Formula Ia or Ib, or a pharmaceutically acceptable salt thereof;
   about 0.14 w/w % to about 0.15 w/w % of the monohydrate of the compound of Formula IIa or IIb; and
   one or more pharmaceutically acceptable excipients.

In some embodiments, the method comprises:
i) administering to the patient an initiation dose comprising two tablets on day 1, and two tablets on day 2, wherein each tablet of the initiation dose comprises:
   about 26.67 w/w % of the spray dried dispersion of the compound of Formula Ia or Ib, or a pharmaceutically acceptable salt thereof;
   about 0.0355 w/w % of the monohydrate of the compound of Formula IIa or IIb; and
   one or more pharmaceutically acceptable excipients; and
ii) administering to the patient a maintenance dose comprising one tablet, once weekly, beginning on day 8, wherein the tablet comprises:
   about 26.67 w/w % of the spray dried dispersion of the compound of Formula Ia or Ib, or a pharmaceutically acceptable salt thereof;
   about 0.142 w/w % of the monohydrate of the compound of Formula IIa or IIb; and
   one or more pharmaceutically acceptable excipients.

In some embodiments, the method comprises:
i) administering to the patient an initiation dose comprising two tablets on day 1, and two tablets on day 2, wherein each tablet of the initiation dose comprises:
   about 26.6 w/w % to about 26.7 w/w % of the spray dried dispersion of the compound of Formula Ia or Ib, or a pharmaceutically acceptable salt thereof;
   about 0.03 w/w % to about 0.04 w/w % of the compound of Formula IIa or IIb, or a pharmaceutically acceptable salt, co-crystal, solvate, or combination thereof;
   about 41 w/w % to about 42 w/w % of mannitol;
   about 22.3 w/w % to about 22.4 w/w % of microcrystalline cellulose;
   about 7.9 w/w % to about 8.1 w/w % of croscarmellose sodium; and
   about 1.4 w/w % to about 1.6 w/w % of magnesium stearate; and
ii) administering to the patient a maintenance dose comprising one tablet, once weekly, beginning on day 8, wherein the tablet comprises:
   about 26.6 w/w % to about 26.7 w/w % of the spray dried dispersion of the compound of Formula Ia or Ib, or a pharmaceutically acceptable salt thereof;
   about 0.14 w/w % to about 0.15 w/w % of the compound of Formula IIa or IIb, or a pharmaceutically acceptable salt, co-crystal, solvate, or combination thereof;
   about 41 w/w % to about 42 w/w % of mannitol;
   about 22.2 w/w % to about 22.3 w/w % of microcrystalline cellulose;
   about 7.9 w/w % to about 8.1 w/w % of croscarmellose sodium; and
   about 1.4 w/w % to about 1.6 w/w % of magnesium stearate.

In some embodiments, the method comprises:
i) administering to the patient an initiation dose comprising two tablets on day 1, and two tablets on day 2, wherein each tablet of the initiation dose comprises:
   about 26.67 w/w % of the spray dried dispersion of the compound of Formula Ia or Ib, or a pharmaceutically acceptable salt thereof;
   about 0.0355 w/w % of the compound of Formula IIa or IIb, or a pharmaceutically acceptable salt, co-crystal, solvate, or combination thereof;
   about 41.40 w/w % of mannitol;
   about 22.3945 w/w % of microcrystalline cellulose;
   about 8.0 w/w % of croscarmellose sodium; and
   about 1.5 w/w % of magnesium stearate; and
ii) administering to the patient a maintenance dose comprising one tablet, once weekly, beginning on day 8, wherein the tablet comprises:
   about 26.67 w/w % of the spray dried dispersion of the compound of Formula Ia or Ib, or a pharmaceutically acceptable salt thereof;
   about 0.142 w/w % of the compound of Formula IIa or IIb, or a pharmaceutically acceptable salt, co-crystal, solvate, or combination thereof;
   about 41.40 w/w % of mannitol;
   about 22.288 w/w % microcrystalline cellulose;
   about 8.0 w/w % of croscarmellose sodium; and
   about 1.5 w/w % of magnesium stearate.

In some embodiments, the method comprises:
i) administering to the patient an initiation dose comprising two tablets on day 1, and two tablets on day 2, wherein each tablet of the initiation dose comprises:
   about 400 mg of the spray dried dispersion of the compound of Formula Ia or Ib, or a pharmaceutically acceptable salt thereof; and
   about 0.5 mg to about 0.6 mg of the compound of Formula IIa or IIb, or a pharmaceutically acceptable salt, co-crystal, solvate, or combination thereof, and
ii) administering to the patient a maintenance dose comprising one tablet, once weekly, beginning on day 8, wherein the tablet comprises:
   about 400 mg of the spray dried dispersion of the compound of Formula Ia or Ib, or a pharmaceutically acceptable salt thereof; and
   about 2.1 mg to about 2.2 mg of the compound of Formula IIa or IIb, or a pharmaceutically acceptable salt, co-crystal, solvate, or combination thereof.

In some embodiments, the method comprises:
i) administering to the patient an initiation dose comprising two tablets on day 1, and two tablets on day 2, wherein each tablet of the initiation dose comprises:
   about 400 mg of the spray dried dispersion of the compound of Formula Ia or Ib, or a pharmaceutically acceptable salt thereof; and
   about 0.5325 mg of the compound of Formula IIa or IIb, or a pharmaceutically acceptable salt, co-crystal, solvate, or combination thereof; and
ii) administering to the patient a maintenance dose comprising one tablet, once weekly, beginning on day 8, wherein the tablet comprises:
   about 400 mg of the spray dried dispersion of the compound of Formula Ia or Ib, or a pharmaceutically acceptable salt thereof; and
   about 2.130 mg of the compound of Formula IIa or IIb, or a pharmaceutically acceptable salt, co-crystal, solvate, or combination thereof.

In some embodiments, the method comprises:
i) administering to the patient an initiation dose comprising two tablets on day 1, and two tablets on day 2, wherein each tablet of the initiation dose comprises:
   about 400 mg of the spray dried dispersion of the compound of Formula Ia or Ib, or a pharmaceutically acceptable salt thereof; and
   about 0.5 mg of the compound of Formula IIa or IIb, or a pharmaceutically acceptable salt, co-crystal, solvate, or combination thereof,
on a free base basis; and
ii) administering to the patient a maintenance dose comprising one tablet, once weekly, beginning on day 8, wherein the tablet comprises:
   about 400 mg of the spray dried dispersion of the compound of Formula Ia or Ib, or a pharmaceutically acceptable salt thereof; and
   about 2.0 mg of the compound of Formula IIa or IIb, or a pharmaceutically acceptable salt, co-crystal, solvate, or combination thereof,
on a free base basis.

In some embodiments, the method comprises:
i) administering to the patient an initiation dose comprising two tablets on day 1, and two tablets on day 2, wherein each tablet of the initiation dose comprises:
   about 400 mg of the spray dried dispersion of the compound of Formula Ia or Ib, or a pharmaceutically acceptable salt thereof;
   about 0.5 mg to about 0.6 mg of the compound of Formula IIa or IIb, or a pharmaceutically acceptable salt, co-crystal, solvate, or combination thereof;
   about 615 mg to about 625 mg of mannitol;
   about 330 mg to about 340 mg of microcrystalline cellulose;
   about 115 mg to about 125 mg croscarmellose sodium; and
   about 20 mg to about 25 mg magnesium stearate; and
ii) administering to the patient a maintenance dose comprising one tablet, once weekly, beginning on day 8, wherein the tablet comprises:
   about 400 mg of the spray dried dispersion of the compound of Formula Ia or Ib, or a pharmaceutically acceptable salt thereof;
   about 2.1 mg to about 2.2 mg of the compound of Formula IIa or IIb, or a pharmaceutically acceptable salt, co-crystal, solvate, or combination thereof;
   about 615 mg to about 625 mg of mannitol;
   about 330 mg to about 340 mg of microcrystalline cellulose;
   about 115 mg to about 125 mg croscarmellose sodium; and
   about 20 mg to about 25 mg magnesium stearate.

In some embodiments, the method comprises:
i) administering to the patient an initiation dose comprising two tablets on day 1, and two tablets on day 2, wherein each tablet of the initiation dose comprises:
   about 400 mg of the spray dried dispersion of the compound of Formula Ia or Ib, or a pharmaceutically acceptable salt thereof;
   about 0.5325 mg of the compound of Formula IIa or IIb, or a pharmaceutically acceptable salt, co-crystal, solvate, or combination thereof;
   about 621 mg of mannitol;
   about 335.9175 mg of microcrystalline cellulose;
   about 120 mg croscarmellose sodium; and
   about 22.5 mg magnesium stearate; and
ii) administering to the patient a maintenance dose comprising one tablet, once weekly, beginning on day 8, wherein the tablet comprises:
   about 400 mg of the spray dried dispersion of the compound of Formula Ia or Ib, or a pharmaceutically acceptable salt thereof;
   about 2.130 mg of the compound of Formula IIa or IIb, or a pharmaceutically acceptable salt, co-crystal, solvate, or combination thereof;
   about 621 mg of mannitol;
   about 334.32 mg of microcrystalline cellulose;
   about 120 mg croscarmellose sodium; and
   about 22.5 mg magnesium stearate.

In some embodiments, the method comprises:
i) administering to the patient an initiation dose comprising two tablets on day 1, and two tablets on day 2, wherein each tablet of the initiation dose comprises:
   about 400 mg of the spray dried dispersion of the compound of Formula Ia or Ib, or a pharmaceutically acceptable salt thereof;
   about 0.5 mg of the compound of Formula IIa or IIb, or a pharmaceutically acceptable salt, co-crystal, solvate, or combination thereof,
on a free base basis;
   about 621 mg of mannitol;
   about 335.9175 mg of microcrystalline cellulose;
   about 120 mg croscarmellose sodium; and
   about 22.5 mg magnesium stearate; and
ii) administering to the patient a maintenance dose comprising one tablet, once weekly, beginning on day 8, wherein the tablet comprises:
   about 400 mg of the spray dried dispersion of the compound of Formula Ia or Ib, or a pharmaceutically acceptable salt thereof;
   about 2.0 mg of the compound of Formula IIa or IIb, or a pharmaceutically acceptable salt, co-crystal, solvate, or combination thereof,
on a free base basis;
   about 621 mg of mannitol;
   about 334.32 mg of microcrystalline cellulose;
   about 120 mg croscarmellose sodium; and
   about 22.5 mg magnesium stearate.

In some embodiments, the method comprises:
i) administering to the patient an initiation dose comprising two tablets on day 1, and two tablets on day 2, wherein each tablet of the initiation dose comprises:
   about 26.6 w/w % to about 26.7 w/w % of the spray dried dispersion of the sodium salt of the compound of Formula Ia or Ib;
   about 0.03 w/w % to about 0.04 w/w % of the compound of Formula IIa or IIb, or a pharmaceutically acceptable salt, co-crystal, solvate, or combination thereof, and
   one or more pharmaceutically acceptable excipients; and
ii) administering to the patient a maintenance dose comprising one tablet, once weekly, beginning on day 8, wherein the tablet comprises:
   about 26.6 w/w % to about 26.7 w/w % of the spray dried dispersion of the sodium salt of the compound of Formula Ia or Ib;
   about 0.14 w/w % to about 0.15 w/w % of the compound of Formula IIa or IIb, or a pharmaceutically acceptable salt, co-crystal, solvate, or combination thereof, and
   one or more pharmaceutically acceptable excipients.

In some embodiments, the method comprises:
i) administering to the patient an initiation dose comprising two tablets on day 1, and two tablets on day 2, wherein each tablet of the initiation dose comprises:
   about 26.67 w/w % of the spray dried dispersion of the sodium salt of the compound of Formula Ia or Ib;
   about 0.0355 w/w % of the compound of Formula IIa or IIb, or a pharmaceutically acceptable salt, co-crystal, solvate, or combination thereof; and
one or more pharmaceutically acceptable excipients; and
ii) administering to the patient a maintenance dose comprising one tablet, once weekly, beginning on day 8, wherein the tablet comprises:
   about 26.67 w/w % of the spray dried dispersion of the sodium salt of the compound of Formula Ia or Ib;
   about 0.142 w/w % of the compound of Formula IIa or IIb, or a pharmaceutically acceptable salt, co-crystal, solvate, or combination thereof; and
one or more pharmaceutically acceptable excipients.

In some embodiments, the method comprises:
i) administering to the patient an initiation dose comprising two tablets on day 1, and two tablets on day 2, wherein each tablet of the initiation dose comprises:
   about 26.6 w/w % to about 26.7 w/w % of the spray dried dispersion of the sodium salt of the compound of Formula Ia or Ib;
   about 0.03 w/w % to about 0.04 w/w % of the compound of Formula IIa or IIb, or a solvate thereof; and
   one or more pharmaceutically acceptable excipients; and
ii) administering to the patient a maintenance dose comprising one tablet, once weekly, beginning on day 8, wherein the tablet comprises:
   about 26.6 w/w % to about 26.7 w/w % of the spray dried dispersion of the sodium salt of the compound of Formula Ia or Ib;
   about 0.14 w/w % to about 0.15 w/w % of the compound of Formula IIa or IIb, or a solvate thereof; and
   one or more pharmaceutically acceptable excipients.

In some embodiments, the method comprises:
i) administering to the patient an initiation dose comprising two tablets on day 1, and two tablets on day 2, wherein each tablet of the initiation dose comprises:
   about 26.67 w/w % of the spray dried dispersion of the sodium salt of the compound of Formula Ia or Ib;
   about 0.0355 w/w % of the compound of Formula IIa or IIb, or a solvate thereof, and
   one or more pharmaceutically acceptable excipients; and
ii) administering to the patient a maintenance dose comprising one tablet, once weekly, beginning on day 8, wherein the tablet comprises:
   about 26.67 w/w % of the spray dried dispersion of the sodium salt of the compound of Formula Ia or Ib;
   about 0.142 w/w % of the compound of Formula IIa or IIb, or a solvate thereof, and
   one or more pharmaceutically acceptable excipients.

In some embodiments, the method comprises:
i) administering to the patient an initiation dose comprising two tablets on day 1, and two tablets on day 2, wherein each tablet of the initiation dose comprises:
   about 26.6 w/w % to about 26.7 w/w % of the spray dried dispersion of the sodium salt of the compound of Formula Ia or Ib;
   about 0.03 w/w % to about 0.04 w/w % of the monohydrate of the compound of Formula IIa or IIb; and
   one or more pharmaceutically acceptable excipients; and
ii) administering to the patient a maintenance dose comprising one tablet, once weekly, beginning on day 8, wherein the tablet comprises:
   about 26.6 w/w % to about 26.7 w/w % of the spray dried dispersion of the sodium salt of the compound of Formula Ia or Ib;
   about 0.14 w/w % to about 0.15 w/w % of the monohydrate of the compound of Formula IIa or IIb; and
   one or more pharmaceutically acceptable excipients.

In some embodiments, the method comprises:
i) administering to the patient an initiation dose comprising two tablets on day 1, and two tablets on day 2, wherein each tablet of the initiation dose comprises:
   about 26.67 w/w % of the spray dried dispersion of the sodium salt of the compound of Formula Ia or Ib;
   about 0.0355 w/w % of the monohydrate of the compound of Formula IIa or IIb; and
   one or more pharmaceutically acceptable excipients; and
ii) administering to the patient a maintenance dose comprising one tablet, once weekly, beginning on day 8, wherein the tablet comprises:
   about 26.67 w/w % of the spray dried dispersion of the sodium salt of the compound of Formula Ia or Ib;
   about 0.142 w/w % of the monohydrate of the compound of Formula IIa or IIb; and
   one or more pharmaceutically acceptable excipients.

In some embodiments, the method comprises:
i) administering to the patient an initiation dose comprising two tablets on day 1, and two tablets on day 2, wherein each tablet of the initiation dose comprises:
   about 26.6 w/w % to about 26.7 w/w % of the spray dried dispersion of the sodium salt of the compound of Formula Ia or Ib;
   about 0.03 w/w % to about 0.04 w/w % of the compound of Formula IIa or IIb, or a pharmaceutically acceptable salt, co-crystal, solvate, or combination thereof;
   about 41 w/w % to about 42 w/w % of mannitol;
   about 22.3 w/w % to about 22.4 w/w % of microcrystalline cellulose;
   about 7.9 w/w % to about 8.1 w/w % of croscarmellose sodium; and
   about 1.4 w/w % to about 1.6 w/w % of magnesium stearate; and
ii) administering to the patient a maintenance dose comprising one tablet, once weekly, beginning on day 8, wherein the tablet comprises:
   about 26.6 w/w % to about 26.7 w/w % of the spray dried dispersion of the sodium salt of the compound of Formula Ia or Ib;
   about 0.14 w/w % to about 0.15 w/w % of the compound of Formula IIa or IIb, or a pharmaceutically acceptable salt, co-crystal, solvate, or combination thereof;
   about 41 w/w % to about 42 w/w % of mannitol;
   about 22.2 w/w % to about 22.3 w/w % of microcrystalline cellulose;
   about 7.9 w/w % to about 8.1 w/w % of croscarmellose sodium; and
   about 1.4 w/w % to about 1.6 w/w % of magnesium stearate.

In some embodiments, the method comprises:
i) administering to the patient an initiation dose comprising two tablets on day 1, and two tablets on day 2, wherein each tablet of the initiation dose comprises:
   about 26.67 w/w % of the spray dried dispersion of the sodium salt of the compound of Formula Ia or Ib;
   about 0.0355 w/w % of the compound of Formula IIa or IIb, or a pharmaceutically acceptable salt, co-crystal, solvate, or combination thereof;
   about 41.40 w/w % of mannitol;
   about 22.3945 w/w % of microcrystalline cellulose;
   about 8.0 w/w % of croscarmellose sodium; and
   about 1.5 w/w % of magnesium stearate; and
ii) administering to the patient a maintenance dose comprising one tablet, once weekly, beginning on day 8, wherein the tablet comprises:
   about 26.67 w/w % of the spray dried dispersion of the sodium salt of the compound of Formula Ia or Ib;
   about 0.142 w/w % of the compound of Formula IIa or IIb, or a pharmaceutically acceptable salt, co-crystal, solvate, or combination thereof;
   about 41.40 w/w % of mannitol;
   about 22.288 w/w % microcrystalline cellulose;
   about 8.0 w/w % of croscarmellose sodium; and
   about 1.5 w/w % of magnesium stearate.

In some embodiments, the method comprises:
i) administering to the patient an initiation dose comprising two tablets on day 1, and two tablets on day 2, wherein each tablet of the initiation dose comprises:
   about 400 mg of the spray dried dispersion of the sodium salt of the compound of Formula Ia or Ib; and
   about 0.5 mg to about 0.6 mg of the compound of Formula IIa or IIb, or a pharmaceutically acceptable salt, co-crystal, solvate, or combination thereof, and
ii) administering to the patient a maintenance dose comprising one tablet, once weekly, beginning on day 8, wherein the tablet comprises:
   about 400 mg of the spray dried dispersion of the sodium salt of the compound of Formula Ia or Ib; and
   about 2.1 mg to about 2.2 mg of the compound of Formula IIa or IIb, or a pharmaceutically acceptable salt, co-crystal, solvate, or combination thereof.

In some embodiments, the method comprises:
i) administering to the patient an initiation dose comprising two tablets on day 1, and two tablets on day 2, wherein each tablet of the initiation dose comprises:
   about 400 mg of the spray dried dispersion of the sodium salt of the compound of Formula Ia or Ib; and
   about 0.5325 mg of the compound of Formula IIa or IIb, or a pharmaceutically acceptable salt, co-crystal, solvate, or combination thereof; and
ii) administering to the patient a maintenance dose comprising one tablet, once weekly, beginning on day 8, wherein the tablet comprises:
   about 400 mg of the spray dried dispersion of the sodium salt of the compound of Formula Ia or Ib; and
   about 2.130 mg of the compound of Formula IIa or IIb, or a pharmaceutically acceptable salt, co-crystal, solvate, or combination thereof.

In some embodiments, the method comprises:
i) administering to the patient an initiation dose comprising two tablets on day 1, and two tablets on day 2, wherein each tablet of the initiation dose comprises:
   about 400 mg of the spray dried dispersion of the sodium salt of the compound of Formula Ia or Ib; and
   about 0.5 mg of the compound of Formula IIa or IIb, or a pharmaceutically acceptable salt, co-crystal, solvate, or combination thereof,
on a free base basis; and
ii) administering to the patient a maintenance dose comprising one tablet, once weekly, beginning on day 8, wherein the tablet comprises:
   about 400 mg of the spray dried dispersion of the sodium salt of the compound of Formula Ia or Ib; and
   about 2.0 mg of the compound of Formula IIa or IIb, or a pharmaceutically acceptable salt, co-crystal, solvate, or combination thereof,
on a free base basis.

In some embodiments, the method comprises:
i) administering to the patient an initiation dose comprising two tablets on day 1, and two tablets on day 2, wherein each tablet of the initiation dose comprises:
   about 400 mg of the spray dried dispersion of the sodium salt of the compound of Formula Ia or Ib;
   about 0.5 mg to about 0.6 mg of the compound of Formula IIa or IIb, or a pharmaceutically acceptable salt, co-crystal, solvate, or combination thereof;
   about 615 mg to about 625 mg of mannitol;
   about 330 mg to about 340 mg of microcrystalline cellulose;
   about 115 mg to about 125 mg croscarmellose sodium; and
   about 20 mg to about 25 mg magnesium stearate; and
ii) administering to the patient a maintenance dose comprising one tablet, once weekly, beginning on day 8, wherein the tablet comprises:
   about 400 mg of the spray dried dispersion of the sodium salt of the compound of Formula Ia or Ib;
   about 2.1 mg to about 2.2 mg of the compound of Formula IIa or IIb, or a pharmaceutically acceptable salt, co-crystal, solvate, or combination thereof;
   about 615 mg to about 625 mg of mannitol;
   about 330 mg to about 340 mg of microcrystalline cellulose;
   about 115 mg to about 125 mg croscarmellose sodium; and
   about 20 mg to about 25 mg magnesium stearate.

In some embodiments, the method comprises:
i) administering to the patient an initiation dose comprising two tablets on day 1, and two tablets on day 2, wherein each tablet of the initiation dose comprises:
   about 400 mg of the spray dried dispersion of the sodium salt of the compound of Formula Ia or Ib;
   about 0.5325 mg of the compound of Formula IIa or IIb, or a pharmaceutically acceptable salt, co-crystal, solvate, or combination thereof;
   about 621 mg of mannitol;
   about 335.9175 mg of microcrystalline cellulose;
   about 120 mg croscarmellose sodium; and
   about 22.5 mg magnesium stearate; and
ii) administering to the patient a maintenance dose comprising one tablet, once weekly, beginning on day 8, wherein the tablet comprises:
   about 400 mg of the spray dried dispersion of the sodium salt of the compound of Formula Ia or Ib;
   about 2.130 mg of the compound of Formula IIa or IIb, or a pharmaceutically acceptable salt, co-crystal, solvate, or combination thereof;
   about 621 mg of mannitol;
   about 334.32 mg of microcrystalline cellulose;
   about 120 mg croscarmellose sodium; and
   about 22.5 mg magnesium stearate.

In some embodiments, the method comprises:

i) administering to the patient an initiation dose comprising two tablets on day 1, and two tablets on day 2, wherein each tablet of the initiation dose comprises:
   about 400 mg of the spray dried dispersion of the sodium salt of the compound of Formula Ia or Ib;
   about 0.5 mg of the compound of Formula IIa or IIb, or a pharmaceutically acceptable salt, co-crystal, solvate, or combination thereof,
on a free base basis;
   about 621 mg of mannitol;
   about 335.9175 mg of microcrystalline cellulose;
   about 120 mg croscarmellose sodium; and
   about 22.5 mg magnesium stearate; and
ii) administering to the patient a maintenance dose comprising one tablet, once weekly, beginning on day 8, wherein the tablet comprises:
   about 400 mg of the spray dried dispersion of the sodium salt of the compound of Formula Ia or Ib;
   about 2.0 mg of the compound of Formula IIa or IIb, or a pharmaceutically acceptable salt, co-crystal, solvate, or combination thereof,
on a free base basis;
   about 621 mg of mannitol;
   about 334.32 mg of microcrystalline cellulose;
   about 120 mg croscarmellose sodium; and
   about 22.5 mg magnesium stearate.

In some embodiments, the method comprises:
i) administering to the patient an initiation dose comprising two tablets on day 1, and two tablets on day 2, wherein each tablet of the initiation dose comprises:
   about 26.6 w/w % to about 26.7 w/w % of the spray dried dispersion of the sodium salt of the compound of Formula Ia;
   about 0.03 w/w % to about 0.04 w/w % of the compound of Formula IIa or IIb, or a pharmaceutically acceptable salt, co-crystal, solvate, or combination thereof, and
   one or more pharmaceutically acceptable excipients; and
ii) administering to the patient a maintenance dose comprising one tablet, once weekly, beginning on day 8, wherein the tablet comprises:
   about 26.6 w/w % to about 26.7 w/w % of the spray dried dispersion of the sodium salt of the compound of Formula Ia;
   about 0.14 w/w % to about 0.15 w/w % of the compound of Formula IIa or IIb, or a pharmaceutically acceptable salt, co-crystal, solvate, or combination thereof, and
   one or more pharmaceutically acceptable excipients.

In some embodiments, the method comprises:
i) administering to the patient an initiation dose comprising two tablets on day 1, and two tablets on day 2, wherein each tablet of the initiation dose comprises:
   about 26.67 w/w % of the spray dried dispersion of the sodium salt of the compound of Formula Ia;
   about 0.0355 w/w % of the compound of Formula IIa or IIb, or a pharmaceutically acceptable salt, co-crystal, solvate, or combination thereof; and
   one or more pharmaceutically acceptable excipients; and
ii) administering to the patient a maintenance dose comprising one tablet, once weekly, beginning on day 8, wherein the tablet comprises:
   about 26.67 w/w % of the spray dried dispersion of the sodium salt of the compound of Formula Ia;
   about 0.142 w/w % of the compound of Formula IIa or IIb, or a pharmaceutically acceptable salt, co-crystal, solvate, or combination thereof; and
one or more pharmaceutically acceptable excipients.

In some embodiments, the method comprises:
i) administering to the patient an initiation dose comprising two tablets on day 1, and two tablets on day 2, wherein each tablet of the initiation dose comprises:
   about 26.6 w/w % to about 26.7 w/w % of the spray dried dispersion of the sodium salt of the compound of Formula Ia;
   about 0.03 w/w % to about 0.04 w/w % of the compound of Formula IIa or IIb, or a solvate thereof; and
   one or more pharmaceutically acceptable excipients; and
ii) administering to the patient a maintenance dose comprising one tablet, once weekly, beginning on day 8, wherein the tablet comprises:
   about 26.6 w/w % to about 26.7 w/w % of the spray dried dispersion of the sodium salt of the compound of Formula Ia;
   about 0.14 w/w % to about 0.15 w/w % of the compound of Formula IIa or IIb, or a solvate thereof; and
   one or more pharmaceutically acceptable excipients.

In some embodiments, the method comprises:
i) administering to the patient an initiation dose comprising two tablets on day 1, and two tablets on day 2, wherein each tablet of the initiation dose comprises:
   about 26.67 w/w % of the spray dried dispersion of the sodium salt of the compound of Formula Ia;
   about 0.0355 w/w % of the compound of Formula IIa or IIb, or a solvate thereof, and
   one or more pharmaceutically acceptable excipients; and
ii) administering to the patient a maintenance dose comprising one tablet, once weekly, beginning on day 8, wherein the tablet comprises:
   about 26.67 w/w % of the spray dried dispersion of the sodium salt of the compound of Formula Ia;
   about 0.142 w/w % of the compound of Formula IIa or IIb, or a solvate thereof, and
   one or more pharmaceutically acceptable excipients.

In some embodiments, the method comprises:
i) administering to the patient an initiation dose comprising two tablets on day 1, and two tablets on day 2, wherein each tablet of the initiation dose comprises:
   about 26.6 w/w % to about 26.7 w/w % of the spray dried dispersion of the sodium salt of the compound of Formula Ia;
   about 0.03 w/w % to about 0.04 w/w % of the monohydrate of the compound of Formula IIa or IIb; and
   one or more pharmaceutically acceptable excipients; and
ii) administering to the patient a maintenance dose comprising one tablet, once weekly, beginning on day 8, wherein the tablet comprises:
   about 26.6 w/w % to about 26.7 w/w % of the spray dried dispersion of the sodium salt of the compound of Formula Ia;
   about 0.14 w/w % to about 0.15 w/w % of the monohydrate of the compound of Formula IIa or IIb; and
   one or more pharmaceutically acceptable excipients.

In some embodiments, the method comprises:
i) administering to the patient an initiation dose comprising two tablets on day 1, and two tablets on day 2, wherein each tablet of the initiation dose comprises:
   about 26.67 w/w % of the spray dried dispersion of the sodium salt of the compound of Formula Ia;
   about 0.0355 w/w % of the monohydrate of the compound of Formula IIa or IIb; and
   one or more pharmaceutically acceptable excipients; and
ii) administering to the patient a maintenance dose comprising one tablet, once weekly, beginning on day 8, wherein the tablet comprises:
   about 26.67 w/w % of the spray dried dispersion of the sodium salt of the compound of Formula Ia;
   about 0.142 w/w % of the monohydrate of the compound of Formula IIa or IIb; and
   one or more pharmaceutically acceptable excipients.

In some embodiments, the method comprises:
i) administering to the patient an initiation dose comprising two tablets on day 1, and two tablets on day 2, wherein each tablet of the initiation dose comprises:
   about 26.6 w/w % to about 26.7 w/w % of the spray dried dispersion of the sodium salt of the compound of Formula Ia;
   about 0.03 w/w % to about 0.04 w/w % of the compound of Formula IIa or IIb, or a pharmaceutically acceptable salt, co-crystal, solvate, or combination thereof;
   about 41 w/w % to about 42 w/w % of mannitol;
   about 22.3 w/w % to about 22.4 w/w % of microcrystalline cellulose;
   about 7.9 w/w % to about 8.1 w/w % of croscarmellose sodium; and
   about 1.4 w/w % to about 1.6 w/w % of magnesium stearate; and
ii) administering to the patient a maintenance dose comprising one tablet, once weekly, beginning on day 8, wherein the tablet comprises:
   about 26.6 w/w % to about 26.7 w/w % of the spray dried dispersion of the sodium salt of the compound of Formula Ia;
   about 0.14 w/w % to about 0.15 w/w % of the compound of Formula IIa or IIb, or a pharmaceutically acceptable salt, co-crystal, solvate, or combination thereof;
   about 41 w/w % to about 42 w/w % of mannitol;
   about 22.2 w/w % to about 22.3 w/w % of microcrystalline cellulose;
   about 7.9 w/w % to about 8.1 w/w % of croscarmellose sodium; and
   about 1.4 w/w % to about 1.6 w/w % of magnesium stearate.

In some embodiments, the method comprises:
i) administering to the patient an initiation dose comprising two tablets on day 1, and two tablets on day 2, wherein each tablet of the initiation dose comprises:
   about 26.67 w/w % of the spray dried dispersion of the sodium salt of the compound of Formula Ia;
   about 0.0355 w/w % of the compound of Formula IIa or IIb, or a pharmaceutically acceptable salt, co-crystal, solvate, or combination thereof;
   about 41.40 w/w % of mannitol;
   about 22.3945 w/w % of microcrystalline cellulose;
   about 8.0 w/w % of croscarmellose sodium; and
   about 1.5 w/w % of magnesium stearate; and
ii) administering to the patient a maintenance dose comprising one tablet, once weekly, beginning on day 8, wherein the tablet comprises:
   about 26.67 w/w % of the spray dried dispersion of the sodium salt of the compound of Formula Ia;
   about 0.142 w/w % of the compound of Formula IIa or IIb, or a pharmaceutically acceptable salt, co-crystal, solvate, or combination thereof;
   about 41.40 w/w % of mannitol;
   about 22.288 w/w % microcrystalline cellulose;
   about 8.0 w/w % of croscarmellose sodium; and
   about 1.5 w/w % of magnesium stearate.

In some embodiments, the method comprises:
i) administering to the patient an initiation dose comprising two tablets on day 1, and two tablets on day 2, wherein each tablet of the initiation dose comprises:
   about 400 mg of the spray dried dispersion of the sodium salt of the compound of Formula Ia; and
   about 0.5 mg to about 0.6 mg of the compound of Formula IIa or IIb, or a pharmaceutically acceptable salt, co-crystal, solvate, or combination thereof, and
ii) administering to the patient a maintenance dose comprising one tablet, once weekly, beginning on day 8, wherein the tablet comprises:
   about 400 mg of the spray dried dispersion of the sodium salt of the compound of Formula Ia; and
   about 2.1 mg to about 2.2 mg of the compound of Formula IIa or IIb, or a pharmaceutically acceptable salt, co-crystal, solvate, or combination thereof.

In some embodiments, the method comprises:
i) administering to the patient an initiation dose comprising two tablets on day 1, and two tablets on day 2, wherein each tablet of the initiation dose comprises:
   about 400 mg of the spray dried dispersion of the sodium salt of the compound of Formula Ia; and
   about 0.5325 mg of the compound of Formula IIa or IIb, or a pharmaceutically acceptable salt, co-crystal, solvate, or combination thereof; and
ii) administering to the patient a maintenance dose comprising one tablet, once weekly, beginning on day 8, wherein the tablet comprises:
   about 400 mg of the spray dried dispersion of the sodium salt of the compound of Formula Ia; and
   about 2.130 mg of the compound of Formula IIa or IIb, or a pharmaceutically acceptable salt, co-crystal, solvate, or combination thereof.

In some embodiments, the method comprises:
i) administering to the patient an initiation dose comprising two tablets on day 1, and two tablets on day 2, wherein each tablet of the initiation dose comprises:
   about 400 mg of the spray dried dispersion of the sodium salt of the compound of Formula Ia; and
   about 0.5 mg of the compound of Formula IIa or IIb, or a pharmaceutically acceptable salt, co-crystal, solvate, or combination thereof,
on a free base basis; and
ii) administering to the patient a maintenance dose comprising one tablet, once weekly, beginning on day 8, wherein the tablet comprises:
   about 400 mg of the spray dried dispersion of the sodium salt of the compound of Formula Ia; and
   about 2.0 mg of the compound of Formula IIa or IIb, or a pharmaceutically acceptable salt, co-crystal, solvate, or combination thereof,
on a free base basis.

In some embodiments, the method comprises:
i) administering to the patient an initiation dose comprising two tablets on day 1, and two tablets on day 2, wherein each tablet of the initiation dose comprises:
   about 400 mg of the spray dried dispersion of the sodium salt of the compound of Formula Ia;
   about 0.5 mg to about 0.6 mg of the compound of Formula IIa or IIb, or a pharmaceutically acceptable salt, co-crystal, solvate, or combination thereof;
   about 615 mg to about 625 mg of mannitol;
   about 330 mg to about 340 mg of microcrystalline cellulose;
   about 115 mg to about 125 mg croscarmellose sodium; and
   about 20 mg to about 25 mg magnesium stearate; and
ii) administering to the patient a maintenance dose comprising one tablet, once weekly, beginning on day 8, wherein the tablet comprises:
   about 400 mg of the spray dried dispersion of the sodium salt of the compound of Formula Ia;
   about 2.1 mg to about 2.2 mg of the compound of Formula IIa or IIb, or a pharmaceutically acceptable salt, co-crystal, solvate, or combination thereof;
   about 615 mg to about 625 mg of mannitol;
   about 330 mg to about 340 mg of microcrystalline cellulose;
   about 115 mg to about 125 mg croscarmellose sodium; and
   about 20 mg to about 25 mg magnesium stearate.

In some embodiments, the method comprises:
i) administering to the patient an initiation dose comprising two tablets on day 1, and two tablets on day 2, wherein each tablet of the initiation dose comprises:
   about 400 mg of the spray dried dispersion of the sodium salt of the compound of Formula Ia;
   about 0.5325 mg of the compound of Formula IIa or IIb, or a pharmaceutically acceptable salt, co-crystal, solvate, or combination thereof;
   about 621 mg of mannitol;
   about 335.9175 mg of microcrystalline cellulose;
   about 120 mg croscarmellose sodium; and
   about 22.5 mg magnesium stearate; and
ii) administering to the patient a maintenance dose comprising one tablet, once weekly, beginning on day 8, wherein the tablet comprises:
   about 400 mg of the spray dried dispersion of the sodium salt of the compound of Formula Ia;
   about 2.130 mg of the compound of Formula IIa or IIb, or a pharmaceutically acceptable salt, co-crystal, solvate, or combination thereof;
   about 621 mg of mannitol;
   about 334.32 mg of microcrystalline cellulose;
   about 120 mg croscarmellose sodium; and
   about 22.5 mg magnesium stearate.

In some embodiments, the method comprises:
i) administering to the patient an initiation dose comprising two tablets on day 1, and two tablets on day 2, wherein each tablet of the initiation dose comprises:
   about 400 mg of the spray dried dispersion of the sodium salt of the compound of Formula Ia;
   about 0.5 mg of the compound of Formula IIa or IIb, or a pharmaceutically acceptable salt, co-crystal, solvate, or combination thereof,
on a free base basis;
   about 621 mg of mannitol;
   about 335.9175 mg of microcrystalline cellulose;
   about 120 mg croscarmellose sodium; and
   about 22.5 mg magnesium stearate; and
ii) administering to the patient a maintenance dose comprising one tablet, once weekly, beginning on day 8, wherein the tablet comprises:
   about 400 mg of the spray dried dispersion of the sodium salt of the compound of Formula Ia;
   about 2.0 mg of the compound of Formula IIa or IIb, or a pharmaceutically acceptable salt, co-crystal, solvate, or combination thereof,
on a free base basis;
   about 621 mg of mannitol;
   about 334.32 mg of microcrystalline cellulose;
   about 120 mg croscarmellose sodium; and
   about 22.5 mg magnesium stearate.

In some embodiments, the method comprises:
i) administering to the patient an initiation dose comprising two tablets on day 1, and two tablets on day 2, wherein each tablet of the initiation dose comprises:
   about 26.6 w/w % to about 26.7 w/w % of the lenacapavir spray dried dispersion;
   about 0.03 w/w % to about 0.04 w/w % of the compound of Formula IIa or IIb, or a pharmaceutically acceptable salt, co-crystal, solvate, or combination thereof, and
   one or more pharmaceutically acceptable excipients; and
ii) administering to the patient a maintenance dose comprising one tablet, once weekly, beginning on day 8, wherein the tablet comprises:
   about 26.6 w/w % to about 26.7 w/w % of the lenacapavir spray dried dispersion;
   about 0.14 w/w % to about 0.15 w/w % of the compound of Formula IIa or IIb, or a pharmaceutically acceptable salt, co-crystal, solvate, or combination thereof, and
   one or more pharmaceutically acceptable excipients.

In some embodiments, the method comprises:
i) administering to the patient an initiation dose comprising two tablets on day 1, and two tablets on day 2, wherein each tablet of the initiation dose comprises:
   about 26.67 w/w % of the lenacapavir spray dried dispersion;
   about 0.0355 w/w % of the compound of Formula IIa or IIb, or a pharmaceutically acceptable salt, co-crystal, solvate, or combination thereof; and
   one or more pharmaceutically acceptable excipients; and
ii) administering to the patient a maintenance dose comprising one tablet, once weekly, beginning on day 8, wherein the tablet comprises:
   about 26.67 w/w % of the lenacapavir spray dried dispersion;
   about 0.142 w/w % of the compound of Formula IIa or IIb, or a pharmaceutically acceptable salt, co-crystal, solvate, or combination thereof; and
   one or more pharmaceutically acceptable excipients.

In some embodiments, the method comprises:
i) administering to the patient an initiation dose comprising two tablets on day 1, and two tablets on day 2, wherein each tablet of the initiation dose comprises:
   about 26.6 w/w % to about 26.7 w/w % of the lenacapavir spray dried dispersion;
   about 0.03 w/w % to about 0.04 w/w % of the compound of Formula IIa or IIb, or a solvate thereof; and
   one or more pharmaceutically acceptable excipients; and
ii) administering to the patient a maintenance dose comprising one tablet, once weekly, beginning on day 8, wherein the tablet comprises:
   about 26.6 w/w % to about 26.7 w/w % of the lenacapavir spray dried dispersion;
   about 0.14 w/w % to about 0.15 w/w % of the compound of Formula IIa or IIb, or a solvate thereof; and
   one or more pharmaceutically acceptable excipients.

In some embodiments, the method comprises:
i) administering to the patient an initiation dose comprising two tablets on day 1, and two tablets on day 2, wherein each tablet of the initiation dose comprises:
   about 26.67 w/w % of the lenacapavir spray dried dispersion;
   about 0.0355 w/w % of the compound of Formula IIa or IIb, or a solvate thereof, and
   one or more pharmaceutically acceptable excipients; and
ii) administering to the patient a maintenance dose comprising one tablet, once weekly, beginning on day 8, wherein the tablet comprises:
   about 26.67 w/w % of the lenacapavir spray dried dispersion;
   about 0.142 w/w % of the compound of Formula IIa or IIb, or a solvate thereof, and
   one or more pharmaceutically acceptable excipients.

In some embodiments, the method comprises:
i) administering to the patient an initiation dose comprising two tablets on day 1, and two tablets on day 2, wherein each tablet of the initiation dose comprises:
   about 26.6 w/w % to about 26.7 w/w % of the lenacapavir spray dried dispersion;
   about 0.03 w/w % to about 0.04 w/w % of the monohydrate of the compound of Formula IIa or IIb; and
   one or more pharmaceutically acceptable excipients; and
ii) administering to the patient a maintenance dose comprising one tablet, once weekly, beginning on day 8, wherein the tablet comprises:
   about 26.6 w/w % to about 26.7 w/w % of the lenacapavir spray dried dispersion;
   about 0.14 w/w % to about 0.15 w/w % of the monohydrate of the compound of Formula IIa or IIb; and
   one or more pharmaceutically acceptable excipients.

In some embodiments, the method comprises:
i) administering to the patient an initiation dose comprising two tablets on day 1, and two tablets on day 2, wherein each tablet of the initiation dose comprises:
   about 26.67 w/w % of the lenacapavir spray dried dispersion;
   about 0.0355 w/w % of the monohydrate of the compound of Formula IIa or IIb; and
   one or more pharmaceutically acceptable excipients; and
ii) administering to the patient a maintenance dose comprising one tablet, once weekly, beginning on day 8, wherein the tablet comprises:
   about 26.67 w/w % of the lenacapavir spray dried dispersion;
   about 0.142 w/w % of the monohydrate of the compound of Formula IIa or IIb; and
   one or more pharmaceutically acceptable excipients.

In some embodiments, the method comprises:
i) administering to the patient an initiation dose comprising two tablets on day 1, and two tablets on day 2, wherein each tablet of the initiation dose comprises:
   about 26.6 w/w % to about 26.7 w/w % of the lenacapavir spray dried dispersion;
   about 0.03 w/w % to about 0.04 w/w % of the compound of Formula IIa or IIb, or a pharmaceutically acceptable salt, co-crystal, solvate, or combination thereof;
   about 41 w/w % to about 42 w/w % of mannitol;
   about 22.3 w/w % to about 22.4 w/w % of microcrystalline cellulose;
   about 7.9 w/w % to about 8.1 w/w % of croscarmellose sodium; and
   about 1.4 w/w % to about 1.6 w/w % of magnesium stearate; and
ii) administering to the patient a maintenance dose comprising one tablet, once weekly, beginning on day 8, wherein the tablet comprises:
   about 26.6 w/w % to about 26.7 w/w % of the lenacapavir spray dried dispersion;
   about 0.14 w/w % to about 0.15 w/w % of the compound of Formula IIa or IIb, or a pharmaceutically acceptable salt, co-crystal, solvate, or combination thereof;
   about 41 w/w % to about 42 w/w % of mannitol;
   about 22.2 w/w % to about 22.3 w/w % of microcrystalline cellulose;
   about 7.9 w/w % to about 8.1 w/w % of croscarmellose sodium; and
   about 1.4 w/w % to about 1.6 w/w % of magnesium stearate.

In some embodiments, the method comprises:
i) administering to the patient an initiation dose comprising two tablets on day 1, and two tablets on day 2, wherein each tablet of the initiation dose comprises:
   about 26.67 w/w % of the lenacapavir spray dried dispersion;
   about 0.0355 w/w % of the compound of Formula IIa or IIb, or a pharmaceutically acceptable salt, co-crystal, solvate, or combination thereof;
   about 41.40 w/w % of mannitol;
   about 22.3945 w/w % of microcrystalline cellulose;
   about 8.0 w/w % of croscarmellose sodium; and
   about 1.5 w/w % of magnesium stearate; and
ii) administering to the patient a maintenance dose comprising one tablet, once weekly, beginning on day 8, wherein the tablet comprises:
   about 26.67 w/w % of the lenacapavir spray dried dispersion;
   about 0.142 w/w % of the compound of Formula IIa or IIb, or a pharmaceutically acceptable salt, co-crystal, solvate, or combination thereof;
   about 41.40 w/w % of mannitol;
   about 22.288 w/w % microcrystalline cellulose;
   about 8.0 w/w % of croscarmellose sodium; and
   about 1.5 w/w % of magnesium stearate.

In some embodiments, the method comprises:
i) administering to the patient an initiation dose comprising two tablets on day 1, and two tablets on day 2, wherein each tablet of the initiation dose comprises:
   about 400 mg of the lenacapavir spray dried dispersion; and
   about 0.5 mg to about 0.6 mg of the compound of Formula IIa or IIb, or a pharmaceutically acceptable salt, co-crystal, solvate, or combination thereof, and
ii) administering to the patient a maintenance dose comprising one tablet, once weekly, beginning on day 8, wherein the tablet comprises:
   about 400 mg of the lenacapavir spray dried dispersion; and
   about 2.1 mg to about 2.2 mg of the compound of Formula IIa or IIb, or a pharmaceutically acceptable salt, co-crystal, solvate, or combination thereof.

In some embodiments, the method comprises:
i) administering to the patient an initiation dose comprising two tablets on day 1, and two tablets on day 2, wherein each tablet of the initiation dose comprises:
   about 400 mg of the lenacapavir spray dried dispersion; and
   about 0.5325 mg of the compound of Formula IIa or IIb, or a pharmaceutically acceptable salt, co-crystal, solvate, or combination thereof; and
ii) administering to the patient a maintenance dose comprising one tablet, once weekly, beginning on day 8, wherein the tablet comprises:
   about 400 mg of the lenacapavir spray dried dispersion; and
   about 2.130 mg of the compound of Formula IIa or IIb, or a pharmaceutically acceptable salt, co-crystal, solvate, or combination thereof.

In some embodiments, the method comprises:
i) administering to the patient an initiation dose comprising two tablets on day 1, and two tablets on day 2, wherein each tablet of the initiation dose comprises:
   about 400 mg of the lenacapavir spray dried dispersion; and
   about 0.5 mg of the compound of Formula IIa or IIb, or a pharmaceutically acceptable salt, co-crystal, solvate, or combination thereof,
on a free base basis; and
ii) administering to the patient a maintenance dose comprising one tablet, once weekly, beginning on day 8, wherein the tablet comprises:
   about 400 mg of the lenacapavir spray dried dispersion; and
   about 2.0 mg of the compound of Formula IIa or IIb, or a pharmaceutically acceptable salt, co-crystal, solvate, or combination thereof,
on a free base basis.

In some embodiments, the method comprises:
i) administering to the patient an initiation dose comprising two tablets on day 1, and two tablets on day 2, wherein each tablet of the initiation dose comprises:
   about 400 mg of the lenacapavir spray dried dispersion;
   about 0.5 mg to about 0.6 mg of the compound of Formula IIa or IIb, or a pharmaceutically acceptable salt, co-crystal, solvate, or combination thereof;
   about 615 mg to about 625 mg of mannitol;
   about 330 mg to about 340 mg of microcrystalline cellulose;
   about 115 mg to about 125 mg croscarmellose sodium; and
   about 20 mg to about 25 mg magnesium stearate; and
ii) administering to the patient a maintenance dose comprising one tablet, once weekly, beginning on day 8, wherein the tablet comprises:
   about 400 mg of the lenacapavir spray dried dispersion;
   about 2.1 mg to about 2.2 mg of the compound of Formula IIa or IIb, or a pharmaceutically acceptable salt, co-crystal, solvate, or combination thereof;
   about 615 mg to about 625 mg of mannitol;
   about 330 mg to about 340 mg of microcrystalline cellulose;
   about 115 mg to about 125 mg croscarmellose sodium; and
   about 20 mg to about 25 mg magnesium stearate.

In some embodiments, the method comprises:
i) administering to the patient an initiation dose comprising two tablets on day 1, and two tablets on day 2, wherein each tablet of the initiation dose comprises:
   about 400 mg of the lenacapavir spray dried dispersion;
   about 0.5325 mg of the compound of Formula IIa or IIb, or a pharmaceutically acceptable salt, co-crystal, solvate, or combination thereof;
   about 621 mg of mannitol;
   about 335.9175 mg of microcrystalline cellulose;
   about 120 mg croscarmellose sodium; and
   about 22.5 mg magnesium stearate; and
ii) administering to the patient a maintenance dose comprising one tablet, once weekly, beginning on day 8, wherein the tablet comprises:
   about 400 mg of the lenacapavir spray dried dispersion;
   about 2.130 mg of the compound of Formula IIa or IIb, or a pharmaceutically acceptable salt, co-crystal, solvate, or combination thereof;
   about 621 mg of mannitol;
   about 334.32 mg of microcrystalline cellulose;
   about 120 mg croscarmellose sodium; and
   about 22.5 mg magnesium stearate.

In some embodiments, the method comprises:
i) administering to the patient an initiation dose comprising two tablets on day 1, and two tablets on day 2, wherein each tablet of the initiation dose comprises:
   about 400 mg of the lenacapavir spray dried dispersion;
   about 0.5 mg of the compound of Formula IIa or IIb, or a pharmaceutically acceptable salt, co-crystal, solvate, or combination thereof,
on a free base basis;
   about 621 mg of mannitol;
   about 335.9175 mg of microcrystalline cellulose;
   about 120 mg croscarmellose sodium; and
   about 22.5 mg magnesium stearate; and
ii) administering to the patient a maintenance dose comprising one tablet, once weekly, beginning on day 8, wherein the tablet comprises:
   about 400 mg of the lenacapavir spray dried dispersion;
   about 2.0 mg of the compound of Formula IIa or IIb, or a pharmaceutically acceptable salt, co-crystal, solvate, or combination thereof,
on a free base basis;
   about 621 mg of mannitol;
   about 334.32 mg of microcrystalline cellulose;
   about 120 mg croscarmellose sodium; and
   about 22.5 mg magnesium stearate.

In some embodiments, the method comprises:
i) administering to the patient an initiation dose comprising two tablets on day 1, and two tablets on day 2, wherein each tablet of the initiation dose comprises:
   about 300 mg of the compound of Formula Ia or Ib, or a pharmaceutically acceptable salt, co-crystal, solvate, or combination thereof,
on a free acid basis; and
   about 0.5 mg to about 0.6 mg of the compound of Formula IIa or IIb, or a pharmaceutically acceptable salt, co-crystal, solvate, or combination thereof, and
ii) administering to the patient a maintenance dose comprising one tablet, once weekly, beginning on day 8, wherein the tablet comprises:
   about 300 mg of the compound of Formula Ia or Ib, or a pharmaceutically acceptable salt, co-crystal, solvate, or combination thereof,
on a free acid basis; and
   about 2.1 mg to about 2.2 mg of the compound of Formula IIa or IIb, or a pharmaceutically acceptable salt, co-crystal, solvate, or combination thereof.

In some embodiments, the method comprises:
i) administering to the patient an initiation dose comprising two tablets on day 1, and two tablets on day 2, wherein each tablet of the initiation dose comprises:
   about 300 mg of the compound of Formula Ia or Ib, or a pharmaceutically acceptable salt, co-crystal, solvate, or combination thereof,
on a free acid basis; and
   about 0.5325 mg of the compound of Formula IIa or IIb, or a pharmaceutically acceptable salt, co-crystal, solvate, or combination thereof; and
ii) administering to the patient a maintenance dose comprising one tablet, once weekly, beginning on day 8, wherein the tablet comprises:
   about 300 mg of the compound of Formula Ia or Ib, or a pharmaceutically acceptable salt, co-crystal, solvate, or combination thereof,
on a free acid basis; and
   about 2.130 mg of the compound of Formula IIa or IIb, or a pharmaceutically acceptable salt, co-crystal, solvate, or combination thereof.

In some embodiments, the method comprises:
i) administering to the patient an initiation dose comprising two tablets on day 1, and two tablets on day 2, wherein each tablet of the initiation dose comprises:
   about 300 mg of the compound of Formula Ia or Ib, or a pharmaceutically acceptable salt, co-crystal, solvate, or combination thereof,
on a free acid basis;
   about 0.5 mg of the compound of Formula IIa or IIb, or a pharmaceutically acceptable salt, co-crystal, solvate, or combination thereof,
on a free base basis; and
ii) administering to the patient a maintenance dose comprising one tablet, once weekly, beginning on day 8, wherein the tablet comprises:
   about 300 mg of the compound of Formula Ia or Ib, or a pharmaceutically acceptable salt, co-crystal, solvate, or combination thereof,
on a free acid basis; and
   about 2.0 mg of the compound of Formula IIa or IIb, or a pharmaceutically acceptable salt, co-crystal, solvate, or combination thereof,
on a free base basis.

In some embodiments, the method comprises:
i) administering to the patient an initiation dose comprising two tablets on day 1, and two tablets on day 2, wherein each tablet of the initiation dose comprises:
   about 300 mg of the compound of Formula Ia or Ib, or a pharmaceutically acceptable salt, co-crystal, solvate, or combination thereof,
on a free acid basis;
   about 0.5 mg to about 0.6 mg of the compound of Formula IIa or IIb, or a pharmaceutically acceptable salt, co-crystal, solvate, or combination thereof;
   about 615 mg to about 625 mg of mannitol;
   about 330 mg to about 340 mg of microcrystalline cellulose;
   about 115 mg to about 125 mg croscarmellose sodium; and
   about 20 mg to about 25 mg magnesium stearate; and
ii) administering to the patient a maintenance dose comprising one tablet, once weekly, beginning on day 8, wherein the tablet comprises:
   about 300 mg of the compound of Formula Ia or Ib, or a pharmaceutically acceptable salt, co-crystal, solvate, or combination thereof,
on a free acid basis;
   about 2.1 mg to about 2.2 mg of the compound of Formula IIa or IIb, or a pharmaceutically acceptable salt, co-crystal, solvate, or combination thereof;
   about 615 mg to about 625 mg of mannitol;
   about 330 mg to about 340 mg of microcrystalline cellulose;
   about 115 mg to about 125 mg croscarmellose sodium; and
   about 20 mg to about 25 mg magnesium stearate.

In some embodiments, the method comprises:
i) administering to the patient an initiation dose comprising two tablets on day 1, and two tablets on day 2, wherein each tablet of the initiation dose comprises:
   about 300 mg of the compound of Formula Ia or Ib, or a pharmaceutically acceptable salt, co-crystal, solvate, or combination thereof,
on a free acid basis;
   about 0.5325 mg of the compound of Formula IIa or IIb, or a pharmaceutically acceptable salt, co-crystal, solvate, or combination thereof;
   about 621 mg of mannitol;
   about 335.9175 mg of microcrystalline cellulose;
   about 120 mg croscarmellose sodium; and
   about 22.5 mg magnesium stearate; and
ii) administering to the patient a maintenance dose comprising one tablet, once weekly, beginning on day 8, wherein the tablet comprises:
   about 300 mg of the compound of Formula Ia or Ib, or a pharmaceutically acceptable salt, co-crystal, solvate, or combination thereof,
on a free acid basis;
   about 2.130 mg of the compound of Formula IIa or IIb, or a pharmaceutically acceptable salt, co-crystal, solvate, or combination thereof;
   about 621 mg of mannitol;
   about 334.32 mg of microcrystalline cellulose;
   about 120 mg croscarmellose sodium; and
   about 22.5 mg magnesium stearate.

In some embodiments, the method comprises:
i) administering to the patient an initiation dose comprising two tablets on day 1, and two tablets on day 2, wherein each tablet of the initiation dose comprises:
   about 300 mg of the compound of Formula Ia or Ib, or a pharmaceutically acceptable salt, co-crystal, solvate, or combination thereof,
on a free acid basis;
   about 0.5 mg of the compound of Formula IIa or IIb, or a pharmaceutically acceptable salt, co-crystal, solvate, or combination thereof,
on a free base basis;
   about 621 mg of mannitol;
   about 335.9175 mg of microcrystalline cellulose;
   about 120 mg croscarmellose sodium; and
   about 22.5 mg magnesium stearate; and
ii) administering to the patient a maintenance dose comprising one tablet, once weekly, beginning on day 8, wherein the tablet comprises:
   about 300 mg of the compound of Formula Ia or Ib, or a pharmaceutically acceptable salt, co-crystal, solvate, or combination thereof,
on a free acid basis;
   about 2.0 mg of the compound of Formula IIa or IIb, or a pharmaceutically acceptable salt, co-crystal, solvate, or combination thereof,
on a free base basis;
   about 621 mg of mannitol;
   about 334.32 mg of microcrystalline cellulose;
   about 120 mg croscarmellose sodium; and
   about 22.5 mg magnesium stearate.

In some embodiments, the methods provided herein comprise:
(i) administering to the patient an initiation dosage for a first period of time, as described herein;
(ii) administering to the patient one or more maintenance dosages, for a second period of time, as described herein, wherein the second period of time occurs after the first period of time;
wherein if the patient misses a maintenance dosage during the second period of time; and
wherein about 16 days or less have elapsed since administration of a prior maintenance dosage;
the method further comprises restarting administration of the one or more maintenance dosages of step (ii).

In some embodiments, if the patient misses a maintenance dosage during the second period of time; and
wherein about 15 days or less have elapsed since administration of a prior maintenance dosage;
the method further comprises restarting administration of the one or more maintenance dosages of step (ii).

In some embodiments, if the patient misses a maintenance dosage during the second period of time; and
wherein about 14 days or less have elapsed since administration of a prior maintenance dosage;
the method further comprises restarting administration of the one or more maintenance dosages of step (ii).

In some embodiments, if the patient misses a maintenance dosage during the second period of time; and
wherein about 13 days or less have elapsed since administration of a prior maintenance dosage;
the method further comprises restarting administration of the one or more maintenance dosages of step (ii).

In some embodiments, if the patient misses a maintenance dosage during the second period of time; and
wherein about 12 days or less have elapsed since administration of a prior maintenance dosage;
the method further comprises restarting administration of the one or more maintenance dosages of step (ii).

In some embodiments, if the patient misses a maintenance dosage during the second period of time; and
wherein about 11 days or less have elapsed since administration of a prior maintenance dosage;
the method further comprises restarting administration of the one or more maintenance dosages of step (ii).

In some embodiments, if the patient misses a maintenance dosage during the second period of time; and
wherein about 10 days or less have elapsed since administration of a prior maintenance dosage;
the method further comprises restarting administration of the one or more maintenance dosages of step (ii).

In some embodiments, if the patient misses a maintenance dosage during the second period of time; and
wherein about 9 days or less have elapsed since administration of a prior maintenance dosage;
the method further comprises restarting administration of the one or more maintenance dosages of step (ii).

In some embodiments, if the patient misses a maintenance dosage during the second period of time; and
wherein about 8 days or less have elapsed since administration of a prior maintenance dosage;
the method further comprises restarting administration of the one or more maintenance dosages of step (ii).

In some embodiments, if the patient misses a maintenance dosage during the second period of time; and
wherein about 7 days have elapsed since administration of a prior maintenance dosage;
the method further comprises restarting administration of the one or more maintenance dosages of step (ii).

In some embodiments, the methods provided herein comprise:
(i) administering to the patient an initiation dosage for a first period of time, as described herein;
(ii) administering to the patient one or more maintenance dosages, for a second period of time, as described herein, wherein the second period of time occurs after the first period of time;
wherein if the patient misses a maintenance dosage during the second period of time; and
wherein between about 7 days to about 16 days have elapsed since administration of a prior maintenance dosage;
the method further comprises restarting administration of the one or more maintenance dosages of step (ii).

In some embodiments, the method comprises:
i) administering to the patient an initiation dose comprising two tablets on day 1, and two tablets on day 2, wherein each tablet of the initiation dose comprises:
   about 26.6 w/w % to about 26.7 w/w % of the spray dried dispersion of the compound of Formula Ia or Ib, or a pharmaceutically acceptable salt, co-crystal, solvate, or combination thereof;
   about 0.03 w/w % to about 0.04 w/w % of the compound of Formula IIa or IIb, or a pharmaceutically acceptable salt, co-crystal, solvate, or combination thereof, and
   one or more pharmaceutically acceptable excipients; and
ii) administering to the patient a maintenance dose comprising one tablet, once weekly, beginning on day 8, wherein the tablet comprises:
   about 26.6 w/w % to about 26.7 w/w % of the spray dried dispersion of the compound of Formula Ia or Ib, or a pharmaceutically acceptable salt, co-crystal, solvate, or combination thereof;
   about 0.14 w/w % to about 0.15 w/w % of the compound of Formula IIa or IIb, or a pharmaceutically acceptable salt, co-crystal, solvate, or combination thereof; and
   one or more pharmaceutically acceptable excipients;
wherein if the patient misses a maintenance dosage; and
wherein between about 7 days to about 16 days have elapsed since administration of a prior maintenance dosage;
the method further comprises restarting administration of the one or more maintenance dosages of step (ii).

In some embodiments, the method comprises:
i) administering to the patient an initiation dose comprising two tablets on day 1, and two tablets on day 2, wherein each tablet of the initiation dose comprises:
   about 26.67 w/w % of the spray dried dispersion of the compound of Formula Ia or Ib, or a pharmaceutically acceptable salt, co-crystal, solvate, or combination thereof;
   about 0.0355 w/w % of the compound of Formula IIa or IIb, or a pharmaceutically acceptable salt, co-crystal, solvate, or combination thereof; and
   one or more pharmaceutically acceptable excipients; and
ii) administering to the patient a maintenance dose comprising one tablet, once weekly, beginning on day 8, wherein the tablet comprises:
   about 26.67 w/w % of the spray dried dispersion of the compound of Formula Ia or Ib, or a pharmaceutically acceptable salt, co-crystal, solvate, or combination thereof;
   about 0.142 w/w % of the compound of Formula IIa or IIb, or a pharmaceutically acceptable salt, co-crystal, solvate, or combination thereof; and
   one or more pharmaceutically acceptable excipients;
wherein if the patient misses a maintenance dosage; and
wherein between about 7 days to about 16 days have elapsed since administration of a prior maintenance dosage;
the method further comprises restarting administration of the one or more maintenance dosages of step (ii).

In some embodiments, the method comprises:
i) administering to the patient an initiation dose comprising two tablets on day 1, and two tablets on day 2, wherein each tablet of the initiation dose comprises:
   about 26.6 w/w % to about 26.7 w/w % of the spray dried dispersion of the compound of Formula Ia or Ib, or a pharmaceutically acceptable salt, co-crystal, solvate, or combination thereof;
   about 0.03 w/w % to about 0.04 w/w % of the compound of Formula IIa or IIb, or a solvate thereof; and
   one or more pharmaceutically acceptable excipients; and
ii) administering to the patient a maintenance dose comprising one tablet, once weekly, beginning on day 8, wherein the tablet comprises:
   about 26.6 w/w % to about 26.7 w/w % of the spray dried dispersion of the compound of Formula Ia or Ib, or a pharmaceutically acceptable salt, co-crystal, solvate, or combination thereof;
   about 0.14 w/w % to about 0.15 w/w % of the compound of Formula IIa or IIb, or a solvate thereof; and
   one or more pharmaceutically acceptable excipients;
wherein if the patient misses a maintenance dosage; and
wherein between about 7 days to about 16 days have elapsed since administration of a prior maintenance dosage;
the method further comprises restarting administration of the one or more maintenance dosages of step (ii).

In some embodiments, the method comprises:
i) administering to the patient an initiation dose comprising two tablets on day 1, and two tablets on day 2, wherein each tablet of the initiation dose comprises:
   about 26.67 w/w % of the spray dried dispersion of the compound of Formula Ia or Ib, or a pharmaceutically acceptable salt, co-crystal, solvate, or combination thereof;
   about 0.0355 w/w % of the compound of Formula IIa or IIb, or a solvate thereof, and
   one or more pharmaceutically acceptable excipients; and
ii) administering to the patient a maintenance dose comprising one tablet, once weekly, beginning on day 8, wherein the tablet comprises:
   about 26.67 w/w % of the spray dried dispersion of the compound of Formula Ia or Ib, or a pharmaceutically acceptable salt, co-crystal, solvate, or combination thereof;
   about 0.142 w/w % of the compound of Formula IIa or IIb, or a solvate thereof, and
   one or more pharmaceutically acceptable excipients;
wherein if the patient misses a maintenance dosage; and
wherein between about 7 days to about 16 days have elapsed since administration of a prior maintenance dosage;
the method further comprises restarting administration of the one or more maintenance dosages of step (ii).

In some embodiments, the method comprises:
i) administering to the patient an initiation dose comprising two tablets on day 1, and two tablets on day 2, wherein each tablet of the initiation dose comprises:
   about 26.6 w/w % to about 26.7 w/w % of the spray dried dispersion of the compound of Formula Ia or Ib, or a pharmaceutically acceptable salt, co-crystal, solvate, or combination thereof;
   about 0.03 w/w % to about 0.04 w/w % of the monohydrate of the compound of Formula IIa or IIb; and
   one or more pharmaceutically acceptable excipients; and
ii) administering to the patient a maintenance dose comprising one tablet, once weekly, beginning on day 8, wherein the tablet comprises:
   about 26.6 w/w % to about 26.7 w/w % of the spray dried dispersion of the compound of Formula Ia or Ib, or a pharmaceutically acceptable salt, co-crystal, solvate, or combination thereof;
   about 0.14 w/w % to about 0.15 w/w % of the monohydrate of the compound of Formula IIa or IIb; and
   one or more pharmaceutically acceptable excipients;
wherein if the patient misses a maintenance dosage; and
wherein between about 7 days to about 16 days have elapsed since administration of a prior maintenance dosage;
the method further comprises restarting administration of the one or more maintenance dosages of step (ii).

In some embodiments, the method comprises:
i) administering to the patient an initiation dose comprising two tablets on day 1, and two tablets on day 2, wherein each tablet of the initiation dose comprises:
   about 26.67 w/w % of the spray dried dispersion of the compound of Formula Ia or Ib, or a pharmaceutically acceptable salt, co-crystal, solvate, or combination thereof;
   about 0.0355 w/w % of the monohydrate of the compound of Formula IIa or IIb; and
   one or more pharmaceutically acceptable excipients; and
ii) administering to the patient a maintenance dose comprising one tablet, once weekly, beginning on day 8, wherein the tablet comprises:
   about 26.67 w/w % of the spray dried dispersion of the compound of Formula Ia or Ib, or a pharmaceutically acceptable salt, co-crystal, solvate, or combination thereof;
   about 0.142 w/w % of the monohydrate of the compound of Formula IIa or IIb; and
   one or more pharmaceutically acceptable excipients;
wherein if the patient misses a maintenance dosage; and
wherein between about 7 days to about 16 days have elapsed since administration of a prior maintenance dosage;
the method further comprises restarting administration of the one or more maintenance dosages of step (ii).

In some embodiments, the method comprises:
i) administering to the patient an initiation dose comprising two tablets on day 1, and two tablets on day 2, wherein each tablet of the initiation dose comprises:
   about 26.6 w/w % to about 26.7 w/w % of the spray dried dispersion of the compound of Formula Ia or Ib, or a pharmaceutically acceptable salt, co-crystal, solvate, or combination thereof;
   about 0.03 w/w % to about 0.04 w/w % of the compound of Formula IIa or IIb, or a pharmaceutically acceptable salt, co-crystal, solvate, or combination thereof;
   about 41 w/w % to about 42 w/w % of mannitol;
   about 22.3 w/w % to about 22.4 w/w % of microcrystalline cellulose;
   about 7.9 w/w % to about 8.1 w/w % of croscarmellose sodium; and
   about 1.4 w/w % to about 1.6 w/w % of magnesium stearate; and
ii) administering to the patient a maintenance dose comprising one tablet, once weekly, beginning on day 8, wherein the tablet comprises:
   about 26.6 w/w % to about 26.7 w/w % of the spray dried dispersion of the compound of Formula Ia or Ib, or a pharmaceutically acceptable salt, co-crystal, solvate, or combination thereof;
   about 0.14 w/w % to about 0.15 w/w % of the compound of Formula IIa or IIb, or a pharmaceutically acceptable salt, co-crystal, solvate, or combination thereof;
   about 41 w/w % to about 42 w/w % of mannitol;
   about 22.2 w/w % to about 22.3 w/w % of microcrystalline cellulose;
   about 7.9 w/w % to about 8.1 w/w % of croscarmellose sodium; and
   about 1.4 w/w % to about 1.6 w/w % of magnesium stearate;
wherein if the patient misses a maintenance dosage; and
wherein between about 7 days to about 16 days have elapsed since administration of a prior maintenance dosage;
the method further comprises restarting administration of the one or more maintenance dosages of step (ii).

In some embodiments, the method comprises:
i) administering to the patient an initiation dose comprising two tablets on day 1, and two tablets on day 2, wherein each tablet of the initiation dose comprises:
   about 26.67 w/w % of the spray dried dispersion of the compound of Formula Ia or Ib, or a pharmaceutically acceptable salt, co-crystal, solvate, or combination thereof;
   about 0.0355 w/w % of the compound of Formula IIa or IIb, or a pharmaceutically acceptable salt, co-crystal, solvate, or combination thereof;
   about 41.40 w/w % of mannitol;
   about 22.3945 w/w % of microcrystalline cellulose;
   about 8.0 w/w % of croscarmellose sodium; and
   about 1.5 w/w % of magnesium stearate; and
ii) administering to the patient a maintenance dose comprising one tablet, once weekly, beginning on day 8, wherein the tablet comprises:
   about 26.67 w/w % of the spray dried dispersion of the compound of Formula Ia or Ib, or a pharmaceutically acceptable salt, co-crystal, solvate, or combination thereof;
   about 0.142 w/w % of the compound of Formula IIa or IIb, or a pharmaceutically acceptable salt, co-crystal, solvate, or combination thereof;
   about 41.40 w/w % of mannitol;
   about 22.288 w/w % microcrystalline cellulose;
   about 8.0 w/w % of croscarmellose sodium; and
   about 1.5 w/w % of magnesium stearate;
wherein if the patient misses a maintenance dosage; and
wherein between about 7 days to about 16 days have elapsed since administration of a prior maintenance dosage;
the method further comprises restarting administration of the one or more maintenance dosages of step (ii).

In some embodiments, the method comprises:
i) administering to the patient an initiation dose comprising two tablets on day 1, and two tablets on day 2, wherein each tablet of the initiation dose comprises:
   about 400 mg of the spray dried dispersion of the compound of Formula Ia or Ib, or a pharmaceutically acceptable salt, co-crystal, solvate, or combination thereof, and
about 0.5 mg to about 0.6 mg of the compound of Formula IIa or IIb, or a pharmaceutically acceptable salt, co-crystal, solvate, or combination thereof, and
ii) administering to the patient a maintenance dose comprising one tablet, once weekly, beginning on day 8, wherein the tablet comprises:
   about 400 mg of the spray dried dispersion of the compound of Formula Ia or Ib, or a pharmaceutically acceptable salt, co-crystal, solvate, or combination thereof, and
about 2.1 mg to about 2.2 mg of the compound of Formula IIa or IIb, or a pharmaceutically acceptable salt, co-crystal, solvate, or combination thereof;
wherein if the patient misses a maintenance dosage; and
wherein between about 7 days to about 16 days have elapsed since administration of a prior maintenance dosage;
the method further comprises restarting administration of the one or more maintenance dosages of step (ii).

In some embodiments, the method comprises:
i) administering to the patient an initiation dose comprising two tablets on day 1, and two tablets on day 2, wherein each tablet of the initiation dose comprises:
   about 400 mg of the spray dried dispersion of the compound of Formula Ia or Ib, or a pharmaceutically acceptable salt, co-crystal, solvate, or combination thereof, and
   about 0.5325 mg of the compound of Formula IIa or IIb, or a pharmaceutically acceptable salt, co-crystal, solvate, or combination thereof; and
ii) administering to the patient a maintenance dose comprising one tablet, once weekly, beginning on day 8, wherein the tablet comprises:
   about 400 mg of the spray dried dispersion of the compound of Formula Ia or Ib, or a pharmaceutically acceptable salt, co-crystal, solvate, or combination thereof; and
   about 2.130 mg of the compound of Formula IIa or IIb, or a pharmaceutically acceptable salt, co-crystal, solvate, or combination thereof;
wherein if the patient misses a maintenance dosage; and
wherein between about 7 days to about 16 days have elapsed since administration of a prior maintenance dosage;
the method further comprises restarting administration of the one or more maintenance dosages of step (ii).

In some embodiments, the method comprises:
i) administering to the patient an initiation dose comprising two tablets on day 1, and two tablets on day 2, wherein each tablet of the initiation dose comprises:
   about 400 mg of the spray dried dispersion of the compound of Formula Ia or Ib, or a pharmaceutically acceptable salt, co-crystal, solvate, or combination thereof, and
   about 0.5 mg of the compound of Formula IIa or IIb, or a pharmaceutically acceptable salt, co-crystal, solvate, or combination thereof,
on a free base basis; and
ii) administering to the patient a maintenance dose comprising one tablet, once weekly, beginning on day 8, wherein the tablet comprises:
   about 400 mg of the spray dried dispersion of the compound of Formula Ia or Ib, or a pharmaceutically acceptable salt, co-crystal, solvate, or combination thereof, and
   about 2.0 mg of the compound of Formula IIa or IIb, or a pharmaceutically acceptable salt, co-crystal, solvate, or combination thereof,
on a free base basis;
wherein if the patient misses a maintenance dosage; and
wherein between about 7 days to about 16 days have elapsed since administration of a prior maintenance dosage;
the method further comprises restarting administration of the one or more maintenance dosages of step (ii).

In some embodiments, the method comprises:
i) administering to the patient an initiation dose comprising two tablets on day 1, and two tablets on day 2, wherein each tablet of the initiation dose comprises:
   about 400 mg of the spray dried dispersion of the compound of Formula Ia or Ib, or a pharmaceutically acceptable salt, co-crystal, solvate, or combination thereof;
   about 0.5 mg to about 0.6 mg of the compound of Formula IIa or IIb, or a pharmaceutically acceptable salt, co-crystal, solvate, or combination thereof;
   about 615 mg to about 625 mg of mannitol;
   about 330 mg to about 340 mg of microcrystalline cellulose;
   about 115 mg to about 125 mg croscarmellose sodium; and
   about 20 mg to about 25 mg magnesium stearate; and
ii) administering to the patient a maintenance dose comprising one tablet, once weekly, beginning on day 8, wherein the tablet comprises:
   about 400 mg of the spray dried dispersion of the compound of Formula Ia or Ib, or a pharmaceutically acceptable salt, co-crystal, solvate, or combination thereof;
   about 2.1 mg to about 2.2 mg of the compound of Formula IIa or IIb, or a pharmaceutically acceptable salt, co-crystal, solvate, or combination thereof;
   about 615 mg to about 625 mg of mannitol;
   about 330 mg to about 340 mg of microcrystalline cellulose;
   about 115 mg to about 125 mg croscarmellose sodium; and
   about 20 mg to about 25 mg magnesium stearate;
wherein if the patient misses a maintenance dosage; and
wherein between about 7 days to about 16 days have elapsed since administration of a prior maintenance dosage;
the method further comprises restarting administration of the one or more maintenance dosages of step (ii).

In some embodiments, the method comprises:
i) administering to the patient an initiation dose comprising two tablets on day 1, and two tablets on day 2, wherein each tablet of the initiation dose comprises:
   about 400 mg of the spray dried dispersion of the compound of Formula Ia or Ib, or a pharmaceutically acceptable salt, co-crystal, solvate, or combination thereof;
   about 0.5325 mg of the compound of Formula IIa or IIb, or a pharmaceutically acceptable salt, co-crystal, solvate, or combination thereof;
   about 621 mg of mannitol;
   about 335.9175 mg of microcrystalline cellulose;
   about 120 mg croscarmellose sodium; and
   about 22.5 mg magnesium stearate; and
ii) administering to the patient a maintenance dose comprising one tablet, once weekly, beginning on day 8, wherein the tablet comprises:
   about 400 mg of the spray dried dispersion of the compound of Formula Ia or Ib, or a pharmaceutically acceptable salt, co-crystal, solvate, or combination thereof;
   about 2.130 mg of the compound of Formula IIa or IIb, or a pharmaceutically acceptable salt, co-crystal, solvate, or combination thereof;
   about 621 mg of mannitol;
   about 334.32 mg of microcrystalline cellulose;
   about 120 mg croscarmellose sodium; and
   about 22.5 mg magnesium stearate;
wherein if the patient misses a maintenance dosage; and
wherein between about 7 days to about 16 days have elapsed since administration of a prior maintenance dosage;
the method further comprises restarting administration of the one or more maintenance dosages of step (ii).

In some embodiments, the method comprises:
i) administering to the patient an initiation dose comprising two tablets on day 1, and two tablets on day 2, wherein each tablet of the initiation dose comprises:
   about 400 mg of the spray dried dispersion of the compound of Formula Ia or Ib, or a pharmaceutically acceptable salt, co-crystal, solvate, or combination thereof;
   about 0.5 mg of the compound of Formula IIa or IIb, or a pharmaceutically acceptable salt, co-crystal, solvate, or combination thereof,
on a free base basis;
   about 621 mg of mannitol;
   about 335.9175 mg of microcrystalline cellulose;
   about 120 mg croscarmellose sodium; and
   about 22.5 mg magnesium stearate; and
ii) administering to the patient a maintenance dose comprising one tablet, once weekly, beginning on day 8, wherein the tablet comprises:
   about 400 mg of the spray dried dispersion of the compound of Formula Ia or Ib, or a pharmaceutically acceptable salt, co-crystal, solvate, or combination thereof;
   about 2.0 mg of the compound of Formula IIa or IIb, or a pharmaceutically acceptable salt, co-crystal, solvate, or combination thereof,
on a free base basis;
   about 621 mg of mannitol;
   about 334.32 mg of microcrystalline cellulose;
   about 120 mg croscarmellose sodium; and
   about 22.5 mg magnesium stearate;
wherein if the patient misses a maintenance dosage; and
wherein between about 7 days to about 16 days have elapsed since administration of a prior maintenance dosage;
the method further comprises restarting administration of the one or more maintenance dosages of step (ii).

In some embodiments, the method comprises:
i) administering to the patient an initiation dose comprising two tablets on day 1, and two tablets on day 2, wherein each tablet of the initiation dose comprises:
   about 26.6 w/w % to about 26.7 w/w % of the spray dried dispersion of the compound of Formula Ia or Ib, or a pharmaceutically acceptable salt thereof;
   about 0.03 w/w % to about 0.04 w/w % of the compound of Formula IIa or IIb, or a pharmaceutically acceptable salt, co-crystal, solvate, or combination thereof, and
   one or more pharmaceutically acceptable excipients; and
ii) administering to the patient a maintenance dose comprising one tablet, once weekly, beginning on day 8, wherein the tablet comprises:
   about 26.6 w/w % to about 26.7 w/w % of the spray dried dispersion of the compound of Formula Ia or Ib, or a pharmaceutically acceptable salt thereof;
   about 0.14 w/w % to about 0.15 w/w % of the compound of Formula IIa or IIb, or a pharmaceutically acceptable salt, co-crystal, solvate, or combination thereof, and
   one or more pharmaceutically acceptable excipients;
wherein if the patient misses a maintenance dosage; and
wherein between about 7 days to about 16 days have elapsed since administration of a prior maintenance dosage;
the method further comprises restarting administration of the one or more maintenance dosages of step (ii).

In some embodiments, the method comprises:
i) administering to the patient an initiation dose comprising two tablets on day 1, and two tablets on day 2, wherein each tablet of the initiation dose comprises:
   about 26.67 w/w % of the spray dried dispersion of the compound of Formula Ia or Ib, or a pharmaceutically acceptable salt thereof;
   about 0.0355 w/w % of the compound of Formula IIa or IIb, or a pharmaceutically acceptable salt, co-crystal, solvate, or combination thereof; and
   one or more pharmaceutically acceptable excipients; and
ii) administering to the patient a maintenance dose comprising one tablet, once weekly, beginning on day 8, wherein the tablet comprises:
   about 26.67 w/w % of the spray dried dispersion of the compound of Formula Ia or Ib, or a pharmaceutically acceptable salt thereof;
   about 0.142 w/w % of the compound of Formula IIa or IIb, or a pharmaceutically acceptable salt, co-crystal, solvate, or combination thereof; and
   one or more pharmaceutically acceptable excipients;
wherein if the patient misses a maintenance dosage; and
wherein between about 7 days to about 16 days have elapsed since administration of a prior maintenance dosage;
the method further comprises restarting administration of the one or more maintenance dosages of step (ii).

In some embodiments, the method comprises:
i) administering to the patient an initiation dose comprising two tablets on day 1, and two tablets on day 2, wherein each tablet of the initiation dose comprises:
   about 26.6 w/w % to about 26.7 w/w % of the spray dried dispersion of the compound of Formula Ia or Ib, or a pharmaceutically acceptable salt thereof;
   about 0.03 w/w % to about 0.04 w/w % of the compound of Formula IIa or IIb, or a solvate thereof; and
   one or more pharmaceutically acceptable excipients; and
ii) administering to the patient a maintenance dose comprising one tablet, once weekly, beginning on day 8, wherein the tablet comprises:
   about 26.6 w/w % to about 26.7 w/w % of the spray dried dispersion of the compound of Formula Ia or Ib, or a pharmaceutically acceptable salt thereof;
   about 0.14 w/w % to about 0.15 w/w % of the compound of Formula IIa or IIb, or a solvate thereof; and
   one or more pharmaceutically acceptable excipients;
wherein if the patient misses a maintenance dosage; and
wherein between about 7 days to about 16 days have elapsed since administration of a prior maintenance dosage;
the method further comprises restarting administration of the one or more maintenance dosages of step (ii).

In some embodiments, the method comprises:
i) administering to the patient an initiation dose comprising two tablets on day 1, and two tablets on day 2, wherein each tablet of the initiation dose comprises:
   about 26.67 w/w % of the spray dried dispersion of the compound of Formula Ia or Ib, or a pharmaceutically acceptable salt thereof;
   about 0.0355 w/w % of the compound of Formula IIa or IIb, or a solvate thereof, and
   one or more pharmaceutically acceptable excipients; and
ii) administering to the patient a maintenance dose comprising one tablet, once weekly, beginning on day 8, wherein the tablet comprises:
   about 26.67 w/w % of the spray dried dispersion of the compound of Formula Ia or Ib, or a pharmaceutically acceptable salt thereof;
   about 0.142 w/w % of the compound of Formula IIa or IIb, or a solvate thereof, and
   one or more pharmaceutically acceptable excipients;
wherein if the patient misses a maintenance dosage; and
wherein between about 7 days to about 16 days have elapsed since administration of a prior maintenance dosage;
the method further comprises restarting administration of the one or more maintenance dosages of step (ii).

In some embodiments, the method comprises:
i) administering to the patient an initiation dose comprising two tablets on day 1, and two tablets on day 2, wherein each tablet of the initiation dose comprises:
   about 26.6 w/w % to about 26.7 w/w % of the spray dried dispersion of the compound of Formula Ia or Ib, or a pharmaceutically acceptable salt thereof;
   about 0.03 w/w % to about 0.04 w/w % of the monohydrate of the compound of Formula IIa or IIb; and
   one or more pharmaceutically acceptable excipients; and
ii) administering to the patient a maintenance dose comprising one tablet, once weekly, beginning on day 8, wherein the tablet comprises:
   about 26.6 w/w % to about 26.7 w/w % of the spray dried dispersion of the compound of Formula Ia or Ib, or a pharmaceutically acceptable salt thereof;
   about 0.14 w/w % to about 0.15 w/w % of the monohydrate of the compound of Formula IIa or IIb; and
   one or more pharmaceutically acceptable excipients;
wherein if the patient misses a maintenance dosage; and
wherein between about 7 days to about 16 days have elapsed since administration of a prior maintenance dosage;
the method further comprises restarting administration of the one or more maintenance dosages of step (ii).

In some embodiments, the method comprises:
i) administering to the patient an initiation dose comprising two tablets on day 1, and two tablets on day 2, wherein each tablet of the initiation dose comprises:
   about 26.67 w/w % of the spray dried dispersion of the compound of Formula Ia or Ib, or a pharmaceutically acceptable salt thereof;
   about 0.0355 w/w % of the monohydrate of the compound of Formula IIa or IIb; and
   one or more pharmaceutically acceptable excipients; and
ii) administering to the patient a maintenance dose comprising one tablet, once weekly, beginning on day 8, wherein the tablet comprises:
   about 26.67 w/w % of the spray dried dispersion of the compound of Formula Ia or Ib, or a pharmaceutically acceptable salt thereof;
   about 0.142 w/w % of the monohydrate of the compound of Formula IIa or IIb; and
   one or more pharmaceutically acceptable excipients;
wherein if the patient misses a maintenance dosage; and
wherein between about 7 days to about 16 days have elapsed since administration of a prior maintenance dosage;
the method further comprises restarting administration of the one or more maintenance dosages of step (ii).

In some embodiments, the method comprises:
i) administering to the patient an initiation dose comprising two tablets on day 1, and two tablets on day 2, wherein each tablet of the initiation dose comprises:
   about 26.6 w/w % to about 26.7 w/w % of the spray dried dispersion of the compound of Formula Ia or Ib, or a pharmaceutically acceptable salt thereof;
   about 0.03 w/w % to about 0.04 w/w % of the compound of Formula IIa or IIb, or a pharmaceutically acceptable salt, co-crystal, solvate, or combination thereof;
   about 41 w/w % to about 42 w/w % of mannitol;
   about 22.3 w/w % to about 22.4 w/w % of microcrystalline cellulose;
   about 7.9 w/w % to about 8.1 w/w % of croscarmellose sodium; and
   about 1.4 w/w % to about 1.6 w/w % of magnesium stearate; and
ii) administering to the patient a maintenance dose comprising one tablet, once weekly, beginning on day 8, wherein the tablet comprises:
   about 26.6 w/w % to about 26.7 w/w % of the spray dried dispersion of the compound of Formula Ia or Ib, or a pharmaceutically acceptable salt thereof;
   about 0.14 w/w % to about 0.15 w/w % of the compound of Formula IIa or IIb, or a pharmaceutically acceptable salt, co-crystal, solvate, or combination thereof;
   about 41 w/w % to about 42 w/w % of mannitol;
   about 22.2 w/w % to about 22.3 w/w % of microcrystalline cellulose;
   about 7.9 w/w % to about 8.1 w/w % of croscarmellose sodium; and
   about 1.4 w/w % to about 1.6 w/w % of magnesium stearate;
wherein if the patient misses a maintenance dosage; and
wherein between about 7 days to about 16 days have elapsed since administration of a prior maintenance dosage;
the method further comprises restarting administration of the one or more maintenance dosages of step (ii).

In some embodiments, the method comprises:
i) administering to the patient an initiation dose comprising two tablets on day 1, and two tablets on day 2, wherein each tablet of the initiation dose comprises:
   about 26.67 w/w % of the spray dried dispersion of the compound of Formula Ia or Ib, or a pharmaceutically acceptable salt thereof;
   about 0.0355 w/w % of the compound of Formula IIa or IIb, or a pharmaceutically acceptable salt, co-crystal, solvate, or combination thereof;
   about 41.40 w/w % of mannitol;
   about 22.3945 w/w % of microcrystalline cellulose;
   about 8.0 w/w % of croscarmellose sodium; and
   about 1.5 w/w % of magnesium stearate; and
ii) administering to the patient a maintenance dose comprising one tablet, once weekly, beginning on day 8, wherein the tablet comprises:
   about 26.67 w/w % of the spray dried dispersion of the compound of Formula Ia or Ib, or a pharmaceutically acceptable salt thereof;
   about 0.142 w/w % of the compound of Formula IIa or IIb, or a pharmaceutically acceptable salt, co-crystal, solvate, or combination thereof;
   about 41.40 w/w % of mannitol;
   about 22.288 w/w % microcrystalline cellulose;
   about 8.0 w/w % of croscarmellose sodium; and
   about 1.5 w/w % of magnesium stearate;
wherein if the patient misses a maintenance dosage; and
wherein between about 7 days to about 16 days have elapsed since administration of a prior maintenance dosage;
the method further comprises restarting administration of the one or more maintenance dosages of step (ii).

In some embodiments, the method comprises:
i) administering to the patient an initiation dose comprising two tablets on day 1, and two tablets on day 2, wherein each tablet of the initiation dose comprises:
   about 400 mg of the spray dried dispersion of the compound of Formula Ia or Ib, or a pharmaceutically acceptable salt thereof; and
   about 0.5 mg to about 0.6 mg of the compound of Formula IIa or IIb, or a pharmaceutically acceptable salt, co-crystal, solvate, or combination thereof, and
ii) administering to the patient a maintenance dose comprising one tablet, once weekly, beginning on day 8, wherein the tablet comprises:
   about 400 mg of the spray dried dispersion of the compound of Formula Ia or Ib, or a pharmaceutically acceptable salt thereof; and
   about 2.1 mg to about 2.2 mg of the compound of Formula IIa or IIb, or a pharmaceutically acceptable salt, co-crystal, solvate, or combination thereof;
wherein if the patient misses a maintenance dosage; and
wherein between about 7 days to about 16 days have elapsed since administration of a prior maintenance dosage;
the method further comprises restarting administration of the one or more maintenance dosages of step (ii).

In some embodiments, the method comprises:
i) administering to the patient an initiation dose comprising two tablets on day 1, and two tablets on day 2, wherein each tablet of the initiation dose comprises:
   about 400 mg of the spray dried dispersion of the compound of Formula Ia or Ib, or a pharmaceutically acceptable salt thereof; and
   about 0.5325 mg of the compound of Formula IIa or IIb, or a pharmaceutically acceptable salt, co-crystal, solvate, or combination thereof; and
ii) administering to the patient a maintenance dose comprising one tablet, once weekly, beginning on day 8, wherein the tablet comprises:
   about 400 mg of the spray dried dispersion of the compound of Formula Ia or Ib, or a pharmaceutically acceptable salt thereof; and
   about 2.130 mg of the compound of Formula IIa or IIb, or a pharmaceutically acceptable salt, co-crystal, solvate, or combination thereof;
wherein if the patient misses a once weekly maintenance dosage; and
wherein between about 7 days to about 16 days have elapsed since administration of a prior maintenance dosage;
the method further comprises restarting administration of the one or more maintenance dosages of step (ii).

In some embodiments, the method comprises:
i) administering to the patient an initiation dose comprising two tablets on day 1, and two tablets on day 2, wherein each tablet of the initiation dose comprises:
   about 400 mg of the spray dried dispersion of the compound of Formula Ia or Ib, or a pharmaceutically acceptable salt thereof; and
   about 0.5 mg of the compound of Formula IIa or IIb, or a pharmaceutically acceptable salt, co-crystal, solvate, or combination thereof,
on a free base basis; and
ii) administering to the patient a maintenance dose comprising one tablet, once weekly, beginning on day 8, wherein the tablet comprises:
   about 400 mg of the spray dried dispersion of the compound of Formula Ia or Ib, or a pharmaceutically acceptable salt thereof, and
   about 2.0 mg of the compound of Formula IIa or IIb, or a pharmaceutically acceptable salt, co-crystal, solvate, or combination thereof,
on a free base basis;
wherein if the patient misses a maintenance dosage; and
wherein between about 7 days to about 16 days have elapsed since administration of a prior maintenance dosage;
the method further comprises restarting administration of the one or more maintenance dosages of step (ii).

In some embodiments, the method comprises:
i) administering to the patient an initiation dose comprising two tablets on day 1, and two tablets on day 2, wherein each tablet of the initiation dose comprises:
   about 400 mg of the spray dried dispersion of the compound of Formula Ia or Ib, or a pharmaceutically acceptable salt thereof;
   about 0.5 mg to about 0.6 mg of the compound of Formula IIa or IIb, or a pharmaceutically acceptable salt, co-crystal, solvate, or combination thereof;
   about 615 mg to about 625 mg of mannitol;
   about 330 mg to about 340 mg of microcrystalline cellulose;
   about 115 mg to about 125 mg croscarmellose sodium; and
   about 20 mg to about 25 mg magnesium stearate; and
ii) administering to the patient a maintenance dose comprising one tablet, once weekly, beginning on day 8, wherein the tablet comprises:
   about 400 mg of the spray dried dispersion of the compound of Formula Ia or Ib, or a pharmaceutically acceptable salt thereof;
   about 2.1 mg to about 2.2 mg of the compound of Formula IIa or IIb, or a pharmaceutically acceptable salt, co-crystal, solvate, or combination thereof;
   about 615 mg to about 625 mg of mannitol;
   about 330 mg to about 340 mg of microcrystalline cellulose;
   about 115 mg to about 125 mg croscarmellose sodium; and
   about 20 mg to about 25 mg magnesium stearate;
wherein if the patient misses a maintenance dosage; and
wherein between about 7 days to about 16 days have elapsed since administration of a prior maintenance dosage;
the method further comprises restarting administration of the one or more maintenance dosages of step (ii).

In some embodiments, the method comprises:
i) administering to the patient an initiation dose comprising two tablets on day 1, and two tablets on day 2, wherein each tablet of the initiation dose comprises:
   about 400 mg of the spray dried dispersion of the compound of Formula Ia or Ib, or a pharmaceutically acceptable salt thereof;
   about 0.5325 mg of the compound of Formula IIa or IIb, or a pharmaceutically acceptable salt, co-crystal, solvate, or combination thereof;
   about 621 mg of mannitol;
   about 335.9175 mg of microcrystalline cellulose;
   about 120 mg croscarmellose sodium; and
   about 22.5 mg magnesium stearate; and
ii) administering to the patient a maintenance dose comprising one tablet, once weekly, beginning on day 8, wherein the tablet comprises:
   about 400 mg of the spray dried dispersion of the compound of Formula Ia or Ib, or a pharmaceutically acceptable salt thereof;
   about 2.130 mg of the compound of Formula IIa or IIb, or a pharmaceutically acceptable salt, co-crystal, solvate, or combination thereof;
   about 621 mg of mannitol;
   about 334.32 mg of microcrystalline cellulose;
   about 120 mg croscarmellose sodium; and
   about 22.5 mg magnesium stearate;
wherein if the patient misses a maintenance dosage;
wherein between about 7 days to about 16 days have elapsed since administration of a prior maintenance dosage;
the method further comprises restarting administration of the one or more maintenance dosages of step (ii).

In some embodiments, the method comprises:
i) administering to the patient an initiation dose comprising two tablets on day 1, and two tablets on day 2, wherein each tablet of the initiation dose comprises:
   about 400 mg of the spray dried dispersion of the compound of Formula Ia or Ib, or a pharmaceutically acceptable salt thereof;
   about 0.5 mg of the compound of Formula IIa or IIb, or a pharmaceutically acceptable salt, co-crystal, solvate, or combination thereof,
on a free base basis;
   about 621 mg of mannitol;
   about 335.9175 mg of microcrystalline cellulose;
   about 120 mg croscarmellose sodium; and
   about 22.5 mg magnesium stearate; and
ii) administering to the patient a maintenance dose comprising one tablet, once weekly, beginning on day 8, wherein the tablet comprises:
   about 400 mg of the spray dried dispersion of the compound of Formula Ia or Ib, or a pharmaceutically acceptable salt thereof;
   about 2.0 mg of the compound of Formula IIa or IIb, or a pharmaceutically acceptable salt, co-crystal, solvate, or combination thereof,
on a free base basis;
   about 621 mg of mannitol;
   about 334.32 mg of microcrystalline cellulose;
   about 120 mg croscarmellose sodium; and
   about 22.5 mg magnesium stearate;
wherein if the patient misses a maintenance dosage; and
wherein between about 7 days to about 16 days have elapsed since administration of a prior maintenance dosage;
the method further comprises restarting administration of the one or more maintenance dosages of step (ii).

In some embodiments, the method comprises:
i) administering to the patient an initiation dose comprising two tablets on day 1, and two tablets on day 2, wherein each tablet of the initiation dose comprises:
   about 26.6 w/w % to about 26.7 w/w % of the spray dried dispersion of the sodium salt of the compound of Formula Ia or Ib;
   about 0.03 w/w % to about 0.04 w/w % of the compound of Formula IIa or IIb, or a pharmaceutically acceptable salt, co-crystal, solvate, or combination thereof, and
   one or more pharmaceutically acceptable excipients; and
ii) administering to the patient a maintenance dose comprising one tablet, once weekly, beginning on day 8, wherein the tablet comprises:
   about 26.6 w/w % to about 26.7 w/w % of the spray dried dispersion of the sodium salt of the compound of Formula Ia or Ib;
   about 0.14 w/w % to about 0.15 w/w % of the compound of Formula IIa or IIb, or a pharmaceutically acceptable salt, co-crystal, solvate, or combination thereof, and
   one or more pharmaceutically acceptable excipients;
wherein if the patient misses a maintenance dosage; and
wherein between about 7 days to about 16 days have elapsed since administration of a prior maintenance dosage;
the method further comprises restarting administration of the one or more maintenance dosages of step (ii).

In some embodiments, the method comprises:
i) administering to the patient an initiation dose comprising two tablets on day 1, and two tablets on day 2, wherein each tablet of the initiation dose comprises:
   about 26.67 w/w % of the spray dried dispersion of the sodium salt of the compound of Formula Ia or Ib;
   about 0.0355 w/w % of the compound of Formula IIa or IIb, or a pharmaceutically acceptable salt, co-crystal, solvate, or combination thereof; and
   one or more pharmaceutically acceptable excipients; and
ii) administering to the patient a maintenance dose comprising one tablet, once weekly, beginning on day 8, wherein the tablet comprises:
   about 26.67 w/w % of the spray dried dispersion of the sodium salt of the compound of Formula Ia or Ib;
   about 0.142 w/w % of the compound of Formula IIa or IIb, or a pharmaceutically acceptable salt, co-crystal, solvate, or combination thereof; and
   one or more pharmaceutically acceptable excipients;
wherein if the patient misses a maintenance dosage; and
wherein between about 7 days to about 16 days have elapsed since administration of a prior maintenance dosage;
the method further comprises restarting administration of the one or more maintenance dosages of step (ii).

In some embodiments, the method comprises:
i) administering to the patient an initiation dose comprising two tablets on day 1, and two tablets on day 2, wherein each tablet of the initiation dose comprises:
   about 26.6 w/w % to about 26.7 w/w % of the spray dried dispersion of the sodium salt of the compound of Formula Ia or Ib;
   about 0.03 w/w % to about 0.04 w/w % of the compound of Formula IIa or IIb, or a solvate thereof; and
   one or more pharmaceutically acceptable excipients; and
ii) administering to the patient a maintenance dose comprising one tablet, once weekly, beginning on day 8, wherein the tablet comprises:
   about 26.6 w/w % to about 26.7 w/w % of the spray dried dispersion of the sodium salt of the compound of Formula Ia or Ib;
   about 0.14 w/w % to about 0.15 w/w % of the compound of Formula IIa or IIb, or a solvate thereof; and
   one or more pharmaceutically acceptable excipients;
wherein if the patient misses a maintenance dosage; and
wherein between about 7 days to about 16 days have elapsed since administration of a prior maintenance dosage;
the method further comprises restarting administration of the one or more maintenance dosages of step (ii).

In some embodiments, the method comprises:
i) administering to the patient an initiation dose comprising two tablets on day 1, and two tablets on day 2, wherein each tablet of the initiation dose comprises:
   about 26.67 w/w % of the spray dried dispersion of the sodium salt of the compound of Formula Ia or Ib;
   about 0.0355 w/w % of the compound of Formula IIa or IIb, or a solvate thereof, and
   one or more pharmaceutically acceptable excipients; and
ii) administering to the patient a maintenance dose comprising one tablet, once weekly, beginning on day 8, wherein the tablet comprises:
   about 26.67 w/w % of the spray dried dispersion of the sodium salt of the compound of Formula Ia or Ib;
   about 0.142 w/w % of the compound of Formula IIa or IIb, or a solvate thereof, and
   one or more pharmaceutically acceptable excipients;
wherein if the patient misses a maintenance dosage; and
wherein between about 7 days to about 16 days have elapsed since administration of a prior maintenance dosage;
the method further comprises restarting administration of the one or more maintenance dosages of step (ii).

In some embodiments, the method comprises:
i) administering to the patient an initiation dose comprising two tablets on day 1, and two tablets on day 2, wherein each tablet of the initiation dose comprises:
   about 26.6 w/w % to about 26.7 w/w % of the spray dried dispersion of the sodium salt of the compound of Formula Ia or Ib;
   about 0.03 w/w % to about 0.04 w/w % of the monohydrate of the compound of Formula IIa or IIb; and
   one or more pharmaceutically acceptable excipients; and
ii) administering to the patient a maintenance dose comprising one tablet, once weekly, beginning on day 8, wherein the tablet comprises:
   about 26.6 w/w % to about 26.7 w/w % of the spray dried dispersion of the sodium salt of the compound of Formula Ia or Ib;
   about 0.14 w/w % to about 0.15 w/w % of the monohydrate of the compound of Formula IIa or IIb; and
   one or more pharmaceutically acceptable excipients;
wherein if the patient misses a maintenance dosage; and
wherein between about 7 days to about 16 days have elapsed since administration of a prior maintenance dosage;
the method further comprises restarting administration of the one or more maintenance dosages of step (ii).

In some embodiments, the method comprises:
i) administering to the patient an initiation dose comprising two tablets on day 1, and two tablets on day 2, wherein each tablet of the initiation dose comprises:
   about 26.67 w/w % of the spray dried dispersion of the sodium salt of the compound of Formula Ia or Ib;
   about 0.0355 w/w % of the monohydrate of the compound of Formula IIa or IIb; and
   one or more pharmaceutically acceptable excipients; and
ii) administering to the patient a maintenance dose comprising one tablet, once weekly, beginning on day 8, wherein the tablet comprises:
   about 26.67 w/w % of the spray dried dispersion of the sodium salt of the compound of Formula Ia or Ib;
   about 0.142 w/w % of the monohydrate of the compound of Formula IIa or IIb; and
   one or more pharmaceutically acceptable excipients;
wherein if the patient misses a maintenance dosage; and
wherein between about 7 days to about 16 days have elapsed since administration of a prior maintenance dosage;
the method further comprises restarting administration of the one or more maintenance dosages of step (ii).

In some embodiments, the method comprises:
i) administering to the patient an initiation dose comprising two tablets on day 1, and two tablets on day 2, wherein each tablet of the initiation dose comprises:
   about 26.6 w/w % to about 26.7 w/w % of the spray dried dispersion of the sodium salt of the compound of Formula Ia or Ib;
   about 0.03 w/w % to about 0.04 w/w % of the compound of Formula IIa or IIb, or a pharmaceutically acceptable salt, co-crystal, solvate, or combination thereof;
   about 41 w/w % to about 42 w/w % of mannitol;
   about 22.3 w/w % to about 22.4 w/w % of microcrystalline cellulose;
   about 7.9 w/w % to about 8.1 w/w % of croscarmellose sodium; and
   about 1.4 w/w % to about 1.6 w/w % of magnesium stearate; and
ii) administering to the patient a maintenance dose comprising one tablet, once weekly, beginning on day 8, wherein the tablet comprises:
   about 26.6 w/w % to about 26.7 w/w % of the spray dried dispersion of the sodium salt of the compound of Formula Ia or Ib;
   about 0.14 w/w % to about 0.15 w/w % of the compound of Formula IIa or IIb, or a pharmaceutically acceptable salt, co-crystal, solvate, or combination thereof;
   about 41 w/w % to about 42 w/w % of mannitol;
   about 22.2 w/w % to about 22.3 w/w % of microcrystalline cellulose;
   about 7.9 w/w % to about 8.1 w/w % of croscarmellose sodium; and
   about 1.4 w/w % to about 1.6 w/w % of magnesium stearate;
wherein if the patient misses a maintenance dosage; and
wherein between about 7 days to about 16 days have elapsed since administration of a prior maintenance dosage;
the method further comprises restarting administration of the one or more maintenance dosages of step (ii).

In some embodiments, the method comprises:
i) administering to the patient an initiation dose comprising two tablets on day 1, and two tablets on day 2, wherein each tablet of the initiation dose comprises:
   about 26.67 w/w % of the spray dried dispersion of the sodium salt of the compound of Formula Ia or Ib;
   about 0.0355 w/w % of the compound of Formula IIa or IIb, or a pharmaceutically acceptable salt, co-crystal, solvate, or combination thereof;
   about 41.40 w/w % of mannitol;
   about 22.3945 w/w % of microcrystalline cellulose;
   about 8.0 w/w % of croscarmellose sodium; and
   about 1.5 w/w % of magnesium stearate; and
ii) administering to the patient a maintenance dose comprising one tablet, once weekly, beginning on day 8, wherein the tablet comprises:
   about 26.67 w/w % of the spray dried dispersion of the sodium salt of the compound of Formula Ia or Ib;
   about 0.142 w/w % of the compound of Formula IIa or IIb, or a pharmaceutically acceptable salt, co-crystal, solvate, or combination thereof;
   about 41.40 w/w % of mannitol;
   about 22.288 w/w % microcrystalline cellulose;
   about 8.0 w/w % of croscarmellose sodium; and
   about 1.5 w/w % of magnesium stearate;
wherein if the patient misses a maintenance dosage; and
wherein between about 7 days to about 16 days have elapsed since administration of a prior maintenance dosage;
the method further comprises restarting administration of the one or more maintenance dosages of step (ii).

In some embodiments, the method comprises:
i) administering to the patient an initiation dose comprising two tablets on day 1, and two tablets on day 2, wherein each tablet of the initiation dose comprises:
   about 400 mg of the spray dried dispersion of the sodium salt of the compound of Formula Ia or Ib; and
   about 0.5 mg to about 0.6 mg of the compound of Formula IIa or IIb, or a pharmaceutically acceptable salt, co-crystal, solvate, or combination thereof, and
ii) administering to the patient a maintenance dose comprising one tablet, once weekly, beginning on day 8, wherein the tablet comprises:
   about 400 mg of the spray dried dispersion of the sodium salt of the compound of Formula Ia or Ib; and
   about 2.1 mg to about 2.2 mg of the compound of Formula IIa or IIb, or a pharmaceutically acceptable salt, co-crystal, solvate, or combination thereof;
wherein if the patient misses a maintenance dosage; and
wherein between about 7 days to about 16 days have elapsed since administration of a prior maintenance dosage;
the method further comprises restarting administration of the one or more maintenance dosages of step (ii).

In some embodiments, the method comprises:
i) administering to the patient an initiation dose comprising two tablets on day 1, and two tablets on day 2, wherein each tablet of the initiation dose comprises:
   about 400 mg of the spray dried dispersion of the sodium salt of the compound of Formula Ia or Ib; and
   about 0.5325 mg of the compound of Formula IIa or IIb, or a pharmaceutically acceptable salt, co-crystal, solvate, or combination thereof; and
ii) administering to the patient a maintenance dose comprising one tablet, once weekly, beginning on day 8, wherein the tablet comprises:
   about 400 mg of the spray dried dispersion of the sodium salt of the compound of Formula Ia or Ib; and
   about 2.130 mg of the compound of Formula IIa or IIb, or a pharmaceutically acceptable salt, co-crystal, solvate, or combination thereof;
wherein if the patient misses a maintenance dosage; and
wherein between about 7 days to about 16 days have elapsed since administration of a prior maintenance dosage;
the method further comprises restarting administration of the one or more maintenance dosages of step (ii).

In some embodiments, the method comprises:
i) administering to the patient an initiation dose comprising two tablets on day 1, and two tablets on day 2, wherein each tablet of the initiation dose comprises:
   about 400 mg of the spray dried dispersion of the sodium salt of the compound of Formula Ia or Ib; and
   about 0.5 mg of the compound of Formula IIa or IIb, or a pharmaceutically acceptable salt, co-crystal, solvate, or combination thereof,
on a free base basis; and
ii) administering to the patient a maintenance dose comprising one tablet, once weekly, beginning on day 8, wherein the tablet comprises:
   about 400 mg of the spray dried dispersion of the sodium salt of the compound of Formula Ia or Ib; and
   about 2.0 mg of the compound of Formula IIa or IIb, or a pharmaceutically acceptable salt, co-crystal, solvate, or combination thereof,
on a free base basis;
wherein if the patient misses a maintenance dosage; and
wherein between about 7 days to about 16 days have elapsed since administration of a prior maintenance dosage;
the method further comprises restarting administration of the one or more maintenance dosages of step (ii).

In some embodiments, the method comprises:
i) administering to the patient an initiation dose comprising two tablets on day 1, and two tablets on day 2, wherein each tablet of the initiation dose comprises:
   about 400 mg of the spray dried dispersion of the sodium salt of the compound of Formula Ia or Ib;
   about 0.5 mg to about 0.6 mg of the compound of Formula IIa or IIb, or a pharmaceutically acceptable salt, co-crystal, solvate, or combination thereof;
   about 615 mg to about 625 mg of mannitol;
   about 330 mg to about 340 mg of microcrystalline cellulose;
   about 115 mg to about 125 mg croscarmellose sodium; and
   about 20 mg to about 25 mg magnesium stearate; and
ii) administering to the patient a maintenance dose comprising one tablet, once weekly, beginning on day 8, wherein the tablet comprises:
   about 400 mg of the spray dried dispersion of the sodium salt of the compound of Formula Ia or Ib;
   about 2.1 mg to about 2.2 mg of the compound of Formula IIa or IIb, or a pharmaceutically acceptable salt, co-crystal, solvate, or combination thereof;
   about 615 mg to about 625 mg of mannitol;
   about 330 mg to about 340 mg of microcrystalline cellulose;
   about 115 mg to about 125 mg croscarmellose sodium; and
   about 20 mg to about 25 mg magnesium stearate;
wherein if the patient misses a maintenance dosage; and
wherein between about 7 days to about 16 days have elapsed since administration of a prior maintenance dosage;
the method further comprises restarting administration of the one or more maintenance dosages of step (ii).

In some embodiments, the method comprises:
i) administering to the patient an initiation dose comprising two tablets on day 1, and two tablets on day 2, wherein each tablet of the initiation dose comprises:
   about 400 mg of the spray dried dispersion of the sodium salt of the compound of Formula Ia or Ib;
   about 0.5325 mg of the compound of Formula IIa or IIb, or a pharmaceutically acceptable salt, co-crystal, solvate, or combination thereof;
   about 621 mg of mannitol;
   about 335.9175 mg of microcrystalline cellulose;
   about 120 mg croscarmellose sodium; and
   about 22.5 mg magnesium stearate; and
ii) administering to the patient a maintenance dose comprising one tablet, once weekly, beginning on day 8, wherein the tablet comprises:
   about 400 mg of the spray dried dispersion of the sodium salt of the compound of Formula Ia or Ib;
   about 2.130 mg of the compound of Formula IIa or IIb, or a pharmaceutically acceptable salt, co-crystal, solvate, or combination thereof;
   about 621 mg of mannitol;
   about 334.32 mg of microcrystalline cellulose;
   about 120 mg croscarmellose sodium; and
   about 22.5 mg magnesium stearate;
wherein if the patient misses a maintenance dosage; and
wherein between about 7 days to about 16 days have elapsed since administration of a prior maintenance dosage;
the method further comprises restarting administration of the one or more maintenance dosages of step (ii).

In some embodiments, the method comprises:

i) administering to the patient an initiation dose comprising two tablets on day 1, and two tablets on day 2, wherein each tablet of the initiation dose comprises:
   about 400 mg of the spray dried dispersion of the sodium salt of the compound of Formula Ia or Ib;
   about 0.5 mg of the compound of Formula IIa or IIb, or a pharmaceutically acceptable salt, co-crystal, solvate, or combination thereof,
on a free base basis;
   about 621 mg of mannitol;
   about 335.9175 mg of microcrystalline cellulose;
   about 120 mg croscarmellose sodium; and
   about 22.5 mg magnesium stearate; and
ii) administering to the patient a maintenance dose comprising one tablet, once weekly, beginning on day 8, wherein the tablet comprises:
   about 400 mg of the spray dried dispersion of the sodium salt of the compound of Formula Ia or Ib;
   about 2.0 mg of the compound of Formula IIa or IIb, or a pharmaceutically acceptable salt, co-crystal, solvate, or combination thereof,
on a free base basis;
   about 621 mg of mannitol;
   about 334.32 mg of microcrystalline cellulose;
   about 120 mg croscarmellose sodium; and
   about 22.5 mg magnesium stearate;
wherein if the patient misses a maintenance dosage; and
wherein between about 7 days to about 16 days have elapsed since administration of a prior maintenance dosage;
the method further comprises restarting administration of the one or more maintenance dosages of step (ii).

In some embodiments, the method comprises:
i) administering to the patient an initiation dose comprising two tablets on day 1, and two tablets on day 2, wherein each tablet of the initiation dose comprises:
   about 26.6 w/w % to about 26.7 w/w % of the spray dried dispersion of the sodium salt of the compound of Formula Ia;
   about 0.03 w/w % to about 0.04 w/w % of the compound of Formula IIa or IIb, or a pharmaceutically acceptable salt, co-crystal, solvate, or combination thereof, and
   one or more pharmaceutically acceptable excipients; and
ii) administering to the patient a maintenance dose comprising one tablet, once weekly, beginning on day 8, wherein the tablet comprises:
   about 26.6 w/w % to about 26.7 w/w % of the spray dried dispersion of the sodium salt of the compound of Formula Ia;
   about 0.14 w/w % to about 0.15 w/w % of the compound of Formula IIa or IIb, or a pharmaceutically acceptable salt, co-crystal, solvate, or combination thereof, and
   one or more pharmaceutically acceptable excipients;
wherein if the patient misses a maintenance dosage; and
wherein between about 7 days to about 16 days have elapsed since administration of a prior maintenance dosage;
the method further comprises restarting administration of the one or more maintenance dosages of step (ii).

In some embodiments, the method comprises:
i) administering to the patient an initiation dose comprising two tablets on day 1, and two tablets on day 2, wherein each tablet of the initiation dose comprises:
   about 26.67 w/w % of the spray dried dispersion of the sodium salt of the compound of Formula Ia;
   about 0.0355 w/w % of the compound of Formula IIa or IIb, or a pharmaceutically acceptable salt, co-crystal, solvate, or combination thereof; and
   one or more pharmaceutically acceptable excipients; and
ii) administering to the patient a maintenance dose comprising one tablet, once weekly, beginning on day 8, wherein the tablet comprises:
   about 26.67 w/w % of the spray dried dispersion of the sodium salt of the compound of Formula Ia;
   about 0.142 w/w % of the compound of Formula IIa or IIb, or a pharmaceutically acceptable salt, co-crystal, solvate, or combination thereof; and
   one or more pharmaceutically acceptable excipients;
wherein if the patient misses a maintenance dosage; and
wherein between about 7 days to about 16 days have elapsed since administration of a prior maintenance dosage;
the method further comprises restarting administration of the one or more maintenance dosages of step (ii).

In some embodiments, the method comprises:
i) administering to the patient an initiation dose comprising two tablets on day 1, and two tablets on day 2, wherein each tablet of the initiation dose comprises:
   about 26.6 w/w % to about 26.7 w/w % of the spray dried dispersion of the sodium salt of the compound of Formula Ia;
   about 0.03 w/w % to about 0.04 w/w % of the compound of Formula IIa or IIb, or a solvate thereof; and
   one or more pharmaceutically acceptable excipients; and
ii) administering to the patient a maintenance dose comprising one tablet, once weekly, beginning on day 8, wherein the tablet comprises:
   about 26.6 w/w % to about 26.7 w/w % of the spray dried dispersion of the sodium salt of the compound of Formula Ia;
   about 0.14 w/w % to about 0.15 w/w % of the compound of Formula IIa or IIb, or a solvate thereof; and
   one or more pharmaceutically acceptable excipients;
wherein if the patient misses a maintenance dosage; and
wherein between about 7 days to about 16 days have elapsed since administration of a prior maintenance dosage;
the method further comprises restarting administration of the one or more maintenance dosages of step (ii).

In some embodiments, the method comprises:
i) administering to the patient an initiation dose comprising two tablets on day 1, and two tablets on day 2, wherein each tablet of the initiation dose comprises:
   about 26.67 w/w % of the spray dried dispersion of the sodium salt of the compound of Formula Ia;
   about 0.0355 w/w % of the compound of Formula IIa or IIb, or a solvate thereof, and
   one or more pharmaceutically acceptable excipients; and
ii) administering to the patient a maintenance dose comprising one tablet, once weekly, beginning on day 8, wherein the tablet comprises:
   about 26.67 w/w % of the spray dried dispersion of the sodium salt of the compound of Formula Ia;
   about 0.142 w/w % of the compound of Formula IIa or IIb, or a solvate thereof, and
   one or more pharmaceutically acceptable excipients;
wherein if the patient misses a maintenance dosage; and
wherein between about 7 days to about 16 days have elapsed since administration of a prior maintenance dosage;
the method further comprises restarting administration of the one or more maintenance dosages of step (ii).

In some embodiments, the method comprises:
i) administering to the patient an initiation dose comprising two tablets on day 1, and two tablets on day 2, wherein each tablet of the initiation dose comprises:
   about 26.6 w/w % to about 26.7 w/w % of the spray dried dispersion of the sodium salt of the compound of Formula Ia;
   about 0.03 w/w % to about 0.04 w/w % of the monohydrate of the compound of Formula IIa or IIb; and
   one or more pharmaceutically acceptable excipients; and
ii) administering to the patient a maintenance dose comprising one tablet, once weekly, beginning on day 8, wherein the tablet comprises:
   about 26.6 w/w % to about 26.7 w/w % of the spray dried dispersion of the sodium salt of the compound of Formula Ia;
   about 0.14 w/w % to about 0.15 w/w % of the monohydrate of the compound of Formula IIa or IIb; and
   one or more pharmaceutically acceptable excipients;
wherein if the patient misses a maintenance dosage; and
wherein between about 7 days to about 16 days have elapsed since administration of a prior maintenance dosage;
the method further comprises restarting administration of the one or more maintenance dosages of step (ii).

In some embodiments, the method comprises:
i) administering to the patient an initiation dose comprising two tablets on day 1, and two tablets on day 2, wherein each tablet of the initiation dose comprises:
   about 26.67 w/w % of the spray dried dispersion of the sodium salt of the compound of Formula Ia;
   about 0.0355 w/w % of the monohydrate of the compound of Formula IIa or IIb; and
   one or more pharmaceutically acceptable excipients; and
ii) administering to the patient a maintenance dose comprising one tablet, once weekly, beginning on day 8, wherein the tablet comprises:
   about 26.67 w/w % of the spray dried dispersion of the sodium salt of the compound of Formula Ia;
   about 0.142 w/w % of the monohydrate of the compound of Formula IIa or IIb; and
   one or more pharmaceutically acceptable excipients;
wherein if the patient misses a maintenance dosage; and
wherein between about 7 days to about 16 days have elapsed since administration of a prior maintenance dosage;
the method further comprises restarting administration of the one or more maintenance dosages of step (ii).

In some embodiments, the method comprises:
i) administering to the patient an initiation dose comprising two tablets on day 1, and two tablets on day 2, wherein each tablet of the initiation dose comprises:
   about 26.6 w/w % to about 26.7 w/w % of the spray dried dispersion of the sodium salt of the compound of Formula Ia;
   about 0.03 w/w % to about 0.04 w/w % of the compound of Formula IIa or IIb, or a pharmaceutically acceptable salt, co-crystal, solvate, or combination thereof;
   about 41 w/w % to about 42 w/w % of mannitol;
   about 22.3 w/w % to about 22.4 w/w % of microcrystalline cellulose;
   about 7.9 w/w % to about 8.1 w/w % of croscarmellose sodium; and
   about 1.4 w/w % to about 1.6 w/w % of magnesium stearate; and
ii) administering to the patient a maintenance dose comprising one tablet, once weekly, beginning on day 8, wherein the tablet comprises:
   about 26.6 w/w % to about 26.7 w/w % of the spray dried dispersion of the sodium salt of the compound of Formula Ia;
   about 0.14 w/w % to about 0.15 w/w % of the compound of Formula IIa or IIb, or a pharmaceutically acceptable salt, co-crystal, solvate, or combination thereof;
   about 41 w/w % to about 42 w/w % of mannitol;
   about 22.2 w/w % to about 22.3 w/w % of microcrystalline cellulose;
   about 7.9 w/w % to about 8.1 w/w % of croscarmellose sodium; and
   about 1.4 w/w % to about 1.6 w/w % of magnesium stearate;
wherein if the patient misses a maintenance dosage; and
wherein between about 7 days to about 16 days have elapsed since administration of a prior maintenance dosage;
the method further comprises restarting administration of the one or more maintenance dosages of step (ii).

In some embodiments, the method comprises:
i) administering to the patient an initiation dose comprising two tablets on day 1, and two tablets on day 2, wherein each tablet of the initiation dose comprises:
   about 26.67 w/w % of the spray dried dispersion of the sodium salt of the compound of Formula Ia;
   about 0.0355 w/w % of the compound of Formula IIa or IIb, or a pharmaceutically acceptable salt, co-crystal, solvate, or combination thereof;
   about 41.40 w/w % of mannitol;
   about 22.3945 w/w % of microcrystalline cellulose;
   about 8.0 w/w % of croscarmellose sodium; and
   about 1.5 w/w % of magnesium stearate; and
ii) administering to the patient a maintenance dose comprising one tablet, once weekly, beginning on day 8, wherein the tablet comprises:
   about 26.67 w/w % of the spray dried dispersion of the sodium salt of the compound of Formula Ia;
   about 0.142 w/w % of the compound of Formula IIa or IIb, or a pharmaceutically acceptable salt, co-crystal, solvate, or combination thereof;
   about 41.40 w/w % of mannitol;
   about 22.288 w/w % microcrystalline cellulose;
   about 8.0 w/w % of croscarmellose sodium; and
   about 1.5 w/w % of magnesium stearate;
wherein if the patient misses a maintenance dosage; and
wherein between about 7 days to about 16 days have elapsed since administration of a prior maintenance dosage;
the method further comprises restarting administration of the one or more maintenance dosages of step (ii).

In some embodiments, the method comprises:
i) administering to the patient an initiation dose comprising two tablets on day 1, and two tablets on day 2, wherein each tablet of the initiation dose comprises:
   about 400 mg of the spray dried dispersion of the sodium salt of the compound of Formula Ia; and
   about 0.5 mg to about 0.6 mg of the compound of Formula IIa or IIb, or a pharmaceutically acceptable salt, co-crystal, solvate, or combination thereof, and
ii) administering to the patient a maintenance dose comprising one tablet, once weekly, beginning on day 8, wherein the tablet comprises:
   about 400 mg of the spray dried dispersion of the sodium salt of the compound of Formula Ia; and
   about 2.1 mg to about 2.2 mg of the compound of Formula IIa or IIb, or a pharmaceutically acceptable salt, co-crystal, solvate, or combination thereof;
wherein if the patient misses a maintenance dosage; and
wherein between about 7 days to about 16 days have elapsed since administration of a prior maintenance dosage;
the method further comprises restarting administration of the one or more maintenance dosages of step (ii).

In some embodiments, the method comprises:
i) administering to the patient an initiation dose comprising two tablets on day 1, and two tablets on day 2, wherein each tablet of the initiation dose comprises:
   about 400 mg of the spray dried dispersion of the sodium salt of the compound of Formula Ia; and
   about 0.5325 mg of the compound of Formula IIa or IIb, or a pharmaceutically acceptable salt, co-crystal, solvate, or combination thereof; and
ii) administering to the patient a maintenance dose comprising one tablet, once weekly, beginning on day 8, wherein the tablet comprises:
   about 400 mg of the spray dried dispersion of the sodium salt of the compound of Formula Ia; and
   about 2.130 mg of the compound of Formula IIa or IIb, or a pharmaceutically acceptable salt, co-crystal, solvate, or combination thereof;
wherein if the patient misses a maintenance dosage; and
wherein between about 7 days to about 16 days have elapsed since administration of a prior maintenance dosage;
the method further comprises restarting administration of the one or more maintenance dosages of step (ii).

In some embodiments, the method comprises:
i) administering to the patient an initiation dose comprising two tablets on day 1, and two tablets on day 2, wherein each tablet of the initiation dose comprises:
   about 400 mg of the spray dried dispersion of the sodium salt of the compound of Formula Ia; and
   about 0.5 mg of the compound of Formula IIa or IIb, or a pharmaceutically acceptable salt, co-crystal, solvate, or combination thereof,
on a free base basis; and
ii) administering to the patient a maintenance dose comprising one tablet, once weekly, beginning on day 8, wherein the tablet comprises:
   about 400 mg of the spray dried dispersion of the sodium salt of the compound of Formula Ia; and
   about 2.0 mg of the compound of Formula IIa or IIb, or a pharmaceutically acceptable salt, co-crystal, solvate, or combination thereof,
on a free base basis;
wherein if the patient misses a maintenance dosage; and
wherein between about 7 days to about 16 days have elapsed since administration of a prior maintenance dosage;
the method further comprises restarting administration of the one or more maintenance dosages of step (ii).

In some embodiments, the method comprises:
i) administering to the patient an initiation dose comprising two tablets on day 1, and two tablets on day 2, wherein each tablet of the initiation dose comprises:
   about 400 mg of the spray dried dispersion of the sodium salt of the compound of Formula Ia;
   about 0.5 mg to about 0.6 mg of the compound of Formula IIa or IIb, or a pharmaceutically acceptable salt, co-crystal, solvate, or combination thereof;
   about 615 mg to about 625 mg of mannitol;
   about 330 mg to about 340 mg of microcrystalline cellulose;
   about 115 mg to about 125 mg croscarmellose sodium; and
   about 20 mg to about 25 mg magnesium stearate; and
ii) administering to the patient a maintenance dose comprising one tablet, once weekly, beginning on day 8, wherein the tablet comprises:
   about 400 mg of the spray dried dispersion of the sodium salt of the compound of Formula Ia;
   about 2.1 mg to about 2.2 mg of the compound of Formula IIa or IIb, or a pharmaceutically acceptable salt, co-crystal, solvate, or combination thereof;
   about 615 mg to about 625 mg of mannitol;
   about 330 mg to about 340 mg of microcrystalline cellulose;
   about 115 mg to about 125 mg croscarmellose sodium; and
   about 20 mg to about 25 mg magnesium stearate;
wherein if the patient misses a maintenance dosage; and
wherein between about 7 days to about 16 days have elapsed since administration of a prior maintenance dosage;
the method further comprises restarting administration of the one or more maintenance dosages of step (ii).

In some embodiments, the method comprises:
i) administering to the patient an initiation dose comprising two tablets on day 1, and two tablets on day 2, wherein each tablet of the initiation dose comprises:
   about 400 mg of the spray dried dispersion of the sodium salt of the compound of Formula Ia;
   about 0.5325 mg of the compound of Formula IIa or IIb, or a pharmaceutically acceptable salt, co-crystal, solvate, or combination thereof;
   about 621 mg of mannitol;
   about 335.9175 mg of microcrystalline cellulose;
   about 120 mg croscarmellose sodium; and
   about 22.5 mg magnesium stearate; and
ii) administering to the patient a maintenance dose comprising one tablet, once weekly, beginning on day 8, wherein the tablet comprises:
   about 400 mg of the spray dried dispersion of the sodium salt of the compound of Formula Ia;
   about 2.130 mg of the compound of Formula IIa or IIb, or a pharmaceutically acceptable salt, co-crystal, solvate, or combination thereof;
   about 621 mg of mannitol;
   about 334.32 mg of microcrystalline cellulose;
   about 120 mg croscarmellose sodium; and
   about 22.5 mg magnesium stearate;
wherein if the patient misses a maintenance dosage; and
wherein between about 7 days to about 16 days have elapsed since administration of a prior maintenance dosage;
the method further comprises restarting administration of the one or more maintenance dosages of step (ii).

In some embodiments, the method comprises:
i) administering to the patient an initiation dose comprising two tablets on day 1, and two tablets on day 2, wherein each tablet of the initiation dose comprises:
   about 400 mg of the spray dried dispersion of the sodium salt of the compound of Formula Ia;
   about 0.5 mg of the compound of Formula IIa or IIb, or a pharmaceutically acceptable salt, co-crystal, solvate, or combination thereof,
on a free base basis;
   about 621 mg of mannitol;
   about 335.9175 mg of microcrystalline cellulose;
   about 120 mg croscarmellose sodium; and
   about 22.5 mg magnesium stearate; and
ii) administering to the patient a maintenance dose comprising one tablet, once weekly, beginning on day 8, wherein the tablet comprises:
   about 400 mg of the spray dried dispersion of the sodium salt of the compound of Formula Ia;
   about 2.0 mg of the compound of Formula IIa or IIb, or a pharmaceutically acceptable salt, co-crystal, solvate, or combination thereof,
on a free base basis;
   about 621 mg of mannitol;
   about 334.32 mg of microcrystalline cellulose;
   about 120 mg croscarmellose sodium; and
   about 22.5 mg magnesium stearate;
wherein if the patient misses a maintenance dosage; and
wherein between about 7 days to about 16 days have elapsed since administration of a prior maintenance dosage;
the method further comprises restarting administration of the one or more maintenance dosages of step (ii).

In some embodiments, the method comprises:
i) administering to the patient an initiation dose comprising two tablets on day 1, and two tablets on day 2, wherein each tablet of the initiation dose comprises:
   about 26.6 w/w % to about 26.7 w/w % of the lenacapavir spray dried dispersion;
   about 0.03 w/w % to about 0.04 w/w % of the compound of Formula IIa or IIb, or a pharmaceutically acceptable salt, co-crystal, solvate, or combination thereof, and
   one or more pharmaceutically acceptable excipients; and
ii) administering to the patient a maintenance dose comprising one tablet, once weekly, beginning on day 8, wherein the tablet comprises:
   about 26.6 w/w % to about 26.7 w/w % of the lenacapavir spray dried dispersion;
   about 0.14 w/w % to about 0.15 w/w % of the compound of Formula IIa or IIb, or a pharmaceutically acceptable salt, co-crystal, solvate, or combination thereof, and
   one or more pharmaceutically acceptable excipients;
wherein if the patient misses a maintenance dosage; and
wherein between about 7 days to about 16 days have elapsed since administration of a prior maintenance dosage;
the method further comprises restarting administration of the one or more maintenance dosages of step (ii).

In some embodiments, the method comprises:
i) administering to the patient an initiation dose comprising two tablets on day 1, and two tablets on day 2, wherein each tablet of the initiation dose comprises:
   about 26.67 w/w % of the lenacapavir spray dried dispersion;
   about 0.0355 w/w % of the compound of Formula IIa or IIb, or a pharmaceutically acceptable salt, co-crystal, solvate, or combination thereof; and
   one or more pharmaceutically acceptable excipients; and
ii) administering to the patient a maintenance dose comprising one tablet, once weekly, beginning on day 8, wherein the tablet comprises:
   about 26.67 w/w % of the lenacapavir spray dried dispersion;
   about 0.142 w/w % of the compound of Formula IIa or IIb, or a pharmaceutically acceptable salt, co-crystal, solvate, or combination thereof; and
   one or more pharmaceutically acceptable excipients;
wherein if the patient misses a maintenance dosage; and
wherein between about 7 days to about 16 days have elapsed since administration of a prior maintenance dosage;
the method further comprises restarting administration of the one or more maintenance dosages of step (ii).

In some embodiments, the method comprises:
i) administering to the patient an initiation dose comprising two tablets on day 1, and two tablets on day 2, wherein each tablet of the initiation dose comprises:
   about 26.6 w/w % to about 26.7 w/w % of the lenacapavir spray dried dispersion;
   about 0.03 w/w % to about 0.04 w/w % of the compound of Formula IIa or IIb, or a solvate thereof; and
   one or more pharmaceutically acceptable excipients; and
ii) administering to the patient a maintenance dose comprising one tablet, once weekly, beginning on day 8, wherein the tablet comprises:
   about 26.6 w/w % to about 26.7 w/w % of the lenacapavir spray dried dispersion;
   about 0.14 w/w % to about 0.15 w/w % of the compound of Formula IIa or IIb, or a solvate thereof; and
   one or more pharmaceutically acceptable excipients;
wherein if the patient misses a maintenance dosage; and
wherein between about 7 days to about 16 days have elapsed since administration of a prior maintenance dosage;
the method further comprises restarting administration of the one or more maintenance dosages of step (ii).

In some embodiments, the method comprises:
i) administering to the patient an initiation dose comprising two tablets on day 1, and two tablets on day 2, wherein each tablet of the initiation dose comprises:
   about 26.67 w/w % of the lenacapavir spray dried dispersion;
   about 0.0355 w/w % of the compound of Formula IIa or IIb, or a solvate thereof, and
   one or more pharmaceutically acceptable excipients; and
ii) administering to the patient a maintenance dose comprising one tablet, once weekly, beginning on day 8, wherein the tablet comprises:
   about 26.67 w/w % of the lenacapavir spray dried dispersion;
   about 0.142 w/w % of the compound of Formula IIa or IIb, or a solvate thereof, and
   one or more pharmaceutically acceptable excipients;
wherein if the patient misses a maintenance dosage; and
wherein between about 7 days to about 16 days have elapsed since administration of a prior maintenance dosage;
the method further comprises restarting administration of the one or more maintenance dosages of step (ii).

In some embodiments, the method comprises:
i) administering to the patient an initiation dose comprising two tablets on day 1, and two tablets on day 2, wherein each tablet of the initiation dose comprises:
   about 26.6 w/w % to about 26.7 w/w % of the lenacapavir spray dried dispersion;
   about 0.03 w/w % to about 0.04 w/w % of the monohydrate of the compound of Formula IIa or IIb; and
   one or more pharmaceutically acceptable excipients; and
ii) administering to the patient a maintenance dose comprising one tablet, once weekly, beginning on day 8, wherein the tablet comprises:
   about 26.6 w/w % to about 26.7 w/w % of the lenacapavir spray dried dispersion;
   about 0.14 w/w % to about 0.15 w/w % of the monohydrate of the compound of Formula IIa or IIb; and
   one or more pharmaceutically acceptable excipients;
wherein if the patient misses a maintenance dosage; and
wherein between about 7 days to about 16 days have elapsed since administration of a prior maintenance dosage;
the method further comprises restarting administration of the one or more maintenance dosages of step (ii).

In some embodiments, the method comprises:
i) administering to the patient an initiation dose comprising two tablets on day 1, and two tablets on day 2, wherein each tablet of the initiation dose comprises:
   about 26.67 w/w % of the lenacapavir spray dried dispersion;
   about 0.0355 w/w % of the monohydrate of the compound of Formula IIa or IIb; and
   one or more pharmaceutically acceptable excipients; and
ii) administering to the patient a maintenance dose comprising one tablet, once weekly, beginning on day 8, wherein the tablet comprises:
   about 26.67 w/w % of the lenacapavir spray dried dispersion;
   about 0.142 w/w % of the monohydrate of the compound of Formula IIa or IIb; and
   one or more pharmaceutically acceptable excipients;
wherein if the patient misses a maintenance dosage; and
wherein between about 7 days to about 16 days have elapsed since administration of a prior maintenance dosage;
the method further comprises restarting administration of the one or more maintenance dosages of step (ii).

In some embodiments, the method comprises:
i) administering to the patient an initiation dose comprising two tablets on day 1, and two tablets on day 2, wherein each tablet of the initiation dose comprises:
   about 26.6 w/w % to about 26.7 w/w % of the lenacapavir spray dried dispersion;
   about 0.03 w/w % to about 0.04 w/w % of the compound of Formula IIa or IIb, or a pharmaceutically acceptable salt, co-crystal, solvate, or combination thereof;
   about 41 w/w % to about 42 w/w % of mannitol;
   about 22.3 w/w % to about 22.4 w/w % of microcrystalline cellulose;
   about 7.9 w/w % to about 8.1 w/w % of croscarmellose sodium; and
   about 1.4 w/w % to about 1.6 w/w % of magnesium stearate; and
ii) administering to the patient a maintenance dose comprising one tablet, once weekly, beginning on day 8, wherein the tablet comprises:
   about 26.6 w/w % to about 26.7 w/w % of the lenacapavir spray dried dispersion;
   about 0.14 w/w % to about 0.15 w/w % of the compound of Formula IIa or IIb, or a pharmaceutically acceptable salt, co-crystal, solvate, or combination thereof;
   about 41 w/w % to about 42 w/w % of mannitol;
   about 22.2 w/w % to about 22.3 w/w % of microcrystalline cellulose;
   about 7.9 w/w % to about 8.1 w/w % of croscarmellose sodium; and
   about 1.4 w/w % to about 1.6 w/w % of magnesium stearate;
wherein if the patient misses a maintenance dosage; and
wherein between about 7 days to about 16 days have elapsed since administration of a prior maintenance dosage;
the method further comprises restarting administration of the one or more maintenance dosages of step (ii).

In some embodiments, the method comprises:
i) administering to the patient an initiation dose comprising two tablets on day 1, and two tablets on day 2, wherein each tablet of the initiation dose comprises:
   about 26.67 w/w % of the lenacapavir spray dried dispersion;
   about 0.0355 w/w % of the compound of Formula IIa or IIb, or a pharmaceutically acceptable salt, co-crystal, solvate, or combination thereof;
   about 41.40 w/w % of mannitol;
   about 22.3945 w/w % of microcrystalline cellulose;
   about 8.0 w/w % of croscarmellose sodium; and
   about 1.5 w/w % of magnesium stearate; and
ii) administering to the patient a maintenance dose comprising one tablet, once weekly, beginning on day 8, wherein the tablet comprises:
   about 26.67 w/w % of the lenacapavir spray dried dispersion;
   about 0.142 w/w % of the compound of Formula IIa or IIb, or a pharmaceutically acceptable salt, co-crystal, solvate, or combination thereof;
   about 41.40 w/w % of mannitol;
   about 22.288 w/w % microcrystalline cellulose;
   about 8.0 w/w % of croscarmellose sodium; and
   about 1.5 w/w % of magnesium stearate;
wherein if the patient misses a maintenance dosage; and
wherein between about 7 days to about 16 days have elapsed since administration of a prior maintenance dosage;
the method further comprises restarting administration of the one or more maintenance dosages of step (ii).

In some embodiments, the method comprises:
i) administering to the patient an initiation dose comprising two tablets on day 1, and two tablets on day 2, wherein each tablet of the initiation dose comprises:
   about 400 mg of the lenacapavir spray dried dispersion; and
   about 0.5 mg to about 0.6 mg of the compound of Formula IIa or IIb, or a pharmaceutically acceptable salt, co-crystal, solvate, or combination thereof, and
ii) administering to the patient a maintenance dose comprising one tablet, once weekly, beginning on day 8, wherein the tablet comprises:
   about 400 mg of the lenacapavir spray dried dispersion; and
   about 2.1 mg to about 2.2 mg of the compound of Formula IIa or IIb, or a pharmaceutically acceptable salt, co-crystal, solvate, or combination thereof;
wherein if the patient misses a maintenance dosage; and
wherein between about 7 days to about 16 days have elapsed since administration of a prior maintenance dosage;
the method further comprises restarting administration of the one or more maintenance dosages of step (ii).

In some embodiments, the method comprises:
i) administering to the patient an initiation dose comprising two tablets on day 1, and two tablets on day 2, wherein each tablet of the initiation dose comprises:
   about 400 mg of the lenacapavir spray dried dispersion; and
   about 0.5325 mg of the compound of Formula IIa or IIb, or a pharmaceutically acceptable salt, co-crystal, solvate, or combination thereof; and
ii) administering to the patient a maintenance dose comprising one tablet, once weekly, beginning on day 8, wherein the tablet comprises:
   about 400 mg of the lenacapavir spray dried dispersion; and
   about 2.130 mg of the compound of Formula IIa or IIb, or a pharmaceutically acceptable salt, co-crystal, solvate, or combination thereof;
wherein if the patient misses a maintenance dosage; and
wherein between about 7 days to about 16 days have elapsed since administration of a prior maintenance dosage;
the method further comprises restarting administration of the one or more maintenance dosages of step (ii).

In some embodiments, the method comprises:
i) administering to the patient an initiation dose comprising two tablets on day 1, and two tablets on day 2, wherein each tablet of the initiation dose comprises:
   about 400 mg of the lenacapavir spray dried dispersion; and
   about 0.5 mg of the compound of Formula IIa or IIb, or a pharmaceutically acceptable salt, co-crystal, solvate, or combination thereof,
on a free base basis; and
ii) administering to the patient a maintenance dose comprising one tablet, once weekly, beginning on day 8, wherein the tablet comprises:
   about 400 mg of the lenacapavir spray dried dispersion; and
   about 2.0 mg of the compound of Formula IIa or IIb, or a pharmaceutically acceptable salt, co-crystal, solvate, or combination thereof,
on a free base basis;
wherein if the patient misses a maintenance dosage; and
wherein between about 7 days to about 16 days have elapsed since administration of a prior maintenance dosage;
the method further comprises restarting administration of the one or more maintenance dosages of step (ii).

In some embodiments, the method comprises:
i) administering to the patient an initiation dose comprising two tablets on day 1, and two tablets on day 2, wherein each tablet of the initiation dose comprises:
   about 400 mg of the lenacapavir spray dried dispersion;
   about 0.5 mg to about 0.6 mg of the compound of Formula IIa or IIb, or a pharmaceutically acceptable salt, co-crystal, solvate, or combination thereof;
   about 615 mg to about 625 mg of mannitol;
   about 330 mg to about 340 mg of microcrystalline cellulose;
   about 115 mg to about 125 mg croscarmellose sodium; and
   about 20 mg to about 25 mg magnesium stearate; and
ii) administering to the patient a maintenance dose comprising one tablet, once weekly, beginning on day 8, wherein the tablet comprises:
   about 400 mg of the lenacapavir spray dried dispersion;
   about 2.1 mg to about 2.2 mg of the compound of Formula IIa or IIb, or a pharmaceutically acceptable salt, co-crystal, solvate, or combination thereof;
   about 615 mg to about 625 mg of mannitol;
   about 330 mg to about 340 mg of microcrystalline cellulose;
   about 115 mg to about 125 mg croscarmellose sodium; and
   about 20 mg to about 25 mg magnesium stearate;
wherein if the patient misses a maintenance dosage; and
wherein between about 7 days to about 16 days have elapsed since administration of a prior maintenance dosage;
the method further comprises restarting administration of the one or more maintenance dosages of step (ii).

In some embodiments, the method comprises:
i) administering to the patient an initiation dose comprising two tablets on day 1, and two tablets on day 2, wherein each tablet of the initiation dose comprises:
   about 400 mg of the lenacapavir spray dried dispersion;
   about 0.5325 mg of the compound of Formula IIa or IIb, or a pharmaceutically acceptable salt, co-crystal, solvate, or combination thereof;
   about 621 mg of mannitol;
   about 335.9175 mg of microcrystalline cellulose;
   about 120 mg croscarmellose sodium; and
   about 22.5 mg magnesium stearate; and
ii) administering to the patient a maintenance dose comprising one tablet, once weekly, beginning on day 8, wherein the tablet comprises:
   about 400 mg of the lenacapavir spray dried dispersion;
   about 2.130 mg of the compound of Formula IIa or IIb, or a pharmaceutically acceptable salt, co-crystal, solvate, or combination thereof;
   about 621 mg of mannitol;
   about 334.32 mg of microcrystalline cellulose;
   about 120 mg croscarmellose sodium; and
   about 22.5 mg magnesium stearate;
wherein if the patient misses a maintenance dosage; and
wherein between about 7 days to about 16 days have elapsed since administration of a prior maintenance dosage;
the method further comprises restarting administration of the one or more maintenance dosages of step (ii).

In some embodiments, the method comprises:
i) administering to the patient an initiation dose comprising two tablets on day 1, and two tablets on day 2, wherein each tablet of the initiation dose comprises:
   about 400 mg of the lenacapavir spray dried dispersion;
   about 0.5 mg of the compound of Formula IIa or IIb, or a pharmaceutically acceptable salt, co-crystal, solvate, or combination thereof,
on a free base basis;
   about 621 mg of mannitol;
   about 335.9175 mg of microcrystalline cellulose;
   about 120 mg croscarmellose sodium; and
   about 22.5 mg magnesium stearate; and
ii) administering to the patient a maintenance dose comprising one tablet, once weekly, beginning on day 8, wherein the tablet comprises:
   about 400 mg of the lenacapavir spray dried dispersion;
   about 2.0 mg of the compound of Formula IIa or IIb, or a pharmaceutically acceptable salt, co-crystal, solvate, or combination thereof,
on a free base basis;
   about 621 mg of mannitol;
   about 334.32 mg of microcrystalline cellulose;
   about 120 mg croscarmellose sodium; and
   about 22.5 mg magnesium stearate;
wherein if the patient misses a maintenance dosage; and
wherein between about 7 days to about 16 days have elapsed since administration of a prior maintenance dosage;
the method further comprises restarting administration of the one or more maintenance dosages of step (ii).

In some embodiments, the method comprises:
i) administering to the patient an initiation dose comprising two tablets on day 1, and two tablets on day 2, wherein each tablet of the initiation dose comprises:
   about 300 mg of the compound of Formula Ia or Ib, or a pharmaceutically acceptable salt, co-crystal, solvate, or combination thereof,
on a free acid basis; and
   about 0.5 mg to about 0.6 mg of the compound of Formula IIa or IIb, or a pharmaceutically acceptable salt, co-crystal, solvate, or combination thereof, and
ii) administering to the patient a maintenance dose comprising one tablet, once weekly, beginning on day 8, wherein the tablet comprises:
   about 300 mg of the compound of Formula Ia or Ib, or a pharmaceutically acceptable salt, co-crystal, solvate, or combination thereof,
on a free acid basis; and
   about 2.1 mg to about 2.2 mg of the compound of Formula IIa or IIb, or a pharmaceutically acceptable salt, co-crystal, solvate, or combination thereof;
wherein if the patient misses a maintenance dosage; and
wherein between about 7 days to about 16 days have elapsed since administration of a prior maintenance dosage;
the method further comprises restarting administration of the one or more maintenance dosages of step (ii).

In some embodiments, the method comprises:
i) administering to the patient an initiation dose comprising two tablets on day 1, and two tablets on day 2, wherein each tablet of the initiation dose comprises:
   about 300 mg of the compound of Formula Ia or Ib, or a pharmaceutically acceptable salt, co-crystal, solvate, or combination thereof,
on a free acid basis; and
   about 0.5325 mg of the compound of Formula IIa or IIb, or a pharmaceutically acceptable salt, co-crystal, solvate, or combination thereof; and
ii) administering to the patient a maintenance dose comprising one tablet, once weekly, beginning on day 8, wherein the tablet comprises:
   about 300 mg of the compound of Formula Ia or Ib, or a pharmaceutically acceptable salt, co-crystal, solvate, or combination thereof,
on a free acid basis; and
   about 2.130 mg of the compound of Formula IIa or IIb, or a pharmaceutically acceptable salt, co-crystal, solvate, or combination thereof;
wherein if the patient misses a maintenance dosage; and
wherein between about 7 days to about 16 days have elapsed since administration of a prior maintenance dosage;
the method further comprises restarting administration of the one or more maintenance dosages of step (ii).

In some embodiments, the method comprises:
i) administering to the patient an initiation dose comprising two tablets on day 1, and two tablets on day 2, wherein each tablet of the initiation dose comprises:
   about 300 mg of the compound of Formula Ia or Ib, or a pharmaceutically acceptable salt, co-crystal, solvate, or combination thereof,
on a free acid basis;
   about 0.5 mg of the compound of Formula IIa or IIb, or a pharmaceutically acceptable salt, co-crystal, solvate, or combination thereof,
on a free base basis; and
ii) administering to the patient a maintenance dose comprising one tablet, once weekly, beginning on day 8, wherein the tablet comprises:
   about 300 mg of the compound of Formula Ia or Ib, or a pharmaceutically acceptable salt, co-crystal, solvate, or combination thereof,
on a free acid basis; and
   about 2.0 mg of the compound of Formula IIa or IIb, or a pharmaceutically acceptable salt, co-crystal, solvate, or combination thereof,
on a free base basis;
wherein if the patient misses a maintenance dosage; and
wherein between about 7 days to about 16 days have elapsed since administration of a prior maintenance dosage;
the method further comprises restarting administration of the one or more maintenance dosages of step (ii).

In some embodiments, the method comprises:
i) administering to the patient an initiation dose comprising two tablets on day 1, and two tablets on day 2, wherein each tablet of the initiation dose comprises:
   about 300 mg of the compound of Formula Ia or Ib, or a pharmaceutically acceptable salt, co-crystal, solvate, or combination thereof,
on a free acid basis;
   about 0.5 mg to about 0.6 mg of the compound of Formula IIa or IIb, or a pharmaceutically acceptable salt, co-crystal, solvate, or combination thereof;
   about 615 mg to about 625 mg of mannitol;
   about 330 mg to about 340 mg of microcrystalline cellulose;
   about 115 mg to about 125 mg croscarmellose sodium; and
   about 20 mg to about 25 mg magnesium stearate; and
ii) administering to the patient a maintenance dose comprising one tablet, once weekly, beginning on day 8, wherein the tablet comprises:
   about 300 mg of the compound of Formula Ia or Ib, or a pharmaceutically acceptable salt, co-crystal, solvate, or combination thereof,
on a free acid basis;
   about 2.1 mg to about 2.2 mg of the compound of Formula IIa or IIb, or a pharmaceutically acceptable salt, co-crystal, solvate, or combination thereof;
   about 615 mg to about 625 mg of mannitol;
   about 330 mg to about 340 mg of microcrystalline cellulose;
   about 115 mg to about 125 mg croscarmellose sodium; and
   about 20 mg to about 25 mg magnesium stearate;
wherein if the patient misses a maintenance dosage; and
wherein between about 7 days to about 16 days have elapsed since administration of a prior maintenance dosage;
the method further comprises restarting administration of the one or more maintenance dosages of step (ii).

In some embodiments, the method comprises:
i) administering to the patient an initiation dose comprising two tablets on day 1, and two tablets on day 2, wherein each tablet of the initiation dose comprises:
   about 300 mg of the compound of Formula Ia or Ib, or a pharmaceutically acceptable salt, co-crystal, solvate, or combination thereof,
on a free acid basis;
   about 0.5325 mg of the compound of Formula IIa or IIb, or a pharmaceutically acceptable salt, co-crystal, solvate, or combination thereof;
   about 621 mg of mannitol;
   about 335.9175 mg of microcrystalline cellulose;
   about 120 mg croscarmellose sodium; and
   about 22.5 mg magnesium stearate; and
ii) administering to the patient a maintenance dose comprising one tablet, once weekly, beginning on day 8, wherein the tablet comprises:
   about 300 mg of the compound of Formula Ia or Ib, or a pharmaceutically acceptable salt, co-crystal, solvate, or combination thereof,
on a free acid basis;
   about 2.130 mg of the compound of Formula IIa or IIb, or a pharmaceutically acceptable salt, co-crystal, solvate, or combination thereof;
   about 621 mg of mannitol;
   about 334.32 mg of microcrystalline cellulose;
   about 120 mg croscarmellose sodium; and
   about 22.5 mg magnesium stearate;
wherein if the patient misses a maintenance dosage; and
wherein between about 7 days to about 16 days have elapsed since administration of a prior maintenance dosage;
the method further comprises restarting administration of the one or more maintenance dosages of step (ii).

In some embodiments, the method comprises:
i) administering to the patient an initiation dose comprising two tablets on day 1, and two tablets on day 2, wherein each tablet of the initiation dose comprises:
   about 300 mg of the compound of Formula Ia or Ib, or a pharmaceutically acceptable salt, co-crystal, solvate, or combination thereof,
on a free acid basis;
   about 0.5 mg of the compound of Formula IIa or IIb, or a pharmaceutically acceptable salt, co-crystal, solvate, or combination thereof,
on a free base basis;
   about 621 mg of mannitol;
   about 335.9175 mg of microcrystalline cellulose;
   about 120 mg croscarmellose sodium; and
   about 22.5 mg magnesium stearate; and
ii) administering to the patient a maintenance dose comprising one tablet, once weekly, beginning on day 8, wherein the tablet comprises:
   about 300 mg of the compound of Formula Ia or Ib, or a pharmaceutically acceptable salt, co-crystal, solvate, or combination thereof,
on a free acid basis;
   about 2.0 mg of the compound of Formula IIa or IIb, or a pharmaceutically acceptable salt, co-crystal, solvate, or combination thereof,
on a free base basis;
   about 621 mg of mannitol;
   about 334.32 mg of microcrystalline cellulose;
   about 120 mg croscarmellose sodium; and
   about 22.5 mg magnesium stearate;
wherein if the patient misses a maintenance dosage; and
wherein between about 7 days to about 16 days have elapsed since administration of a prior maintenance dosage;
the method further comprises restarting administration of the one or more maintenance dosages of step (ii).

In some embodiments, the method comprises:
i) administering to the patient an initiation dose comprising two tablets on day 1, and two tablets on day 2, wherein each tablet of the initiation dose comprises:
   about 300 mg of the compound of Formula Ib, on a free acid basis;
   about 0.5 mg of the compound of Formula IIb, on a free base basis;
   about 621 mg of mannitol;
   about 335.9175 mg of microcrystalline cellulose;
   about 120 mg croscarmellose sodium; and
   about 22.5 mg magnesium stearate;
and
ii) administering to the patient a maintenance dose comprising one tablet, once weekly, beginning on day 8, wherein the tablet comprises:
   about 300 mg of the compound of Formula Ib, on a free acid basis;
   about 2.0 mg of the compound of Formula IIb, on a free base basis;
   about 621 mg of mannitol;
   about 334.32 mg of microcrystalline cellulose;
   about 120 mg croscarmellose sodium; and
   about 22.5 mg magnesium stearate;
wherein if the patient misses a maintenance dosage; and
wherein between about 7 days to about 16 days have elapsed since administration of a prior maintenance dosage;
the method further comprises restarting administration of the one or more maintenance dosages of step (ii) as soon as possible after the missed dose.

In some embodiments, if the patient misses a maintenance dosage (e.g., a once weekly maintenance dosage); and
wherein between about 7 days to about 15 days have elapsed since administration of a prior maintenance dosage;
the method further comprises restarting administration of the one or more maintenance dosages of step (ii).

In some embodiments, if the patient misses a maintenance dosage (e.g., a once weekly maintenance dosage); and
wherein between about 7 days to about 14 days have elapsed since administration of a prior maintenance dosage;
the method further comprises restarting administration of the one or more maintenance dosages of step (ii).

In some embodiments, if the patient misses a maintenance dosage (e.g., a once weekly maintenance dosage); and
wherein between about 7 days to about 13 days have elapsed since administration of a prior maintenance dosage;
the method further comprises restarting administration of the one or more maintenance dosages of step (ii).

In some embodiments, if the patient misses a maintenance dosage (e.g., a once weekly maintenance dosage); and
wherein between about 7 days to about 12 days have elapsed since administration of a prior maintenance dosage;
the method further comprises restarting administration of the one or more maintenance dosages of step (ii).

In some embodiments, if the patient misses a maintenance dosage (e.g., a once weekly maintenance dosage); and
wherein between about 7 days to about 11 days have elapsed since administration of a prior maintenance dosage;
the method further comprises restarting administration of the one or more maintenance dosages of step (ii).

In some embodiments, if the patient misses a maintenance dosage (e.g., a once weekly maintenance dosage); and
wherein between about 7 days to about 10 days have elapsed since administration of a prior maintenance dosage;
the method further comprises restarting administration of the one or more maintenance dosages of step (ii).

In some embodiments, if the patient misses a maintenance dosage (e.g., a once weekly maintenance dosage); and
wherein between about 7 days to about 9 days have elapsed since administration of a prior maintenance dosage;
the method further comprises restarting administration of the one or more maintenance dosages of step (ii).

In some embodiments, if the patient misses a maintenance dosage (e.g., a once weekly maintenance dosage); and
wherein between about 7 days to about 8 days have elapsed since administration of a prior maintenance dosage;
the method further comprises restarting administration of the one or more maintenance dosages of step (ii).

In some embodiments, if the patient misses a maintenance dosage (e.g., a once weekly maintenance dosage); and
wherein between about 8 days to about 15 days have elapsed since administration of a prior maintenance dosage;
the method further comprises restarting administration of the one or more maintenance dosages of step (ii).

In some embodiments, if the patient misses a maintenance dosage (e.g., a once weekly maintenance dosage); and
wherein between about 8 days to about 14 days have elapsed since administration of a prior maintenance dosage;
the method further comprises restarting administration of the one or more maintenance dosages of step (ii).

In some embodiments, if the patient misses a maintenance dosage (e.g., a once weekly maintenance dosage); and
wherein between about 8 days to about 13 days have elapsed since administration of a prior maintenance dosage
the method further comprises restarting administration of the one or more maintenance dosages of step (ii).

In some embodiments, if the patient misses a maintenance dosage (e.g., a once weekly maintenance dosage); and
wherein between about 8 days to about 12 days have elapsed since administration of a prior maintenance dosage;
the method further comprises restarting administration of the one or more maintenance dosages of step (ii).

In some embodiments, if the patient misses a maintenance dosage (e.g., a once weekly maintenance dosage); and
wherein between about 8 days to about 11 days have elapsed since administration of a prior maintenance dosage;
the method further comprises restarting administration of the one or more maintenance dosages of step (ii).

In some embodiments, if the patient misses a maintenance dosage (e.g., a once weekly maintenance dosage); and
wherein between about 8 days to about 10 days have elapsed since administration of a prior maintenance dosage;
the method further comprises restarting administration of the one or more maintenance dosages of step (ii).

In some embodiments, if the patient misses a maintenance dosage (e.g., a once weekly maintenance dosage); and
wherein between about 8 days to about 9 days have elapsed since administration of a prior maintenance dosage;
the method further comprises restarting administration of the one or more maintenance dosages of step (ii).

In some embodiments, administration of the one or more maintenance dosages of step (ii) to the patient is restarted as soon as possible after the missed dose.

### EXAMPLES

The disclosure will be described in greater detail by way of specific examples. The following examples are offered for illustrative purposes, and are not intended to limit the disclosure in any manner. Those of skill in the art will readily recognize a variety of noncritical parameters which can be changed or modified to yield essentially the same results.

### Example 1. Bioavailability of Oral Fixed-Dose Combination Tablet Formulations of Islatravir/Lenacapavir

This study was a Phase 1, open-label, parallel design, single-dose, multiple-cohort study in healthy participants to evaluate the relative bioavailability of islatravir (ISL) and lenacapavir (LEN) in oral fixed-dose combination (FDC) tablet formulation(s) containing ISL (2 mg) and LEN (300 mg), compared with ISL capsules (2×1 mg) and a LEN tablet (300 mg) administered simultaneously under fasted conditions.

Following completion of screening and admission (Day −1) assessments, eligible participants were enrolled to receive study treatments beginning on Day 1. The study treatment cohorts and treatment regimens are summarized in Table 1A.

TABLE 1A

| Cohort^{a} | Treatment Regimen (Day 1) |
|---|---|
| 1 | ISL 2 mg (2 × 1-mg capsules, fasted) + |
| | LEN 300 mg (1 × 300-mg tablet, fasted) |
| 2 | FDC No. 1 (ISL [2 mg] and LEN [300 mg], fasted) |
| 3 | FDC No. 1 (ISL [2 mg] and LEN [300 mg], fed) |

FDC = fixed-dose combination;
ISL = islatravir;
LEN = lenacapavir
^{a}Cohort 1 served as a reference for Cohort 2. Cohort 2 served as reference for Cohort 3.

Cohorts 1 and 2: Study drug was administered on Day 1 with 240 mL of water. On the day of intensive pharmacokinetic (PK) sampling, study treatment was administered in the morning following an overnight fast (no food or drinks, except water, for at least 10 hours). Participants continued to fast until after collection of the 4-hour PK sample, relative to study drug dosing. Additionally, participants were restricted from water consumption from 1 hour before dosing through 2 hours after dosing, except for the 240 mL given with the study treatment

Cohort 3: Study drug was administered on Day 1 with food and with 240 mL of water. Following an overnight fast (no food or drinks, except water, for at least 10 hours), a meal was initiated 30 minutes prior to study drug administration. The study drug dose was administered at or within 5 minutes of the participants completing (100%) the provided high-fat meal containing approximately 800 to 1000 kcal with approximately 50% of the calories from fat. Participants fasted for 4 hours after study drug administration. A meal (standardized lunch) was provided to participants after the 4-hour postdose blood draw. Additionally, water and other fluids were withheld from 1 hour before dosing through 2 hours after dose administration other than the water provided with dosing and beverages provided with the high-fat meal. Participants consumed water following the 2-hour blood draw for the remainder of the collection period.

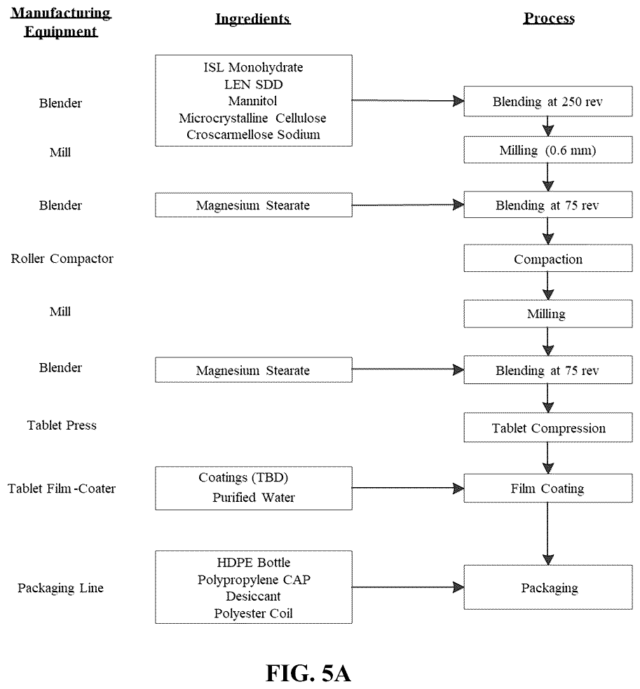

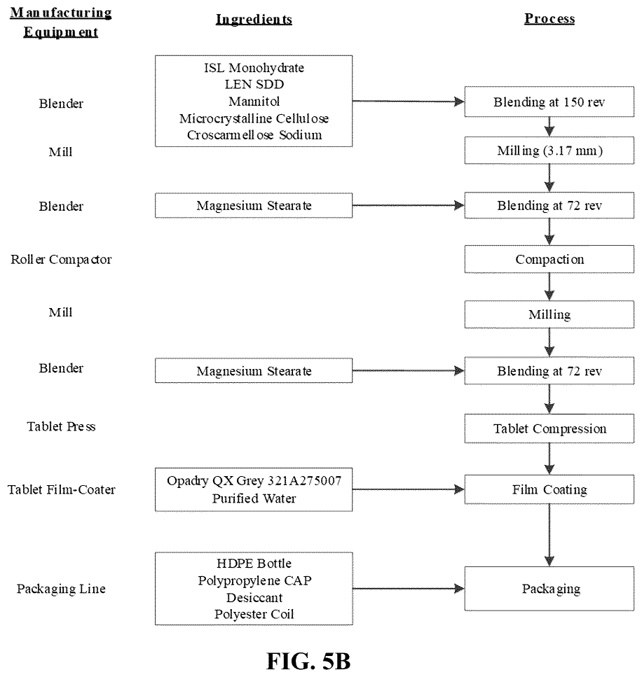

A summary of the FDC tablet administered to Cohorts 2-3 is shown in Table 1B and was prepared according to the scheme as shown in FIG. 5A.

TABLE 1B

ISL/LEN 2/300 mg

| | | |
|---|---|---|
| Components | % w/w | mg/tablet |

| | Intragranular | Islatravir | 0.142 | 2.130^{b} |
|---|---|---|---|---|
| | | Monohydrate | | |
| | | LEN SDD^{a} | 26.67 | 400.0 |
| | | Mannitol | 41.40 | 621.00 |
| | | (Pearlitol 160C) | | |
| | | Microcrystalline | 22.288 | 334.320 |
| | | Cellulose (PH 101) | | |
| | | Croscarmellose | 8 | 120.0 |
| | | Sodium | | |
| | | Magnesium Stearate | 0.5 | 7.5 |
| | Extragranular | Magnesium Stearate | 1.0 | 15.0 |
| Total Tablet Core | | | — | 1500 |
| | Film Coating | Opadry ® Clear | 1.0 | 15.0 |
| | | 03U19298 | | |
| | | Opadry ® TF Pink | 4.0 | 60.6 |
| | | 266U140003 | | |

^{a}Equivalent to 300 mg LEN free acid
^{b}Equivalent to 2 mg ISL free base

### Pharmacokinetic Assessments

### Plasma PK Sampling for ISL

Intensive PK sampling occurred relative to the AM dosing of ISL at the following time points for all cohorts:
Day 1: 0 (predose ≤5 minutes before dose), 0.5, 0.75, 1, 2, 4, 6, 8, 12, 24, 48, and 72 hours postdose.

A single anytime PK sample were collected at approximately the same time of day as the predose sample on Day 1 on the following days:
Days 6, 8, 10, 12, and 14.

### Plasma PK Sampling for LEN

Intensive PK sampling occurred relative to the AM dosing of LEN at the following time points for all cohorts:
Day 1: 0 (≤5 minutes before dose), 1, 2, 4, 6, 8, 12, 24, 48, and 72 hours postdose

A single anytime PK sample were collected at approximately the same time of day as the pre-dose sample on Day 1 on the following days:
Days 6, 8, 10, 12, 14, 16, 20, 24, 29, 36, and 43.

### Analysis of Pharmacokinetics

Relevant PK parameters were estimated using Phoenix WinNonlin® software using standard noncompartmental methods. The linear-up log-down trapezoidal rule was used in conjunction with the appropriate noncompartmental model, with input values for dose level, dosing time, plasma concentration, and corresponding real-time values, based on drug dosing times whenever possible. All pre-dose sample times before time zero were converted to zero.

Relevant PK parameters were generated for all participants for whom those parameters could be derived. The analytes presented in Table 2 were evaluated if data were available.

TABLE 2

| Cohort | Treatment | Analytes |
|---|---|---|
| 1 | ISL (2 × 1 mg) capsules + LEN | ISL and LEN |
| | 300-mg tablet, fasted | |
| 2 | ISL/LEN 2/300 mg FDC 1, fasted | ISL and LEN |
| 3 | ISL/LEN 2/300 mg FDC 1, fed | ISL and LEN |

FDC = fixed-dose combination;
ISL = islatravir;
LEN = lenacapavir

The analytes and parameters presented in Table 3 were used to evaluate the PK objectives of the study (as appropriate). The primary PK parameters were AUC_{inf}, AUCₗₐₛₜ, AUC₀₋₁, and Cₘₐₓ (as applicable) of ISL and LEN.

TABLE 3

| Analyte | Parameters |
|---|---|
| ISL | AUCₗₐₛₜ, AUC_{inf}^{a}, % AUCₑₓₚ, Cₘₐₓ, Tₘₐₓ, Cₗₐₛₜ, |
| | Tₗₐₛₜ, λz, CL/F, V_{z}/F, and t_{1/2} (as applicable) |
| LEN | AUCₗₐₛₜ, AUC_{inf}, % AUCₑₓₚ, Cₘₐₓ, Tₘₐₓ, Cₗₐₛₜ, |
| | Tₗₐₛₜ, λz, CL/F, V_{z}/F, and t_{1/2} (as applicable) |

ISL = islatravir;
LEN = lenacapavir
^{a}Parameter used in sample size and power calculations.

The statistical comparisons of the relevant natural log-transformed PK parameters for each analyte and treatment comparison of interest were performed. The statistical modeling was based on the PK analysis set for the analyte under evaluation. For each analyte, all participants with available data for the PK parameter under evaluation were included in the modeling. For each analyte, and each PK parameter, a parametric (normal theory) mixed-effects analysis of variance model was fitted to the natural log-transformed values of the single-dose PK parameter under evaluation.

### Drug Concentration Measurements

Concentrations of LEN and ISL in plasma samples were determined using fully validated high-performance liquid chromatography-tandem mass spectrometry bioanalytical methods. All samples were analyzed in the time frame supported by frozen stability storage data.

### Baseline Characteristics and Study Participant Disposition

A summary of participant enrollment and disposition was provided by cohort and overall. A by-participant listing of participant disposition including cohort, date of the last dose of study drug(s) (study days), study drug completion status, reason for study drug discontinuation, study completion status, reason for study discontinuation, and PK set status (indicating whether or not a participant is included in a PK analysis set) was provided by participant ID number in ascending order. A participant's extent of exposure to study drug data and meal administration data were generated and listed.

Overall, the median age of participants was 35 years (age range: 18-55). A similar proportion of participants were assigned male at birth (42 participants [45.2%]) and female at birth (51 participants [54.8%]). Most of the participants were White (77 participants [82.8%]), and not Hispanic or Latino (84 participants [90.3%]). Median (first quartile, third quartile) BMI was 26.5 (24.3, 28.3) kg/m².

Participant demographic variables (i.e., age, sex, race, and ethnicity) and baseline characteristics (body weight [in kg], height [in cm], and BMI [in kg/m²]) were summarized by cohort and overall using descriptive statistics for continuous variables and using number and percentage of participants for categorical variables. For baseline body weight, height, and BMI, descriptive statistics were presented by sex in the same table. The summary of demographic data was provided for the All Enrolled Analysis Set.

A total of 93 participants were enrolled and received study drugs (31 each in Cohorts 1, 2, and 3). All enrolled participants (100.0%) completed study drug dosing and 91 participants (97.8%) completed the study. Two participants (2.2%) prematurely discontinued study (both in Cohort 2). The reasons for premature discontinuation from study were lost to follow-up and withdrawal of consent (1 participant each in Cohort 2). The discontinuations had no impact on the overall quality or interpretation of the study data.

### Inclusion Criteria

Participants met all of the following inclusion criteria for eligibility in this study:
Have the ability to understand and sign a written informed consent form (ICF), obtained prior to initiation of study procedures.
Be aged 18 through 55 years, inclusive, at screening.
Be a nonsmoker. The use of nicotine or nicotine-containing products must be discontinued 90 days prior to the first dose of study drug.
Had a calculated body mass index (BMI) of ≥19.0 and ≤30.0 kg/m² at screening.
Had a calculated creatinine clearance (CL_{cr}) of ≥90 mL/min(using the Cockcroft-Gault method (Cockcroft and Gault, Nephron, 1976; 16:31-41) based on serum creatinine and actual body weight as measured at screening and upon admission:
   Participant assigned male at birth:
      (140−Age [years])×(Weight [kg])=CL_{cr} (mL/min)
      72×(Serum Creatinine [mg/dL])
   Participant assigned female at birth:
      (140−Age [years])×(Weight [kg])×0.85=CL_{cr} (mL/min)
      72×(Serum Creatinine [mg/dL])
Participants assigned female at birth and of childbearing potential who engage in heterosexual intercourse agreed to use protocol-specified method(s) of contraception.
Participants have not donated blood within 56 days of study entry or plasma within 7 days of study entry and refrain from blood donation from clinic admission, throughout the study period, and continuing for ≥60 days following the last dose of study drug.
Laboratory evaluations and 12-lead electrocardiogram (ECG) evaluations at screening and/or admission must be without clinically significant abnormalities.
Had liver biometric tests such as ALT, aspartate aminotransferase (AST), alkaline phosphatase, and total bilirubin at or below the upper limit of normal at screening and at admission.
Willing and able to comply with all study requirements.
Be in good health based upon medical history and physical examination, including vital signs.

### Exclusion Criteria

Participants who met any of the following exclusion criteria were not be enrolled in this study:
Positive serum pregnancy test at screening or at admission.
Breastfeeding participant.
Had received any study drug within 30 days prior to study dosing or exposure to LEN or ISL in a prior clinical study.
Had current alcohol or substance abuse judged by the investigator to potentially interfere with participant compliance or participant safety, or a positive drug or alcohol test at screening or baseline.
Had a positive test result for HIV-1/2 antibody, hepatitis B virus (HBV) surface antigen (HBsAg), or hepatitis C virus (HCV) antibody at screening.
Had poor venous access that limits phlebotomy.
Had taken any prescription medications or over-the-counter medications, including herbal products, within 28 days prior to start of study drug dosing, with the exception of vitamins and/or acetaminophen and/or ibuprofen and/or hormonal contraceptive medications.
Had been treated with systemic steroids, immunosuppressant therapies, or chemotherapeutic agents within 3 months prior to screening or is expected to receive these agents during the study (e.g., corticosteroids, immunoglobulins, other immune- or cytokine-based therapies).
Had a history of any of the following:
   Significant drug sensitivity or drug allergy (such as anaphylaxis or hepatoxicity) or food allergy.
   Known hypersensitivity to the study drugs, their metabolites, or to formulation excipients.
   Family history of long QT syndrome, or unexplained death in an otherwise healthy individual between the ages of 1 and 30 years.
   Severe peptic ulcer disease, gastroesophageal reflux disease, or other gastric acid hypersecretory conditions requiring prolonged (≥6 months) medical treatment.
   Medical or surgical treatment that permanently altered gastric absorption (e.g., gastric or intestinal surgery). A history of cholecystectomy was not exclusionary.
   Had any serious or active medical or psychiatric illness (including depression) that, in the opinion of the investigator, would interfere with participant treatment, assessment, or compliance with the protocol. This would include renal, cardiac (including implanted defibrillator or pacemaker), hematological, hepatic (including Gilbert syndrome), pulmonary (including chronic asthma), endocrine (including diabetes), central nervous, gastrointestinal (including an ulcer), vascular, metabolic (thyroid disorders, adrenal disease), immunodeficiency disorders, active infection, or malignancy that are clinically significant or requiring treatment.

### Safety Analysis

The incidence of treatment-emergent adverse events (TEAEs), serious TEAEs, and laboratory abnormalities were summarized by cohort.

### Adverse Events

An AE is any untoward medical occurrence in a clinical study participant administered a study drug that does not necessarily have a causal relationship with the treatment. An AE can, therefore, be any unfavorable and/or unintended sign, symptom, or disease temporally associated with the use of a study drug, whether or not considered related to the study drug. Adverse events may also include pretreatment or posttreatment complications that occur as a result of protocol-specified procedures, or special situations.

An AE does not include the following:
Medical or surgical procedures such as surgery, endoscopy, tooth extraction, and transfusion. The condition that led to the procedure may be an AE and must be reported.
Preexisting diseases, conditions, or laboratory abnormalities present or detected before the screening visit that do not worsen.
Situations where an untoward medical occurrence has not occurred (e.g., hospitalization for elective surgery, social and/or convenience admissions).
Overdose without clinical sequelae.
Any medical condition or clinically significant laboratory abnormality with an onset date before the ICF is signed and not related to a protocol-associated procedure is not an AE but rather considered to be preexisting and should be documented medical history.

Preexisting events that increase in severity or change in nature after study drug initiation or during or as a consequence of participation in the clinical study were also considered AEs.

### Serious Adverse Events

A serious adverse event (SAE) is defined as an event that, at any dose, results in the following:
Death.
A life-threatening situation (the term “life-threatening” in the definition of “serious” refers to an event in which the participant was at risk of death at the time of the event; it does not refer to an event that hypothetically might have caused death if it were more severe.)
Inpatient hospitalization or prolongation of existing hospitalization.
Persistent or significant disability/incapacity.
A congenital anomaly/birth defect.
A medically important event or reaction; such events may not be immediately life-threatening or result in death or hospitalization but may jeopardize the participant or may require intervention to prevent 1 of the other outcomes constituting SAEs. Medical and scientific judgment must be exercised to determine whether such an event is a reportable under expedited reporting rules. Examples of medically important events include intensive treatment in an emergency room or at home for allergic bronchospasm; blood dyscrasias or convulsions that do not result in hospitalization; and development of drug dependency or drug abuse.

All 93 enrolled participants received study drug and were included in the Safety Analysis Set. All 93 participants (31 each in Cohorts 1, 2, and 3) who received study drug and had at least 1 nonmissing concentration value reported by the PK laboratory for the corresponding analytes (ISL or LEN) were included in the ISL or LEN PK Analysis Sets, respectively.

### Pharmacokinetics of Islatravir

### Islatravir Plasma Concentrations

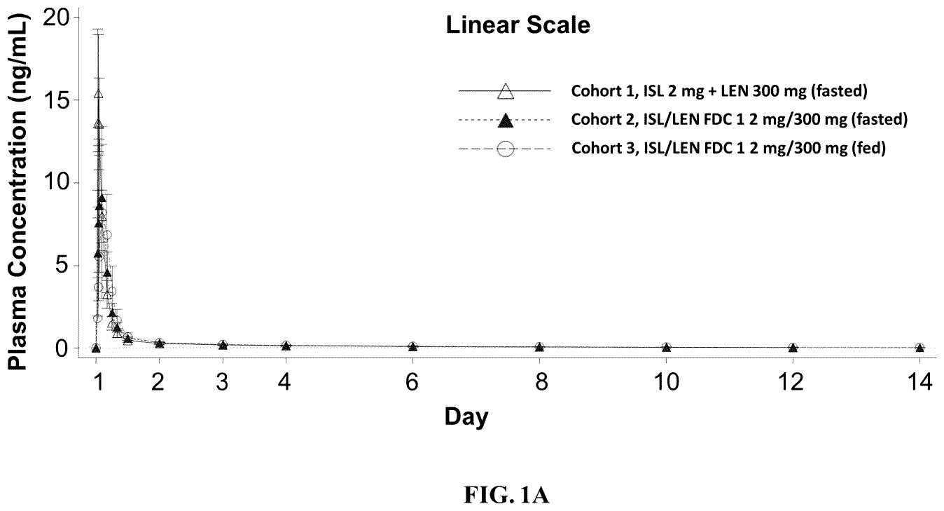

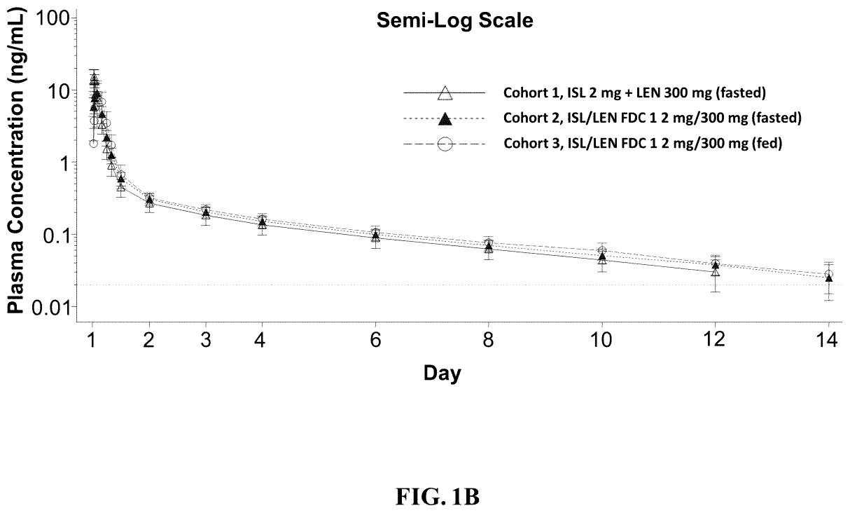

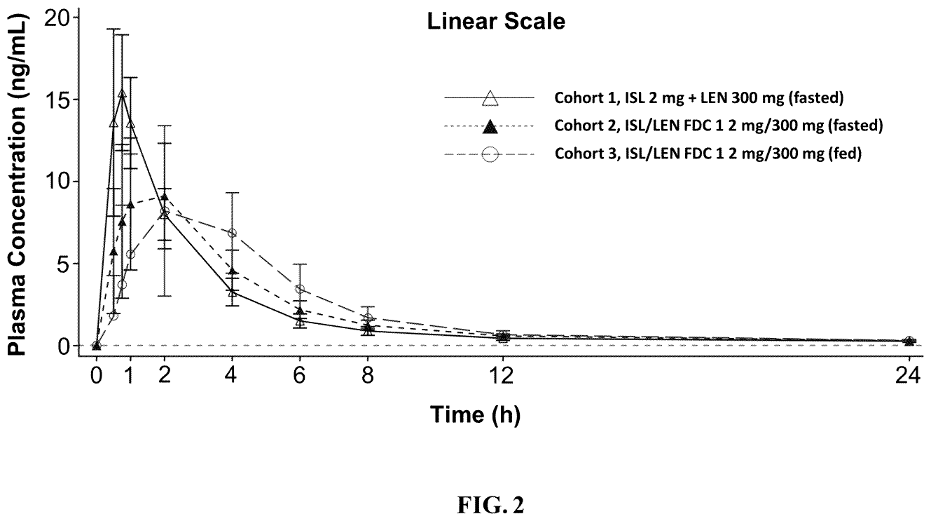

Islatravir plasma concentration-time profiles following coadministration of ISL 2 mg+LEN 300 mg under fasted conditions (Cohort 1), single-dose administration of the ISL/LEN 2/300 mg FDC under fasted (Cohort 2) and fed conditions (Cohort 3) are presented in FIGS. 1A-1B and FIG. 2. Overall, mean ISL plasma concentration-time profiles were similar across all cohorts (FIGS. 1A-1), with lower peak concentrations in ISL/LEN 2/300 mg FDC cohorts compared with single agent coadministration (FIG. 2).

### Islatravir Plasma PK Parameters and Statistical Analysis

### Relative Bioavailability (Formulation Effect) Assessment

Table 4 summarizes ISL PK parameters when administered as an ISL/LEN FDC or coadministered with LEN and the statistical comparisons for ISL Cₘₐₓ, AUC_{inf}, and AUCₗₐₛₜ. Mean AUC_{inf} of ISL in the ISL/LEN 2/300 mg FDC (Cohort 2) were similar to those observed with coadministered ISL 2 mg+LEN 300 mg (Cohort 1) in the reference treatment (AUC_{inf} % GLSM ratio [90% CI] of 107 [97.8, 116]) under fasted conditions. Mean Cₘₐₓ in the ISL/LEN 2/300 mg FDC cohort was approximately 36% lower than those observed with coadministered ISL 2 mg+LEN 300 mg in the reference treatment (Cₘₐₓ % GLSM ratio [90% CI] of 64.4 [56.7, 73.1]).

TABLE 4

| | | | |
|---|---|---|---|
| | | | % GLSM Ratio |
| ISL PK | Test | Reference | (90% CI) Test |
| Parameter | (N = 31) | (N = 31) | vs Reference |

Cohort 2 (ISL/LEN 2/300 mg FDC, Fasted) Test vs Cohort 1
(ISL 2 mg + LEN 300 mg Coadministration, Fasted) Reference

| | | | |
|---|---|---|---|
| Cₘₐₓ (ng/mL) | 11.0 (35.0) | 16.5 (22.0) | 64.4 |
| | | | (56.7, 73.1) |
| AUC_{inf} | 76.7 (17.1)^{a} | 72.6 (22.3) | 107 |
| (h · ng/mL) | | | (97.8, 116) |
| AUCₗₐₛₜ | 72.1 (17.4)^{a} | 68.8 (22.6) | 106 |
| (h · ng/mL) | | | (96.7, 116) |
| % AUCₑₓₚ | 6.08 (24.2)^{a} | 5.27 (19.4) | — |
| Tₘₐₓ (h) | 1.03 | 0.750 | — |
| | (0.750, 2.00) | (0.500, 0.750) | |
| t_{1/2} (h) | 101 | 95.4 | — |
| | (94.4, 110)^{a} | (87.5, 103) | |

% CV = percentage coefficient of variation;
CI = confidence interval;
FDC = fixed-dose combination;
GLSM = geometric least-squares mean;
ISL = islatravir;
LEN = lenacapavir;
PK = pharmacokinetic(s);
Q1 = first quartile;
Q3 = third quartile
^{a}N = 30 due to early discontinuation after Day 3 in 1 participant in Cohort 2.
The PK parameters are presented as mean (% CV) except Tₘₐₓ and t_{1/2}, which are presented as median (Q1, Q3).
N represents the number of participants in the PK analysis set.
Means presented are unadjusted arithmetic means.

### Food Effect Assessment

The food effect on ISL PK for the ISL/LEN 2/300 mg FDC was evaluated with a high-fat meal. Mean ISL AUC_{inf} and Cₘₐₓ were similar in fed versus fasted cohorts (Table 5: AUC_{inf} % GLSM ratio [90% CI] of 107 [99.4, 115], and Cₘₐₓ % GLSM ratio [90% CI] of 106 [90.6, 123]).

TABLE 5

| | | | |
|---|---|---|---|
| | | | % GLSM Ratio |
| ISL PK | Test | Reference | (90% CI) Test |
| Parameter | (N = 31) | (N = 31) | vs Reference |

Cohort 3 (ISL/LEN 2/300 mg FDC, High-Fat Meal) Test vs
Cohort 2 (ISL/LEN 2/300 mg FDC, Fasted) Reference

| | | | | | |
|---|---|---|---|---|---|
| Cₘₐₓ | 11.7 | (37.7) | 11.0 | (35.0) | 106 |
| (ng/mL) | | | | | (90.6, 123) |
| AUC_{inf} | 82.0 | (17.0) | 76.7 | (17.1)^{a} | 107 |
| (h · ng/mL) | | | | | (99.4, 115) |
| AUCₗₐₛₜ | 77.2 | (16.8) | 72.1 | (17.4)^{a} | 107 |
| (h · ng/mL) | | | | | (99.6, 116) |
| % AUCₑₓₚ | 5.80 | (27.2) | 6.08 | (24.2)^{a} | — |

| | | | |
|---|---|---|---|
| Tₘₐₓ (h) | 2.00 | 1.03 | — |
| | (2.00, 4.00) | (0.750, 2.00) | |
| t_{1/2} (h) | 103 | 101 | — |
| | (94.7, 114) | (94.4, 110)^{a} | |

% CV = percentage coefficient of variation;
CI = confidence interval;
FDC = fixed-dose combination;
GLSM = geometric least-squares mean;
ISL = islatravir;
LEN = lenacapavir;
PK = pharmacokinetic(s);
Q1 = first quartile;
Q3 = third quartile
^{a}N = 30 due to early discontinuation after Day 3 in 1 participant in Cohort 2.
The PK parameters are presented as mean (% CV) except Tₘₐₓ and t_{1/2}, which are presented as median (Q1, Q3).
N represents the number of participants in the PK analysis set.
Means presented are unadjusted arithmetic means

### Pharmacokinetics of Lenacapavir

### Lenacapavir Plasma Concentrations

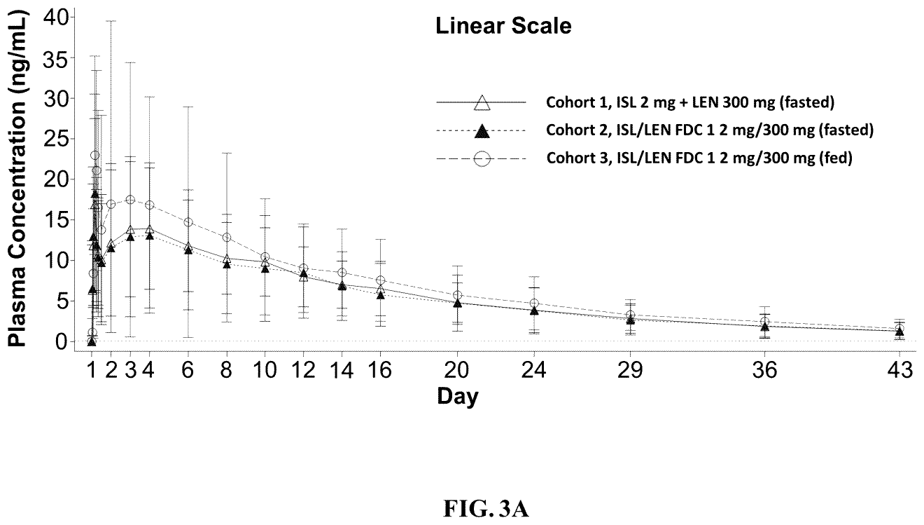

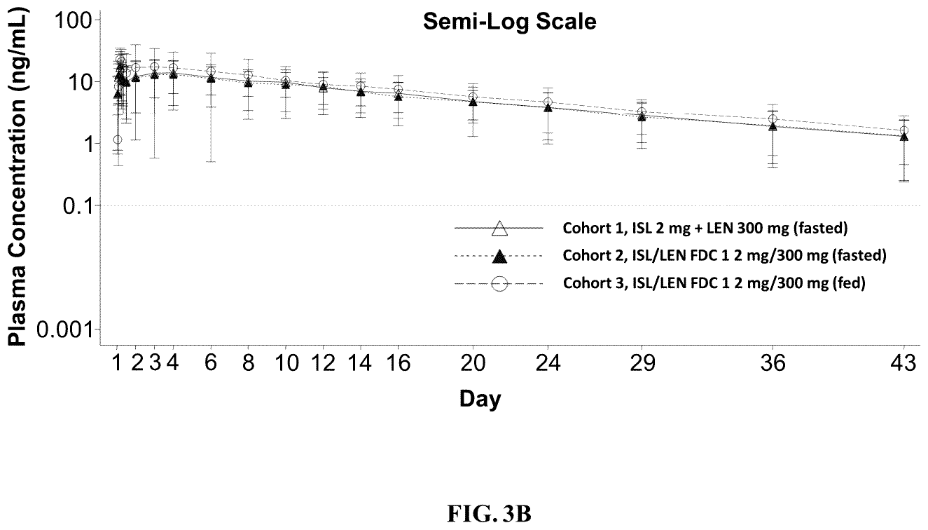

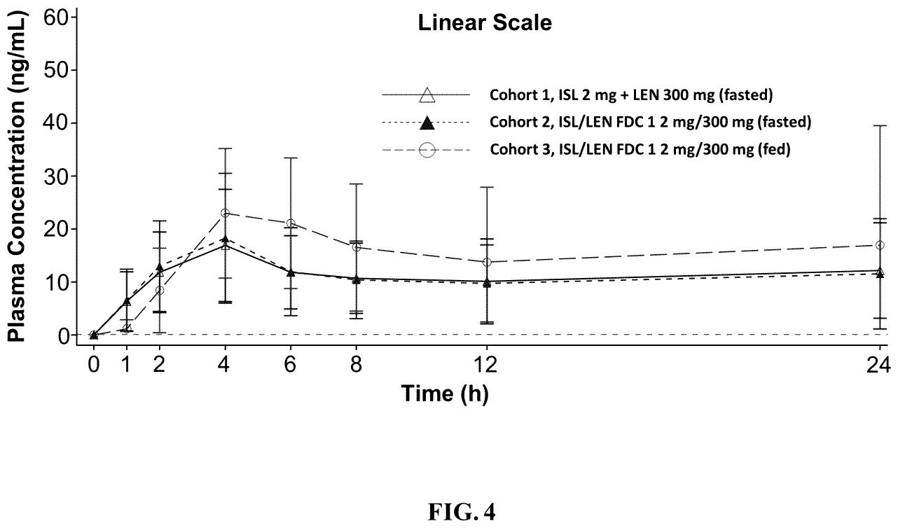

Lenacapavir plasma concentration-time profiles following coadministration of ISL 2 mg+LEN 300 mg under fasted conditions (Cohort 1), single-dose administration of the ISL/LEN 2/300 mg FDC under fasted (Cohort 2) and fed conditions (Cohort 3) are presented in FIGS. 3A-3B and FIG. 4. Lenacapavir exposures in Cohorts 1 and 2 were similar, while the exposures were generally increased (Cohort 3) with a high-fat meal (FIG. 4).

### Lenacapavir Plasma PK Parameters and Statistical Analysis

### Relative Bioavailability (Formulation Effect) Assessment

Table 6 summarizes LEN PK parameters when administered as an FDC or coadministered with ISL and the statistical comparisons for LEN Cₘₐₓ, AUC_{inf}, and AUCₗₐₛₜ. Mean LEN exposures in the ISL/LEN 2/300 mg FDC (Cohort 2) were similar to those observed with coadministered ISL 2 mg+LEN 300 mg (Cohort 1) in the reference treatment under fasted conditions (AUC_{inf} % GLSM ratio [90% CI] of 90.2 [72.1, 113], and Cₘₐₓ % GLSM ratio [90% CI] of 103 [80.6, 132]).

TABLE 6

| | | | |
|---|---|---|---|
| | | | % GLSM Ratio |
| LEN PK | Test | Reference | (90% CI) Test |
| Parameter | (N = 31) | (N = 31) | vs Reference |

Cohort 2 (ISL/LEN 2/300 mg FDC, Fasted) Test vs Cohort 1
(ISL 2 mg + LEN 300 mg Coadministration, Fasted) Reference

| | | | | | |
|---|---|---|---|---|---|
| Cₘₐₓ | 19.4 | (64.9) | 18.1 | (61.3) | 103 |
| (ng/mL) | | | | | (80.6, 132) |
| AUC_{inf} | 6020 | (66.1)^{a} | 6290 | (50.4) | 90.2 |
| (h · ng/mL) | | | | | (72.1, 113) |
| AUCₗₐₛₜ | 5310 | (66.9)^{a} | 5660 | (46.0) | 87.6 |
| (h · ng/mL) | | | | | (70.2, 109) |
| % AUCₑₓₚ | 11.5 | (58.4)^{a} | 8.95 | (64.2) | — |

| | | | |
|---|---|---|---|
| Tₘₐₓ (h) | 4.00 | 4.00 | — |
| | (4.00, 4.00) | (4.00, 24.0) | |
| t_{1/2} (h) | 317 | 279 | — |
| | (246, 371)^{a} | (239, 306) | |

% CV = percentage coefficient of variation;
CI = confidence interval;
FDC = fixed-dose combination;
GLSM = geometric least-squares mean;
ISL = islatravir;
LEN = lenacapavir;
PK = pharmacokinetic(s);
Q1 = first quartile;
Q3 = third quartile
^{a}N = 30 due to early discontinuation after Day 3 in 1 participant in Cohort 2.
The PK parameters are presented as mean (% CV) except Tₘₐₓ and t_{1/2}, which are presented as median (Q1, Q3).
N represents the number of participants in the PK analysis set.
Means presented were unadjusted arithmetic means.

### Food Effect Assessment

The food effect on LEN PK for the ISL/LEN 2/300 mg FDC was evaluated with a high-fat meal. Mean LEN AUC_{inf} and Cₘₐₓ were approximately 27% and 43% higher, respectively, in the fed versus fasted cohorts (Table 7: AUC_{inf} % GLSM ratio [90% CI] of 127 [97.7, 164], and Cₘₐₓ % GLSM ratio [90% CI] of 143 [108, 189]).

TABLE 7

| | | | |
|---|---|---|---|
| | | | % GLSM Ratio |
| LEN PK | Test | Reference | (90% CI) Test |
| Parameter | (N = 31) | (N = 31) | vs Reference |

Cohort 3 (ISL/LEN 2/300 mg FDC, High-Fat Meal) Test vs
Cohort 2 (ISL/LEN 2/300 mg FDC, Fasted) Reference

| | | | | | |
|---|---|---|---|---|---|
| Cₘₐₓ | 28.6 | (75.1) | 19.4 | (64.9) | 143 |
| (ng/mL) | | | | | (108, 189) |
| AUC_{inf} | 7740 | (69.0) | 6020 | (66.1)^{a} | 127 |
| (h · ng/mL) | | | | | (97.7, 164) |
| AUCₗₐₛₜ | 6900 | (71.6) | 5310 | (66.9)^{a} | 127 |
| (h · ng/mL) | | | | | (97.8, 165) |
| % AUCₑₓₚ | 11.1 | (69.7) | 11.5 | (58.4)^{a} | — |

| | | | |
|---|---|---|---|
| Tₘₐₓ (h) | 4.00 | 4.00 | — |
| | (4.00, 6.00) | (4.00, 4.00) | |
| t_{1/2} (h) | 304 | 317 | — |
| | (246, 345) | (246, 371)^{a} | |

% CV = percentage coefficient of variation;
CI = confidence interval;
FDC = fixed-dose combination;
GLSM = geometric least-squares mean;
ISL = islatravir;
LEN = lenacapavir;
PK = pharmacokinetic(s);
Q1 = first quartile;
Q3 = third quartile
^{a}N = 30 due to early discontinuation after Day 3 in 1 participant in Cohort 2.
The PK parameters are presented as mean (% CV) except Tₘₐₓ and t_{1/2}, which are presented as median (Q1, Q3).
N represents the number of participants in the PK analysis set.
Means presented were unadjusted arithmetic means.

Comparison with Historical Data

No clinically relevant drug-drug interactions on the PK of ISL and LEN were observed following ISL and LEN co-administration. Therefore, historical LEN PK data from single agent LEN tablet (300 mg) administered orally under fasted conditions was utilized as additional references (Cohort 7 and Cohort 4 in Table 8) to compare and contextualize the PK findings given the relatively higher variability for LEN. As shown in Table 8, LEN exposures in the cohort of ISL 2 mg+LEN 300 mg coadministration (Cohort 1) were generally comparable with those observed in the additional reference studies. Thus, the data from Cohort 1 and these additional references were pooled to increase sample size for additional assessment of FDC versus single agent comparison for LEN exposures.

Consistent with study-defined comparison (Table 6), LEN exposures in the ISL/LEN 2/300 mg FDC (Cohort 2) were generally similar to those observed in this pooled LEN data as the reference treatment under fasted conditions (Table 9): AUC_{inf} % GLSM ratio [90% CI] of 96.7 [79.2, 118], and Cₘₐₓ % GLSM ratio [90% CI] of 98.3 [78.0, 124]).

TABLE 8

| | | | Reference | | Reference | | | |
|---|---|---|---|---|---|---|---|---|
| | | | Data for LEN | | Data for LEN | | Pooled | |
| LEN PK | Cohort 1^{a} | | Cohort 7^{b} | | Cohort 4^{c} | | LEN Data | |
| Parameter | (N = 31) | | (N = 8) | | (N = 27) | | (N = 66) | |
| Cₘₐₓ | 18.1 | (61.3) | 33.7 | (96.3) | 20.4 | (102) | 20.9 | (91.9) |
| (ng/mL) | | | | | | | | |
| AUC_{inf} | 6290 | (50.4) | 7690 | (57.8) | 5290 | (61.1) | 6050 | (56.2) |
| (h · ng/mL) | | | | | | | | |

| | | | | |
|---|---|---|---|---|
| Tₘₐₓ (h) | 4.00 | 4.00 | 4.00 | 4.00 |
| | (4.00, 24.00) | (4.00, 6.00) | (4.00, 6.00) | (4.00, 6.00) |
| t_{1/2} (h) | 279 | 243 | 329 | 279 |
| | (239, 306) | (204, 281) | (231, 388) | (231, 332) |

ISL = islatravir;
LEN = lenacapavir;
PK = pharmacokinetic(s);
Q1 = first quartile;
Q3 = third quartile
^{a}ISL 2 mg + LEN 300 mg coadministration under fasted conditions.
^{b}LEN 300 mg single agent under fasted conditions.
^{c}LEN 300 mg single agent under fasted conditions.
The PK parameters are presented as mean (% CV) except Tₘₐₓ and t_{1/2}, which are presented as median (Q1, Q3).

TABLE 9

| | | | |
|---|---|---|---|
| | | | % GLSM Ratio |
| LEN PK | Test | Reference | (90% CI) Test |
| Parameter | (N = 31) | (N = 66) | vs Reference |

Cohort 2 (ISL/LEN 2/300 mg FDC, Fasted) Test vs
Pooled LEN data (LEN 300-mg Tablet, Fasted) Reference

| | | | | | |
|---|---|---|---|---|---|
| Cₘₐₓ | 19.4 | (64.9) | 20.9 | (91.9) | 98.3 |
| (ng/mL) | | | | | (78.0, 124) |
| AUC_{inf} | 6020 | (66.1)^{a} | 6050 | (56.2) | 96.7 |
| (h · ng/mL) | | | | | (79.2, 118) |

| | | | |
|---|---|---|---|
| Tₘₐₓ (h) | 4.00 | 4.00 | — |
| | (4.00, 4.00) | (4.00, 6.00) | |
| t_{1/2} (h) | 317 | 279 | — |
| | (246, 371)^{a} | (231, 332) | |

% CV = percentage coefficient of variation;
CI = confidence interval;
FDC = fixed-dose combination;
GLSM = geometric least-squares mean;
ISL = islatravir;
LEN = lenacapavir;
PK = pharmacokinetic(s);
Q1 = first quartile;
Q3 = third quartile
^{a}N = 30 due to early discontinuation after Day 3 in 1 participant in Cohort 2.
The PK parameters are presented as mean (% CV) except Tₘₐₓ and t_{1/2}, which are presented as median (Q1, Q3).
N represents the number of participants in the PK analysis set.
Means presented were unadjusted arithmetic means.

### Safety Evaluation

The key safety results from this study are as follows:
ISL and LEN were safe and generally well tolerated when administered as an FDC.
Headache was the most commonly reported AE in this study. Most AEs were Grade 1 in severity. One participant in Cohort 3 had a Grade 2 AE of urinary tract infection, which was considered not related to study drug.
One participant (3.2%) each in Cohorts 1 and 2, and 5 participants (16.1%) in Cohort 3 had study drug-related AEs.
No deaths, Grade 3 or 4 AEs, SAEs, AEs leading to discontinuation of study drug or study, or pregnancies were reported during the study.
Most of the laboratory abnormalities were Grade 2 or 3 in severity.
No clinically significant changes in vital signs, physical findings, or safety ECG results were observed during this study.

### Extent of Exposure

The median extent of exposure to study drug was similar between cohorts: Cohort 1 (59 days), Cohort 2 (60 days), and Cohort 3 (61 days). All participants (100.0%) in Cohorts 1 and 3 and 96.8% of participants in Cohort 2 experienced a duration of exposure of at least 56 days of treatment.

### Adverse Events

Overall, AEs were reported in 27 participants: 12 of 31 participants (38.7%) in Cohort 1, 5 of 31 participants (16.1%) in Cohort 2, and 10 of 31 participants (32.3%) in Cohort 3. One participant (3.2%) each in Cohorts 1 and 2, and 5 participants (16.1%) in Cohort 3 had study drug-related AEs. All AEs were of Grade 1 or 2 in severity. There were no deaths, SAEs, Grade 3 or higher AEs, or AEs leading to discontinuation of study drug or study.

Most AEs were Grade 1 in severity. One participant in Cohort 3 had a Grade 2 AE of urinary tract infection which was considered not related to study drug. No Grade 3 or higher AEs were reported during the study

The frequency of participants with any AE was relatively lower in Cohort 2 compared with Cohorts 1 and 3. Headache was the most commonly reported AE in this study. The most commonly reported AEs (reported in ≥2 participants) by cohort were as follows:
Cohort 1—headache (5 participants [16.1%]); abdominal pain lower, dysmenorrhea, and ill-defined disorder (each in 2 participants [6.5%])
Cohort 2—headache and nausea (each in 2 participants [6.5%])
Cohort 3—headache (3 participants [9.7%]); constipation, cough, nasal congestion, and nausea (each in 2 participants [6.5%]).

Treatment-related AEs included:
Cohort 1: rash (1 participant [3.2%])
Cohort 2: nausea and vomiting (each in 1 participant [3.2%])
Cohort 3: headache and nausea (each in 2 participants [6.5%]) and cold sweat (1 participant [3.2%])

For the Cohort 1 treatment-related AE of rash, causality of related was applied to both components ISL and LEN; for all Cohorts 2 and 3 treatment-related AEs, causality of related was applied to the combination treatment ISL/LEN FDC.

### CONCLUSIONS

ISL had comparable AUC_{inf} and approximately 36% lower Cₘₐₓ when administered as a single dose of ISL/LEN 2/300 mg FDC (Cohort 2, fasted dosing) relative to the ISL 2 mg+LEN 300 mg coadministration (Cohort 1, fasted dosing). The apparent slower rate of absorption for ISL from FDC compared with single agent capsule, reflected in the lower Cₘₐₓ, is likely due to a relatively slower initial release from the FDC tablet considering the formulation and size difference between the ISL capsule (16×6 mm, 150.1 mg) and the ISL/LEN FDC tablet (21×10 mm, 1500 mg). However, the total extent of absorption (AUC_{inf}) for ISL in the FDC tablet remains similar to the capsule. This indicates that while the initial rate of ISL release seems to be impacted by formulation, the total extent of absorption (AUC_{inf}) is not affected.

ISL exposures (AUC_{inf} and Cₘₐₓ) were generally similar with fed (Cohort 3) versus fasted (Cohort 2) administration of a single dose of ISL/LEN 2/300 mg FDC. These data support the administration of the ISL/LEN FDC without regard to food.

LEN exposures (AUC_{inf} and Cₘₐₓ) were generally similar when administered as a single dose of ISL/LEN 2/300 mg FDC (Cohort 2, fasted dosing) relative to the ISL 2 mg+LEN 300 mg coadministration (Cohort 1, fasted dosing). An additional analysis, with pooled data containing Cohort 1 and additional historical LEN studies to increase sample size for robust comparative assessment of FDC versus single agent, showed comparable findings for LEN exposures.

LEN exposures were generally higher (27% and 43% higher AUC_{inf} and Cₘₐₓ, respectively) with fed (Cohort 3) versus fasted (Cohort 2) administration of a single dose of ISL/LEN 2/300 mg FDC. The increase is not considered clinically relevant and is in agreement with previously observed food effect for LEN as a single agent. These data support the administration of the ISL/LEN FDC without regard to food.

ISL and LEN administered as individual components and FDCs (under fasted and/or fed conditions) were safe and generally well tolerated as demonstrated by no Grade 3 or 4 AEs, and no deaths.

### Example 2. Fixed-Dose Combination Tablet Formulations

The following FDC tablet formulations of Tables 10-16 were prepared according to the scheme as shown in FIG. 5A (Tables 11-16) or FIG. 5B (Table 10).

TABLE 10

| | | ISL/LEN 20/300 mg | |
|---|---|---|---|
| Components | | % w/w | mg/tablet |
| Intragranular | Islatravir Monohydrate | 1.416 | 21.3^{b} |
| | LEN SDD^{a} | 26.67 | 400.0 |
| | Mannitol (Pearlitol 160C) | 41.40 | 621.0 |
| | Microcrystalline | 21.014 | 315.21 |
| | Cellulose (PH 101) | | |
| | Croscarmellose Sodium | 8.00 | 120.0 |
| | Magnesium Stearate | 0.50 | 7.5 |
| Extragranular | Magnesium Stearate | 1.0 | 15.0 |
| Total Tablet Core | | — | 1500 |
| Film Coating | Opadry ® QX Grey | 4.0 | 60 |
| | 321A275007 | | |

^{a}Equivalent to 300 mg LEN free acid
^{b}Equivalent to 20 mg ISL free base

TABLE 11

| | | ISL/LEN 3/300 mg | |
|---|---|---|---|
| Components | | % w/w | mg/tablet |
| Intragranular | Islatravir Monohydrate | 0.213 | 3.195^{b} |
| | LEN SDD^{a} | 26.67 | 400.0 |
| | Mannitol (Pearlitol 160C) | 41.40 | 621.00 |
| | Microcrystalline | 22.217 | 333.255 |
| | Cellulose (PH 101) | | |
| | Croscarmellose Sodium | 8.00 | 120.0 |
| | Magnesium Stearate | 0.50 | 7.5 |
| Extragranular | Magnesium Stearate | 1.0 | 15.0 |
| Total Tablet Core | | — | 1500 |
| Film Coating | Opadry ® QX Grey | 4.0 | 60 |
| | 321A275007 | | |

^{a}Equivalent to 300 mg LEN free acid
^{b}Equivalent to 3 mg ISL free base

TABLE 12

| | | ISL/LEN 2/300 mg | |
|---|---|---|---|
| Components | | % w/w | mg/tablet |
| Intragranular | Islatravir Monohydrate | 0.142 | 2.130^{b} |
| | LEN SDD^{a} | 26.67 | 400.0 |
| | Mannitol (Pearlitol 160C) | 41.40 | 621.00 |
| | Microcrystalline | 22.288 | 334.320 |
| | Cellulose (PH 101) | | |
| | Croscarmellose Sodium | 8 | 120.0 |
| | Magnesium Stearate | 0.5 | 7.5 |
| Extragranular | Magnesium Stearate | 1.0 | 15.0 |
| Total Tablet Core | | — | 1500 |
| Film Coating | Opadry ® QX Pink | 4.0 | 60 |
| | 321A840039 | | |

^{a}Equivalent to 300 mg LEN free acid
^{b}Equivalent to 2 mg ISL free base

TABLE 13

| | | ISL/LEN 2/300 mg | |
|---|---|---|---|
| Components | | % w/w | mg/tablet |
| Intragranular | Islatravir Monohydrate | 0.142 | 2.130^{b} |
| | LEN SDD^{a} | 26.67 | 400.0 |
| | Mannitol (Pearlitol 160C) | 41.40 | 621.00 |
| | Microcrystalline | 22.288 | 334.320 |
| | Cellulose (PH 101) | | |
| | Croscarmellose Sodium | 8 | 120.0 |
| | Magnesium Stearate | 0.5 | 7.5 |
| Extragranular | Magnesium Stearate | 1.0 | 15.0 |
| Total Tablet Core | | — | 1500 |
| Film Coating | Opadry ® Clear 03U19298 | 1.0 | 15.0 |
| | Opadry ® TF Pink | 4.0 | 60.6 |
| | 266U140003 | | |

^{a}Equivalent to 300 mg LEN free acid
^{b}Equivalent to 2 mg ISL free base

TABLE 14

| | | ISL/LEN 0.5/300 mg | |
|---|---|---|---|
| Components | | % w/w | mg/tablet |
| Intragranular | Islatravir Monohydrate | 0.0355 | 0.5325^{b} |
| | LEN SDD^{a} | 26.67 | 400.0 |
| | Mannitol (Pearlitol 160C) | 41.40 | 621.0 |
| | Microcrystalline | 22.3945 | 335.9175 |
| | Cellulose (PH 101) | | |
| | Croscarmellose Sodium | 8.00 | 120.0 |
| | Magnesium Stearate | 0.50 | 7.5 |
| Extragranular | Magnesium Stearate | 1.0 | 15.0 |
| Total Tablet Core | | — | 1500 |
| Film Coating | Opadry ® Clear 03U19298 | 1.0 | 15.0 |
| | Opadry ® TF Grey | 4.00 | 60.0 |
| | 266U175004 | | |

^{a}Equivalent to 300 mg LEN free acid
^{b}Equivalent to 0.5 mg ISL free base

TABLE 15

| | | ISL/LEN 2/300 mg | |
|---|---|---|---|
| Components | | % w/w | mg/tablet |
| Intragranular | Islatravir Monohydrate^{a} | 0.142 | 2.13^{a} |
| | Lenacapavir Sodium^{b} | 20.46 | 306.8^{c} |
| | Copovidone^{b} | 4.88 | 73.20 |
| | Poloxamer 407^{b} | 1.33 | 19.95 |
| | Mannitol (Pearlitol 160C) | 41.40 | 621.0 |
| | Microcrystalline | 22.288 | 334.32 |
| | Cellulose (PH 101) | | |
| | Croscarmellose Sodium | 8.00 | 120.0 |
| | Magnesium Stearate | 0.50 | 7.5 |
| Extragranular | Magnesium Stearate | 1.0 | 15.0 |

| | | |
|---|---|---|
| Total Tablet Core | 100.0 | 1500 |

Sub-Coat

| | | |
|---|---|---|
| Opadry ® Clear 03U19298 | 1.00 | 15.0 |

Film-Coat

| | | |
|---|---|---|
| Opadry TF Pink 266U140003 | 4.00 | 60.6 |

^{a}Equivalent to 2 mg ISL free base
^{b}LEN sodium, copovidone, and poloxamer 407 are combined in LEN SDD, methanol is used during the SDD process and is removed
^{c}Equivalent to 300 mg LEN free acid

TABLE 16

| | | ISL/LEN 0.5/300 mg | |
|---|---|---|---|
| Components | | % w/w | mg/tablet |
| Intragranular | Islatravir Monohydrate^{a} | 0.0355 | 0.53^{a} |
| | Lenacapavir Sodium^{b} | 20.46 | 306.8^{c} |
| | Copovidone^{b} | 4.88 | 73.20 |
| | Poloxamer 407^{b} | 1.33 | 19.95 |
| | Mannitol (Pearlitol 160C) | 41.40 | 621.00 |
| | Microcrystalline | 22.395 | 335.92 |
| | Cellulose (PH 101) | | |
| | Croscarmellose Sodium | 8.00 | 120.00 |
| | Magnesium Stearate | 0.50 | 7.50 |
| Extragranular | Magnesium Stearate | 1.0 | 15.00 |

| | | |
|---|---|---|
| Total Tablet Core | 100.00 | 1500 |

Sub-Coat

| | | |
|---|---|---|
| Opadry ® Clear 03U19298 | 1.00 | 15.0 |

Film-Coat

| | | |
|---|---|---|
| Opadry TF Grey 266U175004 | 4.00 | 60.6 |

^{a}Equivalent to 0.5 mg ISL free base
^{b}LEN sodium, copovidone, and poloxamer 407 are combined in LEN SDD, methanol is used during the SDD process and is removed
^{c}Equivalent to 300 mg LEN free acid

### Example 3. Lenacapavir Spray Dried Dispersion (LEN SDD)

Crystalline LEN sodium, copovidone, and poloxamer 407 were dissolved in methanol to prepare a feed solution for spray drying. In the spray drying process, the feed solution was atomized to form droplets that were quickly dried in a heated gas stream to form solid particles containing a uniform mixture of amorphous LEN sodium, copovidone, and poloxamer 407.

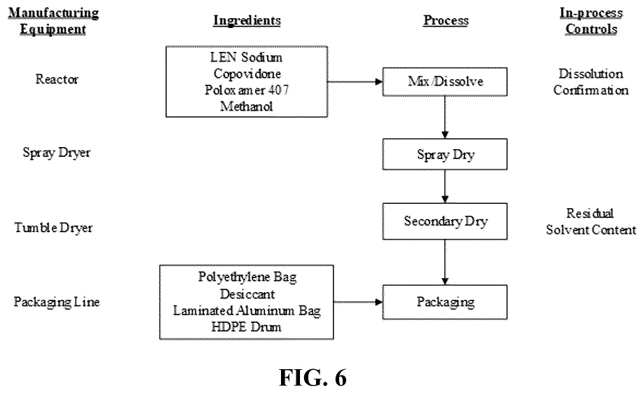

LEN SDD was manufactured using a series of unit operations summarized in the manufacturing process flow diagram shown in FIG. 6. The manufacturing process for LEN SDD consisted of four steps: (1) feed solution preparation by dissolving LEN sodium drug substance, copovidone, and poloxamer 407 in methanol; (2) spray drying the feed solution to yield primary-dried LEN SDD; (3) secondary drying to remove residual solvents to yield LEN SDD; and (4) packaging of LEN SDD. The components of the LEN SDD formulation used in preparing the tablets of Examples 1-2 are provided in Table 17. The column “Amount per tablet” of Table 17 refers to the amount (mg) of each component (e.g., lenacapavir sodium, copovidone, poloxamer 407) in each tablet comprising the LEN SDD.

TABLE 17

| | Composition | Amount per tablet |
|---|---|---|
| Component | (% w/w) | (mg/tablet) |
| lenacapavir sodium^{a} | 76.7 | 306.8^{b} |
| Copovidone^{a} | 18.3 | 73.2 |
| Poloxamer 407 | 5.0 | 20.0 |
| Methanol^{b} | — | — |
| Total Spray-Dried | 100 | 400 |
| Dispersion | | |

^{a}Equivalent to 300 mg of lenacapavir free acid
^{b}Methanol is used during the spray drying process and is removed during

### Example 4. Pharmacokinetic Modeling of Missed Dose Scenario of Oral Weekly Islatravir Plus Lenacapavir

Phase 1 study results indicated that ISL/LEN fixed dose combination (FDC) tablets had no significant effects on overall bioavailability or extent of absorption compared with single-agent tablets, and no drug-drug interaction was detected when the two agents were combined. Therefore, single-agent ISL and LEN models are reasonable for projecting exposure outcomes of various dosing scenarios.

In a Phase 2 study, following starting doses (2 mg ISL on Day 1 and 600 mg LEN on Days 1 and 2), a once-weekly (QW) oral regimen of 2 mg ISL plus 300 mg LEN maintained a high rate (94.2%) of viral suppression (HIV-1 RNA <50 copies/mL) at 48 weeks in PWH who were virologically suppressed. In two Phase 3 studies, following initiation doses (1 mg ISL and 600 mg LEN on Days 1 and 2, administered as two tablets of 0.5/300 mg ISL/LEN FDC on each day), a QW maintenance dose of oral 2/300 mg ISL/LEN FDC (one tablet) starting on Day 8 is being evaluated in virologically-suppressed PWH who have switched from standard-of-care treatment.

Using simulation analyses with existing ISL and LEN population pharmacokinetic (PopPK) models, this Example describes data to support effective drug concentrations within safety and efficacy margins in the event of missed oral QW ISL/LEN doses. PopPK modeling and simulation was utilized to evaluate pharmacokinetics (PK) of ISL and LEN following missed oral QW doses to quantify the degree of forgiveness for missed doses, and to inform make-up dosing strategies across various dosing scenarios.

### Methods

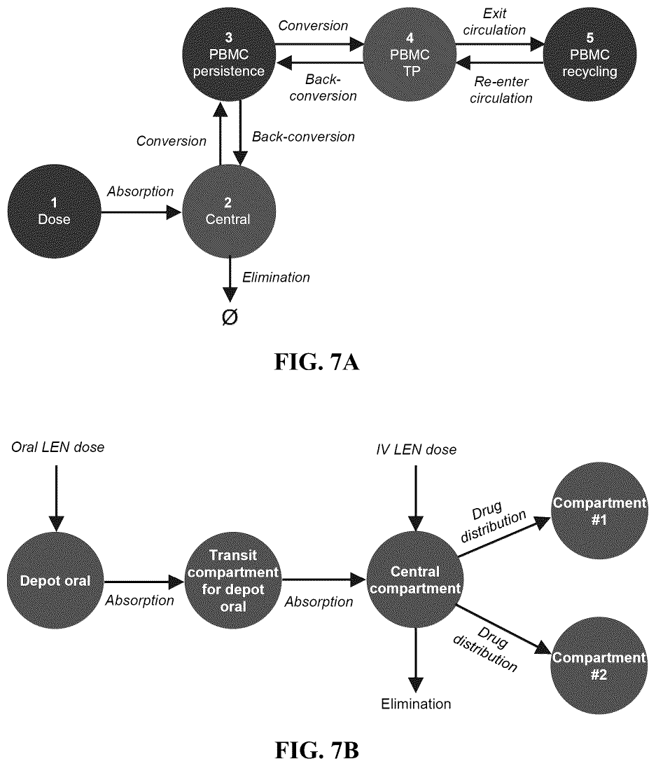

ISL-TP simulations used an existing PopPK model developed using oral ISL PK data from ISL Phase 1-3 studies, as shown in FIG. 7A. LEN simulations used an existing PopPK model developed with intravenous, oral, and subcutaneous LEN PK data from Phase 1-3 studies, as shown in FIG. 7B.

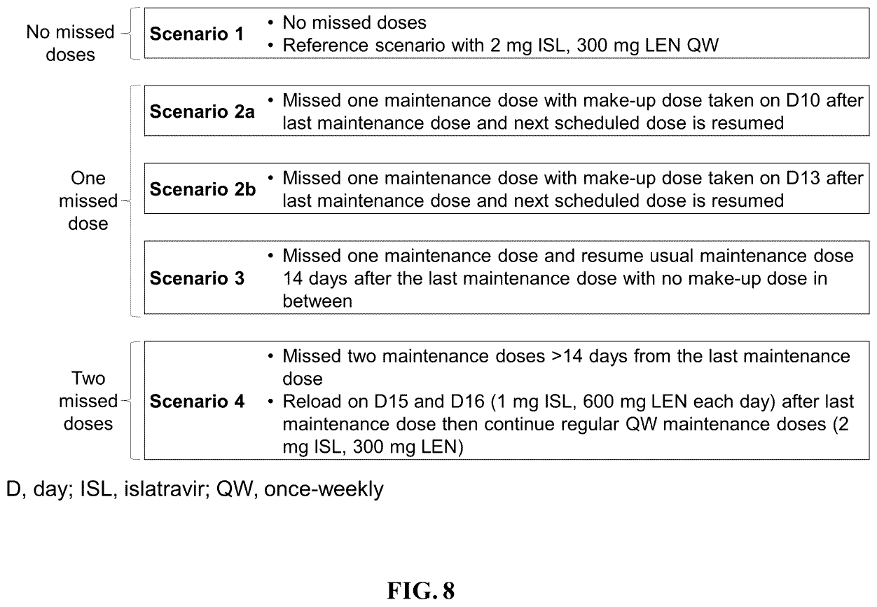

Simulations under steady-state conditions were performed to estimate the impact of various durations of missed doses and make-up-dose strategies. The reference scenario of no missed doses was clinical dosing with starting doses, 1 mg ISL and 600 mg LEN on Days 1 and 2, administered as two tablets of 0.5/300 mg ISL/LEN FDC on each day, followed by 2 mg ISL and 300 mg LEN (one tablet of 2/300 mg ISL/LEN FDC) QW starting on Day 8 as a maintenance dose. Simulation scenarios, shown in FIG. 8, were selected to include the specific scenarios that would provide the largest potential excursions in the maximum concentration (Cₘₐₓ) and the concentration at the end of the dosing interval (C_{trough}) in the event of missed or delayed doses. The same missed dose simulation scenarios were conducted for both ISL and LEN, as Phase 3 studies utilize an ISL/LEN FDC formulation.

Various dosing scenarios under steady-state conditions following missed oral QW doses were compared against the reference scenario of no missed doses (Scenario 1), as well as efficacy and clinically established safety exposure thresholds, as appropriate. For ISL-TP, the target was to ensure that the 95% prediction interval (PI) for median ISL-TP concentrations remained:
above the inhibitory quotient (IQ) 5 (1.25 μM) for M184V/I variants and IQ5 (0.25 μM) for wild-type HIV-1; and
below exposures associated with the 3 mg QW dose predicted in previous modeling and simulation analyses to have no meaningful impact on lymphocyte and CD4+ T-cell counts.

For LEN, the target was to ensure that the 90% confidence interval for mean LEN concentrations remained:
above the IQ4 (15.5 mg/mL); and
below the highest exposure with no associated safety signal, as established in a LEN Thorough-QT study.

### Results

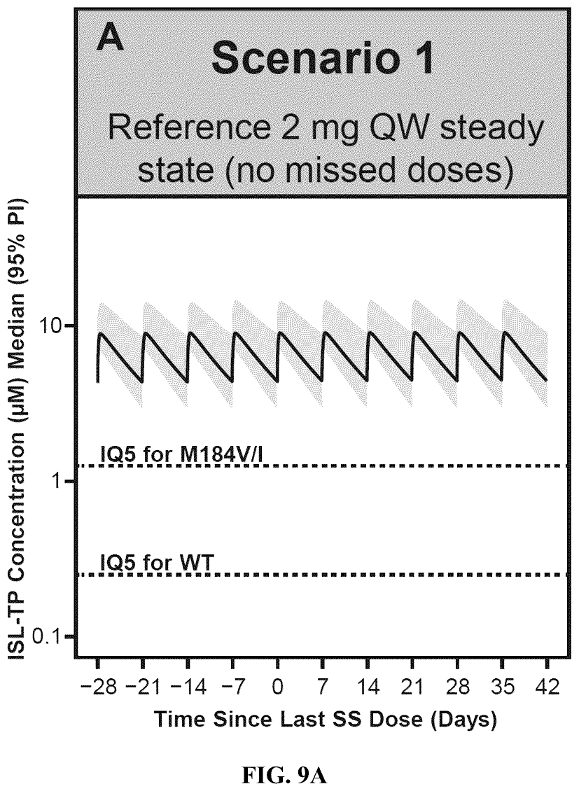

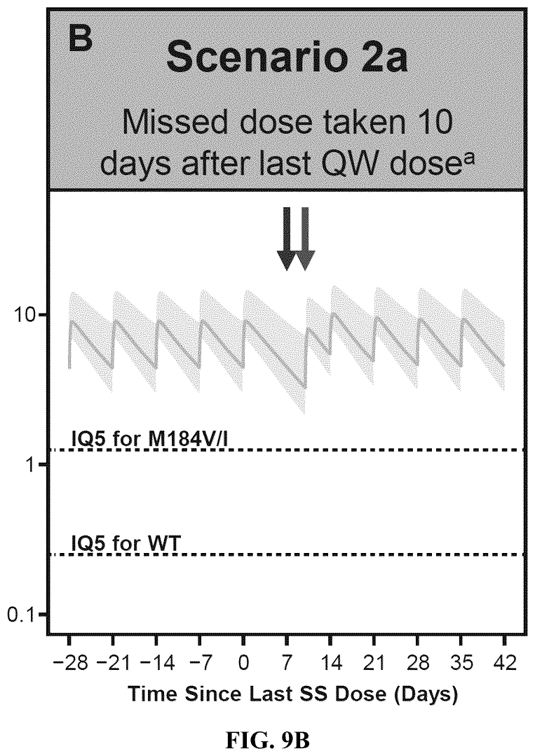

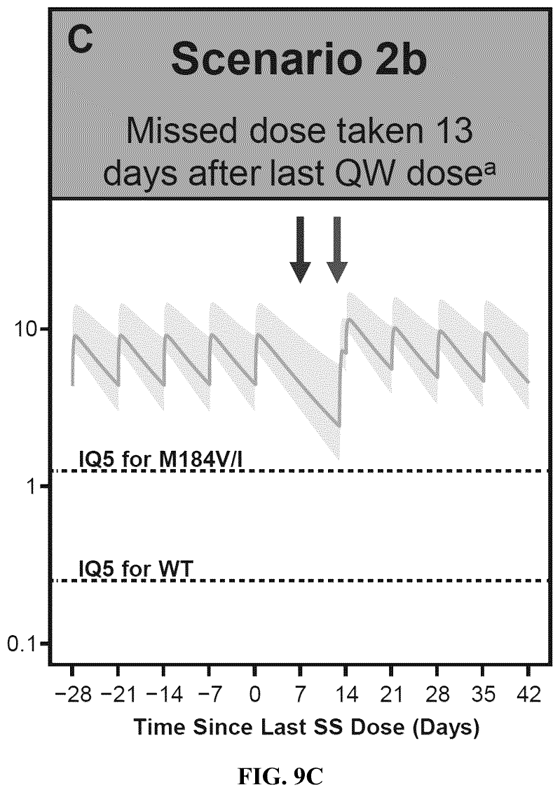

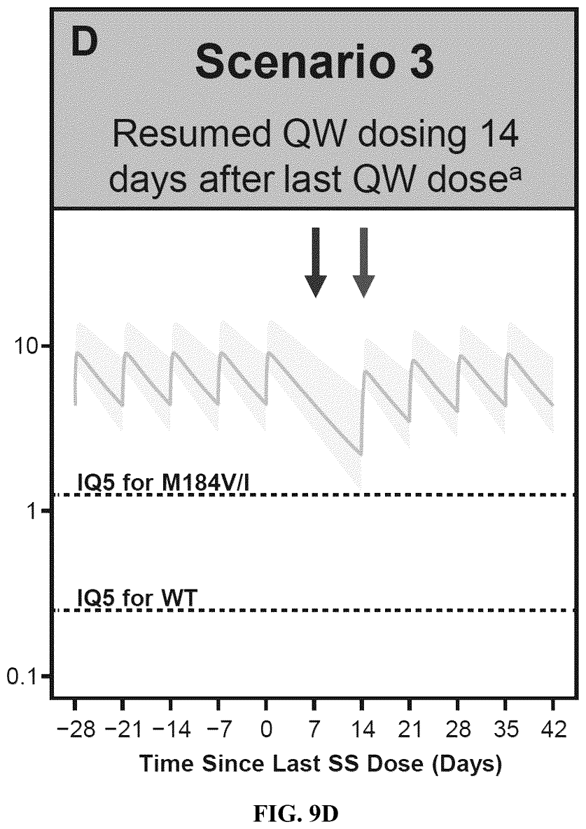

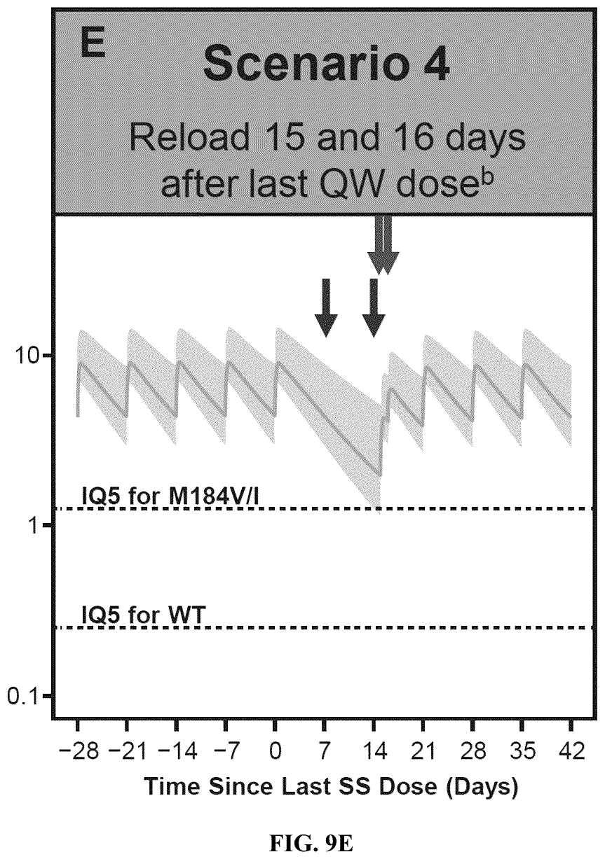

Simulated ISL-TP concentrations for the reference scenario and missed dose scenarios are shown in FIGS. 9A-9E; ISL-TP C_{trough} and Cₘₐₓ are summarized in Table 18. Transient increases in ISL-TP exposures that occurred in Scenario 2a and Scenario 2b, where two 2 mg doses of ISL are taken within a 1-week period, resulted in Cₘₐₓ values above the steady-state Cₘₐₓ with the 2 mg QW reference scenario. It was also well below the steady state Cₘₐₓ values associated with 3 mg QW, the dose predicted in previous modeling and simulation analyses to have no meaningful impact on lymphocyte counts, as shown in FIGS. 9B-9C and Table 18.

ISL-TP maintained effective concentrations 1 week after a missed dose at steady state in Scenario 3, where dosing is resumed 14 days from the last dose with no make-up doses in between, as shown in FIG. 9D and Table 18. Reloading with 1 mg ISL on Day 15 and 1 mg ISL on Day 16, followed by QW maintenance doses in Scenario 4 also maintained effective concentrations, as shown in FIG. 9E and Table 18.

TABLE 18
Predicted ISL-TP C_{trough} and Cₘₐₓ

| Scenario | | |
|---|---|---|
| Median (95% PI), μM | ISL-TP C_{trough} | ISL-TP Cₘₐₓ |
| Scenario 1: Reference 2 mg QW | 4.42 (2.95-9.10) | 9.11 (6.97-15.0) |
| steady state (no missed doses) | | |
| Scenario 2a: Missed dose taken | 3.21 (2.03-7.30) | 10.1 (7.81-16.1) |
| 10 days after last QW dose^{a} | | |
| Scenario 2b: Missed dose taken | 2.38 (1.43-5.91) | 11.5 (9.14-17.5) |
| 13 days after last QW dose^{a} | | |
| Scenario 3: Resumed QW dosing | 2.16 (1.28-5.52) | N/A^{b} |
| 14 days after last QW dose | | |
| Scenario 4: Reload 15 and 16 | 1.97 (1.15-5.17) | N/A^{b} |
| days after last QW dose | | |

For reference, predicted median (95% PI) ISL-TP Cₘₐₓ for 3 mg QW ISL was 13.7 μM (10.5-22.5); no meaningful impact on lymphocyte and CD4+ T cell counts were predicted for 3 mg QW ISL in previous modeling and simulation analyses.
^{a}In these scenarios, the maximum predicted ISL-TP concentration occurs following the “regular” 2 mg ISL dose given after the “make-up” dose.
^{b}Cₘₐₓ values after the make-up dose were below 2 mg QW ISL Cₘₐₓ values.
Cₘₐₓ, maximum concentration; C_{trough}, concentration at the end of the dosing interval; ISL, islatravir; ISL-TP, islatravir-triphosphate; PI, prediction interval; QW, once-weekly.

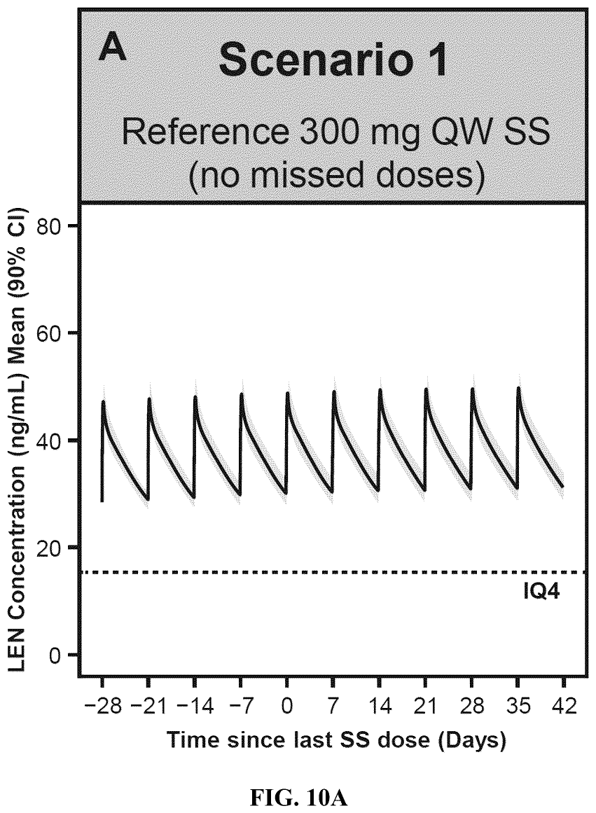

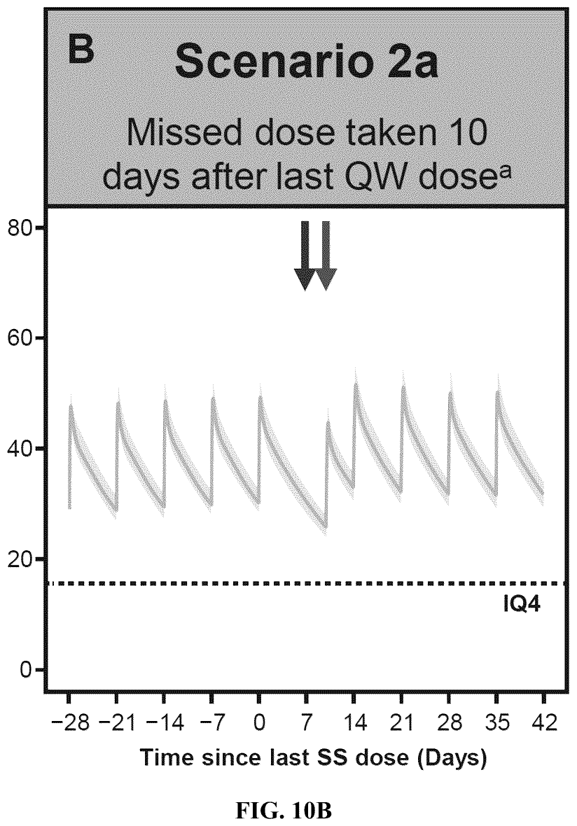

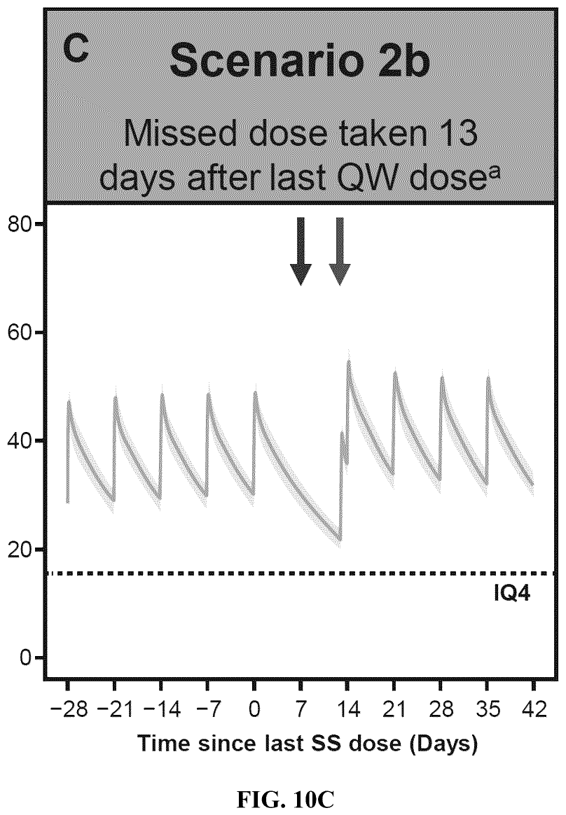

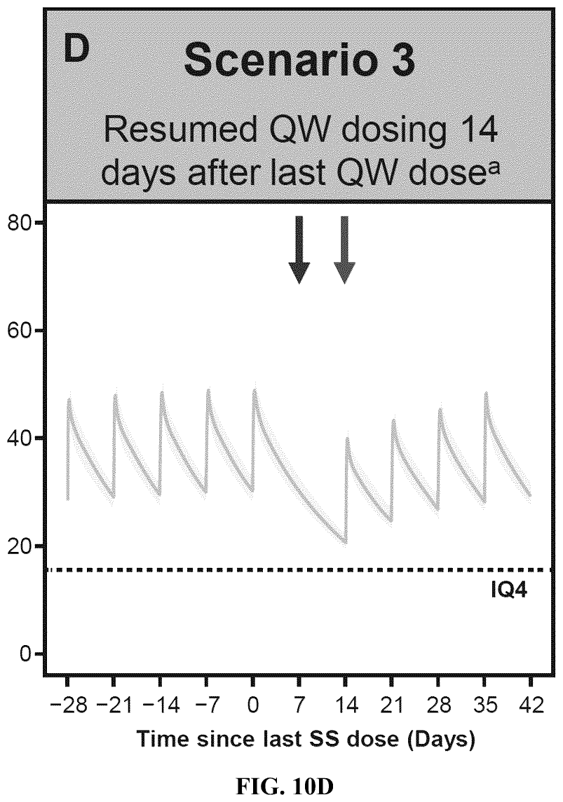

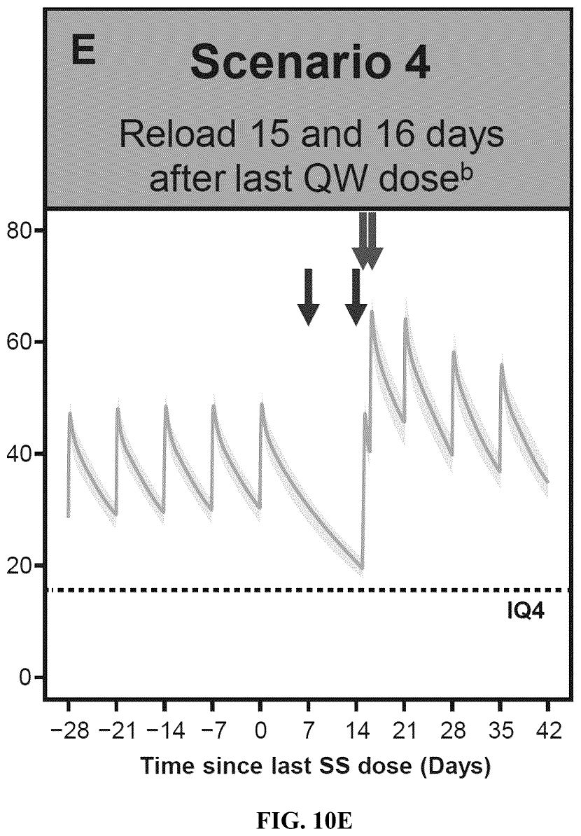

Simulated LEN concentrations for the reference scenario and missed dose scenarios are shown in FIG. 10A-10E; LEN C_{trough} and Cₘₐₓ are summarized in Table 19. Simulated mean C_{trough} concentrations were above the target concentration of 15.5 ng/mL (IQ4) for each scenario, as shown in FIGS. 10A-10E. LEN has been shown to be well tolerated with a wide safety margin, with no clinically relevant prolongation of the QTcF interval at concentrations up to 9-fold higher than therapeutic exposures. Therefore, the potential increases in Cₘₐₓ from various dosing scenarios, including following re-loading as per Scenario 4 (see FIG. 10E), are not anticipated to pose additional safety risks.

TABLE 19
Predicted ISL-TP C_{trough} and Cₘₐₓ

| Scenario | | |
|---|---|---|
| Mean (90% CI), ng/mL | LEN C_{trough} | LEN Cₘₐₓ |
| Scenario 1: Reference 300 mg QW | 29.9 | 48.9 |
| steady state (no missed doses) | (27.7-32.4) | (45.7-53.2) |
| Scenario 2a: Missed dose taken | 25.6 | 52.1 |
| 10 days after last QW dose | (23.7-27.9) | (47.8-56.5)^{a} |
| Scenario 2b: Missed dose taken | 21.9 | 55.6 |
| 13 days after last QW dose | (20.2-24.0) | (51.3-58.7)^{a} |
| Scenario 3: Resumed QW dosing | 20.7 | N/A^{b} |
| 14 days after last QW dose | (19.4-22.9) | |
| Scenario 4: Reload 15 and 16 | 19.6 | 67.7 |
| days after last QW dose | (17.8-21.6) | (62.4-72.4)^{c, d} |

^{a}In these scenarios, predicted LEN Cₘₐₓ occurs following the ‘regular’ 300 mg LEN dose given after the “make-up” dose.
^{b}Cₘₐₓ values after the make-up dose were below LEN 300 mg QW Cₘₐₓ values.
^{c}Cₘₐₓ values after the second 600 mg oral re-loading dose are presented.
^{d}Potential increases in Cₘₐₓ in Scenario 4 are not anticipated to be associated with additional risk given that LEN has been shown to be well-tolerated with a wide safety margin, with no clinically relevant prolongation of the QTcF interval at concentrations up to 9-fold higher than therapeutic exposures (mean LEN C_{trough} and Cₘₐₓ, respectively, are 46.9 and 88.0 ng/mL for Days 1 to 15 [oral loading period] and 32.5 and 86.5 ng/mL for Day 15 to end of Month 6).
CI, confidence interval; Cₘₐₓ, maximum concentration; C_{trough}, concentration at the end of the dosing interval; LEN, lenacapavir; N/A, not applicable; QW, once-weekly.

The simulations described above demonstrate that people with HIV-1 (PWH) who miss one dose of the 2/300 mg islatravir (ISL)/lenacapavir (LEN) fixed-dose combination (FDC) can resume treatment within one week to maintain efficacious drug concentrations, allowing for a one-week forgiveness window.

Various modifications of the invention, in addition to those described herein, will be apparent to those skilled in the art from the foregoing description. Such modifications are also intended to fall within the scope of the appended claims. Each reference, including all patent, patent applications, and publications, cited in the present application is incorporated herein by reference in its entirety.

1. A tablet, comprising a compound of Formula Ia: 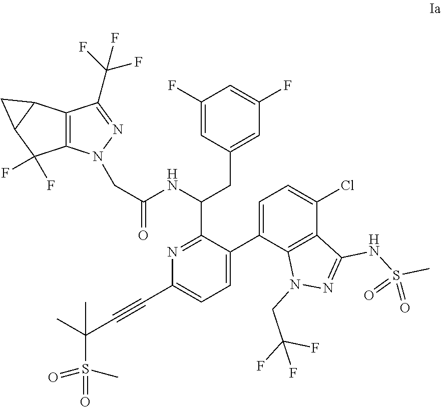 [SMILES: CC(C)(C#Cc1ccc(-c2ccc(Cl)c3c(NS(C)(=O)=O)nn(CC(F)(F)F)c23)c(C(Cc2cc(F)cc(F)c2)NC(=O)Cn2nc(C(F)(F)F)c3c2C(F)(F)C2CC32)n1)S(C)(=O)=O]
or a pharmaceutically acceptable salt, co-crystal, solvate, or combination thereof;
a compound of Formula IIa: 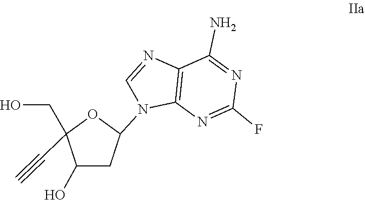 [SMILES: C#CC1(CO)OC(n2cnc3c(N)nc(F)nc32)CC1O]
or a pharmaceutically acceptable salt, co-crystal, solvate, or combination thereof;
and one or more pharmaceutically acceptable excipients.

2. The tablet of claim 1, wherein the tablet comprises a spray dried dispersion of the compound of Formula Ia, or a pharmaceutically acceptable salt, co-crystal, solvate, or combination thereof, wherein the spray dried dispersion comprises about 76.7 w/w % of the compound of Formula Ia, or a pharmaceutically acceptable salt, co-crystal, solvate, or combination thereof, about 18.3 w/w % copovidone, and about 5.0 w/w % poloxamer 407.

3. (canceled)

4. The tablet of claim 2, wherein the tablet comprises about 26.6 w/w % to about 26.7 w/w % of the spray dried dispersion of the compound of Formula Ia, or a pharmaceutically acceptable salt, co-crystal, solvate, or combination thereof.

5. The tablet of claim 1, wherein the tablet comprises about 0.01 w/w % to about 2.0 w/w % of the compound of Formula IIa, or a pharmaceutically acceptable salt, co-crystal, solvate, or combination thereof.

6-10. (canceled)

11. The tablet of claim 1, wherein the tablet comprises about 0.0355 w/w % of the compound of Formula IIa, or a pharmaceutically acceptable salt, co-crystal, solvate, or combination thereof.

12. (canceled)

13. The tablet of claim 1 wherein the tablet comprises mannitol, microcrystalline cellulose, croscarmellose sodium, and magnesium stearate.

14. The tablet of claim 1, wherein the tablet comprises:
about 35 w/w % to about 45 w/w % of mannitol;
about 20 w/w % to about 25 w/w % of microcrystalline cellulose;
about 5 w/w % to about 10 w/w % of croscarmellose sodium; and
about 0.1 w/w % to about 2.0 w/w % of magnesium stearate.

15-29. (canceled)

30. The tablet of claim 1, wherein the tablet comprises:
about 18 w/w % to about 30 w/w % of the compound of Formula Ia, or a pharmaceutically acceptable salt, co-crystal, solvate, or combination thereof;
about 0.01 w/w % to about 2.0 w/w % of the compound of Formula IIa, or a pharmaceutically acceptable salt, co-crystal, solvate, or combination thereof;
about 35 w/w % to about 45 w/w % of mannitol;
about 20 w/w % to about 25 w/w % of microcrystalline cellulose;
about 5 w/w % to about 10 w/w % of croscarmellose sodium; and
about 0.1 w/w % to about 2.0 w/w % of magnesium stearate.

31. The tablet of claim 2, wherein the tablet comprises:
about 26 w/w % to about 27 w/w % of the spray dried dispersion of the compound of Formula Ia, or a pharmaceutically acceptable salt, co-crystal, solvate, or combination thereof;
about 0.02 w/w % to about 0.2 w/w % of the compound of Formula IIa, or a pharmaceutically acceptable salt, co-crystal, solvate, or combination thereof;
about 40 w/w % to about 42 w/w % of mannitol;
about 22 w/w % to about 23 w/w % of microcrystalline cellulose;
about 7 w/w % to about 9 w/w % of croscarmellose sodium; and
about 1.0 w/w % to about 2.0 w/w % of magnesium stearate.

32. The tablet of claim 2, wherein the tablet comprises:
about 26.6 w/w % to about 26.7 w/w % of the spray dried dispersion of the compound of Formula Ia, or a pharmaceutically acceptable salt, co-crystal, solvate, or combination thereof;
about 0.14 w/w % to about 0.15 w/w % of the compound of Formula IIa, or a pharmaceutically acceptable salt, co-crystal, solvate, or combination thereof;
about 41 w/w % to about 42 w/w % of mannitol;
about 22.2 w/w % to about 22.3 w/w % of microcrystalline cellulose;
about 7.9 w/w % to about 8.1 w/w % of croscarmellose sodium; and
about 1.4 w/w % to about 1.6 w/w % of magnesium stearate.

33. The tablet of claim 2, wherein the tablet comprises:
about 26.67 w/w % of the spray dried dispersion of the compound of Formula Ia, or a pharmaceutically acceptable salt, co-crystal, solvate, or combination thereof;
about 0.142 w/w % of the compound of Formula IIa, or a pharmaceutically acceptable salt, co-crystal, solvate, or combination thereof;
about 41.40 w/w % of mannitol;
about 22.288 w/w % microcrystalline cellulose;
about 8.0 w/w % of croscarmellose sodium; and
about 1.5 w/w % of magnesium stearate.

34. The tablet of claim 2, wherein the tablet comprises:
about 20.46 w/w % of the sodium salt of the compound of Formula Ia;
about 0.142 w/w % of the monohydrate of the compound of Formula IIa;
about 4.88 w/w % of copovidone;
about 1.33 w/w % of poloxamer 407;
about 41.40 w/w % of mannitol;
about 22.288 w/w % of microcrystalline cellulose;
about 8.0 w/w % of croscarmellose sodium; and
about 1.5 w/w % of magnesium stearate.

35. The tablet of claim 2, wherein the tablet comprises:
about 26.6 w/w % to about 26.7 w/w % of the spray dried dispersion of the compound of Formula Ia, or a pharmaceutically acceptable salt, co-crystal, solvate, or combination thereof;
about 0.03 w/w % to about 0.04 w/w % of the compound of Formula IIa, or a pharmaceutically acceptable salt, co-crystal, solvate, or combination thereof;
about 41 w/w % to about 42 w/w % of mannitol;
about 22.3 w/w % to about 22.4 w/w % of microcrystalline cellulose;
about 7.9 w/w % to about 8.1 w/w % of croscarmellose sodium; and
about 1.4 w/w % to about 1.6 w/w % of magnesium stearate.

36. The tablet of claim 2, wherein the tablet comprises:
about 26.67 w/w % of the spray dried dispersion of the compound of Formula Ia, or a pharmaceutically acceptable salt, co-crystal, solvate, or combination thereof;
about 0.0355 w/w % of the compound of Formula IIa, or a pharmaceutically acceptable salt, co-crystal, solvate, or combination thereof;
about 41.40 w/w % of mannitol;
about 22.3945 w/w % microcrystalline cellulose;
about 8.0 w/w % of croscarmellose sodium; and
about 1.5 w/w % of magnesium stearate.

37. The tablet of claim 2, wherein the tablet comprises:
about 20.46 w/w % of the sodium salt of the compound of Formula Ia or Ib;
about 0.0355 w/w % of the monohydrate of the compound of Formula IIa or IIb;
about 4.88 w/w % of copovidone;
about 1.33 w/w % of poloxamer 407;
about 41.40 w/w % of mannitol;
about 22.395 w/w % of microcrystalline cellulose;
about 8.0 w/w % of croscarmellose sodium; and
about 1.5 w/w % of magnesium stearate.

38-69. (canceled)

70. The tablet of claim 1, wherein the tablet comprises a sodium salt of the compound of Formula Ia.

71. The tablet of claim 1, wherein the compound of Formula Ia, or a pharmaceutically acceptable salt, co-crystal, solvate, or combination thereof, is a compound of Formula Ib: 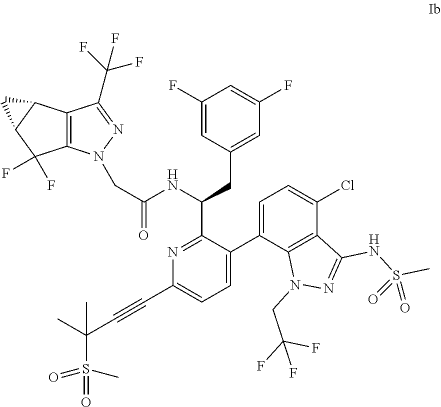 [SMILES: CC(C)(C#Cc1ccc(-c2ccc(Cl)c3c(NS(C)(=O)=O)nn(CC(F)(F)F)c23)c([C@H](Cc2cc(F)cc(F)c2)NC(=O)Cn2nc(C(F)(F)F)c3c2C(F)(F)[C@@H]2C[C@H]32)n1)S(C)(=O)=O]
or a pharmaceutically acceptable salt, co-crystal, solvate, or combination thereof.

72-74. (canceled)

75. The tablet of claim 1, wherein the tablet comprises a monohydrate of the compound of Formula IIa.

76. The tablet of claim 1, wherein the compound of Formula IIa, or a pharmaceutically acceptable salt, co-crystal, solvate, or combination thereof, is a compound of Formula IIb: 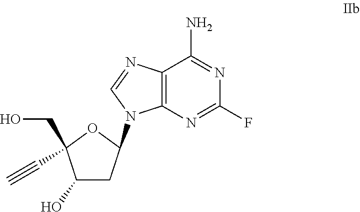 [SMILES: C#C[C@]1(CO)O[C@@H](n2cnc3c(N)nc(F)nc32)C[C@@H]1O]
or a pharmaceutically acceptable salt, co-crystal, solvate, or combination thereof.

77-78. (canceled)

79. A method of treating HIV in a patient, comprising administering to the patient a tablet of claim 1.

80-86. (canceled)

87. The method of claim 79, wherein the method comprises orally administering to the patient an initiation dosage of the tablet for a first period of time; and
orally administering to the patient one or more maintenance dosages of the tablet for a second period of time, wherein the second period of time occurs after the first period of time.

88-108. (canceled)

109. A method of treating HIV in a patient, comprising:
i) administering to the patient an initiation dose comprising two tablets on day 1, and two tablets on day 2, wherein each tablet of the initiation dose comprises:
about 300 mg of the compound of Formula Ib: 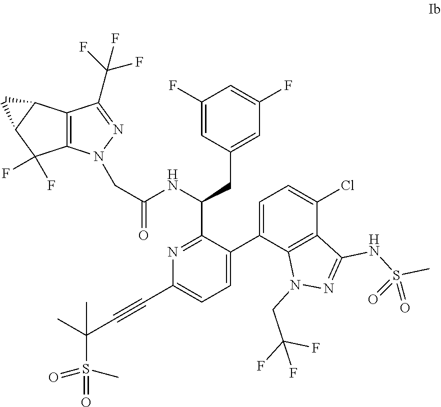 [SMILES: CC(C)(C#Cc1ccc(-c2ccc(Cl)c3c(NS(C)(=O)=O)nn(CC(F)(F)F)c23)c([C@H](Cc2cc(F)cc(F)c2)NC(=O)Cn2nc(C(F)(F)F)c3c2C(F)(F)[C@@H]2C[C@H]32)n1)S(C)(=O)=O]
on a free acid basis;
about 0.5 mg of the compound of Formula IIb: 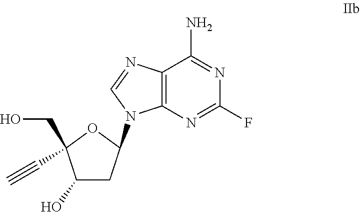 [SMILES: C#C[C@]1(CO)O[C@@H](n2cnc3c(N)nc(F)nc32)C[C@@H]1O]
on a free base basis;
about 621 mg of mannitol;
about 335.9175 mg of microcrystalline cellulose;
about 120 mg croscarmellose sodium; and
about 22.5 mg magnesium stearate; and
ii) administering to the patient a maintenance dose comprising one tablet, once weekly, beginning on day 8, wherein the tablet comprises:
about 300 mg of the compound of Formula Ib, on a free acid basis;
about 2.0 mg of the compound of Formula IIb, on a free base basis;
about 621 mg of mannitol;
about 334.32 mg of microcrystalline cellulose;
about 120 mg croscarmellose sodium; and
about 22.5 mg magnesium stearate;
wherein if the patient misses a maintenance dosage; and
wherein between about 7 days to about 16 days have elapsed since administration of a prior maintenance dosage;
the method further comprises restarting administration of the one or more maintenance dosages of step (ii) as soon as possible after the missed dose.

110. A method of treating HIV in a patient, comprising:
i) administering to the patient an initiation dose comprising two tablets on day 1, and two tablets on day 2, wherein each tablet of the initiation dose comprises:
about 300 mg of the compound of Formula Ib: 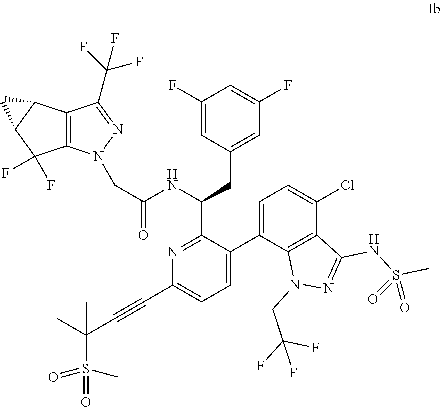 [SMILES: CC(C)(C#Cc1ccc(-c2ccc(Cl)c3c(NS(C)(=O)=O)nn(CC(F)(F)F)c23)c([C@H](Cc2cc(F)cc(F)c2)NC(=O)Cn2nc(C(F)(F)F)c3c2C(F)(F)[C@@H]2C[C@H]32)n1)S(C)(=O)=O]
on a free acid basis;
about 0.5 mg of the compound of Formula IIb: 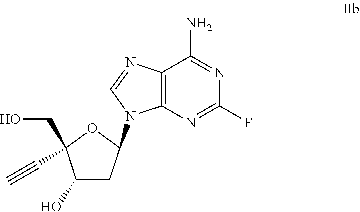 [SMILES: C#C[C@]1(CO)O[C@@H](n2cnc3c(N)nc(F)nc32)C[C@@H]1O]
on a free base basis;
about 73.2 mg of copovidone;
about 19.95 mg of poloxamer 407;
about 621 mg of mannitol;
about 335.92 mg of microcrystalline cellulose;
about 120 mg of croscarmellose sodium;
about 22.5 mg of magnesium stearate; and
ii) administering to the patient a maintenance dose comprising one tablet, once weekly, beginning on day 8, wherein the tablet comprises:
about 300 mg of the compound of Formula Ib, on a free acid basis;
about 2.0 mg of the compound of Formula IIb, on a free base basis;
about 73.2 mg of copovidone;
about 19.95 mg of poloxamer 407;
about 621 mg of mannitol;
about 334.32 mg of microcrystalline cellulose;
about 120 mg of croscarmellose sodium;
about 22.5 mg of magnesium stearate;
wherein if the patient misses a maintenance dosage; and
wherein between about 7 days to about 16 days have elapsed since administration of a prior maintenance dosage;
the method further comprises restarting administration of the one or more maintenance dosages of step (ii) as soon as possible after the missed dose.
